(19) United States
(12) Patent Application Publication
(10) Pub. No.: US 2011/0071150 A1
(43) Pub. Date: Mar. 24, 2011

(54) INDOLE DERIVATIVES AS CRAC MODULATORS

(76) Inventors: Muzaffar Alam, Clifton, NJ (US); Daisy Joe Du Bois, Menlo Park, CA (US); Ronald Charles Hawley, Glen Ridge, NJ (US); Joshua Kennedy-Smith, New York, NY (US); Ana Elena Minatti, Woodland Hills, CA (US); Wylie Solang Palmer, Morristown, NJ (US); Tania Silva, Sunnyvale, CA (US); Robert Stephen Wilhelm, Los Altos, CA (US)

(21) Appl. No.: 12/888,701

(22) Filed: Sep. 23, 2010

Related U.S. Application Data
(60) Provisional application No. 61/245,521, filed on Sep. 24, 2009.
(60) Provisional application No. 61/378,062, filed on Aug. 30, 2010.

(51) Int. Cl. A61K 31/5355; C07D 403/02; C07D 235/04; C07D 417/14; C07D 277/20; C07D 401/14; C07D 263/30; C07D 403/14; C07D 413/14; A61K 31/4178; A61K 31/4155; A61K 31/443; A61K 31/427; A61K 31/444; A61K 31/422; A61K 31/495; A61K 31/506; A61P 11/06; A61P 11/08

(52) U.S. Cl. 514/235.2; 548/364.7; 548/305.1; 546/269.7; 548/202; 546/256; 548/235; 544/333; 544/405; 544/124; 514/406; 514/397; 514/339; 514/365; 514/333; 514/374; 514/256; 514/255.05

(57) ABSTRACT

Compounds of the formula I:

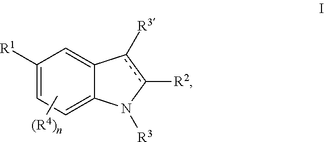

or pharmaceutically acceptable salts thereof,
wherein R¹, R², R³ and R⁴ are as defined herein. Also disclosed are methods of making the compounds and using the compounds for treatment of diseases associated with calcium release-activated calcium channels (CRAC).

### PRIORITY TO RELATED APPLICATION(S)

This application claims the benefit of U.S. Provisional Application No. 61/245,521, filed Sep. 24, 2009, and U.S. Provisional Application No. 61/378,062 filed Aug. 31, 2010. The entire contents of the above-identified applications are hereby incorporated herein by reference.

### FIELD OF THE INVENTION

This invention pertains to compounds useful for treatment of autoimmune and inflammatory diseases associated with IL-2 inhibition via modulation of calcium release-activated calcium channels.

### BACKGROUND OF THE INVENTION

The cytokine interleukin 2 (IL-2) is a T-cell mitogen important for T-cell proliferation and as a B cell growth factor. Because of its effects on T cells and B cells, IL-2 is recognized as an important regulator of immune responses. IL-2 is involved in inflammation, tumor progression and hematopoiesis, and IL-2 affects the production of other cytokines such as TNA alpha, TNF beta, IFN gamma. Inhibition of IL-2 production thus is relevant to immunosuppression therapies and treatment of inflammatory and immune disorders.

T-cell antigen binding in inflammatory events leads to T-cell initiated calcium influx by calcium release-activated calcium channels (CRAC). IL-2 secretion by T-cells occurs in response to calcium ion influx. Modulation of CRAC thus provides a mechanism for control of production of IL-2 and other cytokines associated with inflammation. CRAC inhibition has been recognized as a potential route to therapies for rheumatoid arthritis, asthma, allergic reactions and other inflammatory conditions (see, e.g., Chang et al., Acta Pharmacologica Sinica (2006) Vol. 7, 813-820), and CRAC inhibitors have been shown to prevent antigen-induced airway eosinophilia and late phase asthmatic responses via Th2 cytokine inhibition in animal models (Yoshino et al., Eur. J. Pharm. (2007) Vol. 560(2), 225-233). There is, accordingly, a need for CRAC inhibitors.

### SUMMARY OF THE INVENTION

The invention provides compounds of the formula I:

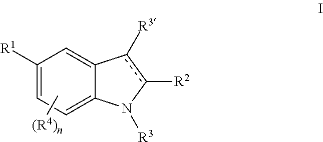

wherein:

### R¹ is:

phenyl substituted one, two or three times with a group or groups independently selected from:
   C₁₋₆alkyl; C₁₋₆alkoxy; halo; halo-C₁₋₆alkyl; halo-C₁₋₆alkoxy; nitrile; acetyl; C₁₋₆alkoxycarbonyl; aminocarbonyl; aminosulfonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; hydroxy; sulfonylmorpholine; sulfonylmethylpiperazine; heterocyclyl; phenyl which may be optionally substituted; or heteroaryl which may be optionally substituted;
pyridinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; heterocyclyl; phenyl which may be optionally substituted; or heteroaryl which may be optionally substituted;
pyrimidinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; heterocyclyl; phenyl which may be optionally substituted; or heteroaryl which may be optionally substituted; or
a five-membered heteroaryl ring optionally substituted one, two or three times with a group or groups independently selected from: C₁₋₆alkyl; C₃₋₆cycloalkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; heterocyclyl; phenyl which may be optionally substituted; and heteroaryl which may be optionally substituted; or two of said substituents together with the atoms to which they are attached may form a phenyl fused to the five-membered heteroaryl ring;

### R² is:

C₃₋₆ cycloalkyl;
phenyl substituted one, two or three times with a group or groups independently selected from: C₁₋₆alkyl; C₁₋₆alkoxy; C₁₋₆alkoxyhydroxy; halo; halo-C₁₋₆alkyl; halo-C₁₋₆alkoxy; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; C₁₋₆alkylcarbonylhydroxy; C₁₋₆alkoxycyano; amino; hydroxy; phenyl which may be optionally substituted; or heteroaryl which may be optionally substituted;
pyridinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; phenyl which may be optionally substituted; or heteroaryl which may be optionally substituted;
pyrimidinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; phenyl which may be optionally substituted; or heteroaryl which may be optionally substituted; or
a five-membered heteroaryl ring optionally substituted once or twice with a group or groups independently selected from: C₁₋₆alkyl; C₁₋₆alkoxy; halo; halo-C₁₋₆alkyl; C₃₋₆cycloalkyl; halo-C₁₋₆alkoxy; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; phenyl which may be optionally substituted; and heteroaryl which may be optionally substituted; or two of said substituents together with the atoms to which they are attached may form a phenyl fused to said five-membered heteroaryl ring;
   R³ is hydrogen
   R^{3′} is hydrogen or C₁₋₆alkyl;
   n is from 0 to 3;
   each R⁴ is independently selected from: hydrogen; C₁₋₆alkyl; C₁₋₆alkoxy; halo; and halo-C₁₋₆alkyl, and
   said dashed line is a bond or absent,
   or a pharmaceutically acceptable salt thereof.

The invention also provides for pharmaceutical compositions comprising the compounds, methods of using the compounds, and methods of preparing the compounds.

### DETAILED DESCRIPTION OF THE INVENTION

### Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

“Alkyl” means the monovalent linear or branched saturated hydrocarbon moiety, consisting solely of carbon and hydrogen atoms, having from one to twelve carbon atoms.

“Lower alkyl” refers to an alkyl group of one to six carbon atoms, i.e. C₁-C₆alkyl. Examples of alkyl groups include, but are not limited to, methyl, ethyl, propyl, isopropyl, isobutyl, sec-butyl, tert-butyl, pentyl, n-hexyl, octyl, dodecyl, and the like.

“Alkenyl” means a linear monovalent hydrocarbon radical of two to six carbon atoms or a branched monovalent hydrocarbon radical of three to six carbon atoms, containing at least one double bond, e.g., ethenyl, propenyl, and the like.

“Alkynyl” means a linear monovalent hydrocarbon radical of two to six carbon atoms or a branched monovalent hydrocarbon radical of three to six carbon atoms, containing at least one triple bond, e.g., ethynyl, propynyl, and the like.

“Alkylene” means a linear saturated divalent hydrocarbon radical of one to six carbon atoms or a branched saturated divalent hydrocarbon radical of three to six carbon atoms, e.g., methylene, ethylene, 2,2-dimethylethylene, propylene, 2-methylpropylene, butylene, pentylene, and the like.

“Alkoxy” and “alkyloxy”, which may be used interchangeably, mean a moiety of the formula —OR, wherein R is an alkyl moiety as defined herein. Examples of alkoxy moieties include, but are not limited to, methoxy, ethoxy, isopropoxy, and the like.

“Alkoxyalkyl” means a moiety of the formula R^{a}—O—R^{b}—, where R^{a} is alkyl and R^{b} is alkylene as defined herein. Exemplary alkoxyalkyl groups include, by way of example, 2-methoxyethyl, 3-methoxypropyl, 1-methyl-2-methoxyethyl, 1-(2-methoxyethyl)-3-methoxypropyl, and 1-(2-methoxyethyl)-3-methoxypropyl.

“Alkoxyalkoxy” means a group of the formula —O—R—R′ wherein R is alkylene and R′ is alkoxy as defined herein.

“Alkylcarbonyl” means a moiety of the formula —C(O)—R, wherein R is alkyl as defined herein.

“Alkoxycarbonyl” means a group of the formula —C(O)—R wherein R is alkoxy as defined herein.

“Alkylcarbonylalkyl” means a group of the formula —R—C(O)—R wherein R is alkylene and R′ is alkyl as defined herein.

“Alkoxycarbonylalkyl” means a group of the formula —R—C(O)—R wherein R is alkylene and R′ is alkoxy as defined herein.

“Alkoxycarbonylalkoxy” means a group of the formula —O—R—C(O)—R′ wherein R is alkylene and R′ is alkoxy as defined herein.

“Hydroxycarbonylalkoxy” means a group of the formula —O—R—C(O)—OH wherein R is alkylene as defined herein.

“Alkylaminocarbonylalkoxy” means a group of the formula —O—R—C(O)—NHR′ wherein R is alkylene and R′ is alkyl as defined herein.

“Dialkylaminocarbonylalkoxy” means a group of the formula —O—R—C(O)—NR′R″ wherein R is alkylene and R′ and R″ are alkyl as defined herein.

“Alkylaminoalkoxy” means a group of the formula —O—R—NHR′ wherein R is alkylene and R′ is alkyl as defined herein.

“Dialkylaminoalkoxy” means a group of the formula —O—R—NR′R′ wherein R is alkylene and R′ and R″ are alkyl as defined herein.

“Alkylsulfonyl” means a moiety of the formula —SO₂—R, wherein R is alkyl as defined herein.

“Alkylsulfonylalkyl means a moiety of the formula —R′—SO₂—R″ where R′ is alkylene and R″ is alkyl as defined herein.

“Alkylsulfonylalkoxy” means a group of the formula —O—R—SO₂—R′ wherein R is alkylene and R′ is alkyl as defined herein.

“Amino means a moiety of the formula —NRR′ wherein R and R′ each independently is hydrogen or alkyl as defined herein. “Amino thus includes “alkylamino (where one of R and R′ is alkyl and the other is hydrogen) and “dialkylamino (where R and R′ are both alkyl.

“Aminocarbonyl” means a group of the formula —C(O)—R wherein R is amino as defined herein.

“Alkoxyamino” means a moiety of the formula —NR—OR′ wherein R is hydrogen or alkyl and R′ is alkyl as defined herein.

“Alkylsulfanyl” means a moiety of the formula —SR wherein R is alkyl as defined herein.

“Aminoalkyl” means a group —R—R′ wherein R′ is amino and R is alkylene as defined herein. “Aminoalkyl” includes aminomethyl, aminoethyl, 1-aminopropyl, 2-aminopropyl, and the like. The amino moiety of “aminoalkyl” may be substituted once or twice with alkyl to provide “alkylaminoalkyl” and “dialkylaminoalkyl” respectively.

“Alkylaminoalkyl” includes methylaminomethyl, methylaminoethyl, methylaminopropyl, ethylaminoethyl and the like. “Dialkylaminoalkyl” includes dimethylaminomethyl, dimethylaminoethyl, dimethylaminopropyl, N-methyl-N-ethylaminoethyl, and the like.

“Aminoalkoxy” means a group —OR—R′ wherein R′ is amino and R is alkylene as defined herein.

“Alkylsulfonylamido” means a moiety of the formula —NR′SO₂—R wherein R is alkyl and R′ is hydrogen or alkyl.

“Aminocarbonyloxyalkyl” or “carbamylalkyl” means a group of the formula —R—O—C(O)—NR′R″ wherein R is alkylene and R′, R″ each independently is hydrogen or alkyl as defined herein.

“Alkynylalkoxy” means a group of the formula —O—R—R′ wherein R is alkylene and R′ is alkynyl as defined herein.

“Aryl” means a monovalent cyclic aromatic hydrocarbon moiety having a mono-, bi- or tricyclic aromatic ring. The aryl group can be optionally substituted as defined herein. Examples of aryl moieties include, but are not limited to, phenyl, naphthyl, phenanthryl, fluorenyl, indenyl, pentalenyl, azulenyl, oxydiphenyl, biphenyl, methylenediphenyl, aminodiphenyl, diphenylsulfidyl, diphenylsulfonyl, diphenylisopropylidenyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like, including partially hydrogenated derivatives thereof, each being optionally substituted.

“Arylalkyl” and “Aralkyl”, which may be used interchangeably, mean a radical-R^{a}R^{b} where R^{a} is an alkylene group and R^{b} is an aryl group as defined herein; e.g., phenylalkyls such as benzyl, phenylethyl, 3-(3-chlorophenyl)-2-methylpentyl, and the like are examples of arylalkyl.

“Arylsulfonyl means a group of the formula —SO₂—R wherein R is aryl as defined herein.

“Aryloxy” means a group of the formula —O—R wherein R is aryl as defined herein. “Aralkyloxy” means a group of the formula —O—R—R″ wherein R is alkylene and R′ is aryl as defined herein.

“Carboxy” or “hydroxycarbonyl”, which may be used interchangeably, means a group of the formula —C(O)—OH.

“Cyanoalkyl” means a moiety of the formula —R′—R″, where R′ is alkylene as defined herein and R″ is cyano or nitrile.

“Cycloalkyl” means a monovalent saturated carbocyclic moiety having mono- or bicyclic rings. Preferred cycloalkyl are unsubstituted or substituted with alkyl. Cycloalkyl can optionally be substituted with one or more substituents, wherein each substituent is independently hydroxy, alkyl, alkoxy, halo, haloalkyl, amino, monoalkylamino, or dialkylamino, unless otherwise specifically indicated. Examples of cycloalkyl moieties include, but are not limited to, cyclopropyl, cyclobutyl, cyclopentyl, cyclohexyl, cycloheptyl, and the like, including partially unsaturated (cycloalkenyl) derivatives thereof.

“Cycloalkylalkyl” means a moiety of the formula —R′—R″, where R′ is alkylene and R″ is cycloalkyl as defined herein.

“Cycloalkylalkoxy” means a group of the formula —O—R—R′ wherein R is alkylene and R′ is cycloalkyl as defined herein.

“Heteroalkyl” means an alkyl radical as defined herein wherein one, two or three hydrogen atoms have been replaced with a substituent independently selected from the group consisting of −OR^{a}, —NR^{b}R^{c} and —S(O)—R^{d} (where n is an integer from 0 to 2), with the understanding that the point of attachment of the heteroalkyl radical is through a carbon atom, wherein R^{a} is hydrogen, acyl, alkyl, cycloalkyl, or cycloalkylalkyl; R^{b} and R^{c} are independently of each other hydrogen, acyl, alkyl, cycloalkyl, or cycloalkylalkyl; and when n is O, R^{d} is hydrogen, alkyl, cycloalkyl, or cycloalkylalkyl, and when n is 1 or 2, R^{d} is alkyl, cycloalkyl, cycloalkylalkyl, amino, acylamino, monoalkylamino, or dialkylamino. Representative examples include, but are not limited to, 2-hydroxyethyl, 3-hydroxypropyl, 2-hydroxy-1-hydroxymethylethyl, 2,3-dihydroxypropyl, 1-hydroxymethylethyl, 3-hydroxybutyl, 2,3-dihydroxybutyl, 2-hydroxy-1-methylpropyl, 2-aminoethyl, 3-aminopropyl, 2-methylsulfonylethyl, aminosulfonylmethyl, aminosulfonylethyl, aminosulfonylpropyl, methylaminosulfonylmethyl, methylaminosulfonylethyl, methylaminosulfonylpropyl, and the like.

“Heteroaryl” means a monocyclic or bicyclic radical of 5 to 12 ring atoms having at least one aromatic ring containing one, two, three or four ring heteroatoms selected from N, O, or S, the remaining ring atoms being C, with the understanding that the attachment point of the heteroaryl radical will be on an aromatic ring. The heteroaryl ring may be optionally substituted as defined herein. Examples of heteroaryl moieties include, but are not limited to, optionally substituted imidazolyl, oxazolyl, isoxazolyl, thiazolyl, isothiazolyl, oxadiazolyl, thiadiazolyl, pyrazinyl, thienyl, benzothienyl, thiophenyl, furanyl, pyranyl, pyridyl, pyrrolyl, pyrazolyl, pyrimidyl, quinolinyl, isoquinolinyl, benzofuryl, benzothiophenyl, benzothiopyranyl, benzimidazolyl, benzooxazolyl, benzooxadiazolyl, benzothiazolyl, benzothiadiazolyl, benzopyranyl, indolyl, isoindolyl, tetrazolyl, triazolyl, triazinyl, quinoxalinyl, purinyl, quinazolinyl, quinolizinyl, naphthyridinyl, pteridinyl, carbazolyl, azepinyl, diazepinyl, acridinyl and the like, including partially hydrogenated derivatives thereof, each optionally substituted.

Heteroarylalkyl” or “heteroaralkyl” means a group of the formula —R—R′ wherein R is alkylene and R′ is heteroaryl as defined herein.

“Heteroarylsulfonyl means a group of the formula —SO₂—R wherein R is heteroaryl as defined herein.

“Heteroaryloxy” means a group of the formula —O—R wherein R is heteroaryl as defined herein.

“Heteroaralkyloxy” means a group of the formula —O—R—R″ wherein R is alkylene and R′ is heteroaryl as defined herein.

The terms “halo”, “halogen” and “halide”, which may be used interchangeably, refer to a substituent fluoro, chloro, bromo, or iodo.

“Haloalkyl” means alkyl as defined herein in which one or more hydrogen has been replaced with same or different halogen. Exemplary haloalkyls include —CH₂Cl, —CH₂CF₃, —CH₂CCl₃, perfluoroalkyl (e.g., —CF₃), and the like.

“Haloalkoxy” means a moiety of the formula —OR, wherein R is a haloalkyl moiety as defined herein. An exemplary haloalkoxy is difluoromethoxy.

“Heterocycloamino” means a saturated ring wherein at least one ring atom is N, NH or N-alkyl and the remaining ring atoms form an alkylene group.

“Heterocyclyl” means a monovalent saturated moiety, having one to three rings, incorporating one, two, or three or four heteroatoms (chosen from nitrogen, oxygen or sulfur). The heterocyclyl ring may be optionally substituted as defined herein. Examples of heterocyclyl moieties include, but are not limited to, optionally substituted piperidinyl, piperazinyl, homopiperazinyl, azepinyl, pyrrolidinyl, pyrazolidinyl, imidazolinyl, imidazolidinyl, pyridinyl, pyridazinyl, pyrimidinyl, oxazolidinyl, isoxazolidinyl, morpholinyl, thiazolidinyl, isothiazolidinyl, quinuclidinyl, quinolinyl, isoquinolinyl, benzimidazolyl, thiadiazolylidinyl, benzothiazolidinyl, benzoazolylidinyl, dihydrofuryl, tetrahydrofuryl, dihydropyranyl, tetrahydropyranyl, thiamorpholinyl, thiamorpholinylsulfoxide, thiamorpholinylsulfone, dihydroquinolinyl, dihydrisoquinolinyl, tetrahydroquinolinyl, tetrahydrisoquinolinyl, and the like.

“Heterocyclylalkyl” means a moiety of the formula —R—R′ wherein R is alkylene and R′ is heterocyclyl as defined herein.

“Heterocyclyloxy” means a moiety of the formula —OR wherein R is heterocyclyl as defined herein.

“Heterocyclylalkoxy” means a moiety of the formula —OR—R′ wherein R is alkylene and R′ is heterocyclyl as defined herein.

“Hydroxyalkoxy” means a moiety of the formula —OR wherein R is hydroxyalkyl as defined herein.

“Hydroxyalkylamino” means a moiety of the formula —NR—R′ wherein R is hydrogen or alkyl and R′ is hydroxyalkyl as defined herein.

“Hydroxyalkylaminoalkyl” means a moiety of the formula —R—NR′—R″ wherein R is alkylene, R′ is hydrogen or alkyl, and R″ is hydroxyalkyl as defined herein.

“Hydroxycarbonylalkyl” or “carboxyalkyl” means a group of the formula —R—(CO)—OH where R is alkylene as defined herein.

“Hydroxycarbonylalkoxy” means a group of the formula —O—R—C(O)—OH wherein R is alkylene as defined herein.

“Hydroxyalkyloxycarbonylalkyl” or “hydroxyalkoxycarbonylalkyl” means a group of the formula —R—C(O)—O—R—OH wherein each R is alkylene and may be the same or different.

“Hydroxyalkyl” means an alkyl moiety as defined herein, substituted with one or more, preferably one, two or three hydroxy groups, provided that the same carbon atom does not carry more than one hydroxy group. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 2-hydroxy-1-hydroxymethylethyl, 2,3-dihydroxybutyl, 3,4-dihydroxybutyl and 2-(hydroxymethyl)-3-hydroxypropyl

“Hydroxycycloalkyl” means a cycloalkyl moiety as defined herein wherein one, two or three hydrogen atoms in the cycloalkyl radical have been replaced with a hydroxy substituent. Representative examples include, but are not limited to, 2-, 3-, or 4-hydroxycyclohexyl, and the like.

“Alkoxy hydroxyalkyl” and “hydroxy alkoxyalkyl”, which may be used interchangeably, means an alkyl as defined herein that is substituted at least once with hydroxy and at least once with alkoxy. “Alkoxy hydroxyalkyl” and “hydroxy alkoxyalkyl” thus encompass, for example, 2-hydroxy-3-methoxy-propan-1-yl and the like.

“Urea” or “ureido” means a group of the formula —NR′—C(O)—NR″R″′ wherein R′, R″ and R″′ each independently is hydrogen or alkyl.

“Carbamate” means a group of the formula —O—C(O)—NR′R″ wherein R′ and R″ each independently is hydrogen or alkyl.

“Carboxy” means a group of the formula —O—C(O)—OH.

“Sulfonamido” means a group of the formula —SO₂—NR′R″ wherein R′, R″ and R″′ each independently is hydrogen or alkyl.

“Optionally substituted”, when used in association with “aryl”, phenyl”, “heteroaryl” “cycloalkyl” or “heterocyclyl”, means an aryl, phenyl, heteroaryl, cycloalkyl or heterocyclyl which is optionally substituted independently with one to four substituents, preferably one or two substituents selected from alkyl, cycloalkyl, cycloalkylalkyl, heteroalkyl, hydroxyalkyl, halo, nitro, cyano, hydroxy, alkoxy, amino, acylamino, mono-alkylamino, di-alkylamino, haloalkyl, haloalkoxy, heteroalkyl, —COR, —SO₂R (where R is hydrogen, alkyl, phenyl or phenylalkyl), —(CR′R″)ₙ—COOR (where n is an integer from 0 to 5, R′ and R″ are independently hydrogen or alkyl, and R is hydrogen, alkyl, cycloalkyl, cycloalkylalkyl, phenyl or phenylalkyl), or —(CR′R″)ₙ—CONR^{a}R^{b} (where n is an integer from 0 to 5, R′ and R″ are independently hydrogen or alkyl, and R^{a} and R^{b} are, independently of each other, hydrogen, alkyl, cycloalkyl, cycloalkylalkyl, phenyl or phenylalkyl). Certain preferred optional substituents for “aryl”, phenyl”, “heteroaryl” “cycloalkyl” or “heterocyclyl” include alkyl, halo, haloalkyl, alkoxy, cyano, amino and alkylsulfonyl. More preferred substituents are methyl, fluoro, chloro, trifluoromethyl, methoxy, amino and methanesulfonyl.

“Leaving group” means the group with the meaning conventionally associated with it in synthetic organic chemistry, i.e., an atom or group displaceable under substitution reaction conditions. Examples of leaving groups include, but are not limited to, halogen, alkane- or arylenesulfonyloxy, such as methanesulfonyloxy, ethanesulfonyloxy, thiomethyl, benzenesulfonyloxy, tosyloxy, and thienyloxy, dihalophosphinoyloxy, optionally substituted benzyloxy, isopropyloxy, acyloxy, and the like.

“Modulator” means a molecule that interacts with a target. The interactions include, but are not limited to, agonist, antagonist, and the like, as defined herein.

“Optional” or “optionally” means that the subsequently described event or circumstance may but need not occur, and that the description includes instances where the event or circumstance occurs and instances in which it does not.

“Disease” and “Disease state” means any disease, condition, symptom, disorder or indication.

“Inert organic solvent” or “inert solvent” means the solvent is inert under the conditions of the reaction being described in conjunction therewith, including for example, benzene, toluene, acetonitrile, tetrahydrofuran, N,N-dimethylformamide, chloroform, methylene chloride or dichloromethane, dichloroethane, diethyl ether, ethyl acetate, acetone, methyl ethyl ketone, methanol, ethanol, propanol, isopropanol, tert-butanol, dioxane, pyridine, and the like. Unless specified to the contrary, the solvents used in the reactions of the present invention are inert solvents.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

“Pharmaceutically acceptable salts” of a compound means salts that are pharmaceutically acceptable, as defined herein, and that possess the desired pharmacological activity of the parent compound. Such salts include:

acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic, camphorsulfonic acid, citric acid, ethanesulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, p-toluenesulfonic acid, trimethylacetic acid, and the like; or
salts formed when an acidic proton present in the parent compound either is replaced by a metal ion, e.g., an alkali metal ion, an alkaline earth ion, or an aluminum ion; or coordinates with an organic or inorganic base. Acceptable organic bases include diethanolamine, ethanolamine, N-methylglucamine, triethanolamine, tromethamine, and the like. Acceptable inorganic bases include aluminum hydroxide, calcium hydroxide, potassium hydroxide, sodium carbonate and sodium hydroxide.

The preferred pharmaceutically acceptable salts are the salts formed from acetic acid, hydrochloric acid, sulphuric acid, methanesulfonic acid, maleic acid, phosphoric acid, tartaric acid, citric acid, sodium, potassium, calcium, zinc, and magnesium.

It should be understood that all references to pharmaceutically acceptable salts include solvent addition forms (solvates) or crystal forms (polymorphs) as defined herein, of the same acid addition salt.

“Protective group” or “protecting group” means the group which selectively blocks one reactive site in a multifunctional compound such that a chemical reaction can be carried out selectively at another unprotected reactive site in the meaning conventionally associated with it in synthetic chemistry. Certain processes of this invention rely upon the protective groups to block reactive nitrogen and/or oxygen atoms present in the reactants. For example, the terms “amino-protecting group” and “nitrogen protecting group” are used interchangeably herein and refer to those organic groups intended to protect the nitrogen atom against undesirable reactions during synthetic procedures. Exemplary nitrogen protecting groups include, but are not limited to, trifluoroacetyl, acetamido, benzyl (Bn), benzyloxycarbonyl (carbobenzyloxy, CBZ), p-methoxybenzyloxycarbonyl, p-nitrobenzyloxycarbonyl, tert-butoxycarbonyl (BOC), and the like. The artisan in the art will know how to chose a group for the ease of removal and for the ability to withstand the following reactions.

“Solvates” means solvent additions forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

“Subject” means mammals and non-mammals. Mammals means any member of the mammalian class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

“Arthritis” means diseases or conditions damage to joints of the body and pain associated with such joint damage. Arthritis includes rheumatoid arthritis, osteoarthritis, psoriatic arthritis, septic arthritis and gouty arthritis.

“Pain” includes, without limitation, inflammatory pain; surgical pain; visceral pain; dental pain; premenstrual pain; central pain; pain due to burns; migraine or cluster headaches; nerve injury; neuritis; neuralgias; poisoning; ischemic injury; interstitial cystitis; cancer pain; viral, parasitic or bacterial infection; post-traumatic injury; or pain associated with irritable bowel syndrome.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The terms “those defined above” and “those defined herein” when referring to a variable incorporates by reference the broad definition of the variable as well as preferred, more preferred and most preferred definitions, if any.

“Treating” or “treatment” of a disease state includes: preventing the disease state, i.e. causing the clinical symptoms of the disease state not to develop in a subject that may be exposed to or predisposed to the disease state, but does not yet experience or display symptoms of the disease state:

inhibiting the disease state, i.e., arresting the development of the disease state or its clinical symptoms, or
relieving the disease state, i.e., causing temporary or permanent regression of the disease state or its clinical symptoms.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

### Nomenclature and Structures

In general, the nomenclature used in this Application is based on AUTONOM™ v.4.0, a Beilstein Institute computerized system for the generation of IUPAC systematic nomenclature. Chemical structures shown herein were prepared using ISIS® version 2.2. Any open valency appearing on a carbon, oxygen sulfur or nitrogen atom in the structures herein indicates the presence of a hydrogen atom unless indicated otherwise. Where a nitrogen-containing heteroaryl ring is shown with an open valency on a nitrogen atom, and variables such as R^{a}, R^{b} or R^{c} are shown on the heteroaryl ring, such variables may be bound or joined to the open valency nitrogen. Where a chiral center exists in a structure but no specific stereochemistry is shown for the chiral center, both enantiomers associated with the chiral center are encompassed by the structure. Where a structure shown herein may exist in multiple tautomeric forms, all such tautomers are encompassed by the structure. The atoms represented in the structures herein are intended to encompass all naturally occurring isotopes of such atoms. Thus, for example, the hydrogen atoms represented herein are meant to include deuterium and tritium, and the carbon atoms are meant to include C¹³ and C¹⁴ isotopes.

All patents and publications identified herein are incorporated herein by reference in their entirety.

### Compounds of the Invention

The invention provides compounds of the formula I:

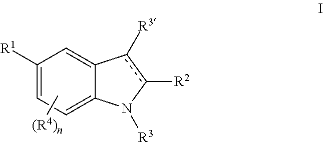

wherein:

### R¹ is:

phenyl substituted one, two or three times with a group or groups independently selected from: C₁₋₆alkyl; C₁₋₆alkoxy; halo; halo-C₁₋₆alkyl; halo-C₁₋₆alkoxy; nitrile; acetyl; C₁₋₆alkoxycarbonyl; aminocarbonyl; aminosulfonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; hydroxy; sulfonylmorpholine; sulfonylmethylpiperazine; heterocyclyl; phenyl which may be optionally substituted; or heteroaryl which may be optionally substituted;
pyridinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; heterocyclyl; phenyl which may be optionally substituted; or heteroaryl which may be optionally substituted;
pyrimidinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; heterocyclyl; phenyl which may be optionally substituted; or heteroaryl which may be optionally substituted; or
a five-membered heteroaryl ring optionally substituted one, two or three times with a group or groups independently selected from: C₁₋₆alkyl; C₃₋₆cycloalkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; heterocyclyl; phenyl which may be optionally substituted; and heteroaryl which may be optionally substituted; or two of said substituents together with the atoms to which they are attached may form a phenyl fused to the five-membered heteroaryl ring;

### R² is:

C₃₋₆cycloalkyl;
phenyl substituted one, two or three times with a group or groups independently selected from: C₁₋₆alkyl; C₁₋₆alkoxy; C₁₋₆alkoxyhydroxy; halo; halo-C₁₋₆alkyl; halo-C₁₋₆alkoxy; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; C₁₋₆alkylcarbonylhydroxy; C₁₋₆alkoxycyano; amino; hydroxy; phenyl which may be optionally substituted; or heteroaryl which may be optionally substituted;
pyridinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; phenyl which may be optionally substituted; or heteroaryl which may be optionally substituted;
pyrimidinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; phenyl which may be optionally substituted; or heteroaryl which may be optionally substituted; or
a five-membered heteroaryl ring optionally substituted once or twice with a group or groups independently selected from: C₁₋₆alkyl; C₁₋₆alkoxy; halo; halo-C₁₋₆alkyl; C₃₋₆cycloalkyl; halo-C₁₋₆alkoxy; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; phenyl which may be optionally substituted; and heteroaryl which may be optionally substituted; or two of said substituents together with the atoms to which they are attached may form a phenyl fused to said five-membered heteroaryl ring;
   R³ is hydrogen;
   R^{3′} is hydrogen or C₁₋₆alkyl;
   n is from 0 to 3;
   each R⁴ is independently selected from: hydrogen; C₁₋₆alkyl; C₁₋₆alkoxy; halo; and halo-C₁₋₆alkyl, and
   said dashed line is a bond or absent,
   or a pharmaceutically acceptable salt thereof.

In certain embodiments of formula I, R^{3′} is hydrogen.

In certain embodiments of formula I, R^{3′} is C₁₋₆alkyl.

In certain embodiments of formula I, R^{3′} is methyl.

In certain embodiments of formula I, n is from 0 to 2.

In certain embodiments of formula I, n is 0 or 1;

In certain embodiments of formula I, n is 0.

In certain embodiments of formula I, R⁴ is halo.

In certain embodiments of formula I, the dashed line is a bond.

In certain embodiments of formula I, R¹ phenyl substituted one, two or three times with a group or groups independently selected from: C₁₋₆alkyl; C₁₋₆alkoxy; halo; halo-C₁₋₆alkyl; halo-C₁₋₆alkoxy; nitrile; acetyl; C₁₋₆alkoxycarbonyl; aminocarbonyl; aminosulfonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; hydroxy; sulfonylmorpholine; sulfonylmethylpiperazine; heterocyclyl; phenyl which may be optionally substituted; or heteroaryl which may be optionally substituted.

In certain embodiments of formula I, R¹ is phenyl substituted one, two or three times with a group or groups independently selected from: C₁₋₆alkyl; C₁₋₆alkoxy; halo; halo-C₁₋₆alkyl; halo-C₁₋₆alkoxy; nitrile; acetyl; C₁₋₆alkoxycarbonyl; aminocarbonyl; aminosulfonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; hydroxy; heterocyclyl; phenyl which may be optionally substituted once or twice with a group or groups independently selected from halo, C₁₋₆alkyl, halo-C₁₋₆alkyl or C₁₋₆alkoxy; and heteroaryl which may be optionally substituted once or twice with a group or groups independently selected from halo, C₁₋₆alkyl, or halo-C₁₋₆alkyl.

In certain embodiments of formula I, R¹ is phenyl substituted one, two or three times with a group or groups independently selected from: C₁₋₆alkyl; C₁₋₆alkoxy; halo; halo-C₁₋₆alkyl; halo-C₁₋₆alkoxy; nitrile; acetyl; C₁₋₆alkoxycarbonyl; aminocarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; hydroxy; heterocyclyl selected from pyrrolidinyl, piperidinyl, piperazinyl, imidazolidinyl or isothiazolidinyl, said heterocyclyl being optionally substituted with oxo or C₁₋₆alkyl; phenyl which may be optionally substituted once or twice with a group or groups independently selected from halo, cyano, C₁₋₆alkyl, halo-C₁₋₆alkyl or C₁₋₆alkoxy; and heteroaryl selected from pyridinyl, pyrimidinyl, pyrazinyl, pyridazinyl, pyrazolyl, imidazolyl, oxazolyl, thiazolyl, isoxazolyl, isothiazolyl, furanyl or thienyl, said heteroaryl being optionally substituted once or twice with a group or groups independently selected from halo, oxo, C₁₋₆alkyl, or halo-C₁₋₆alkyl.

In certain embodiments of formula I, R¹ is phenyl substituted once or twice with a group or groups independently selected from C₁₋₆alkyl, C₁₋₆alkoxy, halo, halo-C₁₋₆alkyl, nitrile, C₁₋₆alkoxycarbonyl, C₁₋₆alkylcarbonylamino, C₁₋₆alkyl-sulfanyl, or a five-membered heteroaryl that is optionally substituted once or twice with a group or groups independently selected from halo, oxo, C₁₋₆alkyl, or halo-C₁₋₆alkyl.

In certain embodiments of formula I, R¹ is phenyl substituted once or twice with a group or groups independently selected from methyl, methoxy, fluoro, chloro, trifluoromethyl, nitrile, methoxycarbonyl, acetamido, methanesulfanyl, oxazolyl and thiazolyl.

In certain embodiments of formula I, R¹ is phenyl substituted once or twice with a group or groups independently selected from halo, nitrile, halo-C₁₋₆alkyl, oxazolyl and thiazolyl.

In certain embodiments of formula I, R¹ is: 2-chloro-5-trifluoromethyl-phenyl, 3-trifluoromethyl-phenyl, 5-methoxycarbonyl-2-methyl-phenyl, 2-methanesulfanyl-phenyl, 4-chloro-phenyl, 3-cyano-phenyl, 3-chloro-4-fluoro-phenyl, 3-methylcarbonyl-amino-phenyl, 4-methoxycarbonyl-phenyl, 2,5-dimethoxy-phenyl, 2-methoxy-5-trifluoromethyl-phenyl, 2-trifluoromethyl-phenyl, 2-methyl-5-thiazol-2-yl-phenyl, 3-oxazol-2-yl-phenyl, 2-chloro-4-methoxycarbonyl-phenyl, 4-amino-2-methyl-phenyl, 2,4-dimethoxy-phenyl, 2-methyl-4-fluoro-phenyl, 2,4-di-trifluoromethyl-phenyl, 2-methyl-4-trifluoromethoxy-phenyl, 4-aminocarbonyl-2-methyl-phenyl, 4-methanesulfonyl-2-trifluoromethyl-phenyl, 4-amino-2-chloro-phenyl, 2-chloro-4-methoxy-phenyl, 2-methyl-4-trifluoromethyl-phenyl, 4-dimethylaminosulfonyl-2-methyl-phenyl, 4-hydroxy-2-methyl-phenyl, 4-methoxy-2-trifluoromethyl-phenyl, 2-chloro-4-trifluoromethyl-phenyl, 4-(2,4-dihydro-[1,2,4]triazol-3-one-1-yl)-2-methyl-phenyl, 2-methyl-4-(5-methyl-tetrazol-1-yl)-phenyl, 2-methyl-4-(pyrrolidin-3-one-1-yl-phenyl, 4-([1,3,5]triazin-2-yl)-2-methyl-phenyl, 2-methyl-4-(tetrazol-1-yl)-phenyl, 4-(1,1-dioxo-1lambda*6*-isothiazolidin-2-yl)-2-methyl-phenyl, 2-methyl-4-(piperidin-2-one-1-yl)-phenyl, 2-methyl-4-(piperidin-4-one-1-yl)-phenyl, 2-methyl-4-(piperidin-2,6-dione-1-yl)-phenyl, 2-methyl-4-(pyrrolidin-2-one-1-yl-phenyl, 2-methyl-4-(pyrrolidin-2,5-dione-1-yl-phenyl, 2-trifluoromethyl-4-(pyrrolidin-1-yl)-phenyl, 2-methyl-5-oxazol-2-yl-phenyl, 3-thiazol-2-yl-phenyl, 4-cyano-2-methyl-phenyl, 4-methoxy-2-methyl-phenyl,2,4-dimethyl-phenyl, 4-methoxycarbonyl-2-methyl-phenyl, 4-chloro-2-methyl-phenyl, 4-cyano-phenyl, 4-methyl-phenyl, or 4-chloro-phenyl.

In certain embodiments of formula I, R¹ is 2-chloro-5-trifluoromethyl-phenyl, 3-trifluoromethyl-phenyl, 5-methoxycarbonyl-2-methyl-phenyl, 2-methanesulfanyl-phenyl, 4-chloro-phenyl, 3-cyano-phenyl, 3-chloro-4-fluoro-phenyl, 3-methylcarbonyl-amino-phenyl, 4-methoxycarbonyl-phenyl, 2,5-dimethoxy-phenyl, 2-methoxy-5-trifluoromethyl-phenyl, 2-trifluoromethyl-phenyl, 2-methyl-5-thiazol-2-yl-phenyl or 3-oxazol-2-yl-phenyl.

In certain embodiments of formula I, R¹ is substituted phenyl of formula A1 or A2

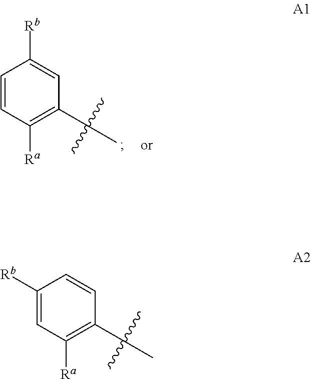

wherein:
R^{a} is: hydrogen; halo; C₁₋₆alkyl; halo-C₁₋₆alkyl; C₁₋₆alkylsulfanyl; or C₁₋₆alkoxy; and
R^{b} is: halo; halo-C₁₋₆alkyl; C₁₋₆alkoxy; halo-C₁₋₆alkoxy; cyano; amino; C₁₋₆alkoxy-carbonyl; amino; aminocarbonyl; aminosulfonyl; hydroxy; heterocyclyl; C₁₋₆alkylsulfonyl; hydroxy; or a 5-membered heteroaryl that is optionally substituted once or twice with a group or groups independently selected from halo, oxo, C₁₋₆alkyl, or halo-C₁₋₆alkyl.

In certain embodiments, R¹ is substituted phenyl of formula A1

In certain embodiments, R¹ is substituted phenyl of formula A2

In certain embodiments of formula A1 or formula A2, R^{b} is: halo; halo-C₁₋₆alkyl; C₁₋₆alkoxy; halo-C₁₋₆alkoxy; amino; C₁₋₆alkoxy-carbonyl; amino; cyano; aminocarbonyl; amino; hydroxy; heterocyclyl selected from pyrrolidinyl, piperidinyl, piperazinyl, imidazolidinyl or isothiazolidinyl, said heterocyclyl being optionally substituted with oxo or C₁₋₆alkyl or a five membered heteroaryl selected from tetrazolyl; triazolyl; oxadiazolyl; thiadiazolyl; pyrazolyl; imidazolyl; thiazolyl; isothiazolyl; oxazolyl; isoxazolyl; pyrrolyl; furanyl; or thienyl; said heteroaryl optionally substituted once or twice with a group or groups independently selected from halo, oxo, C₁₋₆alkyl, C₃₋₆cyclolalkyl, or halo-C₁₋₆alkyl.

In certain embodiments of formula A1 or formula A2, R^{a} is: hydrogen; halo; C₁₋₆alkyl; halo-C₁₋₆alkyl; or C₁₋₆alkoxy.

In certain embodiments of formula A1 or formula A2, R^{a} is: hydrogen; chloro; methyl; trifluoromethyl; or methoxy.

In certain embodiments of formula A1 or formula A2, R^{b} is: halo-C₁₋₆alkyl; C₁₋₆alkoxy; C₁₋₆alkoxy-carbonyl; cyano; oxazolyl; or thiazolyl.

In certain embodiments of formula A1 or formula A2, R^{b} is: trifluoromethyl; methoxy; methoxycarbonyl (carboxylic acid methyl ester); cyano; oxazol-2-yl; or thiazol-2-yl.

In certain embodiments of formula A1 or formula A2, R^{b} is trifluoromethyl.

In certain embodiments of formula A1 or formula A2, R^{a} is chloro.

In certain embodiments of formula A1 or formula A2, R^{a} is methyl.

In certain embodiments of formula A1 or formula A2, R^{a} is methyl, halo or trifluoromethyl and R^{b} is oxazolyl, thiazolyl or pyrazolyl, each optionally substituted with halo or methyl.

In certain embodiments of formula A1 or formula A2, R^{a} is methyl, halo or trifluoromethyl and R^{b} is oxazolyl optionally substituted with halo or methyl.

In certain embodiments of formula A1 or formula A2, R^{a} is methyl, halo or trifluoromethyl and R^{b} is thiazolyl optionally substituted with halo or methyl.

In certain embodiments of formula A1 or formula A2, R^{a} is methyl, halo or trifluoromethyl and R^{b} is pyrazolyl optionally substituted with halo or methyl.

In certain embodiments of formula I, R¹ is pyridinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; heterocyclyl; phenyl which may be optionally substituted; or heteroaryl which may be optionally substituted.

In certain embodiments of formula I, R¹ is pyridinyl optionally substituted once or twice with a group or groups independently selected from C₁₋₆alkyl, C₁₋₆alkoxy, halo, halo-C₁₋₆alkyl, cyano, acetyl, C₁₋₆alkoxycarbonyl, C₁₋₆alkyl-sulfanyl, phenyl which may be optionally substituted with C₁₋₆alkyl, C₁₋₆alkoxy, halo, halo-C₁₋₆alkyl or cyano; or a five-membered heteroaryl which may be optionally substituted with C₁₋₆alkyl, C₁₋₆alkoxy, halo, halo-C₁₋₆alkyl or cyano.

In certain embodiments of formula I, R¹ is:

2-amino-4-methyl-pyridin-5-yl; 4-methyl-2-oxo-pyridin-5-yl; 6-methyl-2-oxo-pyridin-5-yl; 3-methyl-pyridin-4-yl; 3-chloro-4-methyl-pyridin-4-yl; 2,6-dimethoxy-pyridin-5-yl; or 2-methoxy-6-methyl-pyridin-5-yl.

In certain embodiments of formula I, R¹ is pyrimidinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; heterocyclyl; phenyl which may be optionally substituted; or heteroaryl which may be optionally substituted.

In certain embodiments of formula I, R¹ is 2,4-dimethoxy-pyrimidin-5-yl.

In certain embodiments of formula I, R¹ is a five-membered heteroaryl ring optionally substituted once or twice with a group or groups independently selected from: C₁₋₆alkyl; C₃₋₆cycloalkyl; C₁₋₆alkoxy; halo; halo-C₁₋₆alkyl; halo-C₁₋₆alkoxy; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; phenyl which may be optionally substituted; heteroaryl (such as pyridinyl, pyrrolyl, oxazolyl, pyridazyl or pyrimidinyl) which may be optionally substituted; heterocyclyl (such as tetrahydropyranyl, morpholiny, piperidinyl or piperazinyl); or two such substituents together with the atoms to which they are attached may form a phenyl fused to the five-membered heteroaryl ring.

In certain embodiments of formula I, R¹ is a five-membered heteroaryl ring optionally substituted once or twice with a group or groups independently selected from C₁₋₆alkyl, C₃₋₆cycloalkyl, halo, halo-C₁₋₆alkyl, amino, oxo, hydroxy, phenyl which may be optionally substituted, heteroaryl (such as pyridinyl, pyrrolyl, oxazolyl, pyridazyl or pyrimidinyl) which may be optionally substituted, heterocyclyl (such as tetrahydropyranyl, morpholiny, piperidinyl or piperazinyl), or two of said substituents together with the atoms to which they are attached may form a phenyl fused to the five-membered heteroaryl ring.

In certain embodiments of formula I, R¹ is a five-membered heteroaryl ring selected from: tetrazolyl; triazolyl; oxadiazolyl; thiadiazolyl; pyrazolyl; imidazolyl; thiazolyl; isothiazolyl; oxazolyl; isoxazolyl; pyrrolyl; furanyl; or thienyl; each optionally substituted one, two or three times with a group or groups independently selected from C₁₋₆alkyl, C₃₋₆cycloalkyl, C₁₋₆alkoxy, halo, halo-C₁₋₆alkyl, nitrile, acetyl, C₁₋₆alkoxycarbonyl, C₁₋₆alkylcarbonylamino, C₁₋₆alkyl-sulfanyl, C₁₋₆alkyl-sulfonyl, C₁₋₆alkoxy-C₁₋₆alkyl, hydroxy-C₁₋₆alkyl, oxo, phenyl which may be optionally substituted, and heteroaryl (such as pyridinyl) which may be optionally substituted, or two of said substituents together with the atoms to which they are attached may form a phenyl fused to said five-membered heteroaryl ring.

In certain embodiments of formula I, R¹ is a five-membered heteroaryl ring selected from: tetrazolyl; triazolyl; oxadiazolyl; thiadiazolyl; pyrazolyl; imidazolyl; thiazolyl; isothiazolyl; oxazolyl; isoxazolyl; pyrrolyl; furanyl; or thienyl; each optionally substituted once or twice with a group or groups independently selected from C₁₋₆alkyl, C₃₋₆cycloalkyl, halo, halo-C₁₋₆alkyl, oxo, phenyl which may be optionally substituted, heteroaryl (such as pyridinyl or pyrrolyl) which may be optionally substituted, heterocyclyl (such as tetrahydropyranyl), or two of said substituents together with the atoms to which they are attached may form a phenyl fused to the five-membered heteroaryl ring.

In certain embodiments of formula I, R¹ is tetrazolyl; optionally substituted with a group selected from C₁₋₆alkyl, halo, halo-C₁₋₆alkyl, phenyl which may be optionally substituted, or heteroaryl which may be optionally substituted.

In certain embodiments of formula I, R¹ is triazolyl; optionally substituted once or twice with a group or groups independently selected from C₁₋₆alkyl, halo, halo-C₁₋₆alkyl, C₃₋₆cycloalkyl, oxo, phenyl which may be optionally substituted, heteroaryl which may be optionally substituted, or two of said substituents together with the atoms to which they are attached may form a phenyl fused to the triazolyl ring (i.e., benzotriazolyl).

In certain embodiments of formula I, R¹ is oxadiazolyl; optionally substituted once or twice with a group or groups independently selected from C₁₋₆alkyl, halo, halo-C₁₋₆alkyl, C₃₋₆cycloalkyl, oxo, phenyl which may be optionally substituted, or heteroaryl which may be optionally substituted.

In certain embodiments of formula I, R¹ is thiadiazolyl optionally substituted once or twice with a group or groups independently selected from C₁₋₆alkyl, halo, halo-C₁₋₆alkyl, C₃₋₆cycloalkyl, oxo, phenyl which may be optionally substituted, or heteroaryl which may be optionally substituted.

In certain embodiments of formula I, R¹ is pyrazolyl optionally substituted once or twice with a group or groups independently selected from C₁₋₆alkyl, halo, halo-C₁₋₆alkyl, C₃₋₆cycloalkyl, oxo, phenyl which may be optionally substituted, heteroaryl which may be optionally substituted, or two of said substituents together with the atoms to which they are attached may form a phenyl fused to the pyrazolyl ring (i.e., indazolyl).

In certain embodiments of formula I, R¹ is pyrazolyl optionally substituted once or twice with a group or groups independently selected from C₁₋₆alkyl, halo, halo-C₁₋₆alkyl, C₃₋₆cycloalkyl, oxo, phenyl which may be optionally substituted, pyridinyl which may be optionally substituted with C₁₋₆alkyl, pyrrolyl which may be optionally substituted with C₁₋₆alkyl, or two of said substituents together with the atoms to which they are attached may form a phenyl fused to the pyrazolyl ring (i.e., indazolyl).

In certain embodiments of formula I, R¹ is imidazolyl; optionally substituted once or twice with a group or groups independently selected from C₁₋₆alkyl, halo, halo-C₁₋₆alkyl, C₃₋₆cycloalkyl, oxo, phenyl which may be optionally substituted, heteroaryl which may be optionally substituted, or two of said substituents together with the atoms to which they are attached may form a phenyl fused to the imidazolyl ring (i.e., benzimidazolyl).

In certain embodiments of formula I, R¹ is thiazolyl; optionally substituted once or twice with a group or groups independently selected from C₁₋₆alkyl, halo, halo-C₁₋₆alkyl, C₃₋₆cycloalkyl, oxo, phenyl which may be optionally substituted, heteroaryl which may be optionally substituted, or two of said substituents together with the atoms to which they are attached may form a phenyl fused to the thiazolyl ring (i.e., benzothiazolyl).

In certain embodiments of formula I, R¹ is isothiazolyl; optionally substituted once or twice with a group or groups independently selected from C₁₋₆alkyl, halo, halo-C₁₋₆alkyl, C₃₋₆cycloalkyl, oxo, phenyl which may be optionally substituted, or heteroaryl which may be optionally substituted.

In certain embodiments of formula I, R¹ is oxazolyl; optionally substituted once or twice with a group or groups independently selected from C₁₋₆alkyl, halo, halo-C₁₋₆alkyl, C₃₋₆cycloalkyl, oxo, phenyl which may be optionally substituted, heteroaryl which may be optionally substituted, or two of said substituents together with the atoms to which they are attached may form a phenyl fused to the oxazolyl ring (i.e., benzoxazolyl).

In certain embodiments of formula I, R¹ is isoxazolyl; optionally substituted once or twice with a group or groups independently selected from C₁₋₆alkyl, halo, halo-C₁₋₆alkyl, C₃₋₆cycloalkyl, oxo, phenyl which may be optionally substituted, or heteroaryl which may be optionally substituted.

In certain embodiments of formula I, R¹ is pyrrolyl optionally substituted once or twice with a group or groups independently selected from C₁₋₆alkyl, halo, halo-C₁₋₆alkyl, C₃₋₆cycloalkyl, oxo, phenyl which may be optionally substituted, heteroaryl which may be optionally substituted, or two of said substituents together with the atoms to which they are attached may form a phenyl fused to the pyrrolyl ring (i.e., indolyl).

In certain embodiments of formula I, R¹ is furanyl optionally substituted once or twice with a group or groups independently selected from C₁₋₆alkyl, halo, halo-C₁₋₆alkyl, C₃₋₆cycloalkyl, oxo, phenyl which may be optionally substituted, heteroaryl which may be optionally substituted, or two of said substituents together with the atoms to which they are attached may form a phenyl fused to the furanyl ring (i.e., benzofuranyl).

In certain embodiments of formula I, R¹ is thienyl optionally substituted once or twice with a group or groups independently selected from C₁₋₆alkyl, halo, halo-C₁₋₆alkyl, C₃₋₆cycloalkyl, oxo, phenyl which may be optionally substituted, heteroaryl which may be optionally substituted, or two of said substituents together with the atoms to which they are attached may form a phenyl fused to the thienyl ring (i.e., benzothiophenyl).

In certain embodiments of formula I, R¹ is a five membered heteroaryl selected from: pyrazolyl; imidazolyl; thiazolyl; or oxazolyl; each optionally substituted once or twice with a group or groups independently selected from C₁₋₆alkyl, halo, halo-C₁₋₆alkyl, C₃₋₆cycloalkyl, oxo, phenyl which may be optionally substituted, pyridinyl which may be optionally substituted, or two of said substituents together with the atoms to which they are attached may form a phenyl fused to the five-membered heteroaryl ring.

In certain embodiments of formula I, R¹ is a five membered heteroaryl selected from: pyrazolyl; imidazolyl; or thiazolyl; each optionally substituted once or twice with a group or groups independently selected from C₁₋₆alkyl, halo, halo-C₁₋₆alkyl, C₃₋₆cycloalkyl, oxo, phenyl which may be optionally substituted, pyridinyl which may be optionally substituted, or two of said substituents together with the atoms to which they are attached may form a phenyl fused to the five-membered heteroaryl ring.

In certain embodiments of formula I, R¹ is pyrazolyl substituted once or twice with a group or groups independently selected from C₁₋₆alkyl, C₁₋₆alkoxy, halo, halo-C₁₋₆alkyl, nitrile, acetyl, C₁₋₆alkoxycarbonyl, C₁₋₆alkylcarbonylamino, C₁₋₆alkyl-sulfanyl, C₁₋₆alkyl-sulfonyl, C₁₋₆alkoxy-C₁₋₆alkyl, hydroxy-C₁₋₆alkyl, phenyl or pyridinyl, or two of said substituents together with the atoms to which they are attached may form a phenyl fused to said five-membered heteroaryl ring.

In certain embodiments of formula I, R¹ is pyrazolyl substituted once or twice with a group or groups independently selected from C₁₋₆alkyl, halo and halo-C₁₋₆alkyl.

In certain embodiments of formula I, R¹ is pyrazolyl substituted once or twice with a group or groups independently selected from C₁₋₆alkyl and halo-C₁₋₆alkyl.

In certain embodiments of formula I, R¹ is pyrazolyl substituted once or twice with a group or groups independently selected from C₁₋₆alkyl and halo-C₁₋₆alkyl.

In certain embodiments of formula I, R¹ is pyrazol-3-yl substituted once or twice with a group or groups independently selected from C₁₋₆alkyl and halo-C₁₋₆alkyl.

In certain embodiments of formula I, R¹ is pyrazolyl substituted once or twice with a group or groups independently selected from methyl and trifluoromethyl.

In certain embodiments of formula I, R¹ is pyrazol-3-yl substituted once or twice with a group or groups independently selected from methyl and trifluoromethyl.

In certain embodiments of formula I, R¹ is 3,5-bis-trifluoromethyl-pyrazol-1-yl, 2-methyl-5-trifluoromethyl-2H-pyrazol-3-yl or 3-trifluoromethyl-pyrazol-1-yl.

In certain embodiments of formula I, R¹ is 2-methyl-5-trifluoromethyl-2H-pyrazol-3-yl.

In certain embodiments of formula I, R¹ is imidazolyl substituted once or with a group or groups independently selected from C₁₋₆alkyl, C₁₋₆alkoxy, halo, halo-C₁₋₆alkyl, nitrile, acetyl, C₁₋₆alkoxycarbonyl, C₁₋₆alkylcarbonylamino, C₁₋₆alkyl-sulfanyl, C₁₋₆alkyl-sulfonyl, C₁₋₆alkoxy-C₁-6alkyl, hydroxy-C₁₋₆alkyl, phenyl or pyridinyl, or two of said substituents together with the atoms to which they are attached may form a phenyl fused to said five-membered heteroaryl ring.

In certain embodiments of formula I, R¹ is imidazolyl substituted once or with a group or groups independently selected from C₁₋₆alkyl, halo and halo-C₁₋₆alkyl.

In certain embodiments of formula I, R¹ is imidazolyl substituted once or with a group or groups independently selected from C₁₋₆alkyl and halo-C₁₋₆alkyl.

In certain embodiments of formula I, R¹ is imidazolyl substituted once or twice with a group or groups independently selected from methyl and trifluoromethyl.

In certain embodiments of formula I, R¹ is benzimidazolyl substituted once or twice with a group or groups independently selected from C₁₋₆alkyl, C₁₋₆alkoxy, halo and halo-C₁₋₆alkyl.

In certain embodiments of formula I, R¹ is benzimidazolyl substituted once or twice with a group or groups independently selected from C₁₋₆alkyl, C₁₋₆alkoxy and halo-C₁₋₆alkyl.

In certain embodiments of formula I, R¹ is 5-methoxy-2-methyl-1H-benzoimidazole, 2-ethyl-5-methoxy-1H-benzoimidazole, 2-isopropyl-5-methoxy-1H-benzoimidazole, 2-trifluoromethyl-1H-benzoimidazole, 5-methoxy-2-pentafluoroethyl-1H-benzoimidazole, or 5-methoxy-2-trifluoromethyl-1H-benzoimidazole.

In certain embodiments of formula I, R¹ is thiazolyl, oxazolyl or pyrazolyl, each substituted once with C₁₋₆alkyl or halo-C₁₋₆alkyl, and once with phenyl, pyridinyl or pyrimidinyl.

In certain embodiments of formula I, R¹ is thiazolyl or pyrazolyl, each substituted once with either of C₁₋₆alkyl or halo-C₁₋₆alkyl, and once with phenyl, pyridinyl or pyrimidinyl.

In certain embodiments of formula I, R¹ is thiazolyl substituted once with either of C₁₋₆alkyl or halo-C₁₋₆alkyl, and once with phenyl, pyridinyl or pyrimidinyl.

In certain embodiments of formula I, R¹ is pyrazolyl, each substituted once with either of C₁₋₆alkyl or halo-C₁₋₆alkyl, and once with phenyl, pyridinyl or pyrimidinyl.

In certain embodiments of formula I, R¹ is oxazolyl substituted once with either of C₁₋₆alkyl or halo-C₁₋₆alkyl, and once with phenyl, pyridinyl or pyrimidinyl.

In certain embodiments of formula I, R¹ is: 5-methyl-2-pyridin-2-yl-thiazol-4-yl; 4-methyl-2-phenyl-thiazol-5-yl; 5-methyl-2-pyridin-3-yl-thiazol-4-yl; 2-methyl-5-pyridin-2-yl-2H-pyrazol-3-yl; 2-ethyl-5-pyridin-2-yl-2H-pyrazol-3-yl; 2-methyl-5-pyridin-4-yl-2H-pyrazol-3-yl; 2-ethyl-5-phenyl-2H-pyrazol-3-yl; 2-methyl-5-pyridin-3-yl-2H-pyrazol-3-yl; 5-methyl-2-phenyl-thiazol-4-yl; 2-methyl-5-phenyl-2H-pyrazol-3-yl; 2-methyl-5-trifluoromethyl-2H-pyrazol-3-yl; 2-ethyl-5-phenyl-2H-pyrazol-3-yl; 2-ethyl-5-pyridin-3-yl-2H-pyrazol-3-yl; 2-ethyl-5-pyridin-2-yl-2H-pyrazol-3-yl; 2-ethyl-5-pyridin-4-yl-2H-pyrazol-3-yl; 2-methyl-5-phenyl-2H-pyrazol-3-yl; 2-methyl-5-pyridin-2-yl-2H-pyrazol-3-yl; 2-methyl-5-pyridin-3-yl-2H-pyrazol-3-yl; 2-methyl-5-pyridin-4-yl-2H-pyrazol-3-yl; 2-ethyl-5-methyl-thiazol-4-yl; 2-cyclopropyl-5-methyl-thiazol-4-yl; 2-isopropyl-5-methyl-thiazol-4-yl, 5-methyl-2-pyridin-4-yl-thiazol-4-yl, 1,4-dimethyl-1H-imidazol-2-yl, 2-methyl-5-pyridin-2-yl-2H-pyrazol-3-yl, 3-cyano-1-methyl-1H-pyrazol-4-yl, 1-methyl-3-trifluoromethyl-1H-pyrazol-4-yl, 5-methyl-2-oxazol-2-yl-thiazol-4-yl, 5-methyl-2-(tetrahydro-pyran-4-yl, 1,3-dimethyl-1H-pyrazol-4-yl, 5-cyclopropyl-2-methyl-2H-pyrazol-3-yl, 2,5-dimethyl-2H-pyrazol-3-yl, 3,5-bis-trifluoromethyl-pyrazol-1-yl, or 2-methyl-5-pyrimidin-4-yl-2H-pyrazol-3-yl.

In certain embodiments of formula I, R¹ is a group of formula B1

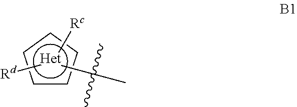

wherein:
Het is a five membered heteroaryl selected from: tetrazolyl; triazolyl; oxadiazolyl; thiadiazolyl; pyrazolyl; imidazolyl; thiazolyl; isothiazolyl; oxazolyl; isoxazolyl; pyrrolyl; furanyl; and thienyl;
R^{c} is: hydrogen; C₁₋₆alkyl; or halo-C₁₋₆alkyl; and
R^{d} is: C₁₋₆alkyl; halo-C₁₋₆alkyl; phenyl; pyridinyl; pyrimidinyl or pyridazinyl; wherein said phenyl, pyridinyl, pyrimidinyl or pyridazinyl each may be optionally substituted once or twice with a group or groups independently selected from halo, C₁₋₆alkyl; halo-C₁₋₆alkyl.

In certain embodiments of formula I, Het is: oxadiazolyl; thiadiazolyl; pyrazolyl; imidazolyl; thiazolyl; isothiazolyl; oxazolyl; or isoxazolyl.

In certain embodiments of formula B1, Het is: oxadiazolyl; thiadiazolyl; or pyrazolyl.

In certain embodiments of formula B1, Het is oxadiazolyl.

In certain embodiments of formula B1, Het is thiadiazolyl.

In certain embodiments of formula B1, Het is pyrazolyl.

In certain embodiments of formula B1, R^{c} is: C₁₋₆alkyl; or halo-C₁₋₆alkyl.

In certain embodiments of formula B¹, R^{c} is C₁₋₆alkyl.

In certain embodiments of formula B¹, R^{c} is halo-C₁₋₆alkyl.

In certain embodiments of formula B1, R^{c} is methyl or trifluoromethyl.

In certain embodiments of formula B1, R^{c} is methyl.

In certain embodiments of formula B1, R^{c} is trifluoromethyl.

In certain embodiments of formula B1, R^{d} is phenyl optionally substituted once or twice with a group or groups independently selected from halo, C₁₋₆alkyl; halo-C₁₋₆alkyl.

In certain embodiments of formula B1, R^{d} is pyridinyl optionally substituted once or twice with a group or groups independently selected from halo, C₁₋₆alkyl; halo-C₁₋₆alkyl.

In certain embodiments of formula B1, R^{d} is pyridin-2-yl.

In certain embodiments of formula B1, R^{d} is pyridin-3-yl.

In certain embodiments of formula B1, R^{d} is pyridin-4-yl.

In certain embodiments of formula B1, R^{d} is pyrimidinyl optionally substituted once or twice with a group or groups independently selected from halo, C₁₋₆alkyl; halo-C₁₋₆alkyl.

In certain embodiments of formula B1, R^{d} is pyrimidin-2-yl.

In certain embodiments of formula B1, R^{d} is pyrimidin-4-yl.

In certain embodiments of formula B1, R^{d} is pyrimidin-5-yl.

In certain embodiments of formula B1, R^{d} is pyridazinyl optionally substituted once or twice with a group or groups independently selected from halo, C₁₋₆alkyl; halo-C₁₋₆alkyl.

In certain embodiments of formula B1, R^{d} is pyridazin-2-yl.

In certain embodiments of formula B1, R^{d} is pyridazin-3-yl.

In certain embodiments of formula I, R² is phenyl substituted one, two or three times with a group or groups independently selected from: C₁₋₆alkyl; C₁₋₆alkoxy; halo; halo-C₁₋₆alkyl; halo-C₁₋₆alkoxy; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; hydroxy; phenyl which may be optionally substituted; or heteroaryl which may be optionally substituted.

In certain embodiments of formula I, R² is phenyl substituted one, two or three times with a group or groups independently selected from C₁₋₆alkyl, C₁₋₆alkoxy, halo, halo-C₁₋₆alkyl, nitrile, acetyl, C₁₋₆alkoxycarbonyl, C₁₋₆alkylcarbonylamino, C₁₋₆alkyl-sulfanyl, C₁₋₆alkyl-sulfonyl, C₁₋₆alkoxy-C₁₋₆alkyl, and hydroxy-C₁₋₆alkyl.

In certain embodiments of formula I, R² is phenyl substituted one, two or three times with a group or groups independently selected from C₁₋₆alkyl, C₁₋₆alkoxy, halo, halo-C₁₋₆alkyl, halo-C₁₋₆alkoxy, nitrile, acetyl, C₁₋₆alkoxycarbonyl, C₁₋₆alkylcarbonylamino, C₁₋₆alkyl-sulfanyl, C₁₋₆alkyl-sulfonyl, C₁₋₆alkoxy-C₁₋₆alkyl, and hydroxy-C₁₋₆alkyl.

In certain embodiments of formula I, R² is phenyl substituted once or twice with a group or groups independently selected from C₁₋₆alkyl, C₁₋₆alkoxy, halo, halo-C₁₋₆alkyl, halo-C₁₋₆alkoxy, nitrile, or C₁₋₆alkyl-sulfanyl.

In certain embodiments of formula I, R² is phenyl substituted once or twice with a group or groups independently selected from halo, halo-C₁₋₆alkyl or halo-C₁₋₆alkoxy.

In certain embodiments of formula I, R² is phenyl substituted once or twice with a group or groups independently selected from fluoro, chloro and trifluoromethoxy.

In certain embodiments of formula I, R² is halo-phenyl or dihalo-phenyl.

In certain embodiments of formula I, R² is 2-halo-phenyl, 2,3-dihalo-phenyl, 2,4-dihalo-phenyl, 2-5-dihalo-phenyl or 2,6-dihalo-phenyl.

In certain embodiments of formula I, R² is 2-halo-phenyl or 2,6-dihalo-phenyl.

In certain embodiments of formula I, R² is 2-halo-phenyl.

In certain embodiments of formula I, R² is 2,6-dihalo-phenyl.

In certain embodiments of formula I, R² is 2,6-difluoro-phenyl, 2-chloro-phenyl, 2-fluoro-phenyl, 4-chloro-phenyl, 2-chloro-6-fluoro-phenyl, 3-chloro-2-fluoro-phenyl, 2,5-dichloro-phenyl, 5-chloro-2-fluoro-phenyl, 2-chloro-4-fluoro-phenyl, 2-chloro-5-fluoro-phenyl, 2,6-dichlorophenyl, 2,3-difluoro-phenyl, 2,3-dichloro-phenyl, 2-methoxy-phenyl, 2-methyl-phenyl, 4-methoxycarbonyl-2-methyl-phenyl, or 4-trifluoromethoxy-phenyl.

In certain embodiments of formula I, R² is 2,6-difluoro-phenyl.

In certain embodiments of formula I, R² is pyridinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; phenyl which may be optionally substituted; or heteroaryl which may be optionally substituted.

In certain embodiments of formula I, R² is pyridinyl substituted once or twice with a group or groups independently selected from C₁₋₆alkyl, C₁₋₆alkoxy, halo, halo-C₁₋₆alkyl, nitrile, acetyl, C₁₋₆alkoxycarbonyl, C₁₋₆alkylcarbonylamino, C₁₋₆alkyl-sulfanyl, C₁₋₆alkyl-sulfonyl, C₁₋₆alkoxy-C₁₋₆alkyl, and hydroxy-C₁₋₆alkyl

In certain embodiments of formula I, R² is pyridinyl optionally substituted once or twice with a group or groups independently selected from fluoro, chloro and trifluoromethoxy.

In certain embodiments of formula I, R² is pyridin-4-yl, 3-fluoro-pyridin-4-yl, 3-methyl-pyridin-4-yl, 2-methyl-pyridin-3-yl, or 2-methoxy-pyridin-3-yl.

In certain embodiments of formula I, R² is pyridin-4-yl.

In certain embodiments of formula I, R² is 2-methyl-pyridin-4-yl, or 2-methyl-pyridin-3-yl.

In certain embodiments of formula I, R² is 2-methyl-pyridin-4-yl.

In certain embodiments of formula I, R² is 2-methyl-pyridin-3-yl.

In certain embodiments of formula I, R² is pyrimidinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; phenyl which may be optionally substituted; or heteroaryl which may be optionally substituted.

In certain embodiments of formula I, R² is pyrimidin-5-yl.

In certain embodiments of formula I, R² is a five-membered heteroaryl ring optionally substituted once or twice with a group or groups independently selected from: C₁₋₆alkyl; C₁₋₆alkoxy; halo; halo-C₁₋₆alkyl; C₃₋₆cycloalkyl; halo-C₁₋₆alkoxy; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; phenyl which may be optionally substituted; and heteroaryl which may be optionally substituted; or two of said substituents together with the atoms to which they are attached may form a phenyl fused to said five-membered heteroaryl ring;

In certain embodiments of formula I, R² is a five-membered heteroaryl ring containing one or two nitrogen atoms and optionally includes a sulfur atom, and which further is optionally substituted once or twice with a group or groups independently selected from C₁₋₆alkyl, C₁₋₆alkoxy, halo, halo-C₁₋₆alkyl, halo-C₁₋₆alkoxy, nitrile, acetyl, C₁₋₆alkoxycarbonyl, C₁₋₆alkylcarbonylamino, C₁₋₆alkyl-sulfanyl, C₁₋₆alkyl-sulfonyl, C₁₋₆alkoxy-C₁₋₆alkyl, and hydroxy-C₁₋₆alkyl, or two of said substituents together with the atoms to which they are attached may form a phenyl fused to said five-membered heteroaryl ring.

In certain embodiments of formula I, R² is pyrazolyl optionally substituted once or with a group or groups independently selected from C₁₋₆alkyl, C₁₋₆alkoxy, halo, halo-C₁₋₆alkyl, nitrile, acetyl, C₁₋₆alkoxycarbonyl, C₁₋₆alkylcarbonylamino, C₁₋₆alkyl-sulfanyl, C₁₋₆alkyl-sulfonyl, C₁₋₆alkoxy-C₁₋₆alkyl, and hydroxy-C₁₋₆alkyl, or two of said substituents together with the atoms to which they are attached may form a phenyl fused to said five-membered heteroaryl ring.

In certain embodiments of formula I, R² is imidazolyl optionally substituted once or with a group or groups independently selected from C₁₋₆alkyl, C₁₋₆alkoxy, halo, halo-C₁₋₆alkyl, nitrile, acetyl, C₁₋₆alkoxycarbonyl, C₁₋₆alkylcarbonylamino, C₁₋₆alkyl-sulfanyl, C₁₋₆alkyl-sulfonyl, C₁₋₆alkoxy-C₁₋₆alkyl, and hydroxy-C₁₋₆alkyl, or two of said substituents together with the atoms to which they are attached may form a phenyl fused to said five-membered heteroaryl ring.

In certain embodiments of formula I, R² is thiadiazolyl optionally substituted once with a group elected from C₁₋₆alkyl, C₁₋₆alkoxy, halo, halo-C₁₋₆alkyl, nitrile, acetyl, C₁₋₆alkoxycarbonyl, C₁₋₆alkylcarbonylamino, C₁₋₆alkyl-sulfanyl, C₁₋₆alkyl-sulfonyl, C₁₋₆alkoxy-C₁₋₆alkyl, and hydroxy-C₁₋₆alkyl.

In certain embodiments of formula I, R² is C₃₋₆cycloalkyl.

In certain embodiments of formula I, R² is 3,6-dihydro-2H-pyran-4-yl.

In certain embodiments of formula I, provided are:
2-(2,6-Difluoro-phenyl)-5-(2-methyl-5-trifluoromethyl-2H-pyrazol-3-yl)-1H-indole;
1-[2-(2,6-Difluoro-phenyl)-1H-indol-5-yl]-5-methoxy-2-trifluoromethyl-1H-benzoimidazole;
5-(2-Methyl-5-trifluoromethyl-2H-pyrazol-3-yl)-2-(4-trifluoromethoxy-phenyl)-1H-indole;
2-(2,6-Difluoro-phenyl)-5-(5-methyl-2-pyridin-2-yl-thiazol-4-yl)-1H-indole;
2-(2-Chloro-phenyl)-5-(5-methyl-2-pyridin-2-yl-thiazol-4-yl)-1H-indole;
5-(5-Methyl-2-pyridin-2-yl-thiazol-4-yl)-2-o-tolyl-1H-indole;
2-(2-Chloro-phenyl)-5-(4-methyl-2-phenyl-thiazol-5-yl)-1H-indole;
5-(4-Methyl-2-phenyl-thiazol-5-yl)-2-(2-methyl-pyridin-3-yl)-1H-indole;
2-(3-Fluoro-pyridin-4-yl)-5-(5-methyl-2-pyridin-2-yl-thiazol-4-yl)-1H-indole;
2-(3-Methyl-pyridin-4-yl)-5-(5-methyl-2-pyridin-2-yl-thiazol-4-yl)-1H-indole;
2-(2-Fluoro-phenyl)-5-(5-methyl-2-pyridin-3-yl-thiazol-4-yl)-1H-indole;
2-(2-Chloro-phenyl)-5-(5-methyl-2-pyridin-3-yl-thiazol-4-yl)-1H-indole;
2-(2,6-Difluoro-phenyl)-5-(5-methyl-2-pyridin-3-yl-thiazol-4-yl)-1H-indole;
2-(2-Fluoro-phenyl)-5-(2-methyl-5-trifluoromethyl-2H-pyrazol-3-yl)-1H-indole;
2-(2,6-difluoro-phenyl)-5-(2-ethyl-5-phenyl-2H-pyrazol-3-yl)-1H-indole;
2-(2,6-Difluoro-phenyl)-5-(2-ethyl-5-pyridin-2-yl-2H-pyrazol-3-yl)-1H-indole;
2-(2,6-Difluoro-phenyl)-5-(2-methyl-5-pyridin-4-yl-2H-pyrazol-3-yl)-1H-indole;
2-(2,6-Difluoro-phenyl)-5-(2-ethyl-5-pyridin-4-yl-2H-pyrazol-3-yl)-1H-indole;
2-(2,6-Difluoro-phenyl)-5-(2-methyl-5-pyridin-3-yl-2H-pyrazol-3-yl)-1H-indole;
2-(2,6-Difluoro-phenyl)-5-(2-ethyl-5-pyridin-3-yl-2H-pyrazol-3-yl)-1H-indole;
2-(2,6-Difluoro-phenyl)-5-(5-methyl-2-pyridin-4-yl-thiazol-4-yl)-1H-indole;
2-(2-Chloro-phenyl)-5-(5-methyl-2-pyridin-4-yl-thiazol-4-yl)-1H-indole;
2-(2,6-Difluoro-phenyl)-5-(2-methyl-5-oxazol-2-yl-phenyl)-1H-indole;
2-(2,6-Difluoro-phenyl)-5-(3-oxazol-2-yl-phenyl)-1H-indole;
2-(2,6-Difluoro-phenyl)-5-(2-methyl-5-thiazol-2-yl-phenyl)-1H-indole;
2-(2,6-Difluoro-phenyl)-5-(2,5-dimethoxy-phenyl)-1H-indole;
4-[2-(2,6-Difluoro-phenyl)-1H-indol-5-yl]-3-methyl-benzonitrile;
2-(2,6-Difluoro-phenyl)-5-(4-methoxy-2-methyl-phenyl)-1H-indole;
2-(2,6-Difluoro-phenyl)-5-(2,4-dimethyl-phenyl)-1H-indole;
4-[2-(2,6-Difluoro-phenyl)-1H-indol-5-yl]-3-methyl-benzoic acid methyl ester;
5-(4-Chloro-2-methyl-phenyl)-2-(2,6-difluoro-phenyl)-1H-indole;
2-(2,6-Difluoro-phenyl)-5-(2-methyl-4-trifluoromethyl-phenyl)-1H-indole;
2-(5-Chloro-2-fluoro-phenyl)-5-(2-methyl-5-trifluoromethyl-2H-pyrazol-3-yl)-1H-indole;
2-(2,4-Dichloro-phenyl)-5-(2-methyl-5-trifluoromethyl-2H-pyrazol-3-yl)-1H-indole;
2-(2-Chloro-4-fluoro-phenyl)-5-(2-methyl-5-trifluoromethyl-2H-pyrazol-3-yl)-1H-indole;
2-(3-Chloro-pyridin-4-yl)-5-(2-methyl-5-trifluoromethyl-2H-pyrazol-3-yl)-1H-indole;
2-(3-Methyl-pyridin-4-yl)-5-(2-methyl-5-trifluoromethyl-2H-pyrazol-3-yl)-1H-indole;
2-(6-Methoxy-2-methyl-pyridin-3-yl)-5-(2-methyl-5-trifluoromethyl-2H-pyrazol-3-yl)-1H-indole;
3-Methyl-4-[5-(2-methyl-5-trifluoromethyl-2H-pyrazol-3-yl)-1H-indol-2-yl]-benzoic acid methyl ester;
3-Methyl-4-[5-(2-methyl-5-trifluoromethyl-2H-pyrazol-3-yl)-1H-indol-2-yl]-benzoic acid methyl ester;
2-(2,3-Dichloro-phenyl)-5-(2-methyl-5-trifluoromethyl-2H-pyrazol-3-yl)-1H-indole;
2-(2-Chloro-5-fluoro-phenyl)-5-(2-methyl-5-trifluoromethyl-2H-pyrazol-3-yl)-1H-indole;
2-(3-Chloro-2-methoxy-pyridin-4-yl)-5-(2-methyl-5-trifluoromethyl-2H-pyrazol-3-yl)-1H-indole;
2-(3-Fluoro-pyridin-4-yl)-5-(2-methyl-5-trifluoromethyl-2H-pyrazol-3-yl)-1H-indole;
2-(3,5-Dimethyl-isoxazol-4-yl)-5-(2-methyl-5-trifluoromethyl-2H-pyrazol-3-yl)-1H-indole;
2-(2,6-Difluoro-phenyl)-5-(2-ethyl-5-trifluoromethyl-2H-pyrazol-3-yl)-1H-indole;
2-(2-Chloro-6-fluoro-phenyl)-5-(2-ethyl-5-trifluoromethyl-2H-pyrazol-3-yl)-1H-indole;
2-(2-Chloro-6-fluoro-phenyl)-5-(2-methyl-5-trifluoromethyl-2H-pyrazol-3-yl)-1H-indole;
2-(2-Chloro-6-fluoro-phenyl)-5-(2-ethyl-5-pyridin-3-yl-2H-pyrazol-3-yl)-1H-indole;
2-(2-Chloro-6-fluoro-phenyl)-5-(5-methyl-2-pyridin-3-yl-thiazol-4-yl)-1H-indole;
2-(2-Chloro-6-fluoro-phenyl)-5-(2-methyl-5-pyridin-4-yl-2H-pyrazol-3-yl)-1H-indole;
2-(2-Chloro-6-fluoro-phenyl)-5-(2-methyl-4-oxazol-2-yl-phenyl)-1H-indole;
4-[2-(2-Chloro-6-fluoro-phenyl)-1H-indol-5-yl]-3-methyl-benzoic acid methyl ester;
2-(2-chloro-6-fluoro-phenyl)-5-(2,4-dimethoxy-phenyl)-1H-indole;
5-(2,4-Bis-trifluoromethyl-phenyl)-2-(2-chloro-6-fluoro-phenyl)-1H-indole;
2-(2-Chloro-6-fluoro-phenyl)-5-(2-chloro-4-trifluoromethyl-phenyl)-1H-indole;
2-(2-Chloro-4-fluoro-phenyl)-5-(5-methyl-2-pyridin-2-yl-thiazol-4-yl)-1H-indole;
2-(2-Chloro-5-fluoro-phenyl)-5-(5-methyl-2-pyridin-2-yl-thiazol-4-yl)-1H-indole;
2-(2-Chloro-phenyl)-5-(2-methyl-5-trifluoromethyl-2H-pyrazol-3-yl)-1H-indole;
5-(2-Methyl-5-trifluoromethyl-2H-pyrazol-3-yl)-2-o-tolyl-1H-indole;
2-(2-Chloro-phenyl)-5-(5-cyclopropyl-2-methyl-2H-pyrazol-3-yl)-1H-indole;
5-(5-Cyclopropyl-2-methyl-2H-pyrazol-3-yl)-2-o-tolyl-1H-indole;
5-(5-Cyclopropyl-2-methyl-2H-pyrazol-3-yl)-2-(2,6-difluoro-phenyl)-1H-indole;
2-(3-Fluoro-pyridin-4-yl)-5-(5-methyl-2-pyridin-3-yl-thiazol-4-yl)-1H-indole;
3-Methyl-4-[5-(5-methyl-2-pyridin-3-yl-thiazol-4-yl)-1H-indol-2-yl]-benzoic acid methyl ester;
2-(2,6-Difluoro-4-methoxy-phenyl)-5-(5-methyl-2-pyridin-3-yl-thiazol-4-yl)-1H-indole;
2-(2-Chloro-4-fluoro-phenyl)-5-(5-methyl-2-pyridin-3-yl-thiazol-4-yl)-1H-indole;
2-(4-Isopropyl-pyrimidin-5-yl)-5-(5-methyl-2-pyridin-3-yl-thiazol-4-yl)-1H-indole;
2-(2-Chloro-phenyl)-5-(2-isopropyl-5-methyl-thiazol-4-yl)-1H-indole;
2-(2,6-Difluoro-phenyl)-5-(2-isopropyl-5-methyl-thiazol-4-yl)-1H-indole;
2-(2,6-Difluoro-phenyl)-5-(2-isopropyl-5-methyl-thiazol-4-yl)-1H-indole;
5-(2-Cyclopropyl-5-methyl-thiazol-4-yl)-2-(2,6-difluoro-phenyl)-1H-indole;
2-(2,6-Difluoro-phenyl)-5-(5-methyl-2-oxazol-2-yl-thiazol-4-yl)-1H-indole;
2-(2,6-Difluoro-phenyl)-5-[5-methyl-2-(tetrahydro-pyran-4-yl)-thiazol-4-yl]-1H-indole;
2-(2-Fluoro-phenyl)-5-(2-methyl-5-pyridin-3-yl-2H-pyrazol-3-yl)-1H-indole;
2-(2-Fluoro-phenyl)-5-(2-ethyl-5-pyridin-3-yl-2H-pyrazol-3-yl)-1H-indole;
2-(2-Chloro-phenyl)-5-(2-methyl-5-pyridin-3-yl-2H-pyrazol-3-yl)-1H-indole;
2-(2-Chloro-phenyl)-5-(2-ethyl-5-pyridin-3-yl-2H-pyrazol-3-yl)-1H-indole;
5-(2-Methyl-5-pyridin-3-yl-2H-pyrazol-3-yl)-2-o-tolyl-1H-indole;
5-(2-Ethyl-5-pyridin-3-yl-2H-pyrazol-3-yl)-2-o-tolyl-1H-indole;
2-(2-Chloro-phenyl)-5-(2-methyl-5-pyridin-2-yl-2H-pyrazol-3-yl)-1H-indole;
2-(2-Chloro-5-fluoro-phenyl)-5-(2-methyl-5-pyridin-2-yl-2H-pyrazol-3-yl)-1H-indole;
2-(2-Chloro-4-fluoro-phenyl)-5-(2-methyl-5-pyridin-2-yl-2H-pyrazol-3-yl)-1H-indole;
2-(2,3-Difluoro-phenyl)-5-(2-methyl-5-pyridin-3-yl-2H-pyrazol-3-yl)-1H-indole;
2-(2,3-Dichloro-phenyl)-5-(2-methyl-5-pyridin-3-yl-2H-pyrazol-3-yl)-1H-indole;
2-(2-Chloro-4-fluoro-phenyl)-5-(2-methyl-5-pyridin-3-yl-2H-pyrazol-3-yl)-1H-indole;
2-(2,5-Dichloro-phenyl)-5-(2-methyl-5-pyridin-3-yl-2H-pyrazol-3-yl)-1H-indole;
4-[2-(2,6-Difluoro-phenyl)-1H-indol-5-yl]-3-chloro-benzoic acid methyl ester;
4-[2-(2,6-Difluoro-phenyl)-1H-indol-5-yl]-3-methyl-benzamide;
2-(2,6-Difluoro-phenyl)-5-(2,4-dimethoxy-phenyl)-1H-indole;
2-(2,6-Difluoro-phenyl)-5-(4-fluoro-2-methyl-phenyl)-1H-indole;
5-(2,4-Bis-trifluoromethyl-phenyl)-2-(2,6-difluoro-phenyl)-1H-indole;
2-(2,6-Difluoro-phenyl)-5-(2,4-dimethoxy-pyrimidin-5-yl)-1H-indole;
5-(2-Chloro-4-trifluoromethyl-phenyl)-2-(2,6-difluoro-phenyl)-1H-indole;
2-(2,6-Difluoro-phenyl)-5-(2,6-dimethoxy-pyridin-3-yl)-1H-indole;
2-(2,6-Difluoro-phenyl)-5-(4-methanesulfonyl-2-trifluoromethyl-phenyl)-1H-indole;
4-[2-(2,6-Difluoro-phenyl)-1H-indol-5-yl]-N,N-dimethyl-3-trifluoromethyl-benzenesulfonamide;
5-(2-Chloro-4-methoxy-phenyl)-2-(2,6-difluoro-phenyl)-1H-indole;
2-(2,6-Difluoro-phenyl)-5-(4-methoxy-2-trifluoromethyl-phenyl)-1H-indole;
2-(2,6-Difluoro-phenyl)-5-(2-methyl-4-trifluoromethoxy-phenyl)-1H-indole;
2-(2,6-Difluoro-phenyl)-5-(6-methoxy-2-methyl-pyridin-3-yl)-1H-indole;
2-(2,6-Difluoro-phenyl)-5-(2-methyl-4-oxazol-2-yl-phenyl)-1H-indole;
2-(2,6-Difluoro-phenyl)-5-(2-methoxy-4-oxazol-2-yl-phenyl)-1H-indole;
2-(2,6-Difluoro-phenyl)-5-(4-methyl-6-piperazin-1-yl-pyridin-3-yl)-1H-indole;
2-(2,6-Difluoro-phenyl)-5-(5-methyl-2-pyridazin-4-yl-thiazol-4-yl)-1H-indole;
2-(2,6-Difluoro-phenyl)-5-(2-iodo-5-methyl-thiazol-4-yl)-1H-indole;
5-{5-[2-(2,6-Difluoro-phenyl)-1H-indol-5-yl]-1-methyl-1H-pyrazol-3-yl}-pyrimidin-2-ylamine;
2-(2,6-Difluoro-phenyl)-5-(1-methyl-1H,1′H-[3,3′]bipyrazolyl-5-yl)-1H-indole;
5-[2-(2-Fluoro-6-methyl-phenyl)-1H-indol-5-yl]-1-methyl-1H-pyrazole-3-carboxylic acid dimethylamide;
2-(2,6-Difluoro-phenyl)-5-(2-methyl-5-oxazol-2-yl-2H-pyrazol-3-yl)-1H-indole;
5-(5-Bromo-2-methyl-2H-pyrazol-3-yl)-2-(2,6-difluoro-phenyl)-1H-indole;
2-(2-Fluoro-phenyl)-5-(6-methoxy-4-methyl-pyridin-3-yl)-1H-indole;
2-(2,6-Difluoro-phenyl)-5-(6-methoxy-4-methyl-pyridin-3-yl)-1H-indole;
2-(2,6-Difluoro-phenyl)-5-(2-methyl-5-pyridin-3-yl-2H-[1,2,4]triazol-3-yl)-1H-indole;
2-(2-Chloro-6-fluoro-phenyl)-5-(2-methyl-4-[1,3,4]oxadiazol-2-yl-phenyl)-1H-indole;
2-(2-Chloro-6-fluoro-phenyl)-5-(2-methyl-5-pyridin-3-yl-2H-[1,2,4]triazol-3-yl)-1H-indole;
5-[2-(2-Chloro-6-fluoro-phenyl)-1H-indol-5-yl]-4-methyl-pyridine-2-carboxylic acid methyl ester;
5-[2-(2-Chloro-6-fluoro-phenyl)-1H-indol-5-yl]-4-methyl-pyridine-2-carboxylic acid methylamide;
2-(2-Chloro-6-fluoro-phenyl)-5-(4-methyl-6-[1,3,4]oxadiazol-2-yl-pyridin-3-yl)-1H-indole;
2-(2-Chloro-6-fluoro-phenyl)-5-(4-methyl-6-(5-[4-methyl-[1,3,4]oxadiazol-2-yl)-pyridin-3-yl]-1H-indole;
2-(2-Chloro-6-fluoro-phenyl)-5-(6-methoxy-4-methyl-pyridin-3-yl)-1H-indole;
2-(2-Chloro-6-fluoro-phenyl)-5-(5-methoxy-3-methyl-pyridin-2-yl)-1H-indole;
2-(2-Chloro-6-fluoro-phenyl)-5-(6-methoxy-2-methyl-pyridin-3-yl)-1H-indole;
2-(2-Chloro-6-fluoro-phenyl)-5-(2-ethyl-5-pyridin-3-yl-2H-[1,2,4]triazol-3-yl)-1H-indole;
2-(2-Chloro-6-fluoro-phenyl)-5-(5-methyl-2-oxazol-2-yl-thiazol-4-yl)-1H-indole;
4-[2-(2-Chloro-phenyl)-1H-indol-5-yl]-3-methyl-benzoic acid methyl ester;
4-[2-(2-Chloro-phenyl)-1H-indol-5-yl]-3,N-dimethyl-benzamide;
4-[2-(2-Chloro-phenyl)-1H-indol-5-yl]-3-methyl-benzamide;
4-[2-(2-Chloro-phenyl)-1H-indol-5-yl]-3-methyl-benzonitrile;
4-[2-(2-Chloro-phenyl)-1H-indol-5-yl]-3,N,N-trimethyl-benzenesulfonamide;
4-[5-(4-carbomethoxy-2-methyl-phenyl)-1H-indol-2-yl]-3-methyl-benzoic acid methyl ester;
4-[2-(2-Chloro-4-methoxy-phenyl)-1H-indol-5-yl]-3-methyl-benzonitrile;
4-[2-(2-Fluoro-4-methanesulfonyl-phenyl)-1H-indol-5-yl]-3-methyl-benzonitrile;
4-[2-(2-Fluoro-3-cyano-phenyl)-1H-indol-5-yl]-3-methyl-benzonitrile;
4-(2-(2,6-difluoro-4-methoxyphenyl)-1H-indol-5-yl)-3-methylbenzonitrile;
4-(2-(2-fluorophenyl)-1H-indol-5-yl)-3-methylbenzonitrile;
4-(2-(4-Cyano-2-methylphenyl)-1H-indol-5-yl)-3-methylbenzonitrile;
4-(2-(2-Chloro-5-cyanophenyl)-1H-indol-5-yl)-3-methylbenzonitrile;
4-(2-(6-methoxy-2-methylpyridin-3-yl)-1H-indol-5-yl)-3-methylbenzonitrile;
4-(2-(3-chloro-2-methoxypyridin-4-yl)-1H-indol-5-yl)-3-methylbenzonitrile;
4-(2-(2,4-difluorophenyl)-1H-indol-5-yl)-3-methylbenzonitrile;
4-(2-(2,6-difluoro-3-methoxyphenyl)-1H-indol-5-yl)-3-methylbenzonitrile;
4-(2-(6-methoxy-4-methylpyridin-3-yl)-1H-indol-5-yl)-3-methylbenzonitrile;
3-methyl-4-(2-(4-methylpyridin-3-yl)-1H-indol-5-yl)benzonitrile;
3-methyl-4-(2-(3-methylpyridin-4-yl)-1H-indol-5-yl)benzonitrile;
3-methyl-4-(2-(3-methylthiophen-2-yl)-1H-indol-5-yl)benzonitrile;
3-methyl-4-(2-(2-methylpyridin-3-yl)-1H-indol-5-yl)benzonitrile;
4-(2-(2,4-dimethylthiazol-5-yl)-1H-indol-5-yl)-3-methylbenzonitrile;
3-methyl-4-(2-(4-methylthiophen-3-yl)-1H-indol-5-yl)benzonitrile;
3-methyl-4-(2-(2-methyl-1H-pyrazol-5-yl)-1H-indol-5-yl)benzonitrile;
4-(2-(3,5-dimethylisoxazol-4-yl)-1H-indol-5-yl)-3-methylbenzonitrile;
2-fluoro-3-(5-(6-methoxy-4-methylpyridin-3-yl)-1H-indol-2-yl)benzonitrile;
4-(2-(2,6-difluoro-4-(2-methoxyethoxy)phenyl)-1H-indol-5-yl)-3-methylbenzonitrile;
4-(2-(2,6-difluoro-4-(2-hydroxyethoxy)phenyl)-1H-indol-5-yl)-3-methylbenzonitrile;
4-(2-(4-(3-cyanopropoxy)-2,6-difluorophenyl)-1H-indol-5-yl)-3-methylbenzonitrile;
4-(2-(2,6-difluoro-4-(3-hydroxypropoxy)phenyl)-1H-indol-5-yl)-3-methylbenzonitrile;
4-(2-(2,6-difluoro-4-hydroxyphenyl)-1H-indol-5-yl)-3-methylbenzonitrile;
4-[2-(2-chloro-6-fluorophenyl)-1H-indol-5-yl]-3-methylbenzonitrile;
4-[2-(2-Chloro-6-fluoro-phenyl)-1H-indol-5-yl]-3,N,N-trimethyl-benzenesulfonamide; or
2-(2-Chloro-6-fluoro-phenyl)-5-(6-chloro-4-methyl-pyridin-3-yl)-1H-indole.
6-(2-(2-chloro-6-fluorophenyl)-1H-indol-5-yl)-5-methylnicotinonitrile;
5-(2-(2-chloro-6-fluorophenyl)-1H-indol-5-yl)-4-methylpicolinonitrile;
2-(2-chloro-6-fluorophenyl)-5-(6-(2-methoxyethoxy)-4-methylpyridin-3-yl)-1H-indole;
2-(2-chloro-6-fluorophenyl)-5-(6-ethoxy-4-methylpyridin-3-yl)-1H-indole;
4-(5-(2-(2-chloro-6-fluorophenyl)-1H-indol-5-yl)-4-methylpyridin-2-yl)morpholine;
5-(2-(2-chloro-6-fluorophenyl)-1H-indol-5-yl)-N,4-dimethylpyridin-2-amine;
6-(2-(2-chloro-6-fluorophenyl)-1H-indol-5-yl)-N,N,5-trimethylpyridine-3-sulfonamide;
4-(2-(2-chloro-6-fluorophenyl)-1H-indol-5-yl)-N,3-dimethylbenzenesulfonamide;
4-(4-(2-(2-chloro-6-fluorophenyl)-1H-indol-5-yl)-3-methylphenylsulfonyl)morpholine;
2-(2-chloro-6-fluorophenyl)-5-(2-methyl-4-(4-methylpiperazin-1-ylsulfonyl)phenyl)-1H-indole;
2-(2-chloro-6-fluorophenyl)-5-(2-methyl-4-(2-methyl-2H-tetrazol-5-yl)phenyl)-1H-indole;
4-[2-(2-Chloro-6-fluoro-phenyl)-1H-indol-5-yl]-3-methoxy-benzonitrile;
2-(2-Chloro-6-fluoro-phenyl)-5-(6-methanesulfonyl-4-methyl-pyridin-3-yl)-1H-indole;
5-(6-Chloro-4-ethyl-pyridin-3-yl)-2-(2-chloro-6-fluoro-phenyl)-1H-indole;
4-[2-(2-chloro-6-fluorophenyl)-1H-indol-5-yl]-5-ethyl-2-(pyridin-3-yl)thiazole;
2-(2-Chloro-6-fluoro-phenyl)-5-(5-methyl-2-pyrazin-2-yl-thiazol-4-yl)-1H-indole;
2-(2-Chloro-6-fluoro-phenyl)-5-(5-methyl-2-pyrimidin-5-yl-thiazol-4-yl)-1H-indole;
2-(2-Chloro-6-fluoro-phenyl)-5-[5-methyl-2-(6-methyl-pyridin-3-yl)-thiazol-4-yl]-1H-indole;
2-(2-Chloro-6-fluoro-phenyl)-5-(5-ethyl-2-pyrazin-2-yl-thiazol-4-yl)-1H-indole;
2-(2-Chloro-6-fluoro-phenyl)-5-(5-isopropyl-2-pyridin-3-yl-thiazol-4-yl)-1H-indole;
2-(2-Chloro-6-fluoro-phenyl)-5-(5-isopropyl-2-pyrazin-2-yl-thiazol-4-yl)-1H-indole;
2-(2-chloro-6-fluoro-phenyl)-5-[2-pyridin-3-yl-5-(2,2,2-trifluoro-1-methyl-ethyl)-thiazol-4-yl]-1H-indole;
2-(2-chloro-6-fluorophenyl)-5-(1-ethyl-3-(pyrazin-2-yl)-1H-pyrazol-5-yl)-1H-indole;
2-(2-Chloro-6-fluoro-phenyl)-5-(2-methyl-5-pyridin-2-yl-2H-[1,2,4]triazol-3-yl)-1H-indole;
2-(2-Chloro-phenyl)-5-(2-ethyl-5-pyridin-3-yl-2H-[1,2,4]triazol-3-yl)-1H-indole;
2-(2,6-Dichloro-phenyl)-5-(2-ethyl-5-pyridin-3-yl-2H-[1,2,4]triazol-3-yl)-1H-indole;
2-(2-Chloro-6-fluoro-phenyl)-5-(2-ethyl-5-pyrazin-2-yl-2H-[1,2,4]triazol-3-yl)-1H-indole;
2-(2-Chloro-6-fluoro-phenyl)-5-(2-methyl-5-pyrimidin-5-yl-2H-[1,2,4]triazol-3-yl)-1H-indole;
5-(1-ethyl-3-(trifluoromethyl)-1H-pyrazol-5-yl)-2-(3-methylpyridin-4-yl)-1H-indole;
5-(1-ethyl-3-(trifluoromethyl)-1H-pyrazol-5-yl)-2-(6-methoxy-4-methylpyridin-3-yl)-1H-indole;
5-(1-ethyl-3-(trifluoromethyl)-1H-pyrazol-5-yl)-2-(4-methylpyridin-3-yl)-1H-indole;
5-(1-ethyl-3-(trifluoromethyl)-1H-pyrazol-5-yl)-2-(3-fluoropyridin-4-yl)-1H-indole;
5-(1-ethyl-3-(trifluoromethyl)-1H-pyrazol-5-yl)-2-(6-methoxy-2-methylpyridin-3-yl)-1H-indole;
2-(3-chloro-2-methoxypyridin-4-yl)-5-(1-ethyl-3-(trifluoromethyl)-1H-pyrazol-5-yl)-1H-indole;
2-cyclohexyl-5-(1-ethyl-3-(trifluoromethyl)-1H-pyrazol-5-yl)-1H-indole;
2-cyclohexenyl-5-(1-ethyl-3-(trifluoromethyl)-1H-pyrazol-5-yl)-1-(trifluoromethylsulfonyl)-1H-indole;
2-Cyclohexyl-5-(1-ethyl-3-(trifluoromethyl)-1H-pyrazol-5-yl)-1H-indole;
[2-(2-Cyclohexyl-ethyl)-4-(2-ethyl-5-trifluoromethyl-2H-pyrazol-3-yl)-phenyl]-methyl-amine;
[2-(2-Cyclohexyl-ethyl)-4-(2-ethyl-5-trifluoromethyl-2H-pyrazol-3-yl)-phenyl]-methyl-amine;
5-(1-ethyl-3-(trifluoromethyl)-1H-pyrazol-5-yl)-2-(tetrahydro-2H-pyran-4-yl)-1H-indole;
5-(1-ethyl-3-(trifluoromethyl)-1H-pyrazol-5-yl)-2-(tetrahydro-2H-pyran-3-yl)-1H-indole;
1-(4-(5-(1-ethyl-3-(trifluoromethyl)-1H-pyrazol-5-yl)-1H-indol-2-yl)piperidin-1-yl)ethanone;
2-(2-chloro-6-fluoro-4-methoxyphenyl)-5-(1-methyl-3-(trifluoromethyl)-1H-pyrazol-5-yl)-1H-indole;
4-(2-(2-chloro-6-fluoro-4-methoxyphenyl)-1H-indol-5-yl)-3-methylbenzonitrile;
2-(2-chloro-6-fluoro-4-methoxyphenyl)-5-(1-ethyl-3-(trifluoromethyl)-1H-pyrazol-5-yl)-1H-indole;
2-(2,6-difluorophenyl)-5-(1-ethyl-3-(pyrazin-2-yl)-1H-pyrazol-5-yl)-1H-indole;
2-(2,6-Difluoro-phenyl)-5-(2-ethyl-5-pyridin-3-yl-2H-[1,2,4]triazol-3-yl)-1H-indole;
2-(2,6-Difluoro-phenyl)-5-(5-methyl-2-pyrazin-2-yl-thiazol-4-yl)-1H-indole;
2-(2,6-Difluoro-phenyl)-5-(5-ethyl-2-pyridin-3-yl-thiazol-4-yl)-1H-indole;
2-(2,6-Difluoro-phenyl)-5-(4-methyl-6-oxazol-2-yl-pyridin-3-yl)-1H-indole;
5-{5-[2-(2,6-Difluoro-phenyl)-1H-indol-5-yl]-4-methyl-pyridin-2-yl}-pyrimidin-2-ylamine;
2-(2,6-Difluoro-phenyl)-5-(4-methyl-6-pyrimidin-5-yl-pyridin-3-yl)-1H-indole;
2-(4-Methyl-pyridin-3-yl)-5-(4,4,5,5-tetramethyl-1,3,2-dioxaborolan-2-yl)-1H-indole;
4-Methyl-5-[2-(4-methyl-pyridin-3-yl)-1H-indol-5-yl]-pyridine-2-carbonitrile;
4-Methoxy-5-[2-(4-methyl-pyridin-3-yl)-1H-indol-5-yl]-pyridine-2-carbonitrile;
5-(6-Methanesulfonyl-4-methyl-pyridin-3-yl)-2-(4-methyl-pyridin-3-yl)1indole;
5-(6-Chloro-4-methyl-pyridin-3-yl)-2-(4-methyl-pyridin-3-yl)-1H-indole;
5-(6-Methoxy-4-methyl-pyridin-3-yl)-2-(4-methyl-pyridin-3-yl)-1H-indole;
2-(2,6-Dichloro-phenyl)-5-(5-methyl-2-pyridin-2-yl-thiazol-4-yl)-1H-indole;
2-(2,6-Dimethyl-phenyl)-5-(5-methyl-2-pyridin-3-yl-thiazol-4-yl)-1H-indole;
2-(2,6-Dimethyl-phenyl)-5-(5-methyl-2-pyridin-2-yl-thiazol-4-yl)-1H-indole;
2-(2-Fluoro-6-methyl-phenyl)-5-(5-methyl-2-pyridin-3-yl-thiazol-4-yl)-1H-indole.
2-(2-Fluoro-6-methyl-phenyl)-5-(5-methyl-2-pyridin-2-yl-thiazol-4-yl)-1H-indole;
2-Cyclohexyl-5-(2,5-dimethyl-2H-pyrazol-3-yl)-1H-indole;
4-(2-cyclohexyl-1H-indol-5-yl)-N,N,3-trimethylbenzenesulfonamide;
2-cyclohexyl-5-(6-methoxy-4-methylpyridin-3-yl)-1H-indole;
4-(2-(2-fluorophenyl)-3-methyl-1H-indol-5-yl)-N,N,3-trimethylbenzenesulfonamide;
N,N,3-trimethyl-4-(3-methyl-2-phenyl-1H-indol-5-yl)benzenesulfonamide;
2-(2,6-Difluoro-phenyl)-5-(2,5-dimethyl-2H-pyrazol-3-yl)-3-methyl-1H-indole;
4-[2-(2,6-Difluoro-phenyl)-3-methyl-1H-indol-5-yl]-3,N,N-trimethyl-benzenesulfonamide; and
2-(2,6-Difluoro-phenyl)-5-(6-methoxy-4-methyl-pyridin-3-yl)-3-methyl-1H-indole.

The invention also provides methods for treating a disease or condition mediated by or otherwise associated with a CRAC receptor, the method comprising administering to a subject in need thereof an effective amount of a compound of the invention.

The invention also provides methods for treating an inflammatory, respiratory or diabetes condition, the method comprising administering to a subject in need thereof an effective amount of a compound of the invention together with an effective amount of a CRAC inhibitor.

The disease may be an inflammatory disease such as arthritis, and more particularly rheumatoid arthritis, osteoarthritis, psoriasis, allergic dermatitis, asthma, chronic obstructive pulmonary disease, airways hyper-responsiveness, septic shock, glomerulonephritis, irritable bowel disease, and Crohn's disease.

The disease may be a pain condition, such as inflammatory pain; surgical pain; visceral pain; dental pain; premenstrual pain; central pain; pain due to burns; migraine or cluster headaches; nerve injury; neuritis; neuralgias; poisoning; ischemic injury; interstitial cystitis; cancer pain; viral, parasitic or bacterial infection; post-traumatic injury; or pain associated with irritable bowel syndrome.

The disease may be a respiratory disorder, such as chronic obstructive pulmonary disorder (COPD), asthma, or bronchospasm, or a gastrointestinal (GI) disorder such as Irritable Bowel Syndrome (IBS), Inflammatory Bowel Disease (IBD), biliary colic and other biliary disorders, renal colic, diarrhea-dominant IBS, pain associated with GI distension.

### Synthesis

Compounds of the present invention can be made by a variety of methods depicted in the illustrative synthetic reaction schemes shown and described below.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40.

The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

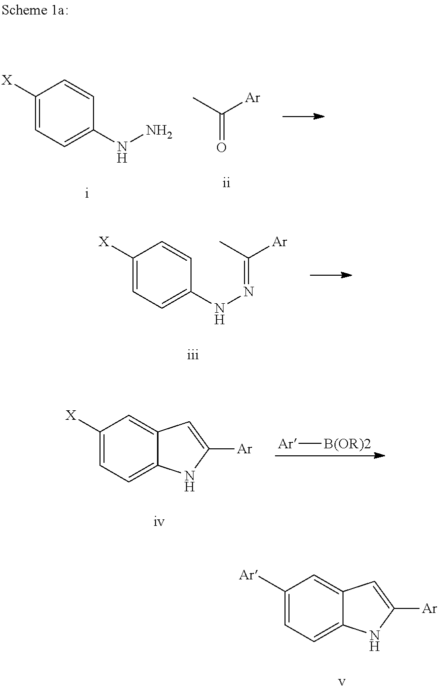

As shown in Scheme 1a, an aryl hydrazine i, where X=halide, can be reached with an appropriate acetophenone ii, to give hydrazone iii. The hydrazone iii can then be reacted in the presence of polyphosphoric acid under Fischer indole synthesis conditions to give a 2-aryl-5-halo-indole iv. Suzuki coupling of indole iv with an appropriate boronic acid or ester then gives 2,5-diaryl-indole v.

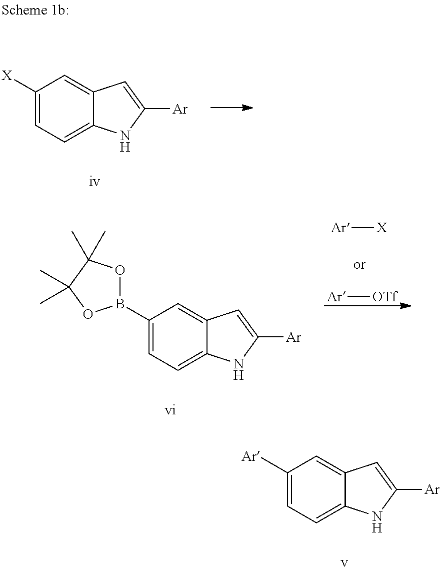

As shown in Scheme 1b, 2-aryl-5-halo-indole iv, can also be converted to the indole-boronic ester vi in the presence of a palladium catalyst and bispinacolatodiborane. Suzuki coupling of indole-boronic ester vi with an appropriate aryl halide or triflate then gives 2,5-diaryl-indole v.

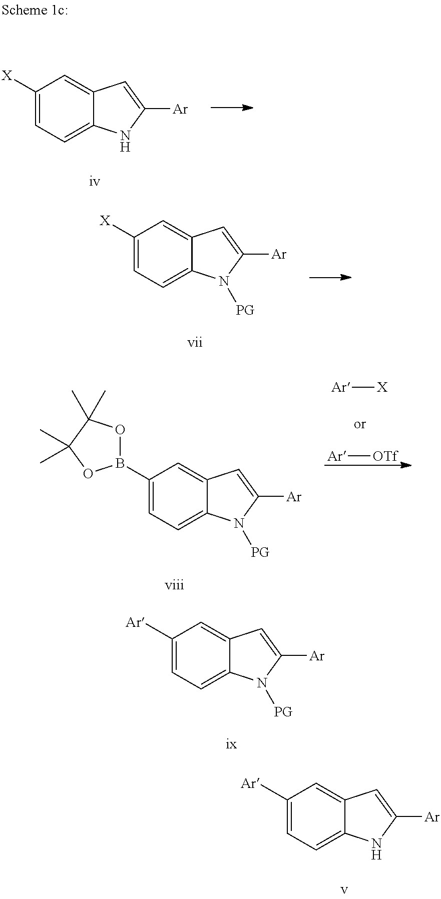

As shown in Scheme 1c, the indole N—H functionality in 2-aryl-5-halo-indole iv can be protected to give protected indole vii. Indole vii can then be converted to the protected indole-boronic ester viii in the presence of a palladium catalyst and bispinacolatodiborane. Suzuki coupling of indole viii with an appropriate aryl halide or triflate then gives protected 2,5-diaryl-indole ix. This indole ix can be deprotected under basic conditions to give 2,5-diaryl-indole v.

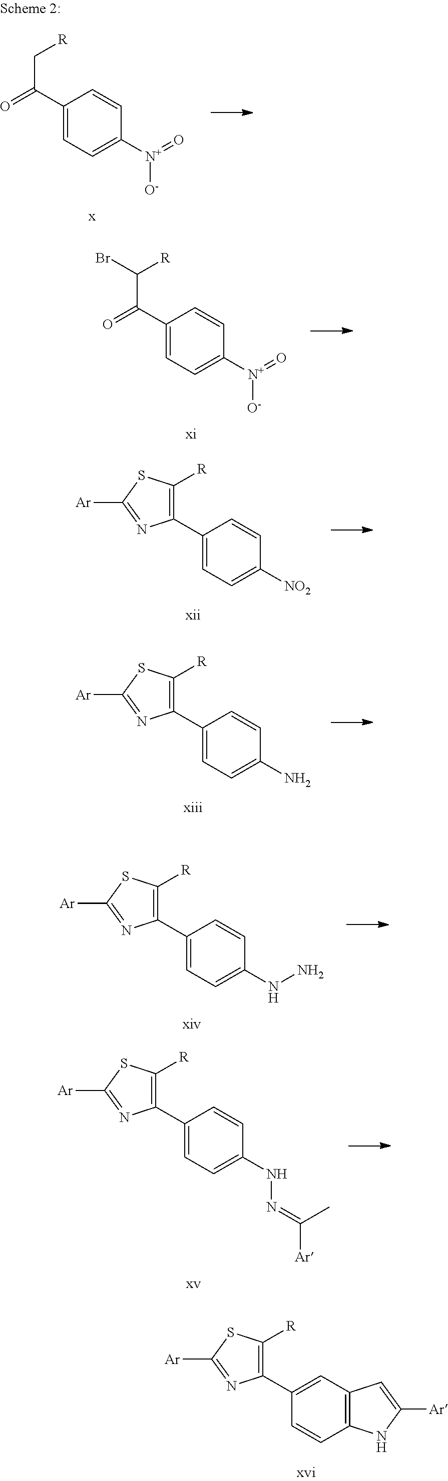

As shown in Scheme 2, nitro ketone x can be brominated to give bromo ketone xi. Reaction of bromo ketone xi with an appropriate thioamide can then produce a nitro-phenyl thiazole xii. Reduction of the nitro-phenyl thiazole xii then gives amino-phenyl thiazole xiii. Conversion of this amino-phenyl thiazole xiii to the aryl-hydrazone xiv can be accomplished via the action of sodium nitrite to produce an intermediate nitroso compound that is subsequently reduced. This aryl hydrazone xiv can be reacted with an appropriate acetophenone, to give hydrazone xv. The hydrazone xv can then be reacted in the presence of polyphosphoric acid under Fischer indole synthesis conditions to give thiazole-indole xvi.

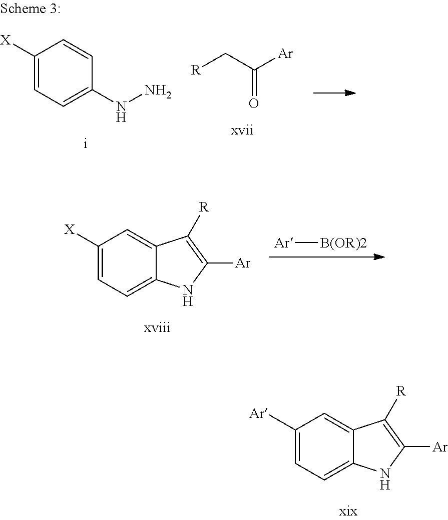

As shown in Scheme 3, an aryl hydrazine i, where X=halide, can be reacted with an appropriate aryl ketone xvii, in the presence of acetic acid under Fischer indole synthesis conditions to give directly a 2-aryl-3-substituted-5-halo-indole xviii. Suzuki coupling of indole xviii with an appropriate boronic acid or ester then gives 2,5-diaryl-indole xix.

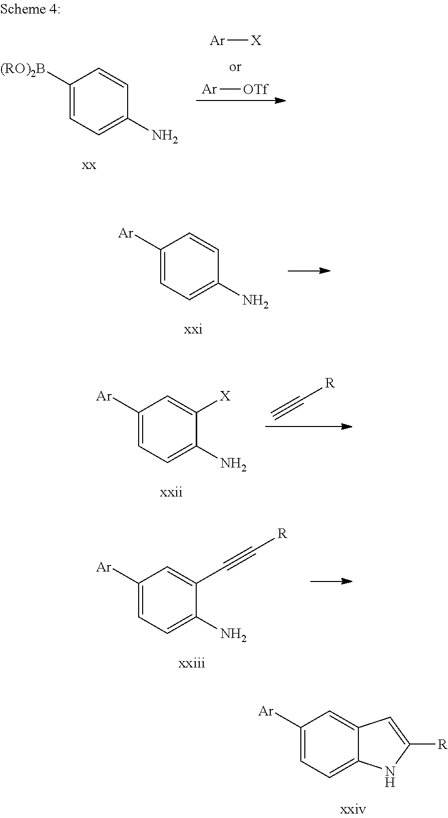

As shown in Scheme 4, the amino-phenyl-boronic acid or ester xx can reacted under Suzuki coupling conditions with an appropriate aryl halide or triflate to aniline xxi. Aniline xxi can be halogenated under electrophilic aromatic substitution conditions to give halide xxii. Sonogashira coupling of an terminal alkyne then gives the alkyne substituted aniline xxiii, where R=aryl, heteroaryl, cycloalkyl, heterocycloalkyl, or alkyl. Conversion of aniline xxiii in the presence of base or a transition metal catalyst then gives 2-substituted-5-aryl-indole xxiv.

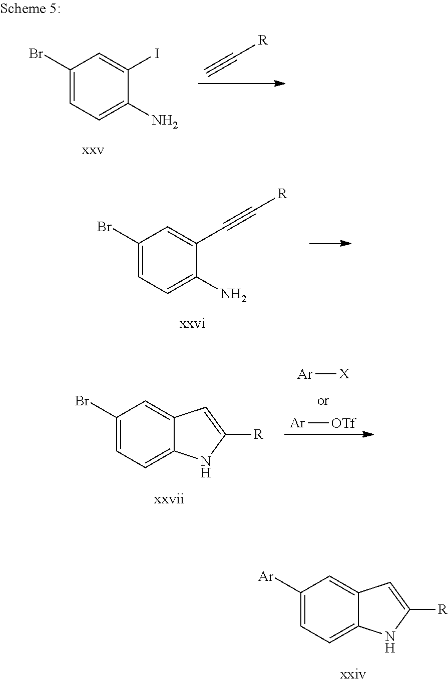

As shown in Scheme 5,4-bromo-2-iodo-aniline xxv can be reacted under Sonogashira coupling conditions with an appropriate terminal alkyne to give the alkyne substituted aniline xxiii, where R=aryl, heteroaryl, cycloalkyl, heterocycloalkyl, or alkyl. Conversion of aniline xxvi in the presence of base or a transition metal catalyst then gives 2-substituted-5-bromo-indole xxvii. Suzuki coupling of indole xxvii with an appropriate boronic acid or ester then gives 2-substituted-5-aryl-indole xxiv

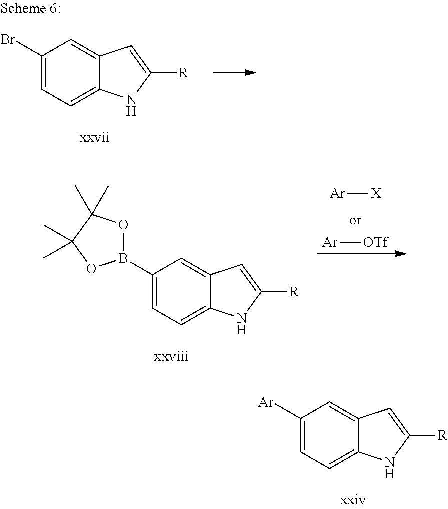

As shown in Scheme 6,2-substituted-5-bromo-indole xxvii, can also be converted to the indole-boronic ester vi in the presence of a palladium catalyst and bispinacolatodiborane. Suzuki coupling of indole boronic ester xxviii with an appropriate aryl halide or triflate then gives 2,5-diaryl-indole xxiv.

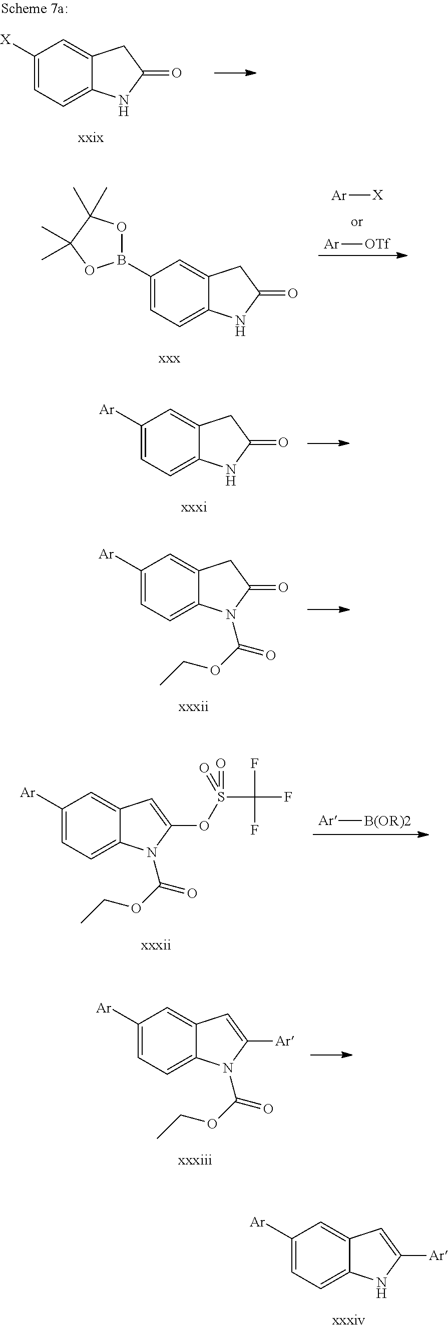

As shown in Scheme 7a, 5-halo-oxindole xxix can be converted the oxindole-boronic ester xxx in the presence of a palladium catalyst and bispinacolatodiborane. Suzuki coupling of oxindole boronic ester xxx with an appropriate aryl halide or triflate then gives the 5-aryl-oxindole xxxi. Conversion of the 5-aryl-oxindole xxxi to the ethyl carbamate xxxii takes places in two steps via the action of ethyl chloroformate and ammonium carbonate. Formation of triflate xxxii can be accomplished with triflic anhydride or phenyltriflamide and an appropriate base. Suzuki coupling of triflate xxxii with an appropriate boronic acid or ester then gives the protected 2,5-diaryl-indole xxxiii. Basic hydrolysis can then produce 2,5-diaryl-indole xxxiv.

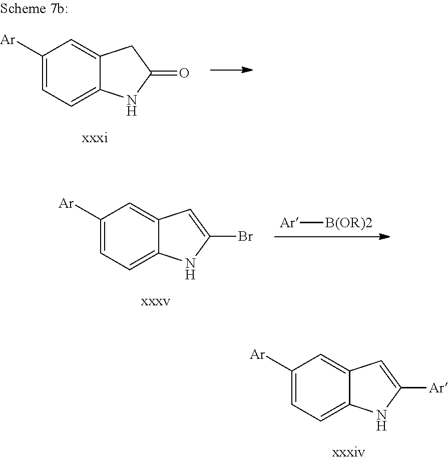

As shown in Scheme 7b, conversion of the 5-aryl-oxindole xxxi to 2-bromoindole xxxv can be accomplished by heating the material in the presence of phosphorus tribromide. Suzuki coupling of 2-bromoindole xxxv with an appropriate boronic acid or ester then gives the 2,5-diaryl-indole xxxiv directly.

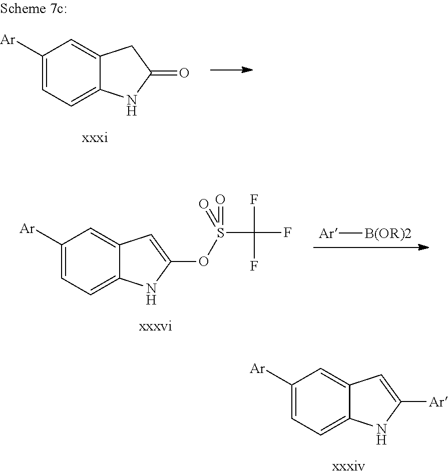

As shown in Scheme 7c, conversion of the 5-aryl-oxindole xxxi to the mono-triflate xxxvi can be accomplished using triflic anhydride, followed by a hydrolytic workup. Suzuki coupling of mono-triflate xxxvi with an appropriate boronic acid or ester then gives the 2,5-diaryl-indole xxxiv directly.

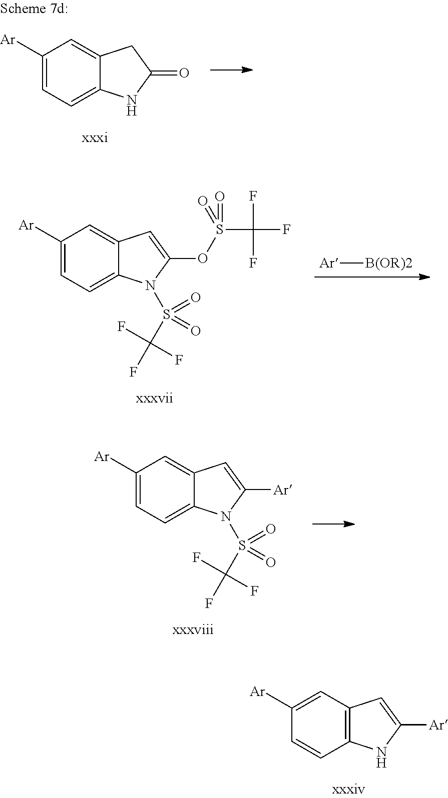

As shown in Scheme 7d, conversion of the 5-aryl-oxindole xxxi to the bis-triflate xxxvii can be accomplished using triflic anhydride. Suzuki coupling of bis-triflate xxxvii with an appropriate boronic acid or ester then gives the triflate protected-2,5-diaryl-indole xxxiv. Deprotection under basic conditions can then furnish the 2,5-diaryl-indole xxxiv.

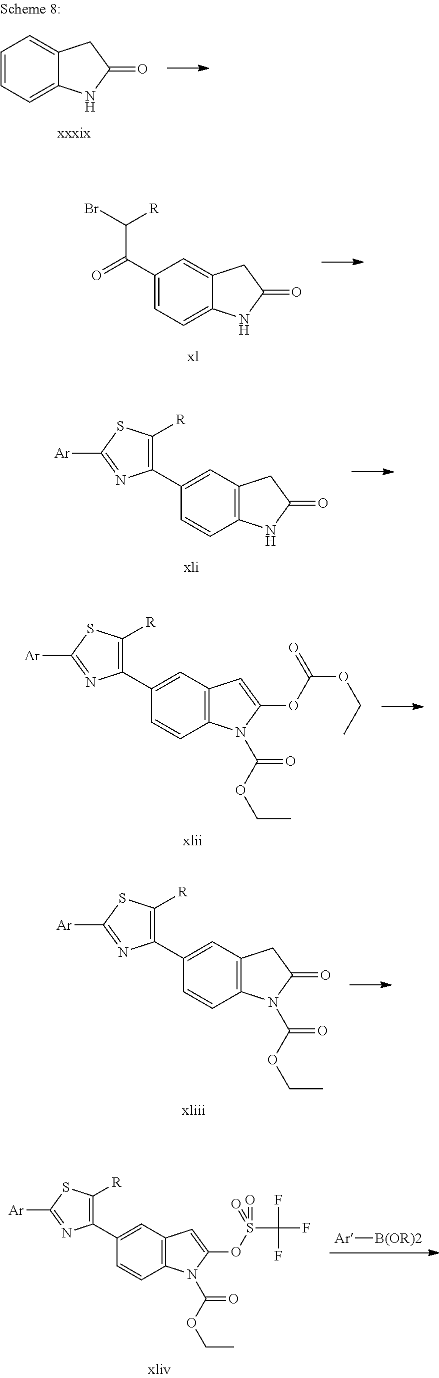

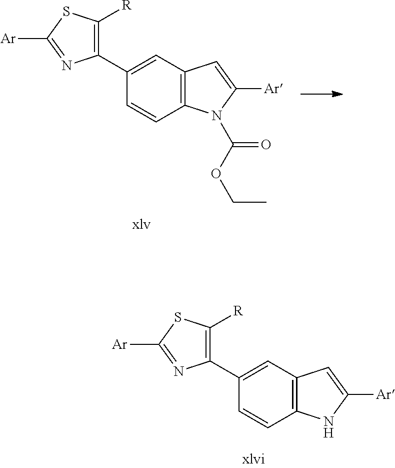

As shown in Scheme 8, conversion of the oxindole xxxix to the bromo ketone xl can be accomplished under Friedel-Crafts acylation conditions with aluminum trichloride and the appropriate acyl chloride. Reaction of the ketone xl with a suitable thioamide can then produce 5-thiazoyl-oxindole xli. Conversion of the 5-thiazoyl-oxindole xli to the ethyl carbamate xliii takes places in two steps via the action of ethyl chloroformate and ammonium carbonate. Formation of triflate xliv can be accomplished with triflic anhydride or phenyltriflamide and an appropriate base. Suzuki coupling of triflate xliv with an appropriate boronic acid or ester then gives the protected 2,5-diaryl-indole xlv. Basic hydrolysis can then produce 2,5-diaryl-indole xlvi.

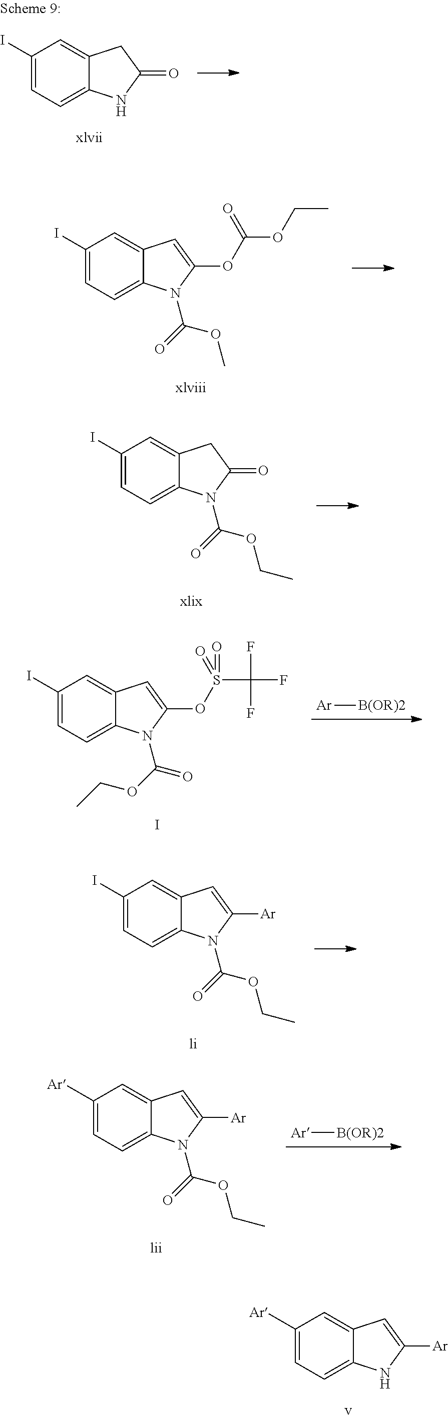

As shown in Scheme 9, conversion of 5-iodooxindole xlvii to the ethyl carbamate xlix takes places in two steps via the action of ethyl chloroformate and ammonium carbonate. Formation of ethyl carbamate protected triflate l can be accomplished with triflic anhydride or phenyltriflamide and an appropriate base. Selective Suzuki coupling of triflate l with an appropriate boronic acid or ester then gives the protected 2-aryl-5-iodo-indole li. Subsequent Suzuki coupling of the iodide li with an appropriate boronic acid or ester then gives the protected 2,5-diaryl-indole lii. Basic hydrolysis can then produce 2,5-diaryl-indole v.

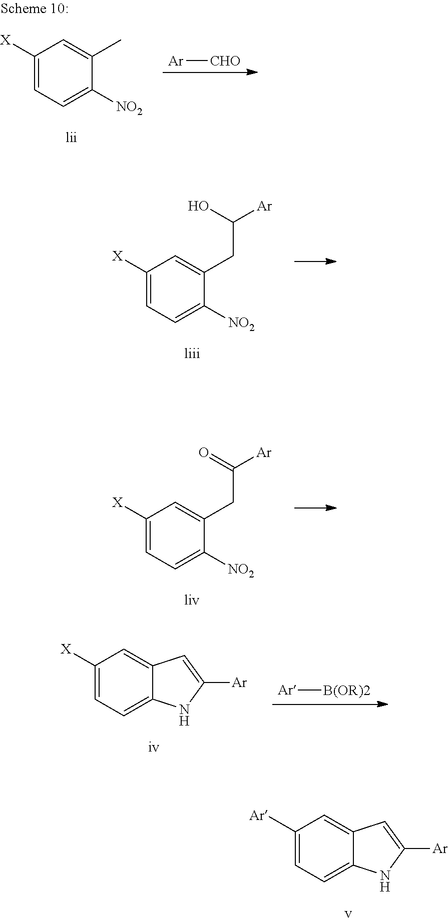

As shown in Scheme 10, 2-methyl-4-halo-nitrobenzene lii can be reacted in the presence of a benzaldehyde and base to form the modified Reissert reaction product liii. Upon oxidation of this alcohol liii with Dess-Martin periodinane the ketone liv can be formed. Nitro reduction with concomitant cyclization then affords 2-aryl-5-halo-indole iv. Suzuki coupling of indole iv with an appropriate boronic acid or ester then gives 2,5-diaryl-indole v.

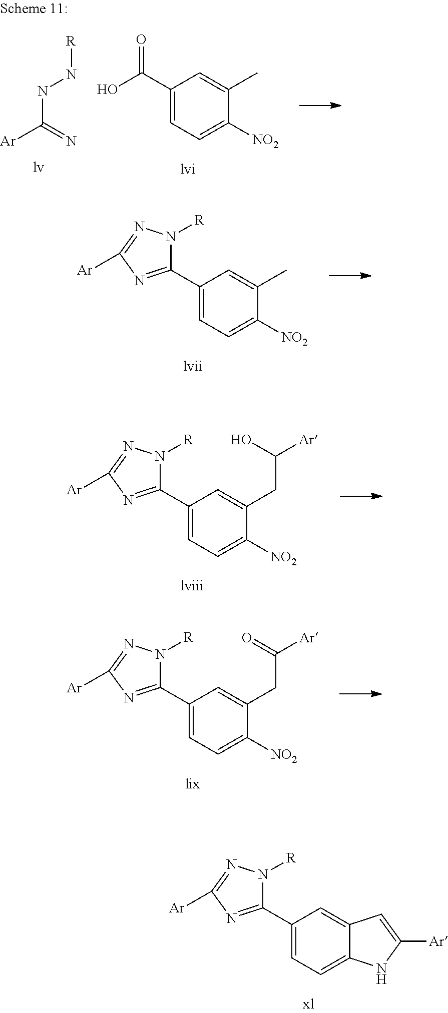

As shown in Scheme 11, amidrazone lv and benzoic acid lvi can be condensed in the presence of carbonyl diimidazole to give to triazole lvii. Triazole lvii can then be reacted in the presence of a benzaldehyde and base to form the modified Reissert reaction product lviii. Upon oxidation of this alcohol lviii with Dess-Martin periodinane the ketone lix can be formed. Nitro reduction with concomitant cyclization then affords 2-aryl-5-triazolo-indole xl.

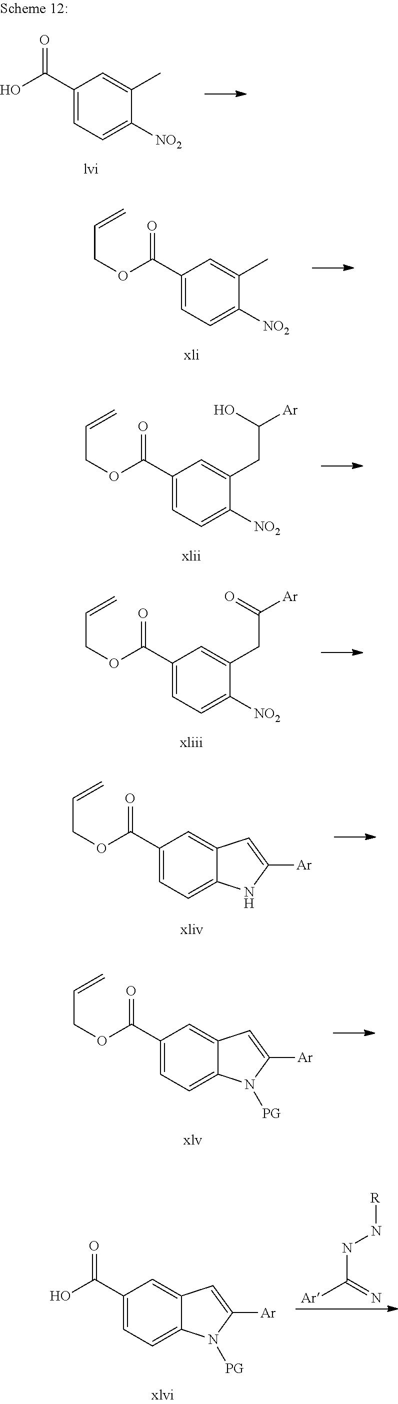

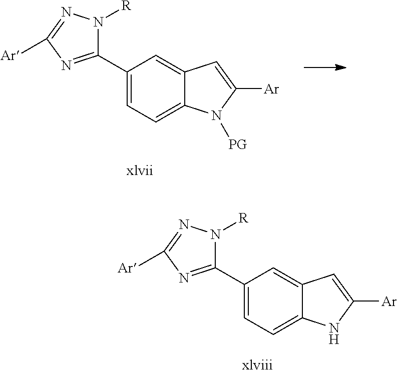

As shown in Scheme 12, benzoic acid lvi can be converted to the ally ester in the presence of potassium carbonate and allyl bromide. Allyl ester vii can then be reacted in the presence of a benzaldehyde and base to form the modified Reissert reaction product xlii. Upon oxidation of this alcohol xlii with Dess-Martin periodinane the ketone xliii can be formed. Nitro reduction with concomitant cyclization then affords 2-aryl-5-ester substituted indole xliv. Protection of the indole N—H group with the appropriate group gives xlv. Deallylation in presence of palladium tetrakis then gives 5-carboxy indole xlvi. Condensation of this material with an amidrazone in the presence of carbonyl diimidazole can then produce triazole xlvii. Subsequent deprotection provides xlviii.

Many variations on the procedure of the above Schemes are possible and will suggest themselves to those skilled in the art. Specific details for producing compounds of the invention are described in the Examples section below.

### Utility

The compounds of the invention are usable for the treatment of a wide range of inflammatory diseases and conditions such as arthritis, including but not limited to, rheumatoid arthritis, spondyloarthropathies, gouty arthritis, osteoarthritis, systemic lupus erythematosus and juvenile arthritis, osteoarthritis, gouty arthritis and other arthritic conditions. The subject compounds would be useful for the treatment of pulmonary disorders or lung inflammation, including adult respiratory distress syndrome, pulmonary sarcoidosis, asthma, silicosis, and chronic pulmonary inflammatory disease.

Further, compounds of the invention are useful for treating respiratory disorders, including chronic obstructive pulmonary disorder (COPD), asthma, bronchospasm, and the like.

### Administration and Pharmaceutical Composition

The invention includes pharmaceutical compositions comprising at least one compound of the present invention, or an individual isomer, racemic or non-racemic mixture of isomers or a pharmaceutically acceptable salt or solvate thereof, together with at least one pharmaceutically acceptable carrier, and optionally other therapeutic and/or prophylactic ingredients.

In general, the compounds of the invention will be administered in a therapeutically effective amount by any of the accepted modes of administration for agents that serve similar utilities. Suitable dosage ranges are typically 1-500 mg daily, preferably 1-100 mg daily, and most preferably 1-30 mg daily, depending upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this Application, to ascertain a therapeutically effective amount of the compounds of the present invention for a given disease.

Compounds of the invention may be administered as pharmaceutical formulations including those suitable for oral (including buccal and sub-lingual), rectal, nasal, topical, pulmonary, vaginal, or parenteral (including intramuscular, intraarterial, intrathecal, subcutaneous and intravenous) administration or in a form suitable for administration by inhalation or insufflation. The preferred manner of administration is generally oral using a convenient daily dosage regimen which can be adjusted according to the degree of affliction.

A compound or compounds of the invention, together with one or more conventional adjuvants, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. Formulations containing about one (1) milligram of active ingredient or, more broadly, about 0.01 to about one hundred (100) milligrams, per tablet, are accordingly suitable representative unit dosage forms.

The compounds of the invention may be formulated in a wide variety of oral administration dosage forms. The pharmaceutical compositions and dosage forms may comprise a compound or compounds of the present invention or pharmaceutically acceptable salts thereof as the active component. The pharmaceutically acceptable carriers may be either solid or liquid. Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges may be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

The compounds of the invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The compounds of the invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatine and glycerine or sucrose and acacia; and mouthwashes comprising the active ingredient in a suitable liquid carrier.

The compounds of the invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the invention may be formulated for vaginal administration. Pessaries, tampons, creams, gels, pastes, foams or sprays containing in addition to the active ingredient such carriers as are known in the art to be appropriate.

The subject compounds may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatine or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylazacycloheptan-2-one). Sustained release delivery systems are inserted subcutaneously into the subdermal layer by surgery or injection. The subdermal implants encapsulate the compound in a lipid soluble membrane, e.g., silicone rubber, or a biodegradable polymer, e.g., polylactic acid.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company, 19th edition, Easton, Pa. Representative pharmaceutical formulations containing a compound of the present invention are described below.

### EXAMPLES

The following preparations and examples are given to enable those skilled in the art to more clearly understand and to practice the present invention. They should not be considered as limiting the scope of the invention, but merely as being illustrative and representative thereof.

Unless otherwise stated, all temperatures including melting points (i.e., MP) are in degrees celsius (° C.). It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The following abbreviations may be used in the Preparations and Examples.

### ABBREVIATIONS

CDI 1,1′-carbonyldiimidazole
DBU 1,8-diazabicyclo[5.4.0]undec-7-ene
DCM dichloromethane/methylene chloride
DME 1,2-dimethoxyethane (glyme)
DMF N,N-dimethylformamide
DMSO dimethyl sulfoxide
dppf 1,1′-Bis(diphenylphosphino)ferrocene
EDCI 1-ethyl-3-(3′-dimethylaminopropyl)carbodiimide
EtOAc ethyl acetate
EtOH ethanol
HOBt N-Hydroxybenzotriazole
hplc high performance liquid chromatography
IPA isopropanol
mCPBA m-chloroperbenzoic acid
MeOH methanol
NB S N-bromo-succinimide
NMP N-methylpyrrolidinone
PPA polyphosphoric acid
TEA triethylamine
THF tetrahydrofuran
TLC thin layer chromatography

### Part 1: Preparation of Preferred Intermediates

### Intermediate 1

### Trifluoro-methanesulfonic acid 5-methyl-2-pyridin-2-yl-thiazol-4-yl ester

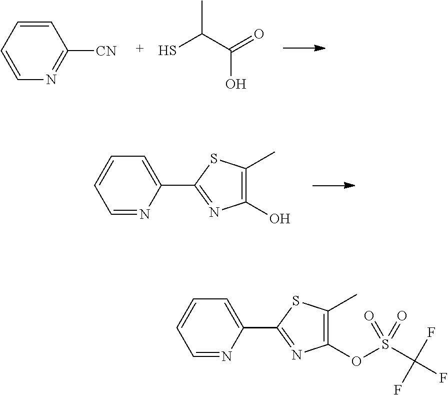

5-Methyl-2-pyridin-2-yl-thiazol-4-ol: To 2-cyanopyridine (5 g, 48 mmol) and thiolactic acid (5.1 g, 48 mmol) was added pyridine (0.97 mL, 12 mmol) and the mixture stirred at 100° C. After 3 h, the mixture was cooled to 25° C. and EtOH (50 mL) was added. After 30 min. the solvent was removed, and the residue washed with diethylether (3×30 mL) to give 5-Methyl-2-pyridin-2-yl-thiazol-4-ol (7 g, 76%).

Trifluoro-methanesulfonic acid 5-methyl-2-pyridin-2-yl-thiazol-4-yl ester: To a solution of 5-Methyl-2-pyridin-2-yl-thiazol-4-ol (500 mg, 2.6 mmol) in THF at 0° C. was added NaH (81.12 mg, 3.38 mmol) followed by N-phenyl bis(trifluoromethanesulfonimide) (1.08 g, 3.02 mmol). The reaction mixture was stirred at 25° C. for 1 h, after which water was added at 0° C. and the entire mixture extracted with EtOAc (3×20 mL). The organic phase was washed with brine, dried over Na₂SO₄, concentrated, and the crude compound was purified by column chromatography (10-20% EtOAc-Hexane) to give Trifluoro-methanesulfonic acid 5-methyl-2-pyridin-2-yl-thiazol-4-yl ester (200 mg, 24%).

### Intermediate 2

### Trifluoro-methanesulfonic acid 2-ethyl-5-phenyl-2H-pyrazol-3-yl ester

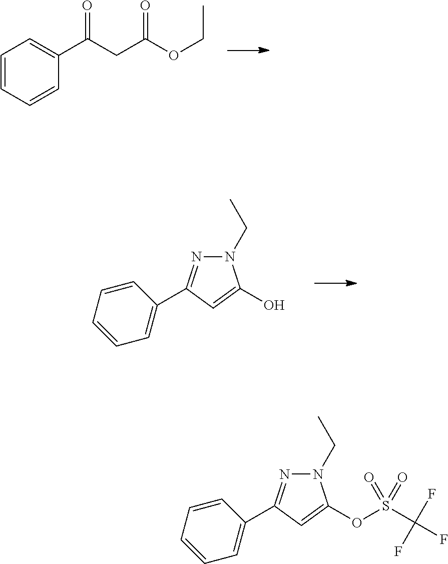

2-Ethyl-5-phenyl-2H-pyrazol-3-ol: To 3-Oxo-3-phenyl-propionic acid ethyl ester (1 g, 5.2 mmol) and ethylhydrazine oxalate (1.17 g, 7.8 mmol) was added AcOH, and the mixture stirred at 110° C. for 24 h. Upon completion of the reaction, aq. Na₂CO₃ was added and the mixture extracted with EtOAc (3×20 mL). The organic phase was washed with brine, dried over Na₂SO₄, and concentrated. The crude compound was purified by column chromatography (35% EtOAc-Hexane) to give 2-Ethyl-5-phenyl-2H-pyrazol-3-ol (0.65 g, 66%).

Trifluoro-methanesulfonic acid 2-ethyl-5-phenyl-2H-pyrazol-3-yl ester: 2-Ethyl-5-phenyl-2H-pyrazol-3-ol (100 mg, 0.53 mmol) in THF was cooled to −78° C. To this was added TEA (271 mg, 2.66 mmol) followed by dropwise addition of Tf₂O (300 mg, 1.06 mmol). The mixture was stirred for 15 min. at this temperature, then allowed to rise to 25° C. and stirred for 1 h. Upon completion, water was added at 0° C. and the mixture extracted with EtOAc (3×20 mL). The organic phase was washed with 1 N HCl, dried over Na₂SO₄ and concentrated. The crude compound was purified by column chromatography (10% EtOAc-Hexane) to give trifluoro-methanesulfonic acid 2-ethyl-5-phenyl-2H-pyrazol-3-yl ester (90 mg, 53%).

### Intermediate 3

### Trifluoro-methanesulfonic acid 2-ethyl-5-pyridin-2-yl-2H-pyrazol-3-yl ester

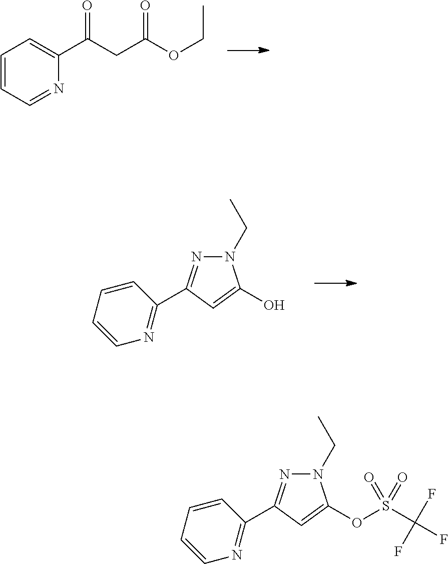

2-Ethyl-5-pyridin-2-yl-2H-pyrazol-3-ol: 3-Oxo-3-pyridin-2-yl-propionic acid ethyl ester (500 mg, 2.59 mmol) and ethylhydrazine oxalate (389 mg, 2.59 mmol) was dissolved in EtOH, and stirred at 80° C. Upon completion, the EtOH was removed and triturated with Et₂O to give 2-Ethyl-5-pyridin-2-yl-2H-pyrazol-3-ol (200 mg, 40%) as a white solid.

Trifluoro-methanesulfonic acid 2-ethyl-5-pyridin-2-yl-2H-pyrazol-3-yl ester: 2-Ethyl-5-pyridin-2-yl-2H-pyrazol-3-ol (200 mg, 1.06 mmol) in THF was cooled to 0° C. and to this solution was added NaH (33 mg, 1.37 mmol) followed by N-phenyl bis(trifluoromethanesulfonimide) (567 mg, 1.58 mmol) and the mixture stirred at 25° C. for 1 h. Upon completion, water was added at 0° C. and the mixture extracted with EtOAc (3×20 mL). The organic phase was washed with 1 N NaOH, dried over Na₂SO₄ and concentrated. The crude compound was purified by column chromatography (20% EtOAc-Hexane) to give trifluoro-methanesulfonic acid 2-ethyl-5-pyridin-2-yl-2H-pyrazol-3-yl ester (90 mg, 27%).

### Intermediate 4

### Trifluoro-methanesulfonic acid 2-methyl-5-pyridin-4-yl-2H-pyrazol-3-yl ester

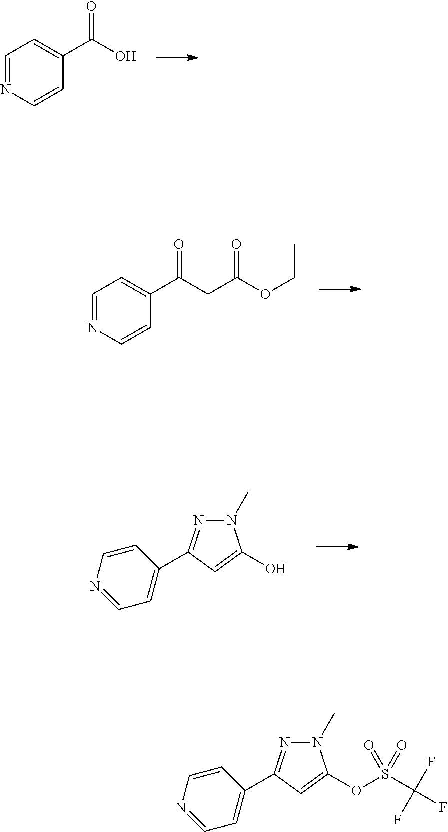

3-Oxo-3-pyridin-4-yl-propionic acid ethyl ester: To ethyl potassium malonate (6.25 g, 36.7 mmol) in THF (30 mL) was added MgCl₂ (2.71 g, 28.4 mmol) and the mixture heated to 50° C. In another flask, CDI (6 g, 36.6 mmol) was added to a solution of isonicotinic acid (3 g, 24.4 mmol) in THF (30 mL) at 10° C. This mixture was stirred at 25° C. for 1 h, after which it was added to the ethyl potassium malonate/MgCl₂ suspension and stirred for 18 h. Upon completion, water was added, and the aqueous mixture extracted with EtOAc (3×50 mL). The organic phase was washed with brine, dried over Na₂SO₄, concentrated, and the crude material purified by column chromatography (30% EtOAc-Hexane) to give 3-Oxo-3-pyridin-4-yl-propionic acid ethyl ester (1.2 g, 25%).

Upon obtaining 3-Oxo-3-pyridin-4-yl-propionic acid ethyl ester the synthesis of Intermediate 4 was identical to that described for Intermediate 3 with substitution of methyl hydrazine in place of ethyl hydrazine oxalate.

### Intermediate 5

### Trifluoro-methanesulfonic acid 2-ethyl-5-pyridin-4-yl-2H-pyrazol-3-yl ester

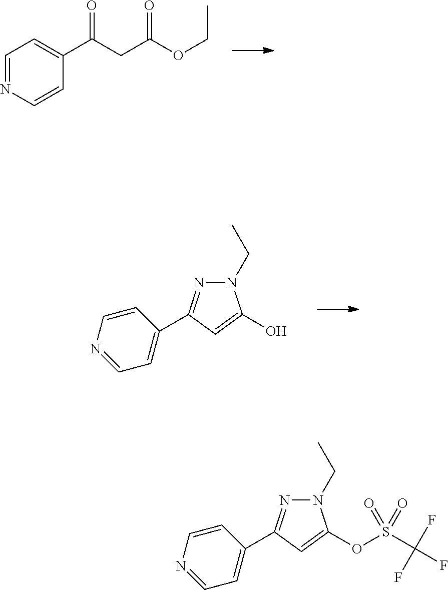

Intermediate 5 was prepared in a manner identical to that used for Intermediate 3.

### Intermediate 6

### Trifluoro-methanesulfonic acid 2-methyl-5-pyridin-3-yl-2H-pyrazol-3-yl ester

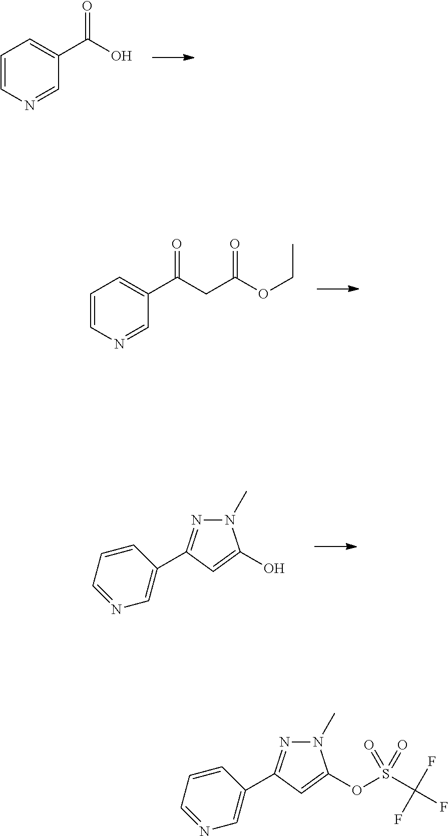

Intermediate 6 was prepared in a manner identical to that used for Intermediate 3.

### Intermediate 7

### Trifluoro-methanesulfonic acid 2-ethyl-5-pyridin-3-yl-2H-pyrazol-3-yl ester

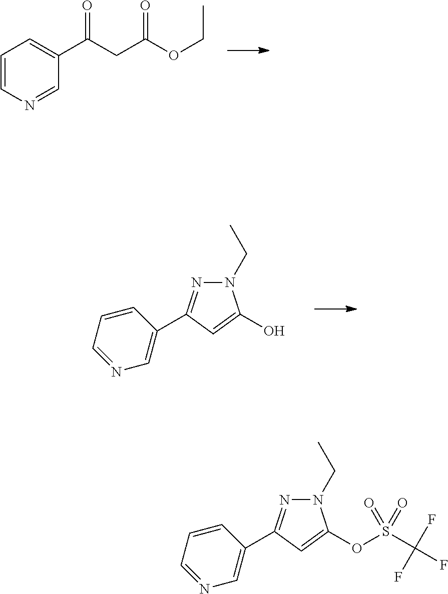

Intermediate 7 was prepared in a manner identical to that used for Intermediate 3.

### Intermediate 8

### Trifluoro-methanesulfonic acid 5-methyl-2-pyridin-4-yl-thiazol-4-yl ester

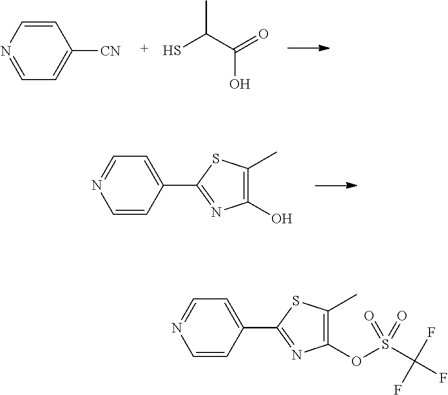

5-Methyl-2-pyridin-4-yl-thaizol-4-ol: To 4-cyanopyridine (5 g, 48 mmol) and thiolactic acid (5.1 g, 48 mmol) was added pyridine (0.97 mL, 12 mmol) and the mixture stirred at 100° C. Upon completion, the mixture was cooled to 25° C. and EtOH (50 mL) was added and stirred for 30 min. The resulting solids were filtered and washed with Et₂O (3×30 mL) to give 5-Methyl-2-pyridin-4-yl-thiazol-4-ol (7 g, 76%).

Trifluoro-methanesulfonic acid-5-methyl-2-pyridin-4-yl-thiazol-4-yl ester: To a solution of 5-Methyl-2-pyridin-4-yl-thiazol-4-ol (4 g, 20.8 mmol) in THF at 0° C. and added NaH (0.65 g, 24.14 mmol) followed by N-phenyl bis(trifluoromethanesulfonimide) (8.62 g, 27.1 mmol). The mixture was stirred at 25° C. for 1 h, after which water was added at 0° C. The mixture was extracted with EtOAc (3×20 mL) and then the organic phase was washed with brine, dried over Na₂SO₄, and concentrated. The crude compound was purified by column chromatography (10-20% EtOAc-Hexane) to give Trifluoro-methanesulfonic acid 5-methyl-2-pyridin-4-yl-thiazol-4-yl ester (4.5 g, 67%).

### Intermediate 9

### 2-(3-Bromo-4-methyl-phenyl)-oxazole

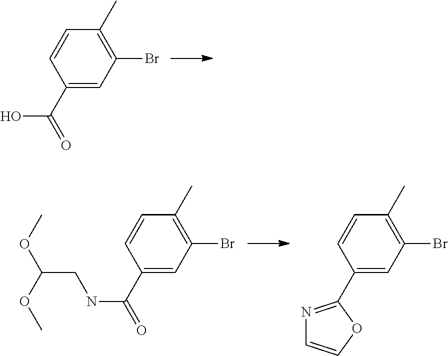

3-Bromo-N-(2,2-dimethoxy-ethyl)-4-methyl-benzamide: To a solution of 3-Bromo-4-methyl-benzoic acid (1 g, 4.65 mmol) in THF was added N-methylmorpholine (0.517 mg, 5.16 mmol) and isopropylchloroformate (0.569 mg, 4.65 mmol), followed by addition of 2,2-dimethoxyethylamine (0.489 mg, 4.65 mmol) at 10° C. The mixture was stirred to ambient temperature overnight, after which it was extracted with EtOAc (3×20 mL). The organic phase was washed with brine, dried over Na₂SO₄, concentrated, and the crude compound purified by column chromatography (10-20% EtOAc-Hexane) to give 3-Bromo-N-(2,2-dimethoxy-ethyl)-4-methyl-benzamide (560 mg, 40%).

2-(3-Bromo-4-methyl-phenyl)-oxazole: A mixture of 3-Bromo-N-(2,2-dimethoxy-ethyl)-4-methyl-benzamide (430 mg, 1.42 mmol) and Eton's reagent (P2O5.MeSO3H) (10.64 g, 37.5 mmol) were stirred at 110° C. After 18 h, the reaction was quenched with ice-water and extracted with EtOAc (3×30 mL). The organic phase was washed with brine, dried over Na₂SO₄, concentrated, and then purified by column chromatography (10-20% EtOAc-Hexane) to give 2-(3-Bromo-4-methyl-phenyl)-oxazole (50 mg, 14%).

### Intermediate 10

### 2-(3-Bromo-4-methyl-phenyl)-thiazole

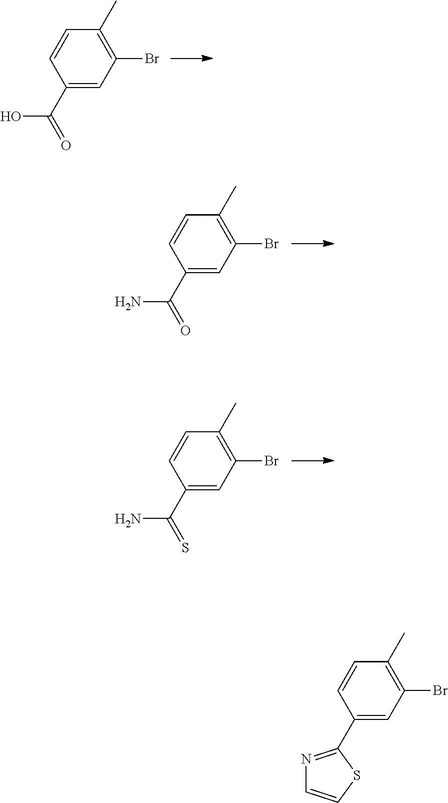

3-Bromo-4-methyl-benzamide: To a solution of 3-Bromo-4-methyl-benzoic acid (1 g, 4.65 mmol) in DCM and dimethylformamide (catalytic) was added oxalyl chloride (0.69 g, 5.44 mmol) at 0° C. The reaction mixture was then stirred at 25° C. for 4 h, after which the solvent was removed and replaced with THF. This solution was then cooled to −78° C. and NH₃ in THF was added. The reaction mixture was then warmed to 25° C. and stirred for an additional 30 min. The solid formed was filtered, and washed with a small amount of THF. The THF filtrate was then evaporated to dryness to give 3-Bromo-4-methyl-benzamide (913 mg, 99%).

3-Bromo-4-methyl-thiobenzamide: To a solution of 3-Bromo-4-methyl-benzamide (200 mg, 0.93 mmol) in DCM was added Lawesson's reagent (180 mg, 0.46 mmol) at 25° C. The reaction mixture was then stirred at this temperature for 48 h, after which the DCM was removed, water was added, and the aqueous mixture extracted with EtOAc (3×20 mL). The organic phase was washed with brine, dried over Na₂SO₄, concentrated, and then purified by column chromatography (30% EtOAc-Hexane) to give 3-Bromo-4-methyl-thiobenzamide (170 mg, 79%).

2-(3-Bromo-4-methyl-phenyl)-thiazole: To a solution of 3-Bromo-4-methyl-thiobenzamide (170 mg, 0.74 mmol) in THF was added 2,2-dimethoxyethylamine (727 mg, 3.69 mmol). The mixture was then heated to 70° C. for 24 h, after which the DCM was removed, water was added, and the aqueous mixture extracted with EtOAc (3×20 mL). The organic phase was washed with brine, dried over Na₂SO₄, concentrated, and then purified by column chromatography (30% EtOAc-Hexane) to give 2-(3-Bromo-4-methyl-phenyl)-thiazole (150 mg, 80%).

### Intermediate 11

### Trifluoro-methanesulfonic acid 2-methyl-5-trifluoromethyl-2H-pyrazol-3-yl ester

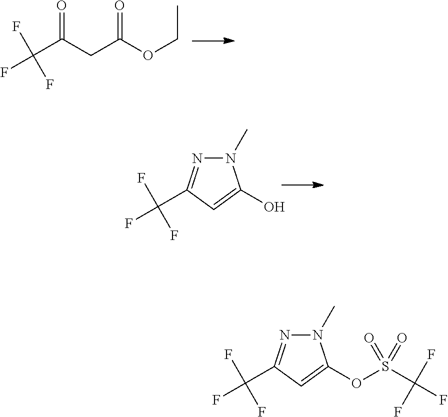

2-Methyl-5-trifluoromethyl-2H-pyrazol-3-ol: To a solution of 4,4,4-Trifluoro-3-oxo-butyric acid ethyl ester (10 g, 54.34 mmol) in EtOH (40 ml) was added methyl hydrazine (2.9 ml, 54.34 mmol) and HCl (2 ml). The mixture was refluxed for 2 days, after which point the EtOH was evaporated and water was added to the reaction mixture. This was then extracted with EtOAc and the organic phase was evaporated to obtain 2-Methyl-5-trifluoromethyl-2H-pyrazol-3-ol (8 g, 89%) as an off-white solid.

Trifluoro-methanesulfonic acid 2-methyl-5-trifluoromethyl-2H-pyrazol-3-yl ester: To a solution of 2-Methyl-5-trifluoromethyl-2H-pyrazol-3-ol (5 g, 30.1 mmol) in DCM (80 mL) at 0° C. was added TEA (8.42 mL, 60.2 mmol), followed by drop wise addition of Tf₂O (7.47 mL, 45.1 mmol). The reaction mixture was allowed to warm to 25° C. and stirred for 1 h. Water was then added to quench the reaction and it was extracted with DCM. The organic phase was then washed with brine, dried over Na₂SO₄, and concentrated in vacuo to give Trifluoro-methanesulfonic acid 2-methyl-5-trifluoromethyl-2H-pyrazol-3-yl ester (5.5 g, 80%) which was sufficiently pure for use in further reactions.

### Intermediate 12

### Trifluoro-methanesulfonic acid 2-ethyl-5-trifluoromethyl-2H-pyrazol-3-yl ester

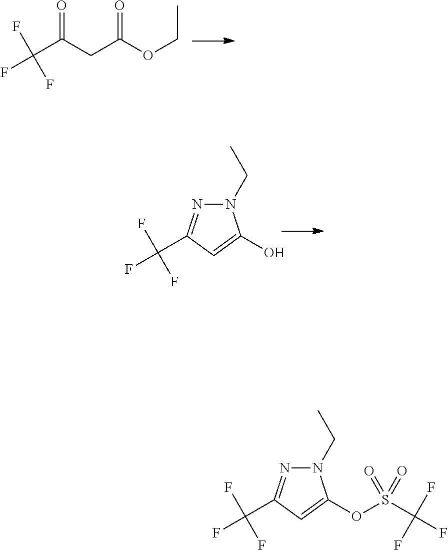

Intermediate 12 was prepared in a manner identical to that used for Intermediate 11 substituting ethyl hydrazine oxalate in the condensation. An alternate procedure is also described here:

ethyl-3-(trifluoromethyl)-1H-pyrazol-5(4H)-one: A mixture of ethyl 4,4,4-trifluoroacetoacetate (11.0 g, 59.7 mmol) and ethyl hydrazine oxalate (8.96 g, 59.7 mmol) in acetic acid (60 ml) was heated at 120° C. in a microwave reactor for 1.5 h. After irradiation the reaction mixture was poured into ice water, extracted with EtOAc. The organic phase was then washed with brine, dried over Na₂SO₄, filtered, concentrated under reduced pressure, and the crude material purified by flash chromatography (5-10% EtOAc/hexanes) to give 2-Ethyl-5-trifluoromethyl-2H-pyrazol-3-ol (4.62 g, 43%) as a yellow solid.

ethyl-3-(trifluoromethyl)-1H-pyrazol-5-yl trifluoromethanesulfonate: To a solution of 2-Ethyl-5-trifluoromethyl-2H-pyrazol-3-ol (4.41 g, 24.5 mmol) in CH₂Cl₂ (100 ml) and DIPEA (4.75 g, 36.7 mmol) at 0° C. was added trifluoromethane sulfonic anhydride (8.98 g, 31.8 mmol) dropwise. The mixture was stirred at 0° C. for 1 hour, then a cold solution of aqueous ammonium chloride and dichloromethane was added. The mixture was partitioned, and the organic phase washed with brine, dried over Na₂SO₄, filtered, concentrated under reduced pressure, and the crude material purified by filtering through a pad of silica (8% EtOAc/Hexanes) to give 1-ethyl-3-(trifluoromethyl)-1H-pyrazol-5-yltrifluoromethanesulfonate (6.12 g, 80%) as a yellow oil.

### Intermediate 13

### Trifluoro-methanesulfonic acid 5-methyl-2-pyridin-3-yl-thiazol-4-yl ester

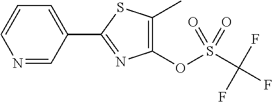

Intermediate 13 was prepared in a manner identical to that used for Intermediate 8.

### Intermediate 14

### 2-(4-Bromo-3-methyl-phenyl)-oxazole

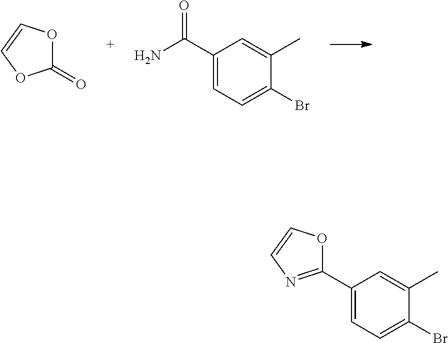

2-(4-Bromo-3-methyl-phenyl)-oxazole: A mixture of 4-Bromo-3-methyl-benzamide (1 g, 4.67 mmol) and vinylene carbonate (0.4 ml, 6.30 mmol) in PPA (15 ml) was heated to 170° C. for 3 h. Upon completion, the reaction was cooled, quenched with water, and extracted with EtOAc. The organic phase was washed with brine, dried over Na₂SO₄, and concentrated. The crude material was purified by column chromatography to give 2-(4-Bromo-3-methyl-phenyl)-oxazole (400 mg, 36%).

### Intermediate 15

### 5-Bromo-1-methyl-3-trifluoromethyl-1H-pyrazole

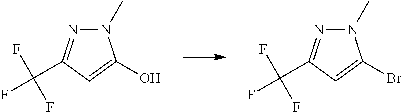

5-Bromo-1-methyl-3-trifluoromethyl-1H-pyrazole: To 2-Methyl-5-trifluoromethyl-2H-pyrazol-3-ol (5 g, 30.12 mmol) was added POBr₃ (8.63 g, 30.12 mmol) and the mixture was heated at 120° C. for 1 h. Upon completion the reaction mixture was cooled to 25° C., ice-water was added, and the pH adjusted to 8-9 with NaOH (1 M), and the mixture was then extracted with EtOAc (3×30 mL). The organic phase was washed with brine, dried over Na₂SO₄, and concentrated to obtain 5-Bromo-1-methyl-3-trifluoromethyl-1H-pyrazole (2.8 g, 41%).

### Intermediate 16

### Trifluoro-methanesulfonic acid 5-cyclopropyl-2-methyl-2H-pyrazol-3-yl ester

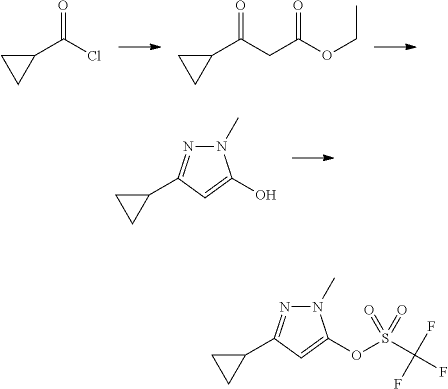

Cyclopropyl-3-oxo-propionic acid ethyl ester: To a solution of ethyl potassium malonate (6.5 g, 38.26 mmol) in acetonitrile was added MgCl₂ (4.55 g, 47.8 mmol) and the mixture stirred for 5 min at 25° C. TEA (10.7 mL, 76.54 mmol) was then added, followed by dropwise addition of cyclopropanecarbonyl chloride (2 g, 19.13 mmol) and stirring was continued at 25° C. for 16 h, after which, the mixture was diluted with water, acidified to pH 3 with 6N HCl, extracted with diethylether (3×40 mL), dried over Na₂SO₄, and concentrated to give 3-Cyclopropyl-3-oxo-propionic acid ethyl ester (1.8 g, 60%).

5-Cyclopropyl-2-methyl-2H-pyrazol-3-ol: To a solution of 3-Cyclopropyl-3-oxo-propionic acid ethyl ester (1.8 g, 11.54 mmol) in EtOH was added methyl hydrazine (0.584 g, 12.7 mmol). This mixture was heated at 80° C., until deemed complete by TLC, after which the EtOH was removed. The solid there obtained was triturated to give 5-cyclopropyl-2-methyl-2H-pyrazol-3-ol (1.3 g, 81.5%) as a white solid.

Trifluoro-methanesulfonic acid 5-cyclopropyl-2-methyl-2H-pyrazol-3-yl ester: To 5-cyclopropyl-2-methyl-2H-pyrazol-3-ol (100 mg, 0.724 mmol) in THF at 0° C. was added NaH (33 mg, 1.37 mmol), followed by N-phenyl bis(trifluoromethanesulfonimide) (310 mg, 0.87 mmol). The mixture was stirred at 25° C. for 1 h, after which water was added at 0° C. The aqueous mixture was extracted with DCM (3×20 mL), the organic phase was then washed with 1 N NaOH, dried over Na₂SO₄, and concentrated to give Trifluoro-methanesulfonic acid 5-cyclopropyl-2-methyl-2H-pyrazol-3-yl ester (90 mg, 46%).

### Intermediate 17

### 2-(5-Bromo-1-methyl-1H-pyrazol-3-yl)-pyridine

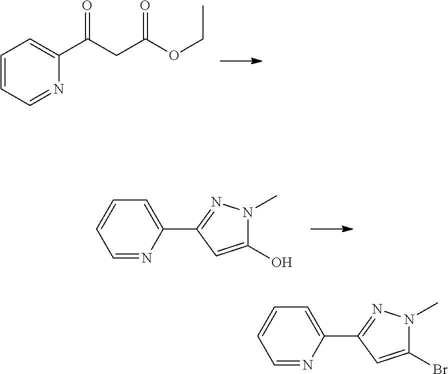

Methyl-5-pyridin-2-yl-2H-pyrazol-3-ol: To a solution of 3-Oxo-3-pyridin-2-yl-propionic acid ethyl ester (5 g, 25.9 mmol) in EtOH (12 ml) was added methyl hydrazine (1.38 ml, 25.9 mmol) and the mixture refluxed for 4 h. Upon completion, the EtOH was evaporated and resultant yellow solid was washed with hexane to give 2-Methyl-5-pyridin-2-yl-2H-pyrazol-3-ol (3.6 g, 79%) as an off-white solid.

2-(5-Bromo-1-methyl-1H-pyrazol-3-yl)-pyridine: A mixture of 2-Methyl-5-pyridin-2-yl-2H-pyrazol-3-ol (1.19 g, 6.8 mmol) and POBr3 (13.64 g, 47.6 mmol) were heated to 120° C. for 1 h. Upon completion, the mixture was cooled, ice-water was then added to quench the reaction, and the aqueous phase extracted with EtOAc. The combined organic layers were then washed with brine, dried over Na₂SO₄, concentrated, and the crude material was purified by column chromatography to give 2-(5-Bromo-1-methyl-1H-pyrazol-3-yl)-pyridine (765 mg, 47%).

### Intermediate 18

### 2-(4-Bromo-3-methoxy-phenyl)-oxazole

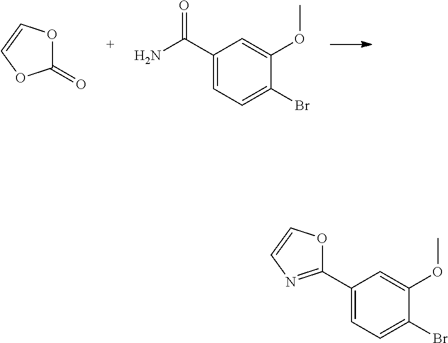

Intermediate 18 was prepared in a manner identical to that used for Intermediate 14.

### Intermediate 19

### Trifluoro-methanesulfonic acid 5-methyl-2-pyridazin-4-yl-thiazol-4-yl ester

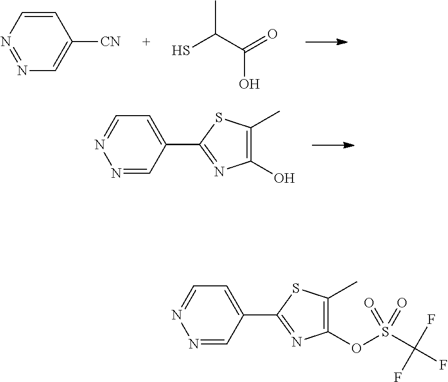

Methyl-2-pyridazin-4-yl-thiazol-4-ol: To 4-cyanopyridazine (100 mg, 0.95 mmol) and thiolactic acid (100 mg, 0.95 mmol) was added pyridine (0.01 ml, 0.24 mmol). The mixture was then heated to 100° C. for 3 h, after which it was cooled, and EtOH (3 ml) was added, stirred for 10 min, filtered and dried to give 5-Methyl-2-pyridazin-4-yl-thiazol-4-ol (150 mg, 81%).

Trifluoro-methanesulfonic acid 5-methyl-2-pyridazin-4-yl-thiazol-4-yl ester: To a solution of 5-Methyl-2-pyridazin-4-yl-thiazol-4-ol (150 mg, 0.777 mmol) in THF (2 ml) cooled to 0° C. was added NaH (24 mg, 1.0 mmol) followed by N-phenyl bis(trifluoromethanesulfonimide) (416 mg, 1.17 mmol). The mixture was then stirred at 25° C. for 1 h, after which water was added at 0° C. and the mixture extracted with EtOAc. The organic phase was separated and washed with NaOH solution (0.1N), brine, dried, concentrated, and purified by column chromatography to give trifluoro-methanesulfonic acid 5-methyl-2-pyridazin-4-yl-thiazol-4-yl ester (100 mg, 40%).

### Intermediate 20

### Bromo-1-methyl-1H-pyrazol-3-ylamine

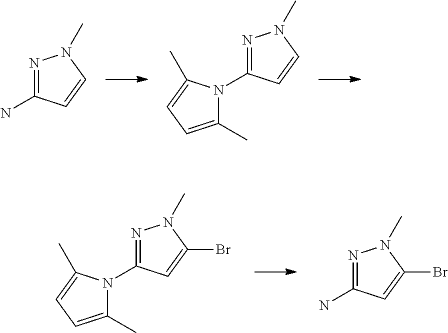

3-(2,5-Dimethyl-pyrrol-1-yl)-1-methyl-1H-pyrazole: To a solution of 1-Methyl-1H-pyrazol-3-ylamine (2 g, 20.59 mmol), hexane-2,5-dione (2.82 g, 24.71 mmol) in toluene (35 ml) was added PTSA.H2O (392 mg, 2.059 mmol). The mixture was refluxed for 20 h, after which the toluene was removed and water was added water. The aqueous layer was then extracted with EtOAc, separated, and the organic phase was washed with brine, dried over Na₂SO₄, concentrated, and the crude material purified by column chromatography to give 3-(2,5-Dimethyl-pyrrol-1-yl)-1-methyl-1H-pyrazole (1.9 g, 52%).

Bromo-3-(2,5-dimethyl-pyrrol-1-yl)-1-methyl-1H-pyrazole: To 3-(2,5-Dimethyl-pyrrol-1-yl)-1-methyl-1H-pyrazole (4.5 g, 25.71 mmol) in dry THF (40 ml) at −78° C. was added n-BuLi (1.7M, 16.4 ml, 28.02 mmol). The reaction mixture was stirred for 2 h at −78° C. before CNBr (2.97 g, 28.02 mmol) dissolved in THF (5 ml) was added. The mixture was allowed to warm to rt, and stirred for an additional 2 h, after which ice-water was added and the aqueous mixture extracted with EtOAc. The organic layer was washed with brine, dried over Na₂SO₄, concentrated and purified by column chromatography to give 5-Bromo-3-(2,5-dimethyl-pyrrol-1-yl)-1-methyl-1H-pyrazole (4.4 g, 68%).

5-Bromo-1-methyl-1H-pyrazol-3-ylamine: To a solution of 5-Bromo-3-(2,5-dimethyl-pyrrol-1-yl)-1-methyl-1H-pyrazole (179 mg, 0.7 mmol) and hydroxylamine hydrochloride (502 mg, 7.0 mmol) in EtOH (2 ml) was added aq. KOH (2.3M, 3 ml). The mixture was refluxed for 65 h, after which it was cooled, the EtOH evaporated, and ice-water added. The mixture was then extracted with EtOAc, and the organic layer was washed with brine, dried over Na₂SO₄, concentrated, and the crude material was purified by column chromatography to obtain 5-Bromo-1-methyl-1H-pyrazol-3-ylamine (90 mg, 71%).

### Intermediate 21

### 3-(5-bromo-1-methyl-1H-[1,2,4]triazol-3-yl)-pyridine

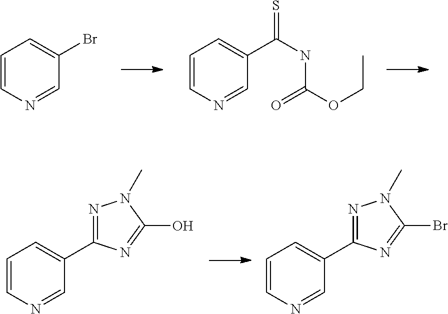

ethyl pyridine-3-carbonothioylcarbamate: n-BuLi (2.5M in THF, 60 mL, 150 mmol, 1 eq) was charged into a 3-neck 2000 ml round bottom flask, attached with a mechanical stirrer and two dropping funnels (one containing a solution of 3-bromopyridine (14.46 mL, 150 mmol, 1 eq) in 220 ml of anhydrous ether and the other one containing O-ethyl carbonisothiocyanatidate (20.4 mL, 180 mmol, 1.2 eq) in 500 mL of anhydrous THF) under argon. The solution was cooled to −78° C. The 3-bromopyridine solution was added dropwise over 45 min and stirred at −7° C. for 30 min. The solution of O-ethyl carbonisothiocyanatidate was added dropwise over 75 min. Stirring was continued and the reaction mixture was allowed to come to RT overnight. 50 mL of saturated ammonium chloride was added and the reaction mixture was concentrated to small volume, diluted with EtOAc, washed with brine, dried over anhydrous magnesium sulfated, filtered and evaporated to a red oil. Flash chromatography on silica gel (600 g) using a gradient of 0-50% EtOAc/hexanes in 60 min gave 5.2 g (16.5%) of ethyl pyridine-3-carbonothioylcarbamate as a yellow solid. LC-MS (ES) calculated for C₉H₁₀N₂O₂S, 210.26; found m/z 211.1 [M+H]⁺.

methyl-3-(pyridin-3-yl)-1H-1,2,4-triazol-5-ol: The solution of ethyl pyridine-3-carbonothioylcarbamate (4.6 g, 21.9 mmol, 1 eq) and methylhydrazine (46 mL, 873 mmol, 39.9 eq) in 46 mL THF was heated at 80° C. in an oil bath for 40 min. The reaction mixture was cooled and evaporated. Flash chromatography on silica gel (240 g) using a gradient of 20-100% EtOAc/hexanes in 60 min gave 2.65 g (69%) of 1-methyl-3-(pyridin-3-yl)-1H-1,2,4-triazol-5-ol as an off-white solid. LC-MS (ES) calculated for C₈H₈N₄O, 176.18; found m/z 177.1 [M+H]⁺.

3-(5-bromo-1-methyl-1H-[1,2,4]triazol-3-yl)-pyridine: 1-methyl-3-(pyridin-3-yl)-1H-1,2,4-triazol-5-ol (1.2 g, 11.33 mmol, 1 eq) and phosphoryl tribromide (14.56 g, 50.84 mmol, 3.98 eq) were combined in a microwave reaction vessel and sealed. The mixture was heated at 120° C. in an oil bath for 2 hrs. The reaction mixture was cooled in acetone/dry ice bath and neutralized carefully with a saturated sodium bicarbonate solution, extracted with EtOAc, dried over anhydrous magnesium, filtered and evaporated. Flash chromatography on silica gel (120 g) using a gradient column of 0-60% EtOAc/hexane in 45 min gave 2.28 g (74%) of 3-(5-bromo-1-methyl-1H-[1,2,4]triazol-3-yl)-pyridine as a white solid. LC-MS (ES) calculated for C₈H₇BrN₄, 239.08; found m/z 240.0 [M+H]⁺.

### Intermediate 22

### 2-(4-Bromo-3-methyl-phenyl)-[1,3,4]oxadiazole

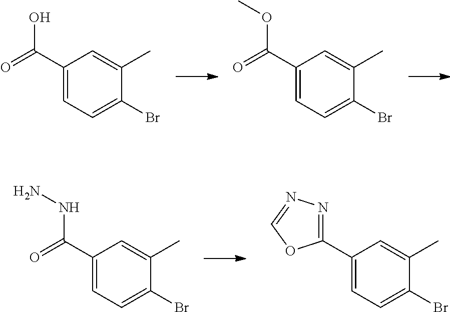

4-Bromo-3-methyl-benzoic acid methyl ester: To a solution of 4-Bromo-3-methyl-benzoic acid (3 g, 13.19 mmol) in MeOH (15 ml) was added conc. H₂SO₄ (0.6 ml). The mixture was refluxed for 14 h, cooled to 0° C., nuetralized with saturated NaHCO₃, and filtered to give a solid. This material was purified by column chromatography to give 4-Bromo-3-methyl-benzoic acid methyl ester (3.1 g, 97%) as a white solid.

4-Bromo-3-methyl-benzoic acid hydrazide: To a solution of 4-Bromo-3-methyl-benzoic acid methyl ester (2 g, 8.73 mmol) in MeOH (20 ml) was added hydrazine hydrate (1.1 ml). The mixture was refluxed for 18 h, cooled to room temperature, concentrated, and purified by column chromatograph to give 4-Bromo-3-methyl-benzoic acid hydrazide (1 gm, 50%) as white solid.

2-(4-Bromo-3-methyl-phenyl)-[1,3,4] oxadiazole: To 4-Bromo-3-methyl-benzoic acid hydrazide (1 g, 4.36 mmol) was added triethyl orthoformate (10 ml). The mixture was refluxed for 18 h, cooled to room temperature, filtered, and purified by column chromatograph to give 2-(4-Bromo-3-methyl-phenyl)-[1,3,4] oxadiazole (900 mg, 90%) as light brown solid.

### Intermediate 23

### 5-Bromo-4-methyl-pyridine-2-carboxylic acid methyl ester

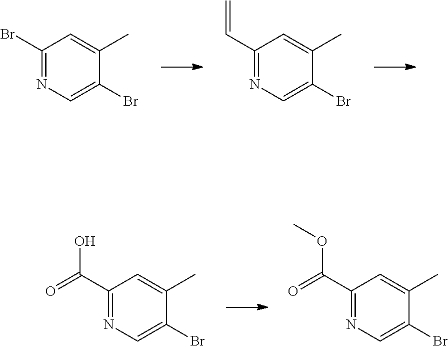

5-Bromo-4-methyl-2-vinyl-pyridine: To a solution of 2,5-Dibromo-4-methyl-pyridine (10 g, 39.8 mmol) and trivinyl cyclotriboroxane (6.44 g, 39.8 mmol) in DME (150 ml) was added K₂CO₃ (5.5 gm, 39.8 mmol) in water (30 mL) followed by Pd(PPh₃)₄ (460 mg, 0.398 mmol). The mixture was stirred at 100° C. for 4 h, after which it was filtered through Celite. The filtrate was diluted with water and extracted with EtOAc. The organic phase was washed with brine, dried, concentrated, and the crude material was purified by column chromatograph to give 5-Bromo-4-methyl-2-vinyl-pyridine (7.04 gm, 70%) as light yellow solid.

5-Bromo-4-methyl-pyridine-2-carboxylic acid: To a solution of 5-Bromo-4-methyl-2-vinyl-pyridine (600 mg, 3 mmol) in acetone-water (1:1, 54 ml) was added KMnO₄ (957 mg, 6 mmol). The mixture was stirred for 3 days at rt, at which point it was filtered, concentrated, and purified by column chromatograph to give 5-Bromo-4-methyl-pyridine-2-carboxylic acid (700 mg, 92%) as white solid.

5-Bromo-4-methyl-pyridine-2-carboxylic acid methyl ester: To a solution of 5-Bromo-4-methyl-pyridine-2-carboxylic acid (650 mg, 3.0 mmol) in MeOH (2 ml) was added conc. H₂SO₄ (0.06 ml). The mixture was refluxed for 14 h, after which it was cooled to 0° C., neutralized with saturated NaHCO₃, filtered, concentrated, and purified by column chromatography to give 5-Bromo-4-methyl-pyridine-2-carboxylic acid methyl ester (340 mg, 49%) as white solid.

### Intermediate 24

### 5-Bromo-4-methyl-pyridine-2-carboxylic acid methylamide

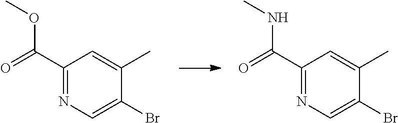

5-Bromo-4-methyl-pyridine-2-carboxylic acid methylamide: To 5-Bromo-4-methyl-pyridine-2-carboxylic acid methyl ester (200 mg, 0.869 mmol) and methylamine (135 mg, 11.34 mmol) was added (CH₃)₃Al (0.6 mg, 0.008 mmol). The mixture was placed in a sealed tube and heated at 100° C. for 1 h, after which the mixture was cooled, quenched with water, and extracted with EtOAc. The organic phase was dried, concentrated, and purified by column chromatograph to give 5-Bromo-4-methyl-pyridine-2-carboxylic acid methylamide (130 mg, 65%) as an off-white solid.

### Intermediate 25

### 5-Bromo-4-methyl-2-[1,3,4]oxadiazol-2-yl-pyridine

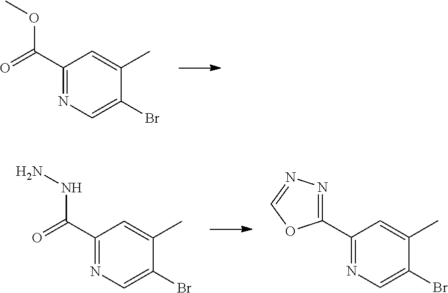

Prepared in a manner identical to Intermediate 22.

5-Bromo-4-methyl-pyridine-2-carboxylic acid hydrazide: 700 mg (70%) as an off-white solid.

5-Bromo-4-methyl-2-[1,3,4]oxadiazol-2-yl-pyridine: 60 mg (20%) as an off-white solid.

### Intermediate 26

### 5-Bromo-4-methyl-2-(5-methyl-[1,3,4]oxadiazol-2-yl)-pyridine

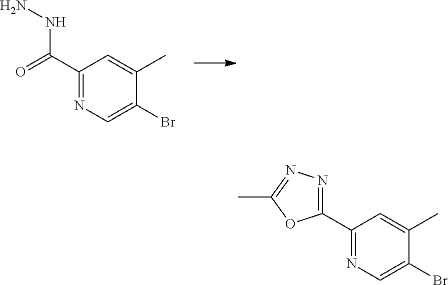

Prepared in a manner identical to Intermediate 22 substituting triethyl orthoacetate in the condensation step.

5-Bromo-4-methyl-2-(5-methyl-[1,3,4]oxadiazol-2-yl)-pyridine: 250 mg (83%) as a white solid.

### Intermediate 27

### 3-(5-Bromo-1-ethyl-1H-[1,2,4]triazol-3-yl)-pyridine

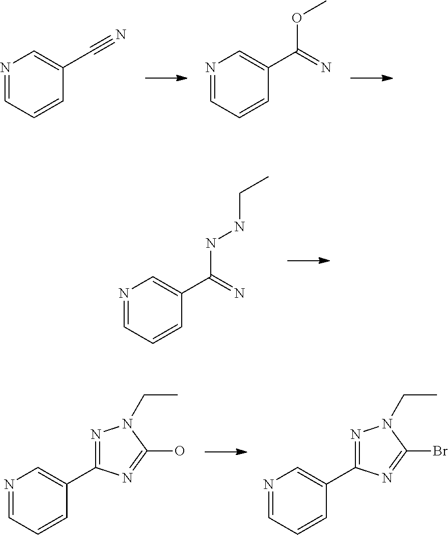

Nicotinimidic acid methyl ester: To a stirred solution of 3-cyanopyridine (5.0 g, 48.07 mmol) in methanol-1,4-dioxane (1:1; 50 ml) was added sodium methoxide (2.85 g, 52.88 mmol) at 0° C. The reaction mixture was stirred for 24 h at rt, after which the solvent was removed, and water (20 mL) was added to the resulting mass. This mixture was extracted with ethyl acetate (2×50), and the organic layers were dried, concentrated in vacuo and purified by column chromatography (20% EtOAc/Hexanes) to give nicotinimidic acid methyl ester (3.6 g, 55%) as light yellow liquid.

N′-ethylnicotinimidohydrazide: To a stirred solution of nicotinimidic acid methyl ester (2.0 g, 14.70 mmol) in dry pyridine (10 mL) was added ethyl hydrazine oxalate (2.34 g, 15.58 mmol) at rt. The mixture was stirred for 12 h, after which the solvent was removed to furnish a crude mass. This material was triturated with diethyl ether to give N′-ethylnicotinimidohydrazide (2.1 g, 87%) as a white solid.

Ethyl-5-pyridin-3-yl-2H-[1,2,4]triazol-3-ol: To a stirred solution of N′-ethylnicotinimidohydrazide (0.500 g, 3.05 mmol) in dry DMF (15 mL) was added CDI (0.524 g, 3.23 mmol) at rt. The mixture was then stirred for 12 h, after which the DMF was removed in vacuo, the material redissolved in methylene dichloride (25 mL), and filtered through a sintered funnel. The filtrate was concentrated under reduced pressure to provide a crude mass that was purified by column chromatography (20% methanol in DCM), to give 2-Ethyl-5-pyridin-3-yl-2H-[1,2,4]triazol-3-ol (0.200 g, 35%) as a white solid.

3-(5-Bromo-1-ethyl-1H-[1,2,4]triazol-3-yl)-pyridine: A solution of 2-Ethyl-5-pyridin-3-yl-2H-[1,2,4]triazol-3-ol (0.240 g, 1.26 mmol) in phosphorus oxybromide (1.44 g, 5.05 mmol) was stirred at 140° C. for 1 h. It was then cooled to 0° C. and the solution was basified to pH ˜9 with an aqueous solution of saturated sodium bicarbonate. The aqueous mixture was extracted with ethyl acetate (3×20 mL), and the organic layers were then dried over anhydrous sodium sulfate, concentrated, and purified by column chromatography (20% EtOAc/Hexanes) to give 3-(5-Bromo-1-ethyl-1H-[1,2,4]triazol-3-yl)-pyridine (0.160 g, 50.19%) as a brown solid.

### Intermediate 28

### Trifluoro-methanesulfonic acid 5-methyl-2-oxazol-2-yl-thiazol-4-yl ester

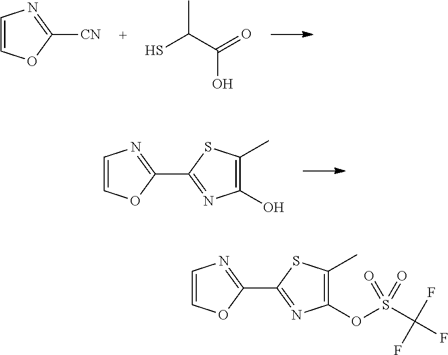

5-Methyl-2-oxazol-2-yl-thiazol-4-ol: To a mixture of 2-cyanooxazole (500 mg, 5.32 mmol) and thiolactic acid (564 mg, 5.32 mmol) was added pyridine (0.1 ml, 1.32 mmol). The mixture was heated to 100° C. for 3 h, after which it was cooled to rt, EtOH (3 ml) was added, and the suspension stirred for 10 min, filtered, and the solid dried. Further purification by column chromatography (30% EtOAc/Hexane) gave 5-Methyl-2-oxazol-2-yl-thiazol-4-ol (492 mg, 51%) as an off white solid.

Trifluoro-methanesulfonic acid 5-methyl-2-oxazol-2-yl-thiazol-4-yl ester: To a solution of 5-Methyl-2-oxazol-2-yl-thiazol-4-ol (492 mg, 2.70 mmol) in THF (35 ml) was added NaH (95 mg, 4.05 mmol) followed by N-phenyl bis(trifluoromethanesulfonimide) (1.32 g, 3.24 mmol) at 0° C. The reaction mixture was stirred at 25° C. for 1 h, at which point water was added at 0° C., and resulting solution extracted with EtOAc. The organic phase was washed with NaOH solution (0.1N), brine, then dried over Na₂SO₄, concentrated, and purified by column chromatography (8% EtOAC-Hexane) to give Trifluoro-methanesulfonic acid 5-methyl-2-oxazol-2-yl-thiazol-4-yl ester (551 mg, 65%) as a white solid.

### Intermediate 29

### 5-bromo-2-ethoxy-4-picoline

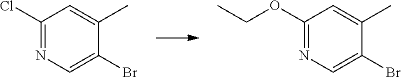

5-bromo-2-ethoxy-4-picoline: To a solution of 5-bromo-2-chloro-4-picoline (0.50 g, 2.4 mmol) in NMP (4 ml), was added a solution of sodium ethoxide (21% in EtOH, 1.2 ml, 3.2 mmol), the mixture was placed in a microwave reactor and heated to 150° C. for 30 minutes, the cooled reaction mixture was partitioned between EtOAc and water, the organic phase was washed with brine, dried over Na₂SO₄, filtered and concentrated under reduced pressure, the crude material was purified by filtering through a pad of silica gel (10% EtOAc/hexanes) to give 5-bromo-2-ethoxy-4-picoline (0.42 g, 80%) as a pale yellow oil.

### Intermediate 30

### Chloro-5-methyl-pyridine-3-sulfonic acid dimethylamide

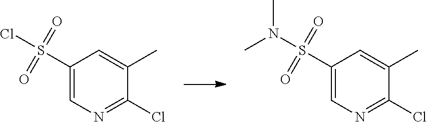

6-chloro-5-methyl-pyridine-3-sulfonic acid dimethylamide: To solution of 6-chloro-5-methylpyridine-3-sulfonyl chloride (1.0 g, 4.4 mmol) and triethylamine (492 mg, 0.68 mL, 4.9 mmol) in CH₂Cl₂ (5 ml) was added dropwise a solution of dimethylamine (2.4 ml, 4.9 mmol) in CH₂Cl₂ (5 ml). The reaction mixture was stirred overnight at room temperature, partitioned between CH₂Cl₂ and water, the organic phase was washed with water and brine, dried over Na₂SO₄, filtered and concentrated under reduced pressure. The crude 6-chloro-5-methyl-pyridine-3-sulfonic acid dimethylamide was used without further purification.

### Intermediate 31

### Bromo-N,3-dimethylbenzenesulfonamide

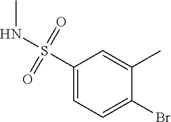

bromo-N,3-dimethylbenzenesulfonamide: Similarly prepared using the above procedure outlined for Intermediate 30, but replacing 6-chloro-5-methylpyridine-3-sulfonyl chloride with 4-bromo-3-methylbenzene-1-sulfonyl chloride and dimethylamine with methylamine hydrochloride to give 4-bromo-N,3-dimethylbenzenesulfonamide, which was used without purification.

### Intermediate 32

### 4-(4-Chloro-3-methyl-benzenesulfonyl)-morpholine

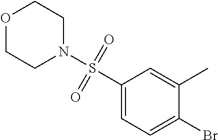

4-(4-Chloro-3-methyl-benzenesulfonyl)-morpholine: Similarly prepared using the above procedure outlined for Intermediate 30, but replacing 6-chloro-5-methylpyridine-3-sulfonyl chloride with 4-bromo-3-methylbenzene-1-sulfonyl chloride and dimethylamine with morpholine to give 4-(4-chloro-3-methyl-benzenesulfonyl)-morpholine, which was used without purification.

### Intermediate 33

### 1-(4-Bromo-3-methyl-benzenesulfonyl)-4-methyl-piperazine

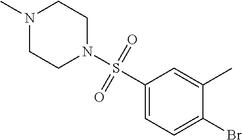

1-(4-Bromo-3-methyl-benzenesulfonyl)-4-methyl-piperazine: Similarly prepared using the above procedure outlined for Intermediate 30, but replacing 6-chloro-5-methylpyridine-3-sulfonyl chloride with 4-bromo-3-methylbenzene-1-sulfonyl chloride and dimethylamine with 1-methylpiperazine to give 1-(4-bromo-3-methyl-benzenesulfonyl)-4-methyl-piperazine, which was used without purification.

### Intermediate 34

### 2-(2-chloro-6-fluorophenyl)-5-(2-methyl-4-(2-methyl-2H-tetrazol-5-yl)phenyl)-1H-indole

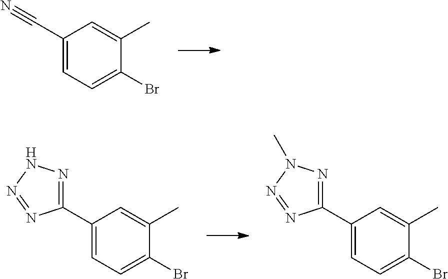

5-(4-bromo-3-methylphenyl)-2H-tetrazole: To a 100 ml round-bottomed flask were added, 4-bromo-3-methylbenzonitrile (2.0 g, 10 mmol), sodium azide (0.86 mg, 13 mmol), triethylamine hydrochloride (1.83 g, 13.3 mmol), and xylenes (20 ml) to give an off-white suspension. The mixture was heated to 140° C. overnight, partitioned between EtOAc and water, and the aqueous solution was adjusted to pH<2 with conc. HCl, the solids were collected, and washed with water three times, dried in a vacuum oven to give 5-(4-bromo-3-methylphenyl)-2H-tetrazole as an off-white solid (2.25 g, 92%).

5-(4-bromo-3-methylphenyl)-2-methyl-2H-tetrazole: To a solution of 5-(4-bromo-3-methylphenyl)-2H-tetrazole (1.02 g, 4.27 mmol) in THF (20 ml), was added dropwise (trimethylsilyl)diazomethane (4.69 ml, 9.39 mmol) at room temperature, the mixture was stirred at room temperature for one hour, water was added, extracted with EtOAc, and the organic phase was washed with brine, dried over Na₂SO₄, filtered and concentrated under reduced pressure. The crude material was purified by filtering through a pad of silica gel (5-10% EtOAc/hexanes) to give 5-(4-bromo-3-methylphenyl)-2-methyl-2H-tetrazole as a white solid (664 mg, 61%).

### Intermediate 35

### Bromo-2-methanesulfonyl-4-methyl-pyridine

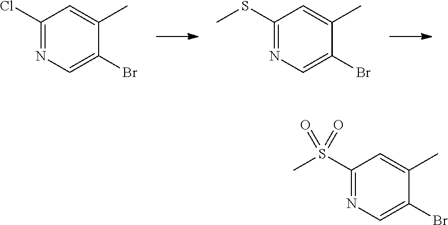

Bromo-4-methyl-2-methylsulfanyl-pyridine: A mixture of 5-bromo-2-chloro-4-methylpyridine (1.81 g, 8.8 mmol), and sodium thiomethoxide (0.68 g, 9.8 mmol) in 10 mL of dioxane was placed in a 110° C. oil bath for 3 hrs., cooled and extracted between ethyl acetate and water, washed organic layer with water, dried over sodium sulfate, filtered and concentrated to give the crude product as a pale-yellow liquid (1.83 g). The crude product was carried onto the oxidation step without further purification.

5-Bromo-2-methanesulfonyl-4-methyl-pyridine: To a 0° C. solution of 5-bromo-4-methyl-2-(methylthio)pyridine (1.83 g, 8.4 mmol) in 25 mL of dichloromethane was added MCPBA (3.50 g, 55% pure, 11 mmol). The reaction mixture was stirred for 1 hr., partitioned between water and dichloromethane, then washed the organic layer twice with aq. sodium bicarbonate, dried over sodium sulfate, filtered and concentrated to give a crude yellow solid. The crude mixture was loaded onto Si-gel and purified by flash chromatography (20:80-1:1 ethyl acetate/hexanes then 100% ethyl acetate) to give the product as a light-yellow solid (0.64 g, 29% over two steps). MS (M+H)=252.

### Intermediate 36

### Chloro-4-ethyl-5-iodo-pyridine

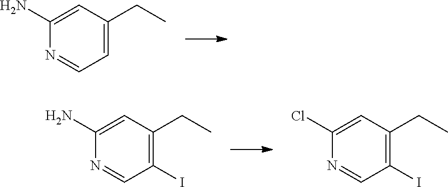

ethyl-5-iodopyridin-2-amine: 4-ethylpyridin-2-amine (2 g, 16.4 mmol, Eq: 1.00) and potassium acetate (1.61 g, 16.4 mmol, Eq: 1.00) were dissolved in 20 mL acetic acid and heated to 80° C. Added a solution of iodine monochloride (2.66 g, 820 μL, 16.4 mmol, Eq: 1.00) in acetic acid (10 mL) and continued to heat at 80° C. for 4 hrs. Quenched reaction with sodium bisulfite, sat (3 mL) and then removed acetic acid in vacuo. Diluted with EtOAc/NaHCO₃. Washed with NaHCO₃ (1×) and water (1×). Dried organic layer onto silica gel for purification using a 10-50% EtOAc/Hex gradient. Obtained 4-ethyl-5-iodopyridin-2-amine (2.58 g, 10.4 mmol, 64% yield) as a white solid.

2-chloro-4-ethyl-5-iodopyridine: 4-ethyl-5-iodopyridin-2-amine (2.58 g, 10.4 mmol, Eq: 1.00) was dissovled in hydrochloric acid (28.8 g, 24 mL, 790 mmol, Eq: 75.9) and cooled to 0° C. sodium nitrite (1.44 g, 20.8 mmol, Eq: 2) was dissolved in water (8 mL) and added dropwise to the solution at 0° C. Stirred at 0° C. for 2 hr. Warmed to r.t. for 1 hr. Continued to stir at r.t. over weekend. Cooled the mixture to 0° C. and added NaOH (sat) until pH-12. Extracted with DCM (2×). Dried onto silica gel for purification using a 10-50% EtOAc/Hex gradient. Obtained 2-chloro-4-ethyl-5-iodopyridine (1.58 g, 57% yield) as a colorless liquid.

### Intermediate 37

### Trifluoro-methanesulfonic acid 5-ethyl-2-pyridin-3-yl-thiazol-4-yl ester

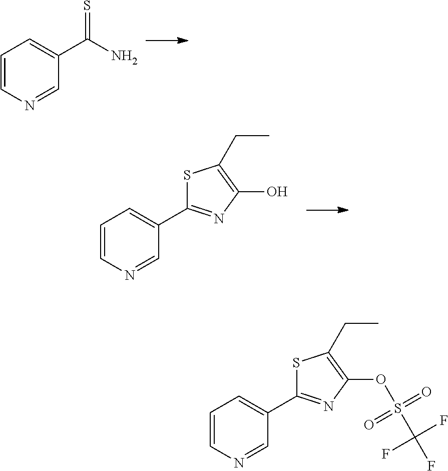

Trifluoro-methanesulfonic acid 5-ethyl-2-pyridin-3-yl-thiazol-4-yl ester: To a solution of pyridine-3-carbothioamide (1 g, 7.24 mmol) in EtOH (15 mL) and pyridine (1 mL, 12.3 mmol) was added methyl 2-bromobutanoate (1 mL, 8.68 mmol). The mixture was heated at reflux for 18 hours, after which it was cooled and concentrated. The crude 5-Ethyl-2-pyridin-3-yl-thiazol-4-ol was then redissolved in DMF (36 mL) at 0° C., and to the mixture was added 60% sodium hydride (751 mg, 18.8 mmol). After stirring for 15 min at rt, 1,1,1-trifluoro-N-phenyl-N-(trifluoromethylsulfonyl)methanesulfonamide (3.87 g, 10.8 mmol) was added. The mixture was reacted for 20 min, quenched with sat. NH4Cl, diluted with diethyl ether. The mixture was washed with water, and then brine. The organic layer was concentrated, and the resulting material chromatographed (5-55% EtOAc/Hexanes to give trifluoro-methanesulfonic acid 5-ethyl-2-pyridin-3-yl-thiazol-4-yl ester (0.85 g) as an orange oil.

### Intermediate 38

### Trifluoro-methanesulfonic acid 5-methyl-2-pyrazin-2-yl-thiazol-4-yl ester

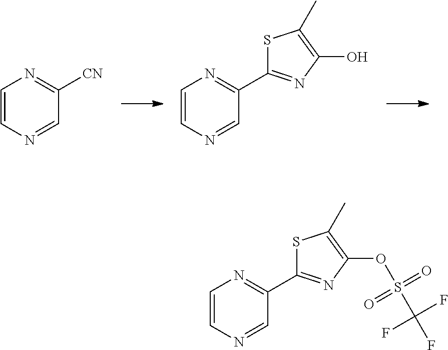

Methyl-2-pyrazin-2-yl-thiazol-4-ol: In a 250 mL round-bottomed flask, pyrazine-2-carbonitrile (10 g, 95.1 mmol), pyridine (2.26 g, 2.33 ml, 28.5 mmol), and 2-mercaptopropionic acid (10.1 g, 95.1 mmol) were combined to give a light yellow solution. The reaction mixture was heated to 100° C. and stirred for 2 h. Upon cooling, the thick yellow mixture was diluted with 100 mL ethanol and stirred for 30 min. The slurry was then filtered, and washed with diethyl ether (2×100 mL) to give 5-methyl-2-pyrazin-2-yl-thiazol-4-ol (17.86 g, 97.1%) as yellow solid which was used directly without further purification.

Trifluoro-methanesulfonic acid 5-methyl-2-pyrazin-2-yl-thiazol-4-yl ester: In a 500 mL round-bottomed flask, 5-methyl-2-(pyrazin-2-yl)thiazol-4-ol (12.24 g, 63.3 mmol) was cooled to 0° C. in THF (110 ml) and stirred for 33 min. 60% sodium hydride (3.32 g, 83.0 mmol) was added followed by N-phenylbis (trifluoromethanesulfonimide) (26.6 g, 72.8 mmol) and the resultant reaction mixture was warmed to 25° C. and stirred for 1 h. The reaction mixture was poured into 50 mL H₂O and extracted with ethyl acetate (3×20 mL). The organic layers were dried over MgSO₄ and concentrated in vacuo. The crude material was purified by flash column chromatography (silica gel, 120 g, 25% to 45% ethyl acetate in hexanes) to give trifluoro-methanesulfonic acid 5-methyl-2-pyrazin-2-yl-thiazol-4-yl ester (7.45 g, 36.2%) as a colorless oil which solidified to an off-white solid.

### Intermediate 39

### Trifluoro-methanesulfonic acid 5-methyl-2-pyrimidin-5-yl-thiazol-4-yl ester

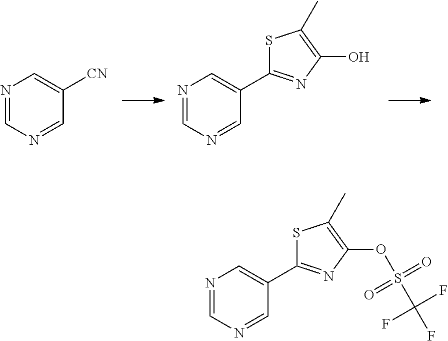

Methyl-2-(pyrimidin-2-yl)-thiazol-4-ol: In a 250 mL round-bottomed flask, pyrimidine-5-carbonitrile (1.5 g, 14.3 mmol), pyridine (0.339 g, 0.35 ml, 28.5 mmol), and 2-mercaptopropionic acid (1.51 g, 14.3 mmol) were combined to give a light yellow solution. The reaction mixture was heated to 100° C. and stirred for 2 h. Upon cooling, the thick yellow mixture was diluted with 100 mL ethanol and stirred for 30 min. The slurry was then filtered, and washed with diethyl ether (2×100 mL) to give 5-Methyl-2-(pyrimidin-2-yl)-thiazol-4-ol (2.33 g, 85%) as yellow solid which was used directly without further purification.

Trifluoro-methanesulfonic acid 5-methyl-2-pyrimidin-5-yl-thiazol-4-yl ester: In a 100 mL round-bottomed flask, 5-Methyl-2-(pyrimidin-2-yl)-thiazol-4-ol (0.74 g, 3.83 mmol) was cooled to 0° C. in DMF (7 ml) and stirred for 33 min. 60% sodium hydride (0.201 g, 5 mmol) was added followed by N-phenylbis (trifluoromethanesulfonimide) (1.61 g, 4.4 mmol) and the resultant reaction mixture was warmed to 25° C. and stirred for 1 h. The reaction mixture was poured into 50 mL water and extracted with ethyl acetate (3×20 mL). The organic layers were dried over MgSO₄ and concentrated in vacuo. The crude material was purified by flash column chromatography (silica gel, 40 g, 25% to 45% ethyl acetate in hexanes) to give trifluoro-methanesulfonic acid 5-methyl-2-pyrimidin-5-yl-thiazol-4-yl ester (0.32 g, 26%) as brown oil.

### Intermediate 40

### Trifluoro-methanesulfonic acid 5-methyl-2-(6-methyl-pyridin-3-yl)-thiazol-4-yl ester

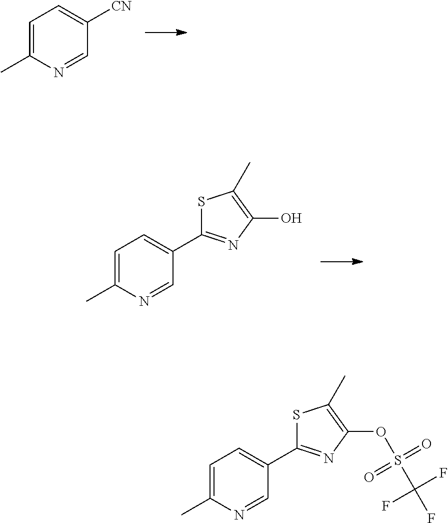

Was prepared in a manner identical to Example 38.

### Intermediate 41

### Trifluoro-methanesulfonic acid 5-ethyl-2-pyrazin-2-yl-thiazol-4-yl ester

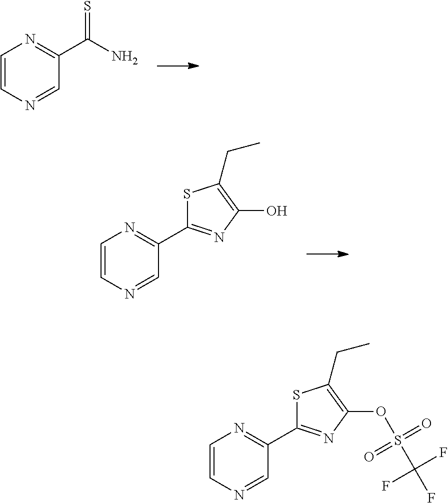

Ethyl-2-pyrazin-2-yl-thiazol-4-ol: A solution of pyrazine-2-carbothioamide (1 g, 7.19 mmol) in ethanol (20 ml) was treated with methyl 2-bromobutyrate (1.56 g, 992 μl, 8.62 mmol) an pyridine (853 mg, 872 μl, 10.8 mmol) and heated to reflux for 2 hours. The reaction mixture was cooled and concentrated to dryness under reduced pressure, and the resulting solid was filtered and washed with diethyl ether to provide 5-ethyl-2-pyrazin-2-yl-thiazol-4-ol (0.740 g, 50%) which was used directly without further purification. MS (M+H)=208.

Trifluoro-methanesulfonic acid 5-ethyl-2-pyrazin-2-yl-thiazol-4-yl ester: In a 100 mL round-bottomed flask, 5-ethyl-2-(pyrazin-2-yl)thiazol-4-ol (0.74 g, 3.57 mmol) was cooled to 0° C. in THF (110 ml) and stirred for 30 min. 60% sodium hydride (0.187 g, 4.68 mmol) was added followed by N-phenylbis (trifluoromethanesulfonimide) (1.5 g, 4.11 mmol) and the resultant reaction mixture was warmed to 25° C. and stirred for 1 h. The reaction mixture was poured into 50 mL H₂O and extracted with ethyl acetate (3×20 mL). The organic layers were dried over MgSO₄ and concentrated in vacuo. The crude material was purified by flash column chromatography (silica gel, 120 g, 20% to 25% ethyl acetate in hexanes) to give trifluoro-methanesulfonic acid 5-ethyl-2-pyrazin-2-yl-thiazol-4-yl ester (0.34 g, 28.1%) as light yellow oil which solidified upon standing.

### Intermediate 42

### Trifluoro-methanesulfonic acid 5-isopropyl-2-pyridin-3-yl-thiazol-4-yl ester

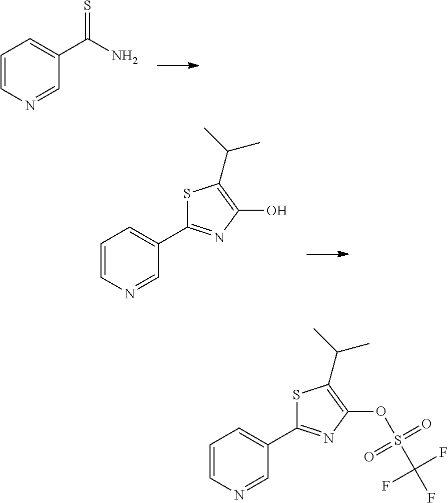

5-isopropyl-2-pyridin-3-yl-thiazol-4-ol: A solution of pyridine-3-carbothioamide (0.2 g, 1.45 mmol) in ethanol (10 ml) was treated with methyl 2-bromoisovalerate (0.423 g, 2.17 mmol) and pyridine (172 mg, 176 μl, 2.17 mmol) is combined to give a dark brown suspension. and heated to 160° C. for 6 hours in a sealed tube. The reaction mixture was cooled and concentrated to dryness under reduced pressure, and the resulting suspension is extracted with ethyl acetate (3×20 mL). The organic layers were combined, washed with saturated NaHCO₃ (1×50 mL), saturated sodium chloride (2×20 mL). The organic layers were dried over Mg50₄ and concentrated in vacuo to give 5-isopropyl-2-pyridin-3-yl-thiazol-4-ol (300 mgs, 94%) which was used directly without further purification.

Trifluoro-methanesulfonic acid 5-isopropyl-2-pyridin-3-yl-thiazol-4-yl ester: In a 100 mL round-bottomed flask, crude 5-isopropyl-2-pyridin-3-yl-thiazol-4-ol (0.30 g, 1.36 mmol) was cooled to 0° C. in DMF (10 ml) and stirred for 30 min. 60% sodium hydride (0.116 g, 2.89 mmol) was added followed by N-phenylbis (trifluoromethanesulfonimide) (0.59 g, 1.66 mmol) and the resultant reaction mixture was warmed to 25° C. and stirred for 16 h. The reaction mixture was poured into 50 mL water and extracted with ethyl acetate (3×20 mL). The organic layers were dried over MgSO₄ and concentrated in vacuo. The crude material was purified by flash column chromatography (silica gel, 40 g, 20% to 25% ethyl acetate in hexanes) to give trifluoro-methanesulfonic acid 5-isopropyl-2-pyridin-3-yl-thiazol-4-yl ester (0.110 g, 22%) as light yellow oil.

### Intermediate 43

### Trifluoro-methanesulfonic acid 5-isopropyl-2-pyrazin-2-yl-thiazol-4-yl ester

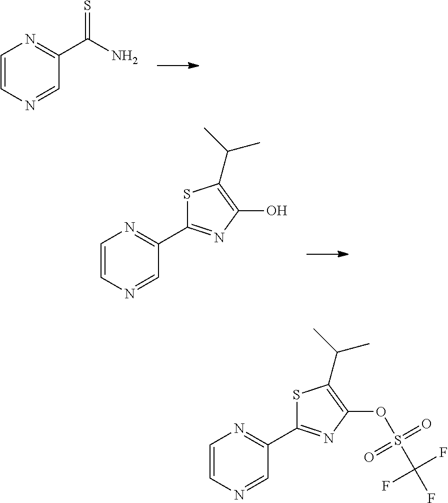

5-isopropyl-2-pyrazin-2-yl-thiazol-4-ol: A solution of pyrazine-2-carbothioamide (1 g, 7.19 mmol) in ethanol (10 ml) was treated with ethyl 2-bromoisovalerate (2.25 g, 10.8 mmol), and pyridine (853 mg, 872 μl, 10.8 mmol) is combined to give a dark brown suspension. and heated to 100° C. for 6 hours in a sealed tube. The reaction mixture was cooled and concentrated to dryness under reduced pressure, and the resulting suspension is extracted with ethyl acetate (3×50 mL). The organic layers were combined, washed with saturated NaHCO₃ (1×50 mL), saturated sodium chloride (2×20 mL). The organic layers were dried over MgSO4 and concentrated in vacuo to give 5-isopropyl-2-pyrazin-2-yl-thiazol-4-ol (260 mgs, 16%) which was used directly without further purification.

Trifluoro-methanesulfonic acid 5-isopropyl-2-pyrazin-2-yl-thiazol-4-yl ester: In a 250 mL pear-shaped flask, 5-isopropyl-2-pyrazin-2-yl-thiazol-4-ol (0.260 g, 1.17 mmol) was cooled to 0° C. in DMF (10 ml) and stirred for 3 min. 60% sodium hydride (0.61.6 g, 1.54 mmol) was added followed by N-phenylbis(trifluoromethane sulfonimide) (0.483 g, 1.35 mmol) and the resultant reaction mixture was warmed to 25° C. and stirred for 2 h. The reaction mixture was poured into 50 mL H₂O and extracted with EtOAc (3×50 mL). The organic layers were dried over MgSO₄ and concentrated in vacuo. The crude material was purified by flash column chromatography (silica gel, 40 g, 10% to 20% ethyl acetate in hexanes). to give trifluoro-methanesulfonic acid 5-isopropyl-2-pyrazin-2-yl-thiazol-4-yl ester (0.225 g, 54%) as colorless oil. MS (M+H)=354.

### Intermediate 44

### Trifluoro-methanesulfonic acid 2-pyridin-3-yl-5-(2,2,2-trifluoro-1-methyl-ethyl)-thiazol-4-yl ester

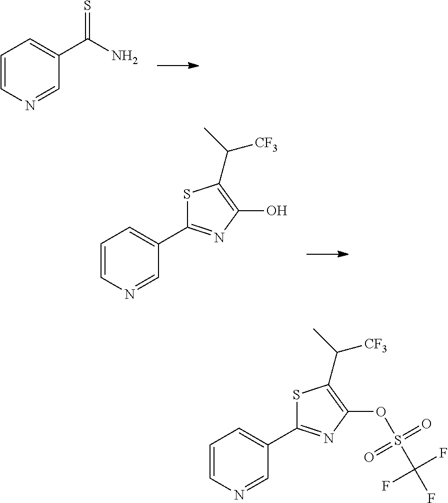

2-(Pyridin-3-yl)-5-(1,1,1-trifluoropropan-2-yl)thiazol-4-ol: A solution of pyridine-3-carbothioamide (1.0 g, 7.24 mmol) in ethanol (7 ml) was treated with ethyl 2-bromo-3-methyl-4,4,4-trifluorobutyrate (3 g, 11.04 mmol) and pyridine (577 mg, 590 μl, 7.29 mmol) is combined to give a dark brown suspension. and heated to 160° C. for 16 hours in a sealed tube. The reaction mixture was cooled and concentrated to dryness under reduced pressure, and the resulting suspension is extracted with ethyl acetate. The organic layers were combined, washed with saturated NaHCO₃ (1×50 mL), saturated sodium chloride (2×20 mL). The organic layers were dried over MgSO₄ and concentrated in vacuo. The crude material was purified by flash column chromatography (silica gel, 40 g, 10% to 30% ethyl acetate in hexanes). to give to give 2-(Pyridin-3-yl)-5-(1,1,1-trifluoropropan-2-yl)thiazol-4-ol (0.273 g, 14%).

Trifluoro-methanesulfonic acid 2-pyridin-3-yl-5-(2,2,2-trifluoro-1-methyl-ethyl)-thiazol-4-yl ester: In a 50 mL round-bottomed flask, 2-(Pyridin-3-yl)-5-(1,1,1-trifluoropropan-2-yl)thiazol-4-ol (0.27 g, 984 μmmol) was cooled to 0° C. in DMF (10 ml) and stirred for 30 min. 60% sodium hydride (0.052 g, 1.29 mmol) was added followed by N-phenylbis(trifluoromethanesulfonimide) (404 mg, 1.13 mmol) and the resultant reaction mixture was warmed to 25° C. and stirred for 1.5 h. The reaction mixture was poured into 50 mL H₂O and extracted with ethyl acetate (3×50 mL). The organic layers were dried over Na₂SO₄ and concentrated in vacuo. The crude material was purified by flash column chromatography (silica gel, 40 g, 10% to 30% ethyl acetate in hexanes). to give Trifluoro-methanesulfonic acid 2-pyridin-3-yl-5-(2,2,2-trifluoro-1-methyl-ethyl)-thiazol-4-yl ester (0.204 g, 51%) as colorless oil. MS (M+H)=407.

### Intermediate 45

### Trifluoro-methanesulfonic acid 2-ethyl-5-pyrazin-2-yl-2H-pyrazol-3-yl ester

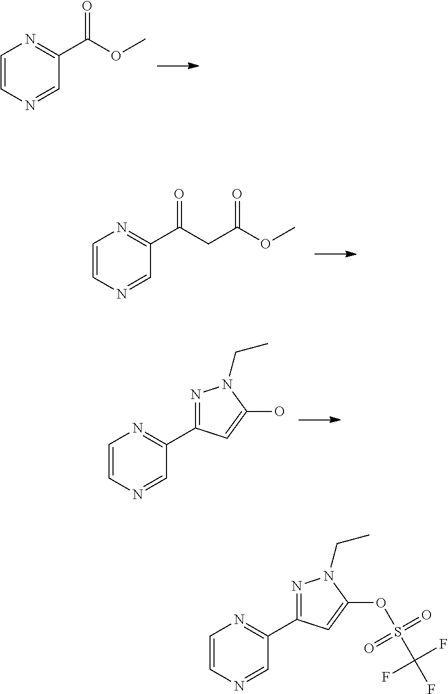

Methyl 3-oxo-3-(pyrazin-2-yl)propanoate: To a stirred solution of sodium methoxide (25% in MeOH, 27.54 mL, 72.4 mmol, 1 eq) in 90 mL of toluene at 110° C. in a 3-neck flask attached with a mechanical stirrer, condenser and dropping funnel was added a solution of methylpyrazine-2-carboxylate (10 g, 72.4 mmol, 1 eq) in 115 mL of methyl acetate, dropwise, over a period of ˜35-40 min. A yellow precipitate was formed. Stirring was continued at 110° C. for 3 hrs. The reaction was cooled and the yellow precipitate was filtered and washed with a small quantity of toluene. This solid was taken into 200 mL of saturated ammonium chloride and 400 mL of EtOAc. The aqueous layer was extracted twice with EtOAc. The combined organic layers were dried over magnesium sulfate, filtered and evaporated to give 6.52 g (50%) of methyl 3-oxo-3-(pyrazin-2-yl)propanoate as a yellow solid.

Ethyl-3-(pyrazin-2-yl)-1H-pyrazol-5-ol: Ethylhydrazine oxalate (6.89 g, 45.9 mmol, 1 eq) was stirred with 450 mL of anhydrous ethanol for 10 min. To this was added methyl 3-oxo-3-(pyrazin-2-yl)propanoate (8.27 g, 45.9 mmol, 1 eq) and the mixture was refluxed for 10 hrs. The reaction was cooled, evaporated, taken into 300 ml of EtOAc, extracted with water and brine, dried over anhydrous magnesium, filtered and evaporated to yield 8.7 g of 1-ethyl-3-(pyrazin-2-yl)-1H-pyrazol-5-ol as a red oil. This material was used without further purification.

Trifluoro-methanesulfonic acid 2-ethyl-5-pyrazin-2-yl-2H-pyrazol-3-yl ester: To a stirred solution of 1-ethyl-3-(pyrazin-2-yl)-1H-pyrazol-5-ol (8.7 g, 45.7 mmol, 1 eq) in 230 mL DMF at 0° C. was added NaH (2.93 g, 73.2 mmol, 1.6 eq). The mixture was allowed to warm to rt and stirred for 1 hr. 1,1,1-Trifluoro-N-phenyl-N-(trifluoromethylsulfonyl)methanesulfonamide (24.5 g, 68.6 mmol, 1.5 eq) was added and stirred at RT for 90 min. The mixture was cooled in an ice bath, quenched with saturated ammonium chloride, evaporated and taken into EtOAc, extracted with water and brine, dried over anhydrous magnesium sulfate, filtered and evaporated to an oil. Flash chromatography on silica gel (400 g) using a gradient of 10-30% EtOAC/hexane gave 9.27 g (62.9%) of trifluoro-methanesulfonic acid 2-ethyl-5-pyrazin-2-yl-2H-pyrazol-3-yl ester as a white solid. LC-MS (ES) calculated for C₁₀H₉F₃N₄O₃S, 322.27; found m/z 322.9 [M+H]⁺.

### Intermediate 46

### Ethynyl-4-methylpyridine

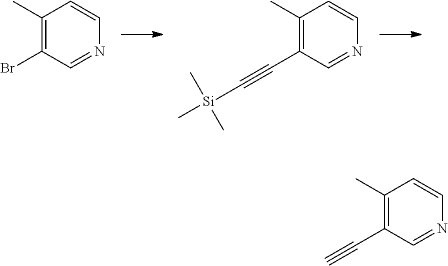

Methyl-3-((trimethylsilyl)ethynyl)pyridine: 3-bromo-4-methylpyridine (9.37 g, 54.5 mmol, Eq: 1.00), bis(triphenylphosphine)palladium(II) chloride (1.91 g, 2.72 mmol, Eq: 0.05), copper(I) iodide (519 mg, 2.72 mmol, Eq: 0.05) were added to anhydrous DMF (93.9 ml). ethynyltrimethylsilane (6.42 g, 9.17 ml, 65.4 mmol, Eq: 1.2) and triethylamine (22.0 g, 30.4 ml, 218 mmol, Eq: 4) was added and heated to 115° C. under N₂ for 16 hrs. Diluted with DCM and water. Washed with water (2×) and brine (1×). Organic layer was dried down and still contained a significant amount of DMF. Diluted with ether and water. Washed with water (2×) and brine (1×). Collected organic layer and dried onto silica gel for purification using a 15-25% EtOAc/Hex gradient. Obtained 4-methyl-3-((trimethylsilyl)ethynyl)pyridine (6.78 g, 35.8 mmol, 66% yield) as a brown oil.

Ethynyl-4-methylpyridine: To a mixture of 4-methyl-3-((trimethylsilyl)ethynyl)pyridine (1 g, 5.28 mmol, Eq: 1.00) in MeOH (35.2 ml) was added potassium carbonate (1.09 g, 7.92 mmol, Eq: 1.5) and stirred at r.t. over night. Diluted with water followed by Et₂O. Washed with water (2×). Dried organic layer over MgSO₄ and removed solvent. Obtained 3-ethynyl-4-methylpyridine (340 mg, 2.9 mmol, 55% yield) as an orange oil.

### Intermediate 47

### 1,3-Dichloro-2-ethynyl-benzene

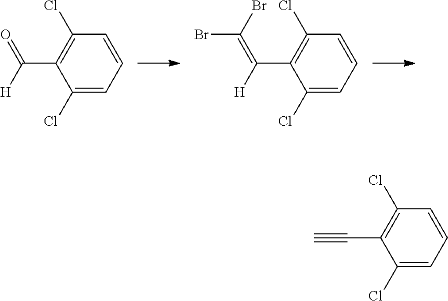

1,3-Dichloro-2-(2,2-dibromo-vinyl)-Benzene: To a stirred solution of 2,6-dichlorobenzaldehyde (2 gm, 11.42 mmol) in DCM (15 ml) was added PPh₃ (6 gm, 22.85 mmol) and CBr₄ (4.16 g, 12.56 mmol) at 0° C. Then the reaction mixture was stirred at rt for 4 hrs, evaporated, and crude was purified by column chromatography (eluting with hexane) to obtain 1,3-Dichloro-2-(2,2-dibromo-vinyl)-benzene (1.5 gm, 40%) as a white solid.

1,3-Dichloro-2-ethynyl-benzene: To a stirred solution of 1,3-Dichloro-2-(2,2-dibromo-vinyl)-benzene (1 gm, 3.03 mmol) in THF (7 ml) was added n-BuLi (1.26M, 5 ml, 6.06 mmol) dropwise under argon at −78° C. The reaction mixture was then stirred for 1.5 hrs at −78° C., after which it was quenched with saturated NH₄Cl, and extracted with EtOAc. The organic phase was then washed with brine, dried, concentrated, and the crude mass purified column chromatography (eluting with hexane) to obtain 1,3-Dichloro-2-ethynyl-benzene (500 mg, 97%) as a white solid.

### Intermediate 48

### 2-Ethynyl-1,3-dimethyl-benzene

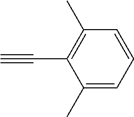

Prepared in a manner identical to Intermediate 47

### Intermediate 49

### 2-Ethynyl-1-fluoro-3-methyl-benzene

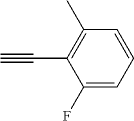

Prepared in a manner identical to Intermediate 47

### Preparation of Preferred Embodiments

### Example 1

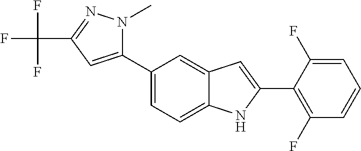

### 2-(2,6-Difluoro-phenyl)-5-(2-methyl-5-trifluoromethyl-2H-pyrazol-3-yl)-1H-indole

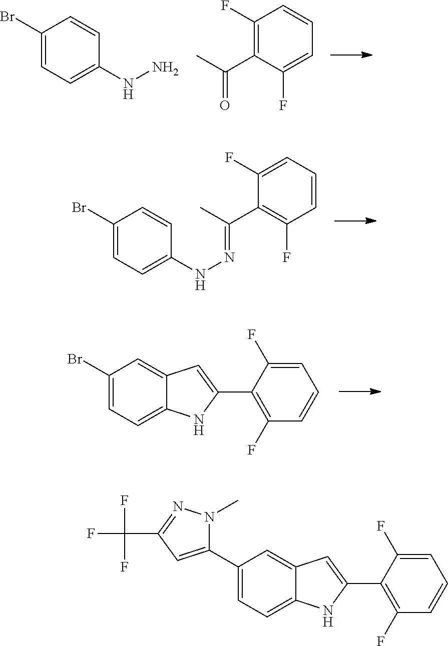

(4-Bromo-phenyl)-[1-(2,6-difluoro-phenyl)eth-(E)-ylidene]-amine: To a solution of 1-(2,6-difluoro-phenyl)-ethanone (1.4 g, 8.95 mmol) and 4-bromo-phenyl hydrazine (2 g, 8.95 mmol) in EtOH was added KOAc (0.88 g, 8.94 mmol). The reaction mixture was stirred at 25° C. for 16 h, then extracted with hexanes. The organic phase was washed with brine, dried over Na₂SO₄ and concentrated under reduced pressure to obtain crude (4-bromo-phenyl)-[1-(2,6-difluoro-phenyl)eth-(E)-ylidene]-amine (2 g, 69%), which was used directly without further purification.

Bromo-2-(2,6-difluoro-phenyl)-1H-indole: Polyphosphoric acid was heated to 70° C., and (4-bromo-phenyl)-[1-(2,6-difluoro-phenyl)eth-(E)-ylidene]-amine (2 g, 6.15 mmol) was added. The reaction mixture was heated to 130° C. for 2 h, then cooled to room temperature and diluted with ice-water. The mixture was extracted with EtOAc, and the organic layer was washed with brine, dried over Na₂SO₄ and concentrated under reduced pressure to give 5-bromo-2-(2,6-difluoro-phenyl)-1H-indole (1.35 g, 72%), which was used directly without further purification.

2-(2,6-Difluoro-phenyl)-5-(2-methyl-5-trifluoromethyl-2H-pyrazol-3-yl)-1H-indole: Bromo-2-(2,6-difluoro-phenyl)-1H-indole (270 mg) was added to dry DMF, under nitrogen atmosphere, followed by 1-methyl-3-trifluoromethyl-1H-pyrazol-3-yl boronic acid (203 mg) and Na₂CO₃ (139.5 mg, 1.5 equiv). The reaction mixture was degassed, and then water (1 mL) was added, followed by Pd(dppf)Cl₂*CH₂Cl₂ (101.26 ug). The reaction mixture was again degassed and then heated to 90° C. for six hours. The reaction mixture was cooled and concentrated under reduced pressure. The residue was diluted with water and EtOAc. The organic layer was separated, dried (Na₂SO₄), filtered and concentrated under reduced pressure. The residue was purified by flash chromatography (30% EtOAc in hexanes) to give 2-(2,6-difluoro-phenyl)-5-(2-methyl-5-trifluoromethyl-2H-pyrazol-3-yl)-1H-indole, MS (M+H)=378.

### Example 2

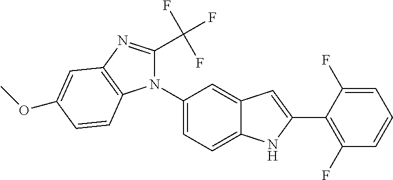

### 1-[2-(2,6-Difluoro-phenyl)-1H-indol-5-yl]-5-methoxy-2-trifluoromethyl-1H-benzoimidazole

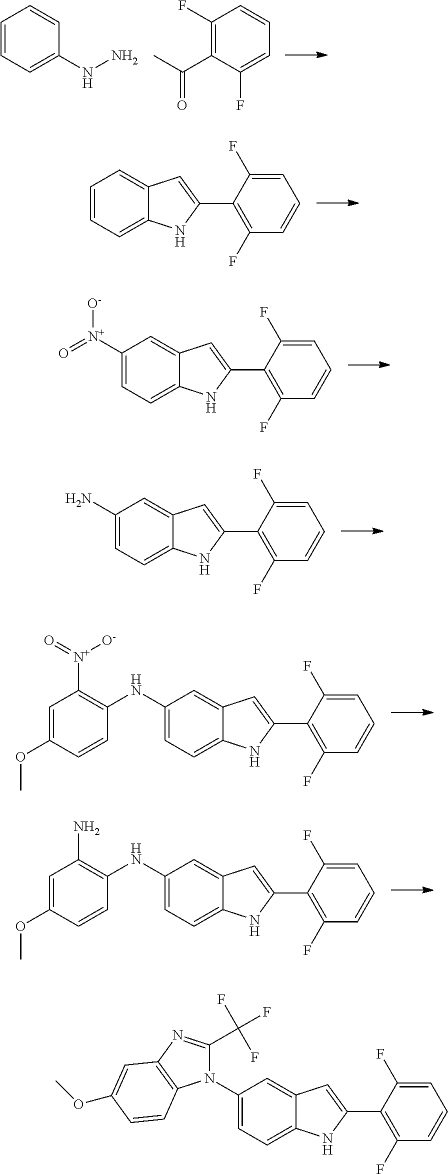

2-(2,6-Difluoro-phenyl)-1H-indole: To a stirred solution of phenylhydrazine (2.4 ml, 1.06 equiv) and 2′,6′-(difluoro)acetophenone (3 ml, 23 mmol) in EtOH (15 ml) and H₂O (6 ml) was added glacial acetic acid (two drops). The reaction mixture was stirred for 2 hours at room temperature, upon which an oil separated, which was extracted into hexane. The organic phase was washed with 1M HCl, water and brine, then dried over MgSO₄ and the solvent was removed under reduce pressure. The resulting oil was added to polyphosphoric acid (70 g) and the mixture was heated to 13° C. for 1 hour. The reaction mixture was poured onto ice water (500 ml). The resulting solid was filtered off and dried to give 2-(2,6-difluoro-phenyl)-1H-indole (3.97 g, 17.3 mmol) as a solid, which was used in the subsequent step without further purification.

2-(2,6-Difluoro-phenyl)-5-nitro-1H-indole: To a solution of 2-(2,6-difluoro-phenyl)-1H-indole (3.97 g, 17.3 mmol) in conc. H₂SO₄ (100 ml) cooled to 5° C., was added a solution of NaNO₃ (1.56 g, 1.06 equiv) in conc. H₂SO₄ (50 ml) at 5° C. The reaction mixture was stirred for 5 min at 5° C. and then poured onto ice (500 ml). The resulting precipitate formed was recovered by filtration and dissolved in EtOAc. The organic phase was washed with brine and dried over MgSO₄. The solvent was removed under reduce pressure and the remaining residue was purified on silica gel by flash chromatography (hexane: EtOAc 10%-80%) to yield 2-(2,6-difluoro-phenyl)-5-nitro-1H-indole (0.9 g) as a yellow solid.

2-(2,6-Difluoro-phenyl)-1H-indol-5-ylamine: To a solution of 2-(2,6-difluoro-phenyl)-5-nitro-1H-indole (0.9 g, 3.28 mmol) in EtOAc (40 ml) was added Pd/C (10%, 150 mg). The reaction mixture was evacuated and backfilled with nitrogen. This procedure was repeated twice. The reaction mixture was then evacuated and backfilled with hydrogen. The flask was fitted with a balloon filled with hydrogen and the reaction mixture was allowed to stir at room temperature for 4 hours. The reaction mixture was filtered through a pad of celite and the filtrate was concentrated under reduced pressure to give 2-(2,6-difluoro-phenyl)-1H-indol-5-ylamine as a yellow solid (quantitative yield).

[2-(2,6-Difluoro-phenyl)-1H-indol-5-yl]-(4-methoxy-2-nitro-phenyl)-amine: 2-(2,6-Difluoro-phenyl)-1H-indol-5-ylamine (329 mg, 1.35 equiv), 4-chloro-3-nitroanisole (169 mg, 0.9 mmol), Pd₂ dba₃ (8.2 mg, 1 mol %), 2-dicyclohexyl-phosphino-2′,4′,6′-triisopropylbiphenyl (22 mg, 5 mol %) and K₂CO₃ (311 mg, 2.5 equiv) were placed in a resealable tube fitted with a rubber septum. The tube was evacuated and backfilled with nitrogen. This procedure was repeated two times. The solids were dissolved in t-BuOH (3 ml) and the reaction mixture was heated to 110° C. for 4 hours. The reaction mixture was cooled to room temperature and filtered through a pad of celite. The solvent was removed under reduced pressure and the remaining residue was purified on silica gel by flash chromatography (hexane: EtOAc 10%-70%) to yield [2-(2,6-difluoro-phenyl)-1H-indol-5-yl]-(4-methoxy-2-nitro-phenyl)-amine (307 mg, 0.78 mmol) as a red solid.

N*1*-[2-(2,6-Difluoro-phenyl)-1H-indol-5-yl]-4-methoxy-benzene-1,2-diamine: To a solution of [2-(2,6-difluoro-phenyl)-1H-indol-5-yl]-(4-methoxy-2-nitro-phenyl)-amine (307 g, 0.78 mmol) in EtOAc (20 ml) was added Pd/C (10%, 150 mg). The reaction mixture was evacuated and backfilled with nitrogen. This procedure was repeated twice. The reaction mixture was then evacuated and backfilled with hydrogen. The flask was fitted with a balloon filled with hydrogen and the reaction mixture was allowed to stir at room temperature for 4 hours. The reaction mixture was filtered through a pad of celite and the solvent was removed under reduced pressure to give N*1*-[2-(2,6-difluoro-phenyl)-1H-indol-5-yl]-4-methoxy-benzene-1,2-diamine as a yellow solid (275 mg, 0.751 mmol).

1-[2-(2,6-Difluoro-phenyl)-1H-indol-5-yl]-5-methoxy-2-trifluoromethyl-1H-benzoimidazole: Trifluoroacetic anhydride (40 μl, 1.5 equiv) was added to a solution of compound N*1*-[2-(2,6-difluoro-phenyl)-1H-indol-5-yl]-4-methoxy-benzene-1,2-diamine (70 mg, 0.19 mmol) in benzene (2 ml) at room temperature. The reaction mixture was stirred for 10 minutes at room temperature. The solvent was removed under reduced pressure and the remaining residue was purified on silica gel by flash chromatography (hexane: EtOAc 10%-70%) to yield compound 1-[2-(2,6-Difluoro-phenyl)-1H-indol-5-yl]-5-methoxy-2-trifluoromethyl-1H-benzoimidazole (64 mg) as an orange solid, MS (M+H)=444.

### Example 3

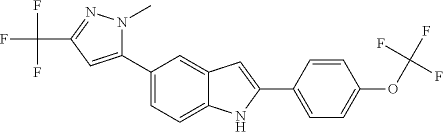

### 5-(2-Methyl-5-trifluoromethyl-2H-pyrazol-3-yl)-2-(4-trifluoromethoxy-phenyl)-1H-indole

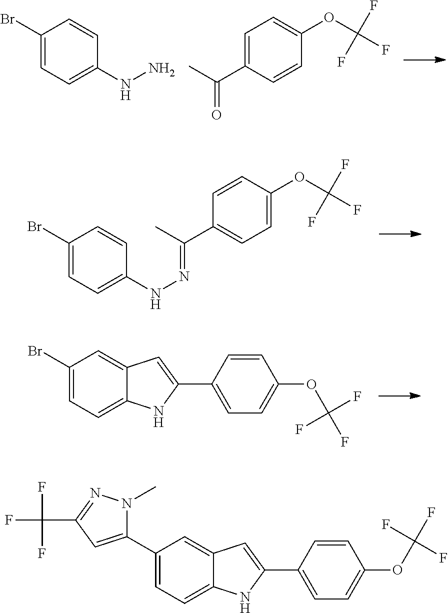

### 5-Bromo-2-(4-trifluoromethoxy-phenyl)-1H-indole

To a stirred solution of p-bromophenylhydrazine monohydrochloride (4.47 g, 20 mmol) and 4′-(trifluoromethoxy)acetophenone (3.19 ml, 1 equiv) in EtOH (200 ml) and H2O (66 ml) was added NaOAc (2.72 g, 1 equiv) in one portion. The reaction mixture was stirred for 12 h at room temperature, then concentrated under reduced pressure. The resulting solid was collected by filtration and dissolved in EtOAc, and the solution was dried over MgSO₄. The solvent was removed under reduce pressure and the residue was added to polyphosphoric acid (70 g). The resulting mixture was heated to 140° C. for 1 h, then poured onto ice water (500 ml). The resulting solid was recovered by filtration and purification on silica gel by flash chromatography (hexane: EtOAc 10%-50%) yielded 5-bromo-2-(4-trifluoromethoxy-phenyl)-1H-indole (3.44 g, 9.65 mmol) as a yellow solid, MS (M+H)=426.

### Example 4

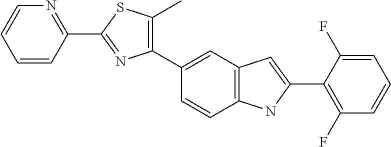

### 2-(2,6-Difluoro-phenyl)-5-(5-methyl-2-pyridin-2-yl-thiazol-4-yl)-1H-indole

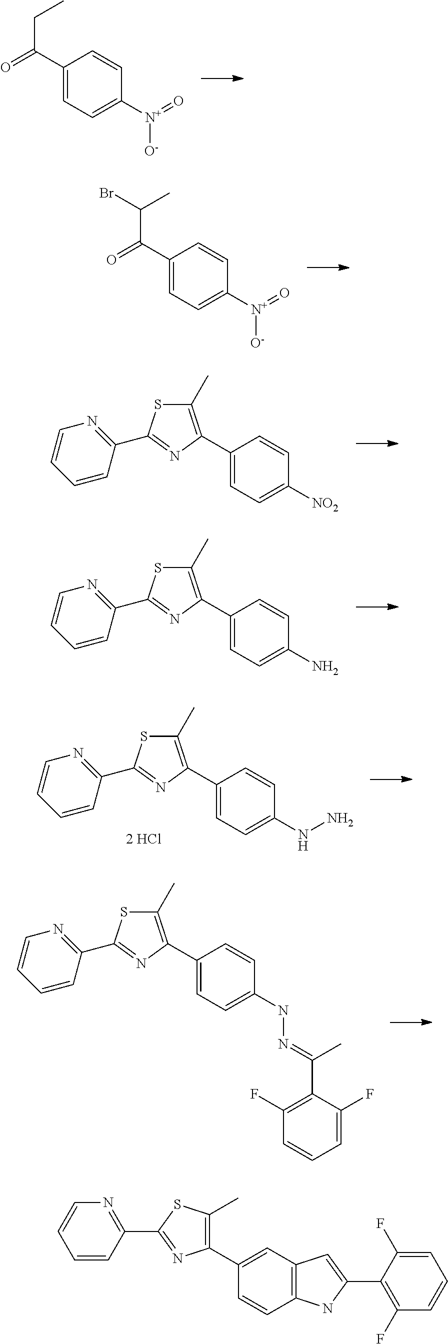

2-Bromo-1-(4-nitro-phenyl)-propan-1-one: To a solution of 1-(4-Nitro-phenyl)-propan-1-one (J. Med. Chem. 2005, 48, 6066-6083-4.37 g, 24.4 mmol) in CCl₄ (32 mL) was added a solution of bromine (3.89 g, 24.4 mmol) in CCl₄ (16 mL) dropwise at room temperature. The mixture was stirred for 1 h at which point it was quenched with 10% sodium thiosulfate. The organic layer was separated, dried with MgSO₄, and concentrated, to give 2-Bromo-1-(4-nitro-phenyl)-propan-1-one (6.13 g, 97% yield).

2-[5-Methyl-4-(4-nitro-phenyl)-thiazol-2-yl]-pyridine: To a solution of 2-Bromo-1-(4-nitro-phenyl)-propan-1-one (6.13 g, 23.75 mmol) in absolute EtOH (200 ml) was added pyridine-2-carbothioic acid amide (3.28 g, 23.75 mmol). The mixture was heated to reflux for 2 h, after which it was concentrated to dryness, and the resulting solid was filtered and washed with Et2O to provide 2-[5-Methyl-4-(4-nitro-phenyl)-thiazol-2-yl]-pyridine (6.08 g, 85%) as a solid.

4-(5-Methyl-2-pyridin-3-yl-thiazol-4-yl)-phenylamine: To a solution of 3-[5-Methyl-4-(4-nitro-phenyl)-thiazol-2-yl]-pyridine (80 mg, 0.27 mmol) in EtOAc (10 ml), was added and 10% Pd/C (20 mg), and the mixture hydrogenated for 18 hours under a hydrogen atmosphere. The reaction mixture was vacuum purged with argon (3×), and filtered through a plug of celite using DCM. The filtrate was concentrated to provide 61 mg (85%) of 4-(5-Methyl-2-pyridin-3-yl-thiazol-4-yl)-phenylamine as a yellow solid.

[4-(5-Methyl-2-pyridin-3-yl-thiazol-4-yl)-phenyl]-hydrazine bis hydrochloride salt: To a solution of conc. HCl (27 ml) was added solid 4-(5-Methyl-2-pyridin-3-yl-thiazol-4-yl)-phenylamine (1.0 g, 3.74 mmol) at 0° C. The resulting red solution was treated dropwise with NaNO₂ (645 mg, 9.35 mmol) in deionized water (1.0 ml) and after stirring for 3 hours at 0° C., SnCl2 (3.19 g, 16.83 mmol) dissolved in 3 ml of conc. HCl was added dropwise to the reaction mixture. The resulting thick yellow reaction mixture was treated with 3 ml of conc. HCl, and allowed to stir at room temperature for 2 days. The resulting yellow solid was filtered, rinsed with hexanes, and dried in the vacuum over at 40° C. for 1 hour affording 4-(5-Methyl-2-pyridin-3-yl-thiazol-4-yl)-phenyl]-hydrazine bis hydrochloride salt 2.2 grams (100%)

N-[1-(2,6-Difluoro-phenyl)-eth-(E)-ylidene]-N′-[4-(5-methyl-2-pyridin-2-yl-thiazol-4-yl)-phenyl]-hydrazine: 4-(5-Methyl-2-pyridin-2-yl-thiazol-4-yl)-phenylamine (393 mg, 1.1 mmol), 1-(2,6-difluoro-phenyl)-ethanone (173 mg, 1.1 mmol), and NaOAc (273 mg, 3.3 mmol) were stirred in EtOH (6.5 ml) and water (2.2 ml) for 2 days. The reaction mixture was partitioned between EtOAc/water and the organic layer was collected, dried over MgSO4, filtered, and concentrated. The crude product was purified by silica gel chromatography using 5-50% EtOAc/Hex as eluant to give N-[1-(2,6-Difluoro-phenyl)-eth-(E)-ylidene]-N′-[1-(5-methyl-2-pyridin-2-yl-thiazol-4-yl)-phenyl]-hydrazine (90 mg, 20%)

2-(2,6-Difluoro-phenyl)-5-(5-methyl-2-pyridin-2-yl-thiazol-4-yl)-1H-indole: To N-[1-(2,6-Difluoro-phenyl)-eth-(E)-ylidene]-N′-[4-(5-methyl-2-pyridin-2-yl-thiazol-4-yl)-phenyl]-hydrazine (90 mg, 0.214 mmol) was added to polyphosphoric acid (−2 g) and the reaction mixture was heated to 130° C. for 2 h. The mixture was then cooled to room temperature, diluted with ice-water, extracted with EtOAc. The organic layer was washed with brine, dried over Na₂SO₄, concentrated under reduced pressure, and the residue purified by chromatorgraphy (5% to 50% EtOAc/Hex) to give 2-(2,6-Difluoro-phenyl)-5-(5-methyl-2-pyridin-2-yl-thiazol-4-yl)-1H-indole (8.1 mg, 9%), MS (M+H)=404.

### Example 5

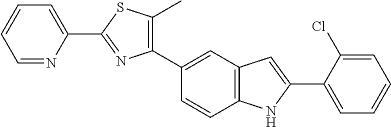

### 2-(2-Chloro-phenyl)-5-(5-methyl-2-pyridin-2-yl-thiazol-4-yl)-1H-indole

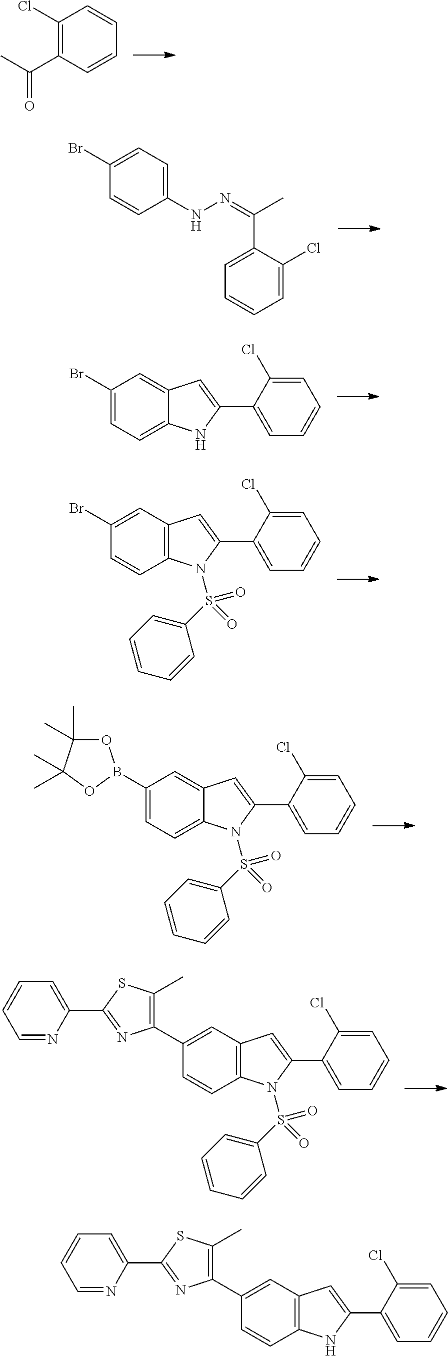

N-(4-Bromo-phenyl)-N′-[1-(2-chloro-phenyl)-eth-(Z)-ylidene]-hydrazine: To a solution of 1-(2-Chloro-phenyl)-ethanone (6.9 g, 44.74 mmol) and 4-bromo-phenylhydrazine hydrochloride (10 g, 44.74 mmol) in EtOH was added KOAc (4.39 g, 44.74 mmol). The mixture was stirred at 25° C. for 16 h, after which it was extracted with hexane (4×70 mL), the organic phase was washed with brine, dried over Na₂SO₄ and concentrated to obtain N-(4-Bromo-phenyl)-N′-[1-(2-chloro-phenyl)-eth-(Z)-ylidene]-hydrazine (11.05 g, 76%).

5-Bromo-2-(2-chloro-phenyl)-1H-indole: To PPA (33.52 g, 0.34 mol) heated to 70° C. was added N-(4-Bromo-phenyl)-N-[1-(2-chloro-phenyl)-eth-(Z)-ylidene]-hydrazine (11.05 g, 0.034 mol). The reaction mixture was then heated to 120° C. for 2 h, after which it was cooled, ice-water was added, and the dark solution extracted with EtOAc (3×25 mL). The organic layer was washed with brine, dried over Na₂SO₄, concentrated to give 5-Bromo-2-(2-chloro-phenyl)-1H-indole (5 g, 48%).

Benzenesulfonyl-5-bromo-2-(2-chloro-phenyl)-1H-indole: To a solution of 5-Bromo-2-(2-chloro-phenyl)-1H-indole (1 g, 3.26 mmol) in DMF at 0° C. was added NaH (0.117 g, 4.9 mmol). The mixture was stirred for 30 min, at which point benzenesulfonylchloride (0.69 g, 3.92 mmol) was added dropwise at 0° C. Stirring was continued to 25° C., and after 2 h, the mixture was quenched with ice-water, extracted with EtOAc (3×50 mL). The organic phase was washed with brine, dried over Na₂SO₄, and purified chromatography to give 1-Benzenesulfonyl-5-bromo-2-(2-chloro-phenyl)-1H-indole (1.05 g, 72%).

Benzenesulfonyl-2-(2-chloro-phenyl)-5-(4,4,5,5-tetramethyl-[1,3,2]dioxaborolan-2-yl)-1H-indole: To a solution of 1-Benzenesulfonyl-5-bromo-2-(2-chloro-phenyl)-1H-indole (2.85 g, 6.39 mmol) in 1,4-dioxane was added bispinacolatodiboron (3.24 g, 12.78 mmol) followed by KOAc (1.56 g, 15.97 mmol). The mixture was degassed and purged with nitrogen (10 min), and Pd(dppf)Cl₂ (10 mol %, 0.521 g) was then added. The reaction mixture was stirred at 100° C. for 14 h, after which it was filtered through Celite. The filtrate was extracted with EtOAc (3×60 mL) and the organic phase was washed with brine, dried over Na₂SO₄, concentrated, and the crude material purified by column chromatography (2% EtOAc-Hexane) to give 1-Benzenesulfonyl-2-(2-chloro-phenyl)-5-(4,4,5,5-tetramethyl-[1,3,2]dioxaborolan-2-yl)-1H-indole (1 g, 32%).

Benzenesulfonyl-2-(2-chloro-phenyl)-5-(5-methyl-2-pyridin-2-yl-thiazol-4-yl)-1H-indole: To a solution of trifluoro-methanesulfonic acid 5-methyl-2-pyridin-2-yl-thiazol-4-yl ester (Intermediate 1, 150 mg, 0.46 mmol) and 1-Benzenesulfonyl-2-(2-chloro-phenyl)-5-(4,4,5,5-tetramethyl-[1,3,2]dioxaborolan-2-yl)-1H-indole (200 mg, 0.41 mmol) in 1,4-dioxane (3 mL) was added aqueous K₂CO₃ (2 M, 0.3 mL) followed by Pd(dppf)Cl₂ (10 mol %, 0.025 g). The mixture degassed, sealed, and stirred at 100° C. for 10 h. Upon cooling the mixture was filtered through Celite, and the filtrate extracted with EtOAc (3×20 mL). The organic phase (EtOAc layer) was washed with brine, dried over Na₂SO₄, concentrated, and purified by column chromatography (10% EtOAc-Hexane) to give 1-Benzenesulfonyl-2-(2-chloro-phenyl)-5-(5-methyl-2-pyridin-2-yl-thiazol-4-yl)-1H-indole (100 mg, 40%).

2-(2-Chloro-phenyl)-5-(5-methyl-2-pyridin-2-yl-thiazol-4-yl)-1H-indole: To a solution of 1-Benzenesulfonyl-2-(2-chloro-phenyl)-5-(5-methyl-2-pyridin-2-yl-thiazol-4-yl)-1H-indole (130 mg, 0.24 mmol) in THF/MeOH (4:3) (6 mL) and was added Cs₂CO₃ (234 mg, 0.72 mmol). The mixture was stirred at 25° C. for 24 h, after which the solvent was removed and replaced with EtOAc. This was washed with brine, dried over Na₂SO₄, concentrated, and the crude material purified by column chromatography (10-20% EtOAc-Hexane) to obtain 2-(2-Chloro-phenyl)-5-(5-methyl-2-pyridin-2-yl-thiazol-4-yl)-1H-indole (15 mg, 16%), MS (M+H)=402.

### Example 6

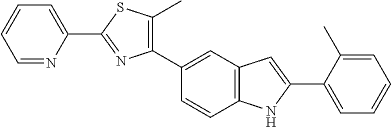

### 5-(5-Methyl-2-pyridin-2-yl-thiazol-4-yl)-2-o-tolyl-1H-indole

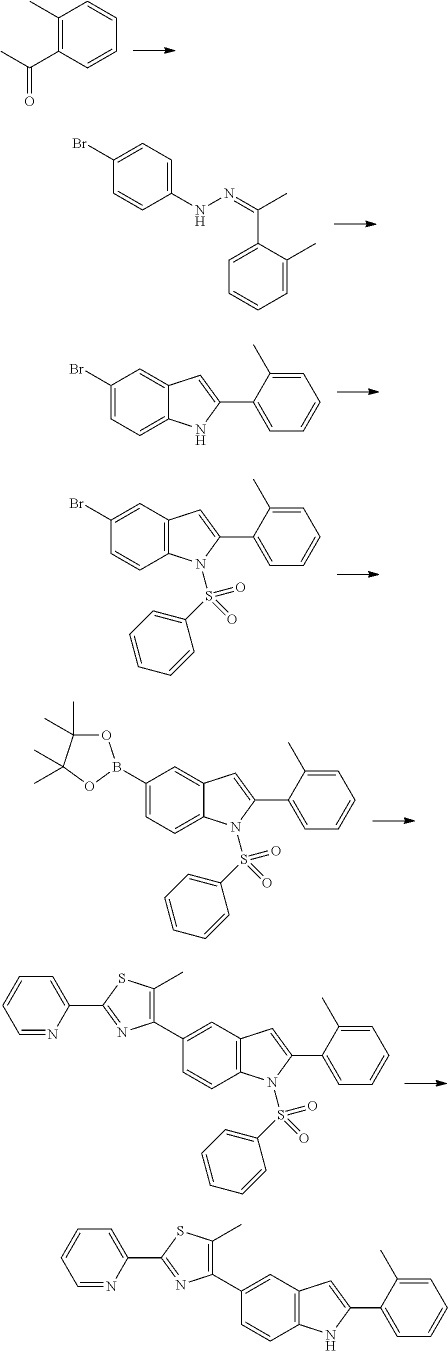

N-(4-Bromo-phenyl)-N′-[1-o-tolyl-eth-(Z)-ylidene]-hydrazine: To a solution of 1-(2-Methyl-phenyl)-ethanone (3 g, 22.37 mmol) and 4-bromo-phenylhydrazine hydrochloride (5 g, 22.37 mmol) in EtOH was added KOAc (2.19 g, 22.37 mmol) and the mixture stirred at 25° C. After 16 h, the mixture was extracted with hexane (3×50 mL), washed with brine, dried over Na₂SO₄, and concentrated to give N-(4-Bromo-phenyl)-N′-[1-o-tolyl-eth-(Z)-ylidene]-hydrazine (5.7 g, 84%).

5-Bromo-2-o-tolyl-1H-indole: To PPA (18.43 g, 0.18 mol) heated to 70° C. was added N-(4-Bromo-phenyl)-N′-[1-o-tolyl-eth-(Z)-ylidene]-hydrazine (5.7 g, 18.81 mmol). The reaction mixture was then heated to 120° C. for 2 h, after which it was cooled, ice-water was added, and the dark solution extracted with EtOAc (4×60 mL). The organic layer was washed with brine, dried over Na₂SO₄, concentrated to give 5-Bromo-2-o-tolyl-1H-indole (2 g, 37%).

Benzenesulfonyl-5-bromo-2-o-tolyl-1H-indole: To a solution of 5-Bromo-2-o-tolyl-1H-indole (1.7 g, 5.94 mmol) in DMF at 0° C. was added NaH (0.213 g, 8.91 mmol). The mixture was stirred for 30 min, after which benzenesulfonylchloride (1.25 g, 7.13 mmol) was added dropwise at 0° C. Stirring was continued to 25° C., and after 2 h, the mixture was quenched with ice-water, extracted with EtOAc (3×50 mL). The organic phase was washed with brine, dried over Na₂SO₄, and purified chromatography to give 1-Benzenesulfonyl-5-bromo-2-o-tolyl-1H-indole (2.3 g, 82%).

1-Benzenesulfonyl-5-(4,4,5,5-tetramethyl-[1,3,2]dioxaborolan-2-yl)-2-o-tolyl-1H-indole: To a solution of 1-Benzenesulfonyl-5-bromo-2-o-tolyl-1H-indole (200 mg, 0.47 mmol) in 1,4-dioxane (6 ml) was added bispinacolatodiboron (237 mg, 0.94 mmol) and KOAc (92 mg, 0.93 mmol). The mixture was degassed and purged with nitrogen (10 min), and Pd(dppf)Cl₂ (10 mol %, 38 mg) was then added. The reaction mixture was stirred at 100° C. for 14 h, after which it was filtered through Celite. The filtrate was extracted with EtOAc (3×60 mL) and the organic phase was washed with brine, dried over Na₂SO₄, concentrated, and the crude material purified by column chromatography (2% EtOAc-Hexane) to obtain 1-Benzenesulfonyl-5-(4,4,5,5-tetramethyl-[1,3,2]dioxaborolan-2-yl)-2-o-tolyl-1H-indole (90 mg, 41%).

Benzenesulfonyl-5-(5-methyl-2-pyridin-2-yl-thiazol-4-yl)-2-o-tolyl-1H-indole: To a solution of trifluoro-methanesulfonic acid 5-methyl-2-pyridin-2-yl-thiazol-4-yl ester (Intermediate 1, 68.5 mg, 0.21 mmol) and 1-Benzenesulfonyl-5-(4,4,5,5-tetramethyl-[1,3,2]dioxaborolan-2-yl)-2-o-tolyl-1H-indole (100 mg, 0.21 mmol) in 1,4-dioxane (2 mL) was added aqueous K₂CO₃ (2 M, 0.31 mL), followed by Pd(dppf)Cl₂ (10 mol %, 17.2 mg). The mixture degassed, sealed, and stirred at 100° C. for 10 h. Upon cooling the mixture was filtered through Celite, and the filtrate extracted with EtOAc (3×20 mL). The organic phase (EtOAc layer) was washed with brine, dried over Na₂SO₄, concentrated, and purified by column chromatography (10% EtOAc-Hexane) to give 1-Benzenesulfonyl-5-(5-methyl-2-pyridin-2-yl-thiazol-4-yl)-2-o-tolyl-1H-indole (40 mg, 36.5%).

5-(5-Methyl-2-pyridin-2-yl-thiazol-4-yl)-2-o-tolyl-1H-indole: To a solution of 1-Benzenesulfonyl-5-(5-methyl-2-pyridin-2-yl-thiazol-4-yl)-2-o-tolyl-1H-indole (100 mg, 0.19 mmol) in THF/MeOH (4:3) (6 mL) was added Cs₂CO₃ (188 mg, 0.58 mmol) at 25° C. The mixture was stirred at 25° C. for 24 h, after which the solvent was removed and replaced with EtOAc. This was washed with brine, dried over Na₂SO₄, concentrated, and the crude material purified by column chromatography (10-20% EtOAc-Hexane) to give 5-(5-Methyl-2-pyridin-2-yl-thiazol-4-yl)-2-o-tolyl-1H-indole (20 mg, 27%), MS (M+H)=382.

### Example 7

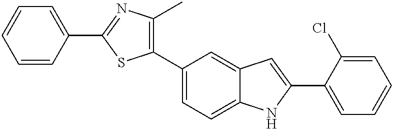

### 2-(2-Chloro-phenyl)-5-(4-methyl-2-phenyl-thiazol-5-yl)-1H-indole

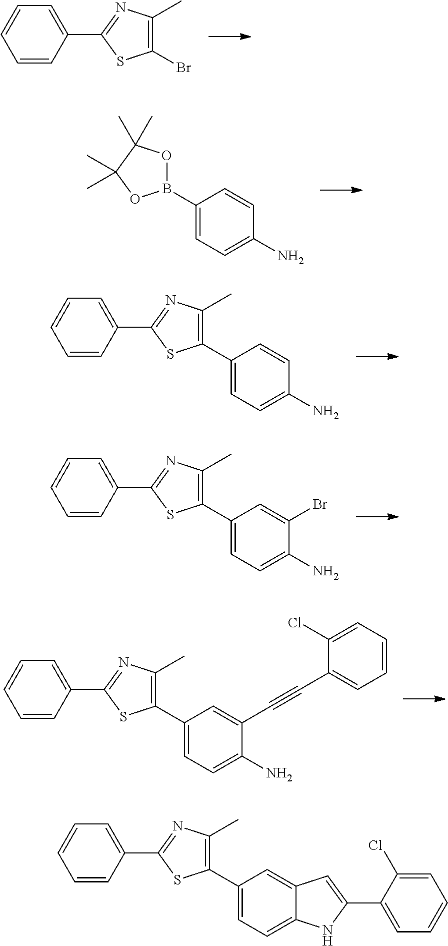

4-(4-Methyl-2-phenyl-thiazol-5-yl)-phenylamine: A suspension of 5-bromo-4-methyl-2-phenyl-thiazole (1.0 g, 3.93 mmol, 1 eq), 4-aminophenyl pinacolatoboronic ester (0.95 g, 4.33 mmol, 1.1 eq), Pd(PPh₃)₄ (0.225 g, 0.20 mmol, 5 mol %), Na₂CO₃ (1.15 g, 10.8 mmol, 2.74 eq) in a mixture of toluene/EtOH/H₂O (40 mL, 40 mL, 20 mL) was heated at 90° C. for 18 h. The mixture was cooled to room temperature, diluted with water, and extracted with EtOAc. The organic layer was separated, dried (MgSO₄), filtered and concentrated under reduced pressure. The residue was flash chromatographed (SiO₂, 27% EtOAc/hexanes) to give 4-(4-Methyl-2-phenyl-thiazol-5-yl)-phenylamine as a yellow solid (0.993 g, 95%).

Bromo-4-(4-methyl-2-phenyl-thiazol-5-yl)-phenylamine: To a suspension of 4-(4-methyl-2-phenyl-thiazol-5-yl)-phenylamine (0.993 g, 3.75 mmol, 1.0 eq) in DCM (25 mL) at 0° C. was added NBS (0.664 g, 3.73 mmol, 1.0 eq). The suspension dissolved, changing color to orange. After 20 minutes solvent was removed under reduced pressure, and the resulting yellow oil was flash chromatographed (25 g SiO₂, 10-15% EtOAc/hexanes) to give 2-bromo-4-(4-methyl-2-phenyl-thiazol-5-yl)-phenylamine (0.427 g, 33%).

2-(2-Chloro-phenylethynyl)-4-(4-methyl-2-phenyl-thiazol-5-yl)-phenylamine: To a solution of 2-bromo-4-(4-methyl-2-phenyl-thiazol-5-yl)-phenylamine (0.200 g, 0.579 mmol, 1.0 eq), 2-chlorophenyl acetylene (0.079 g, 0.070 mL, 0.579 mmol, 1.0 eq), PdCl₂(PPh₃)₂ (0.020 g, 0.029 mmol, 0.05 eq) and CuI (0.011 g, 0.0579 mmol, 0.10 eq) in DMF (1 mL) was added TEA (0.352 g, 0.482 mL, 3.47 mmol, 6 eq). The reaction mixture was heated at 110° C. for 4 h, then cooled and poured into saturated aqueous NH₄Cl. The organic layer was separated, dried (MgSO₄), filtered, and concentrated in vacuo to give an orange solid, which was first flash chromatographed (15-20% EtOAc/hexanes) and then further purified on a prep TLC plate (20% EtOAc/hexanes) to give 2-(2-chloro-phenylethynyl)-4-(4-methyl-2-phenyl-thiazol-5-yl)-phenylamine as an orange oil (0.086 g, 37%).

2-(2-Chloro-phenyl)-5-(4-methyl-2-phenyl-thiazol-5-yl)-1H-indole: A solution of 2-(2-chloro-phenylethynyl)-4-(4-methyl-2-phenyl-thiazol-5-yl)-phenylamine (0.086 g, 0.215 mmol, 1.0 eq) and potassium tert-butoxide (0.072 g, 0.644 mmol, 3.0 eq) in NMP (1 mL) was heated at 70° C. for 3 h. The orange mixture was cooled to room temperature and poured into saturated aqueous NH₄Cl and EtOAc. The organic layer was separated, dried (MgSO₄), filtered, and concentrated in vacuo to give a yellow solid, which was flash chromatographed (20% EtOAc/hexanes) and then repurified on a prep TLC plate (20% EtOAc/hexanes) to give 2-(2-chloro-phenyl)-5-(4-methyl-2-phenyl-thiazol-5-yl)-1H-indole (0.009 g, 10%), MS (M+H)=402.

### Example 8

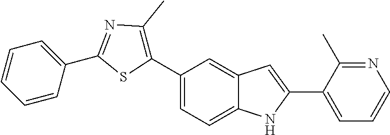

### 5-(4-Methyl-2-phenyl-thiazol-5-yl)-2-(2-methyl-pyridin-3-yl)-1H-indole

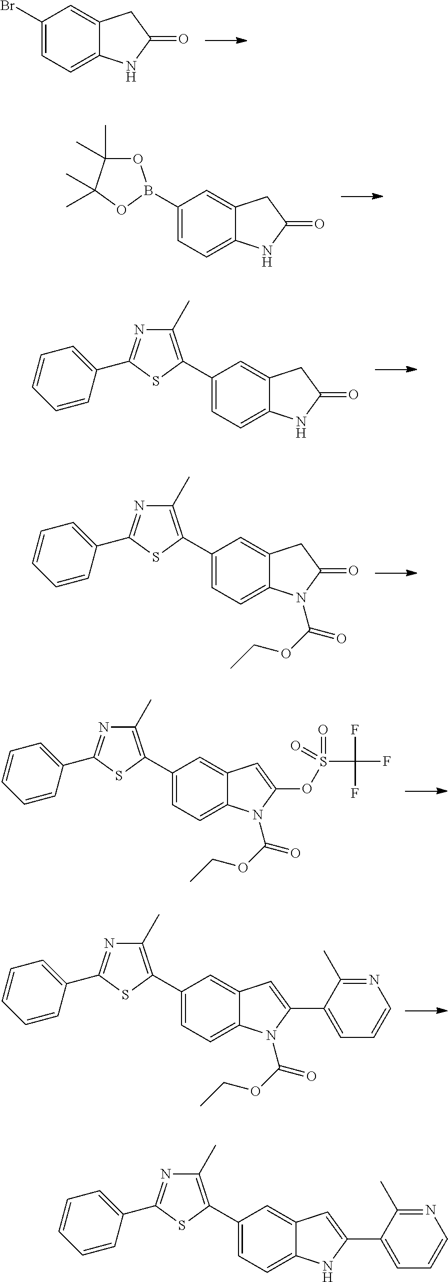

5-(4,4,5,5-Tetramethyl-[1,3,2]dioxaborolan-2-yl)-1,3-dihydro-indol-2-one: To a solution of 5-bromooxindole (4.407 g, 20.7 mmol, 1.0 eq), bispinacolatodiboron (6.33 g, 24.9 mmol, 1.2 eq), PdCl2(dppf)CH₂Cl₂ (1.69 g, 2.07 mmol, 0.10 eq), and KOAc (4.06 g, 41.4 mmol, 2 eq) in dioxane (207 mL, 0.1M) was heated at 90° C. for 18 h. Upon cooling, the mixture was washed with brine, concentrated, and chromatographed (40% EtOAc/Hexanes) to give a solid, which was triturated with Et2O to give 5-(4,4,5,5-Tetramethyl-[1,3,2]dioxaborolan-2-yl)-1,3-dihydro-indol-2-one (3.313 g) as a peach colored solid.

5-(4-Methyl-2-phenyl-thiazol-5-yl)-1,3-dihydro-indol-2-one: To a solution of 5-Bromo-4-methyl-2-phenyl-thiazole (0.100 g, 0.393 mmol, 1 eq) and 5-(4,4,5,5-Tetramethyl-[1,3,2]dioxaborolan-2-yl)-1,3-dihydro-indol-2-one (0.133 g, 0.512 mmol, 1.3 eq) in EtOH/dioxane/H2O (1:1:1 0.6 mL each) was added PdCl2(PPh3)₂ (0.014 g, 0.02 mmol, 5 mol %), 2-(dicyclohexyl phosphino)biphenyl (0.021 g, 0.059 mmol, 0.15 eq), and Na2CO3 (0.062 g, 0.589 mmol, 1.5 eq). The mixture was irradiated in a microwave 30 min at 130° C. After which the dark mixture was partitioned between sat. NH4Cl and EtOAc, and the organic layer was washed with brine, dried, concentrated, and chromatographed (40% EtOAc/Hexanes) to give 5-(4-Methyl-2-phenyl-thiazol-5-yl)-1,3-dihydro-indol-2-one (0.086 g, 71%). Note: this procedure was repeated on 0.500 g scale to give the same product (0.352 g, 58%).

5-(4-Methyl-2-phenyl-thiazol-5-yl)-2-oxo-2,3-dihydro-indole-1-carboxylic acid ethyl ester: To a solution of 5-(4-Methyl-2-phenyl-thiazol-5-yl)-1,3-dihydro-indol-2-one (352 mg, 1.149 mmol) in THF (4.5 mL) and TEA (1 mL, 6.894 mmol) at 0° C. was added Ethylchloroformate (0.547 mL, 5.74 mmol). The reaction mixture was warmed to rt and monitored by LC/MS. Upon full consumption of the starting material, the mixture was concentrated. The material was redissolved in DCM and washed with water and brine. The organic layer is separated, dried over sodium sulfate and concentrated. The oil there obtained was then redissolved in DMF (4 mL) at 0° C., and finely ground ammonium carbonate (110 mg, 1.149 mmol) was added. The mixture is stirred from 0° C. to rt for 2 h at which point the reaction was complete by LC/MS. The mixture was poured into water and extracted with DCM. After washing with brine the organic layer was dried with MgSO4, concentrated, and chromatographed directly (40% EtoAc/hex) to give 5-(4-Methyl-2-phenyl-thiazol-5-yl)-2-oxo-2,3-dihydro-indole-1-carboxylic acid ethyl ester (327 mg, 75%) as a yellow solid.

5-(4-Methyl-2-phenyl-thiazol-5-yl)-2-trifluoromethanesulfonyloxy-indole-1-carboxylic acid ethyl ester: To a solution of 5-(4-Methyl-2-phenyl-thiazol-5-yl)-2-oxo-2,3-dihydro-indole-1-carboxylic acid ethyl ester (47 mg, 0.124 mmol) in DCM (0.750 mL) and DIPEA (32 mg, 0.248 mmol) at 0° C. was added Tf₂O (46 mg, 0.162 mmol). The reaction mixture was stirred at this temperature for 1 h, at which point it was quenched with saturated NH₄Cl. This mixture was then extracted with EtOAc (2×20 mL) and the organic layer washed with brine, dried over Na₂SO₄, and concentrated. The crude compound was then purified by column chromatography (10-30% EtOAc-Hexane) to give 5-(4-Methyl-2-phenyl-thiazol-5-yl)-2-trifluoromethanesulfonyloxy-indole-1-carboxylic acid ethyl ester (41 mg, 65%).

5-(4-Methyl-2-phenyl-thiazol-5-yl)-2-(2-methyl-pyridin-3-yl)-indole-1-carboxylic acid ethyl ester: To a solution of 5-(4-Methyl-2-phenyl-thiazol-5-yl)-2-trifluoromethanesulfonyloxy-indole-1-carboxylic acid ethyl ester (30 mg, 0.061 mmol) and 2-methylpyridine-3-boronic acid (9 mg, 0.067 mmol) in toluene (0.67 mL) was added EtOH (0.44 mL) followed by sat. NaHCO₃ (0.30 mL). The mixture was purged with nitrogen (20 min), and then Pd(PPh₃)₄ (10 mol %, 7 mg) was added. After stirring for 18 h at 100° C. the mixture was filtered through Celite and EtOAc was added (30 mL). This mixture was washed with brine, dried over Na₂SO₄, concentrated, and purified by column chromatography (40% EtOAc-Hexane) to give 5-(4-Methyl-2-phenyl-thiazol-5-yl)-2-(2-methyl-pyridin-3-yl)-indole-1-carboxylic acid ethyl ester (13 mg).

5-(4-Methyl-2-phenyl-thiazol-5-yl)-2-(2-methyl-pyridin-3-yl)-1H-indole: To a solution of 5-(4-Methyl-2-phenyl-thiazol-5-yl)-2-(2-methyl-pyridin-3-yl)-indole-1-carboxylic acid ethyl ester (52 mg, 0.017 mmol) in THF (0.2 mL) andMeOH (0.2 mL) was added solid K₂CO₃ (16 mg, 0.115 mmol) at room temperature. After 1 h the mixture was filtered through Celite and EtOAc (60 mL) was added. This mixture was then washed with brine, dried over Na₂SO₄, concentrated, and the crude material purified by column chromatography (40% EtOAc-Hexane) to give 5-(4-Methyl-2-phenyl-thiazol-5-yl)-2-(2-methyl-pyridin-3-yl)-1H-indole (4 mg), MS (M+H)=382.

### Example 9

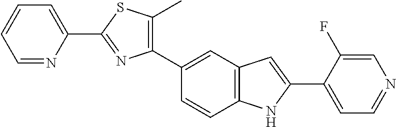

### 2-(3-Fluoro-pyridin-4-yl)-5-(5-methyl-2-pyridin-2-yl-thiazol-4-yl)-1H-indole

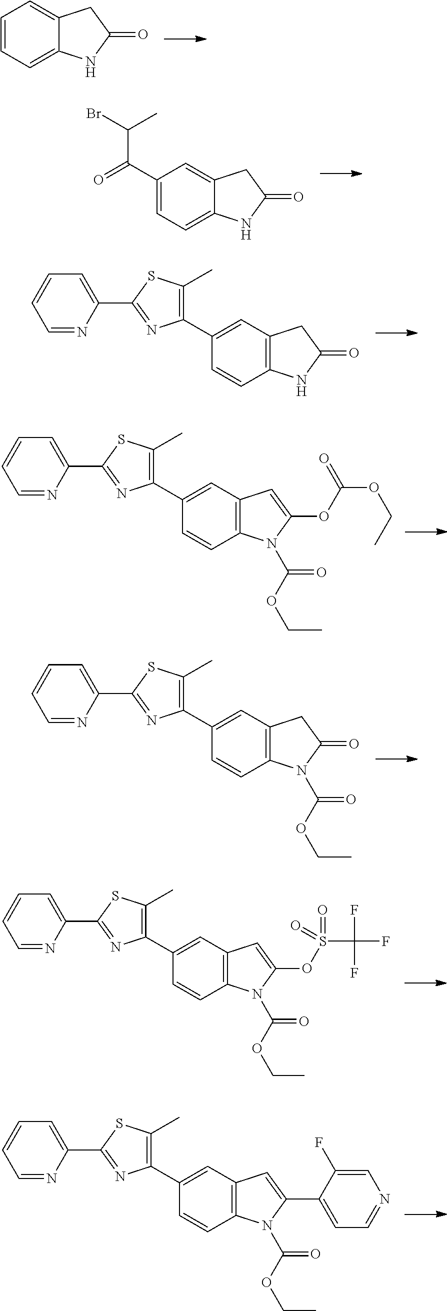

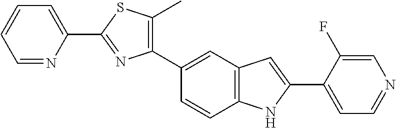

5-(2-Bromo-propionyl)-1,3-dihydro-indol-2-one: To a stirred suspension of oxindole (1 g, 7.51 mmol) and AlCl₃ (3 g, 22.53 mmol) in DCM was added 2-bromo-propionyl chloride (2.5 g, 15.02 mmol). The mixture was refluxed for 6 h, then cooled to room temperature and poured into ice-water. After stirring for 30 min, the solid formed was filtered to give 5-(2-Bromo-propionyl)-1,3-dihydro-indol-2-one (1.5 g, 75%).

5-(5-Methyl-2-pyridin-2-yl-thiazol-4-yl)-1,3-dihydro-indol-2-one: To a solution of 542-Bromo-propionyl)-1,3-dihydro-indol-2-one (3 g, 11.2 mmol) in EtOH was added Pyridine-2-carbothioic acid amide (1.85 g, 13.43 mmol). The mixture was heated to 80° C. for 18 h, after which it was poured into ice-water, and extracted with EtOAc (3×30 mL). The organic phase was washed with brine, dried over Na₂SO₄, and concentrated to give 5-(5-Methyl-2-pyridin-2-yl-thiazol-4-yl)-1,3-dihydro-indol-2-one (3.4 g, 99%).

Ethoxycarbonyloxy-5-(5-methyl-2-pyridin-2-yl-thiazol-4-yl)-indol-1-carboxylic acid ethyl ester: To a solution of 5-(5-Methyl-2-pyridin-2-yl-thiazol-4-yl)-1,3-dihydro-indol-2-one (10 g, 0.033 mol) in THF (130 mL) and triethylamine (27 mL, 0.195 mol) at 0° C. was added ethylchloroformate (15.6 mL, 0.162 mol). The reaction was warmed to room temperature and stirred at this temperature for 20 h. The solvent was then removed and the material redissolved in DCM, washed with water and brine, separated, dried over Na₂SO₄, and concentrated to give 2-Ethoxycarbonyloxy-5-(5-methyl-2-pyridin-2-yl-thiazol-4-yl)-indol-1-carboxylic acid ethyl ester (10.7 g, 73%).

5-(5-Methyl-2-pyridin-2-yl-thiazol-4-yl)-2-oxo-2,3-dihydro-indole-1-carboxylic acid ethyl ester: To a solution of 2-Ethoxycarbonyloxy-5-(5-methyl-2-pyridin-2-yl-thiazol-4-yl)-indol-1-carboxylic acid ethyl ester (3.2 g, 7.08 mmol) in DMF (5 mL) at 0° C. was added (NH₄)₂CO₃ (0.686 g, 7.08 mmol). The mixture was stirred from 0° C. to 25° C. over 3 h. The entire mixture was then poured into water and the solids collected by filtration to give 5-(5-Methyl-2-pyridin-2-yl-thiazol-4-yl)-2-oxo-2,3-dihydro-indole-1-carboxylic acid ethyl ester (1.5 g, 56%).

5-(5-Methyl-2-pyridin-2-yl-thiazol-4-yl)-2-trifluoromethanesulfonyloxy-indole-1-carboxylic acid ethyl ester: To a solution of 5-(5-Methyl-2-pyridin-2-yl-thiazol-4-yl)-2-oxo-2,3-dihydro-indole-1-carboxylic acid ethyl ester (500 mg, 1.32 mmol) in DCM (10 mL) and DIPEA (496 mg, 3.96 mmol) at 0° C. was added Tf₂O (559 mg, 1.98 mmol). The mixture was stirred at this temperature for 1 h, and then was quenched with saturated NH₄Cl. This was then extracted with DCM (2×20 mL) and the organic layer washed with brine, dried over Na₂SO₄, concentrated, and the crude material purified by chromatography (10-30% EtOAc-Hexane) to give 5-(5-Methyl-2-pyridin-2-yl-thiazol-4-yl)-2-trifluoromethanesulfonyloxy-indole-1-carboxylic acid ethyl ester (600 mg, 89%).

2-(3-Fluoro-pyridin-4-yl)-5-(5-methyl-2-pyridin-2-yl-thiazol-4-yl)-indole-1-carboxylic acid ethyl ester: To a solution of 5-(5-Methyl-2-pyridin-2-yl-thiazol-4-yl)-2-trifluoromethanesulfonyloxy-indole-1-carboxylic acid ethyl ester (70 mg, 0.137 mmol) and 3-fluoropyridine-4-boronic acid (21 mg, 0.150 mmol) in toluene (1.5 mL) and EtOH (1 mL) was added sat. NaHCO₃ (0.67 mL). This mixture was purged with nitrogen (20 min) and then Pd(PPh₃)₄ (10 mol %, 16 mg) was added. After stirring at 100° C. for 18 h, the mixture was filtered through Celite, and EtOAc (60 mL) was added. The organic phase was washed with brine, dried over Na₂SO₄, concentrated, and the crude material purified by column chromatography (33-66% EtOAc-Hexane) to give 2-(3-Fluoro-pyridin-4-yl)-5-(5-methyl-2-pyridin-2-yl-thiazol-4-yl)-indole-1-carboxylic acid ethyl ester (8 mg).

2-(3-Fluoro-pyridin-4-yl)-5-(5-methyl-2-pyridin-2-yl-thiazol-4-yl)-1H-indole: To a solution of 2-(3-Fluoro-pyridin-4-yl)-5-(5-methyl-2-pyridin-2-yl-thiazol-4-yl)-indole-1-carboxylic acid ethyl ester (8 mg, 0.017 mmol) in THF (0.2 mL) andMeOH (0.2 mL) was added solid K₂CO₃ at room temperature. After 1 h the mixture was filtered through Celite, and EtOAc (60 mL) added. The organic phase was washed with brine, dried over Na₂SO₄, concentrated, and the crude material purified by column chromatography (50-95% EtOAc-Hexane) to give 2-(3-Fluoro-pyridin-4-yl)-5-(5-methyl-2-pyridin-2-yl-thiazol-4-yl)-1H-indole (4 mg), MS (M+H)=387.

### Example 10

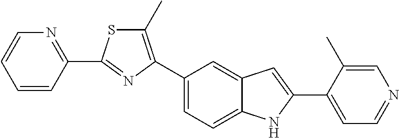

### 2-(3-Methyl-pyridin-4-yl)-5-(5-methyl-2-pyridin-2-yl-thiazol-4-yl)-1H-indole

Was prepared in a manner identical to that described above in Example 9 substituting 3-methyl-pyridine-4-boronic acid in the penultimate step. MS (M+H)=383.

### Example 11

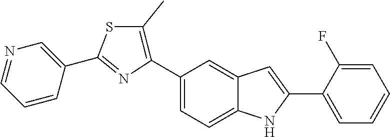

### 2-(2-Fluoro-phenyl)-5-(5-methyl-2-pyridin-3-yl-thiazol-4-yl)-1H-indole

Was prepared in a manner similar to that described above in Example 4 substituting thionicotinamide in the thiazole synthesis and 2′-fluoroacetophenone in the penultimate step. MS (M+H)=386.

### Example 12

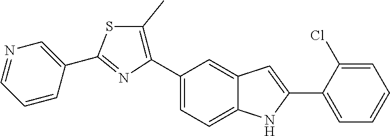

### 2-(2-Chloro-phenyl)-5-(5-methyl-2-pyridin-3-yl-thiazol-4-yl)-1H-indole

Was prepared in a manner similar to that described above in Example 4 substituting thionicotinamide in the thiazole synthesis and 2′-chloroacetophenone in the penultimate step. MS (M+H)=402.

### Example 13

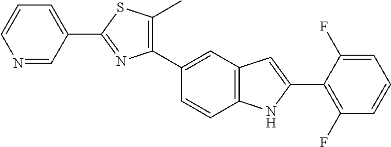

### 2-(2,6-Difluoro-phenyl)-5-(5-methyl-2-pyridin-3-yl-thiazol-4-yl)-1H-indole

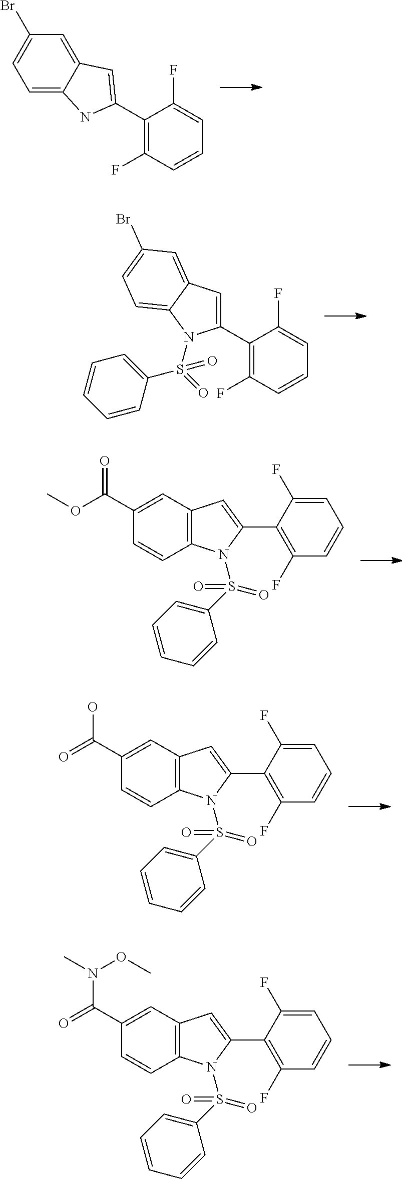

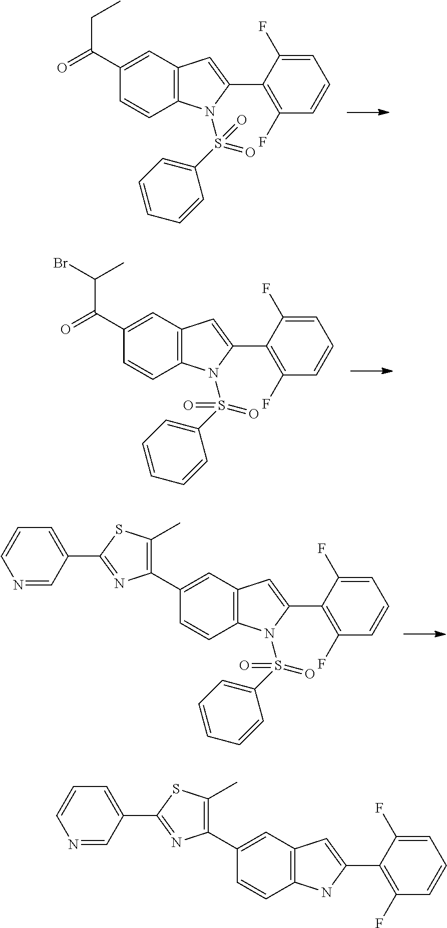

Benzenesulfonyl-5-bromo-2-(2,6-difluoro-phenyl)-1H-indole: To a solution of 5-bromo-2-(2,6-difluoro-phenyl)-1H-indole (2 g, 6.49 mmol) in DMF at 0° C. was added NaH (0.233 g, 9.74 mmol) and stirred for 30 min. Benzenesulfonylchloride (1.37 g, 7.79 mmol) was added dropwise at 0° C. and stirred at 25° C. for 2 h. The reaction was quenched with ice-water, extracted with EtOAc, brine, dried, concentrated and purified by column chromatography to yield 1-benzenesulfonyl-5-bromo-2-(2,6-difluoro-phenyl)-1H-indole (2.1 g, 73%).

Benzenesulfonyl-2-(2,6-difluoro-phenyl)-1H-indole-5-carboxylic acid methyl ester: To a solution of 1-benzenesulfonyl-5-bromo-2-(2,6-difluoro-phenyl)-1H-indole (2 g, 4.45 mmol) in MeOH (50 ml) and triethylamine (0.25 ml, 1.78 mmol), purged with nitrogen for 20 min, was added 1,3-bis(diphenylphosphino)propane (550 mg, 1.33 mmol) and Pd(OAc)₂ (149 mg, 0.668 mmol). The mixture was stirred in autoclave at 220 psi (CO pressure) at 80° C. for 12 h. The reaction mixture was filtered through Celite and the filtrate was concentrated. The crude compound was purified by column chromatography to yield 1-benzenesulfonyl-2-(2,6-difluoro-phenyl)-1H-indole-5-carboxylic acid methyl ester (1.2 g, 63%).

Benzenesulfonyl-2-(2,6-difluoro-phenyl)-1H-indole-5-carboxylic acid: 1-Benzenesulfonyl-2-(2,6-difluoro-phenyl)-1H-indole-5-carboxylic acid methyl ester (1.3 g, 3 mmol) was dissolved in THF-MeOH—H2O (20 ml-10 ml-5 ml) and LiOH.2H₂O (251 mg, 6 mmol) was added. The mixture was stirred at RT for 6 h. After the completion, solvent was removed under vacuum and the residue was acidify with HCl (1M) to pH 1 and extracted with DCM. The organic phase was washed with brine, dried over Na₂SO₄ and concentrated. The crude compound was purified by column chromatography to yield 1-benzenesulfonyl-2-(2,6-difluoro-phenyl)-1H-indole-5-carboxylic acid (800 mg, 64%).

Benzenesulfonyl-2-(2,6-difluoro-phenyl)-1H-indole-5-carboxylic acid methoxy-methyl-amide: To a solution of 1-benzenesulfonyl-2-(2,6-difluoro-phenyl)-1H-indole-5-carboxylic acid (1.1 g, 2.66 mmol) in dry DMF (10 ml) was added EDCI (1.02 g, 5.32 mmol), DMAP (590 mg, 4.84 mmol) and Weinreb amide (363 mg, 3.72 mmol) and stirred for 10 min at RT. Triethylamine (1.35 ml, 9.68 mmol) was added and the mixture was stirred at RT for 16 h. After completion, the reaction was quenched with ice-water and extracted with EtOAc. The organic phase was washed with brine, dried over Na₂SO₄ and concentrate under vacuum. The crude compound was purified by column chromatography to yield 1-benzenesulfonyl-2-(2,6-difluoro-phenyl)-1H-indole-5-carboxylic acid methoxy-methyl-amide (700 mg, 79%).

1-[1-Benzenesulfonyl-2-(2,6-difluoro-phenyl)-1H-indol-5-yl]-propan-1-one: 1-Benzenesulfonyl-2-(2,6-difluoro-phenyl)-1H-indole-5-carboxylic acid methoxy-methyl-amide (3.1 g, 6.79 mmol) was dissolved in dry THF (20 ml). Freshly prepared EtMgBr (4M, 6.79 ml) was added and stirred at 60° C. for 6 h. After the completion, the reaction was quenched with saturated NH4Cl solution and extracted with DCM. The organic phase was washed with brine, dried over Na₂SO₄ and concentrated. The crude compound was purified by column chromatography to yield 1-[1-benzenesulfonyl-2-(2,6-difluoro-phenyl)-1H-indol-5-yl]-propan-1-one (2.4 g, 83%).

1-[1-Benzenesulfonyl-2-(2,6-difluoro-phenyl)-1H-indol-5-yl]-2-bromo-propan-1-one: 1-[1-Benzenesulfonyl-2-(2,6-difluoro-phenyl)-1H-indol-5-yl]-propan-1-one (500 mg, 1.17 mmol) was dissolved in CCl4 (15 ml) and cooled to 0° C. Bromine (0.07 ml, 1.17 mmol) dissolved in CCl4 (5 ml) was added to the reaction mixture and stirred for 12 h at RT. After the completion, the reaction was quenched with aqueous Na2S2O3 solution and extracted with DCM. The organic phase was washed with brine, dried over Na₂SO₄ and concentrated. The crude compound was purified by column chromatography to yield 1-[1-benzenesulfonyl-2-(2,6-difluoro-phenyl)-1H-indol-5-yl]-2-bromo-propan-1-one (410 mg, 69%).

Benzenesulfonyl-2-(2,6-difluoro-phenyl)-5-(5-methyl-2-pyridin-3-yl-thiazol-4-yl)-1H-indole: 1-[1-Benzenesulfonyl-2-(2,6-difluoro-phenyl)-1H-indol-5-yl]-2-bromo-propan-1-one (150 mg, 0.298 mmol) and thionicotinamide (82 mg, 0.595 mmol) was dissolved in EtOH (10 ml) and reflux for 12 h. After the completion, the reaction was concentrated and purified by column chromatography to yield 1-benzenesulfonyl-2-(2,6-difluoro-phenyl)-5-(5-methyl-2-pyridin-3-yl-thiazol-4-yl)-1H-indole (110 mg, 68%).

2-(2,6-Difluoro-phenyl)-5-(5-methyl-2-pyridin-3-yl-thiazol-4-yl)-1H-indole: 1-Benzenesulfonyl-2-(2,6-difluoro-phenyl)-5-(5-methyl-2-pyridin-3-yl-thiazol-4-yl)-1H-indole (76 mg, 0.183 mmol) was dissolved in THF/MeOH (2:1, 3 ml) and added Cs₂CO₃ (120 mg, 0.366 mmol). The above reaction mass was stirred at 25° C. for 24 h. Then the reaction mass was diluted with water and extracted with EtOAc. The organic phase was dried over Na₂SO₄ and concentrated. The crude compound was purified by column chromatography to yield 2-(2,6-difluoro-phenyl)-5-(5-methyl-2-pyridin-3-yl-thiazol-4-yl)-1H-indole (20 mg, 27%), MS (M+H)=404.

### Example 14

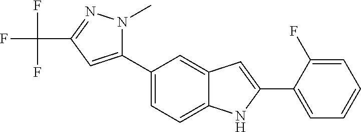

### 2-(2-Fluoro-phenyl)-5-(2-methyl-5-trifluoromethyl-2H-pyrazol-3-yl)-1H-indole

Was prepared in a manner similar to Example 1 except 5-bromo-2-(2-fluoro-phenyl)-1H-indole was substituted for 5-bromo-2-(2,6-difluoro-phenyl)-1H-indole. MS (M+H)=360.

### Example 15

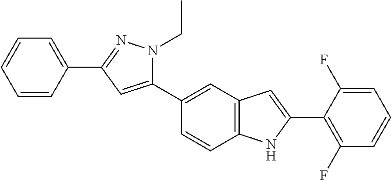

### 2-(2,6-difluoro-phenyl)-5-(2-ethyl-5-phenyl-2H-pyrazol-3-yl)-1H-indole

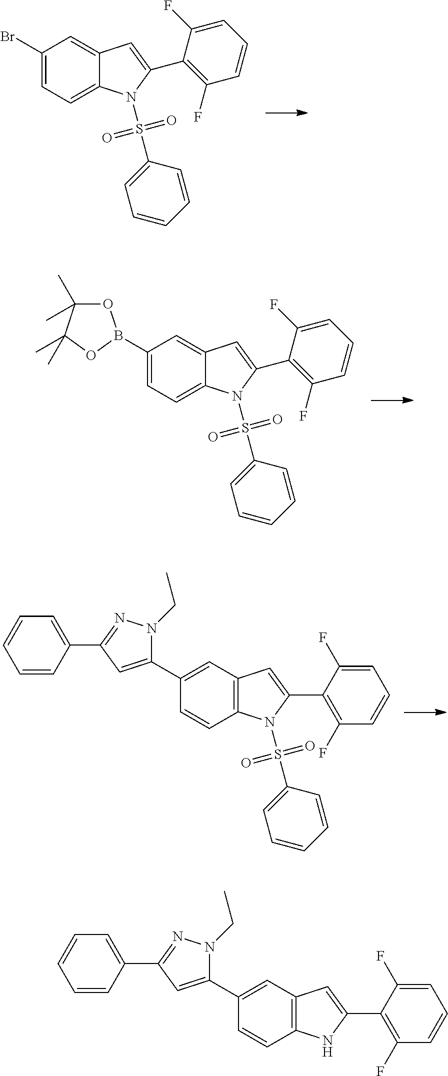

Benzenesulfonyl-2-(2,6-difluoro-phenyl)-5-(4,4,5,5-tetramethyl-[1,3,2]dioxaborolan-2-yl)-1H-indole: To a solution of 1-benzenesulfonyl-5-bromo-2-(2,6-difluoro-phenyl)-1H-indole (2.1 g, 11.68 mmol) in 1,4-dioxane was added bispinacolatodiborane (1.37 g, 5.39 mmol) and K₂CO₃ (1.94 g, 14.06 mmol) at 25° C. The mixture was stirred at 110° C. for 14 h (TLC). After the completion of the reaction, the mixture was extracted with EtOAc (3×50 mL). The organic phase was washed with brine, dried over Na₂SO₄ and concentrated. The crude compound was purified by column chromatography (2% EtOAc-Hexane) to yield 1-benzenesulfonyl-2-(2,6-difluoro-phenyl)-5-(4,4,5,5-tetramethyl-[1,3,2]dioxaborolan-2-yl)-1H-indole (1 g, 44%).

Benzenesulfonyl-2-(2,6-difluoro-phenyl)-5-(2-ethyl-5-phenyl-2H-pyrazol-3-yl)-1H-indole: 1-Benzenesulfonyl-2-(2,6-difluoro-phenyl)-5-(4,4,5,5-tetramethyl-[1,3,2]dioxaborolan-2-yl)-1H-indole (150 mg, 0.30 mmol) was dissolved in 1,4-dioxane. Trifluoro-methanesulfonic acid 2-ethyl-5-phenyl-2H-pyrazol-3-yl ester (Intermediate 2, 87 mg, 0.27 mmol) and aqueous K₂CO₃ (2M, 0.48 mL) were added. The reaction mixture was purged with nitrogen for 10 min, Pd(PPh₃)₄ (35 mg, 0.03 mmol) was added and stirred at 100° C. for 10 h (TLC). The reaction mixture was filtered through Celite and extracted with EtOAc (3×20 mL). The organic phase was dried over Na₂SO₄ and concentrated. The crude compound was purified by column chromatography (20% EtOAc-Hexane) to yield 1-benzenesulfonyl-2-(2,6-difluoro-phenyl)-5-(2-ethyl-5-phenyl-2H-pyrazol-3-yl)-1H-indole (80 mg, 50%).

2-(2,6-Difluoro-phenyl)-5-(2-ethyl-5-phenyl-2H-pyrazol-3-yl)-1H-indole: 1-Benzenesulfonyl-2-(2,6-difluoro-phenyl)-5-(2-ethyl-5-phenyl-2H-pyrazol-3-yl)-1H-indole (105 mg, 0.19 mmol) was dissolved in 1,4-dioxane and aqueous NaOH (5M, 0.8 mL) was added. The reaction mixture was stirred at 100° C. for 4 h (TLC). The pH of the reaction mass was then adjusted to 7 with 5% HCl and extracted with EtOAc (3×20 mL). The combined organic layers were dried over Na₂SO₄ and concentrated. The crude compound was purified by column chromatography (20-30% EtOAc-Hexane) to yield 2-(2,6-difluoro-phenyl)-5-(2-ethyl-5-phenyl-2H-pyrazol-3-yl)-1H-indole (45 mg, 58%), MS (M+H)=400.

### Example 16

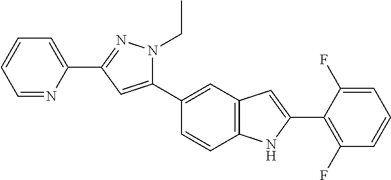

2-(2,6-Difluoro-phenyl)-5-(2-ethyl-5-pyridin-2-yl-2H-pyrazol-3-yl)-1H-indole

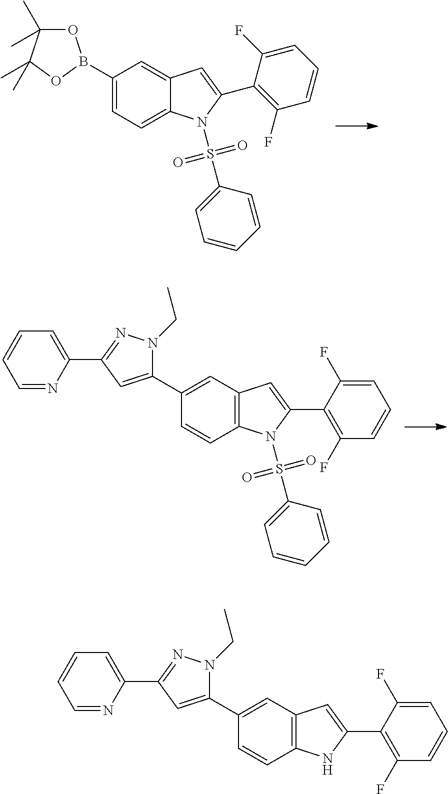

Was prepared as described in Example 15 except trifluoro-methanesulfonic acid 2-ethyl-5-pyridin-2-yl-2H-pyrazol-3-yl ester (Intermediate 3) was coupled to 1-benzenesulfonyl-2-(2,6-difluoro-phenyl)-5-(4,4,5,5-tetramethyl-[1,3,2]dioxaborolan-2-yl)-1H-indole in the Suzuki coupling step.

2-(2,6-Difluoro-phenyl)-5-(2-ethyl-5-pyridin-2-yl-2H-pyrazol-3-yl)-1H-indole: 1-Benzenesulfonyl-2-(2,6-difluoro-phenyl)-5-(2-ethyl-5-pyridin-2-yl-2H-pyrazol-3-yl)-1H-indole (102 mg, 0.19 mmol) was dissolved in THF/MeOH (2:1) and added Cs₂CO₃ (184 mg, 0.57 mmol). The above reaction mass was stirred at 25° C. for 24 h (TLC). The reaction mass was extracted with EtOAc (3×20 mL). The organic phase was dried over Na₂SO₄ and concentrated. The crude compound was purified by column chromatography (20-30% EtOAc-Hexane) to obtain 2-(2,6-difluoro-phenyl)-5-(2-ethyl-5-pyridin-2-yl-2H-pyrazol-3-yl)-1H-indole (15 mg, 20%), MS (M+H)=401.

### Example 17

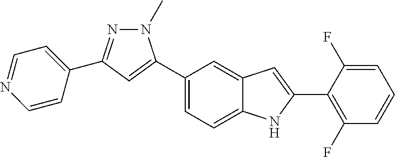

### 2-(2,6-Difluoro-phenyl)-5-(2-methyl-5-pyridin-4-yl-2H-pyrazol-3-yl)-1H-indole

Was prepared as described in Example 16 substituting trifluoro-methanesulfonic acid 2-methyl-5-pyridin-4-yl-2H-pyrazol-3-yl ester (Intermediate 4) in the Suzuki coupling step. MS (M+H)=387.

### Example 18

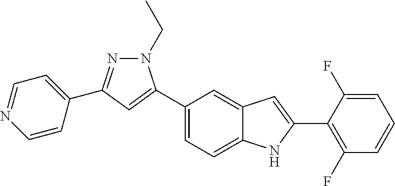

### 2-(2,6-Difluoro-phenyl)-5-(2-ethyl-5-pyridin-4-yl-2H-pyrazol-3-yl)-1H-indole

Was prepared as described in Example 16 substituting trifluoro-methanesulfonic acid 2-ethyl-5-pyridin-4-yl-2H-pyrazol-3-yl ester (Intermediate 5) in the Suzuki coupling step. MS (M+H)=401.

### Example 19

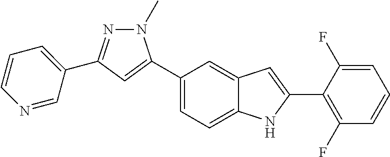

### 2-(2,6-Difluoro-phenyl)-5-(2-methyl-5-pyridin-3-yl-2H-pyrazol-3-yl)-1H-indole

Was prepared as described in Example 16 substituting trifluoro-methanesulfonic acid 2-methyl-5-pyridin-3-yl-2H-pyrazol-3-yl ester (Intermediate 6) in the Suzuki coupling step. MS (M+H)=387.

### Example 20

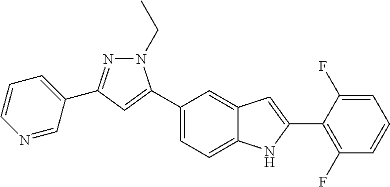

### 2-(2,6-Difluoro-phenyl)-5-(2-ethyl-5-pyridin-3-yl-2H-pyrazol-3-yl)-1H-indole

Was prepared as described in Example 16 substituting trifluoro-methanesulfonic acid 2-ethyl-5-pyridin-3-yl-2H-pyrazol-3-yl ester (Intermediate 7) in the Suzuki coupling step. MS (M+H)=401.

### Example 21

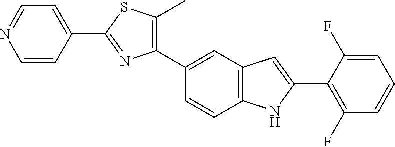

### 2-(2,6-Difluoro-phenyl)-5-(5-methyl-2-pyridin-4-yl-thiazol-4-yl)-1H-indole

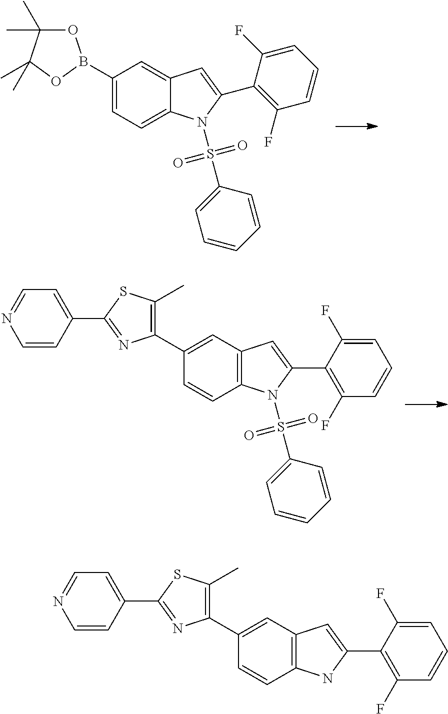

Benzenesulfonyl-2-(2,6-difluoro-phenyl)-5-(5-methyl-2-pyridin-4-yl-thiazol-4-yl)-1H-indole: A solution of 1-benzenesulfonyl-2-(2,6-difluoro-phenyl)-5-(4,4,5,5-tetramethyl-[1,3,2]dioxaborolan-2-yl)-1H-indole (300 mg, 0.81 mmol) and trifluoro-methanesulfonic acid 5-methyl-2-pyridin-4-yl-thiazol-4-yl ester (Intermediate 8, 169 mg, 0.88 mmol) in 1,4-dioxane (2 mL) was purged with nitrogen (10 min) and aqueous K₂CO₃ (2 M, 0.6 mL) was added. The mixture was purged with nitrogen for an additional 20 min. Pd(PPh₃)₄ (10 mol %, 85 mg) was added to the above reaction mixture and stirred at 100° C. After the completion of the reaction (10 h, by TLC), the mixture was filtered through Celite and the filtrate extracted with EtOAc (3×20 mL). The organic phase (EtOAc layer) was washed with brine, dried over Na₂SO₄ and concentrated. The crude product was purified by column chromatography (10% EtOAc-Hexane) to yield 1-benzenesulfonyl-2-(2,6-difluoro-phenyl)-5-(5-methyl-2-pyridin-4-yl-thiazol-4-yl)-1H-indole (123 mg, 37%).

2-(2,6-difluoro-phenyl)-5-(5-methyl-2-pyridin-4-yl-thiazol-4-yl)-1H-indole: To a solution of 1-benzenesulfonyl-2-(2,6-difluoro-phenyl)-5-(5-methyl-2-pyridin-4-yl-thiazol-4-yl)-1H-indole (93 mg, 0.22 mmol) in THF/MeOH (2:1) (6 mL) Cs₂CO₃ (215 mg, 0.66 mmol) was added and stirred at 25° C. for 24 h (TLC). After the completion of the reaction, the solvents were removed and the residue was extracted with EtOAc (3×10 mL). The organic phase (EtOAc layer) was washed with brine, dried over Na₂SO₄ and concentrated. The crude compound was purified by column chromatography (10-20% EtOAc-Hexane) to yield 2-(2,6-difluoro-phenyl)-5-(5-methyl-2-pyridin-4-yl-thiazol-4-yl)-1H-indole (52 mg, 58%), MS (M+H)=404.

### Example 22

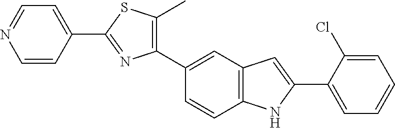

### 2-(2-Chloro-phenyl)-5-(5-methyl-2-pyridin-4-yl-thiazol-4-yl)-1H-indole

Prepared in a manner similar to Example 21 except 1-benzenesulfonyl-2-(2-chloro-phenyl)-5-(4,4,5,5-tetramethyl-[1,3,2]dioxaborolan-2-yl)-1H-indole was substituted for 1-benzenesulfonyl-2-(2,6-difluoro-phenyl)-5-(4,4,5,5-tetramethyl-[1,3,2]dioxaborolan-2-yl)-1H-indole. MS (M+H)=402.

### Example 23

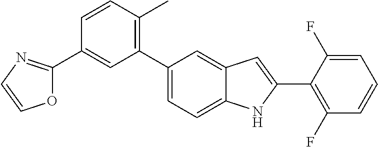

### 2-(2,6-Difluoro-phenyl)-5-(2-methyl-5-oxazol-2-yl-phenyl)-1H-indole

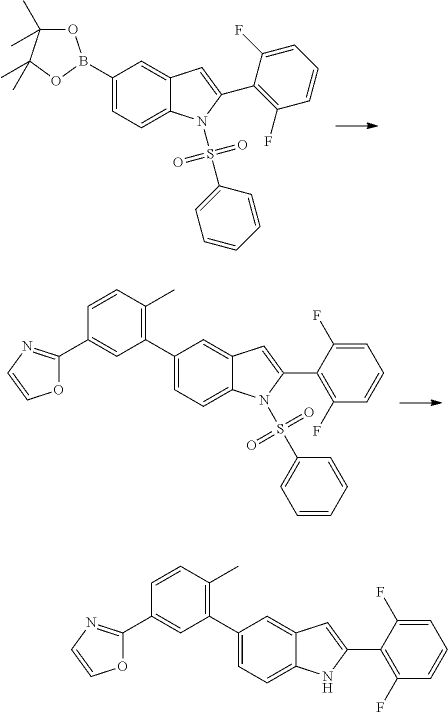

Benzenesulfonyl-2-(2,6-difluoro-phenyl-5-(2-methyl-5-oxazol-2-yl-phenyl)-1H-indole: A solution of 1-benzenesulfonyl-2-(2,6-difluoro-phenyl)-5-(4,4,5,5-tetramethyl-[1,3,2]dioxaborolan-2-yl)-1H-indole (46 mg, 0.191 mmol) and 2-(3-bromo-4-methyl-phenyl)-oxazole (Intermediate 9, 95 mg, 0.191 mmol) in 1,4-dioxane was purged with nitrogen (10 min) and then aqueous K₂CO₃ (2 M, 0.2 mL) was added. The mixture was purged with nitrogen for an additional 20 min. Pd(PPh₃)₄ (10 mol %, 22 mg) was added to the above reaction mixture and stirred at 100° C. After the completion of the reaction (18 h, by TLC), the mixture was filtered through Celite and the filtrate extracted with EtOAc (3×20 mL). The organic phase (EtOAc layer) was washed with brine, dried over Na₂SO₄ and concentrated to yield 1-benzenesulfonyl-2-(2,6-difluoro-phenyl)-5-(2-methyl-5-oxazol-2-yl-phenyl)-1H-indole (20 mg, 20%).

2-(2,6-Difluoro-phenyl)-5-(2-methyl-5-oxazol-2-yl-phenyl)-1H-indole: 1-Benzenesulfonyl-2-(2,6-difluoro-phenyl)-5-(2-methyl-5-oxazol-2-yl-phenyl)-1H-indole (17 mg, 0.032 mmol) was dissolved in THF/MeOH (2:1). Cs₂CO₃ (31 mg, 0.097 mmol) was added and stirred at 25° C. After the completion of the reaction (24 h, by TLC), the solvents were removed and extracted with EtOAc (3×10 mL). The organic phase (EtOAc layer) was washed with brine, dried over Na₂SO₄ and concentrated. The crude compound was purified by combiflash column chromatography (10% EtOAc-Hexane) to yield 2-(2,6-difluoro-phenyl)-5-(2-methyl-5-oxazol-2-yl-phenyl)-1H-indole (7 mg, 56%), MS (M+H)=387.

### Example 24

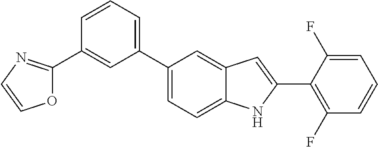

### 2-(2,6-Difluoro-phenyl)-5-(3-oxazol-2-yl-phenyl)-1H-indole

Was prepared in a manner identical to Example 23. MS (M+H)=373.

### Example 25

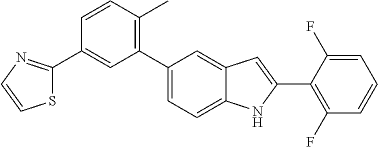

### 2-(2,6-Difluoro-phenyl)-5-(2-methyl-5-thiazol-2-yl-phenyl)-1H-indole

Was prepared in a manner identical to Example 23 substituting Intermediate 10 in the Suzuki coupling step. MS (M+H)=403.

### Example 26

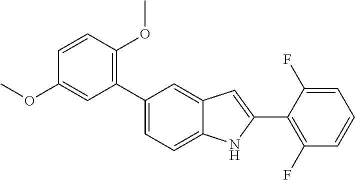

### 2-(2,6-Difluoro-phenyl)-5-(2,5-dimethoxy-phenyl)-1H-indole

Was prepared in a manner identical to Example 23. MS (M+H)=366.

### Example 27

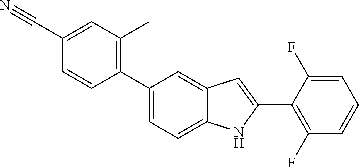

### 4-[2-(2,6-Difluoro-phenyl)-1H-indol-5-yl]-3-methyl-benzonitrile

Was prepared in a manner identical to Example 23. MS (M+H)=345.

### Example 28

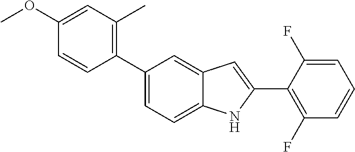

### 2-(2,6-Difluoro-phenyl)-5-(4-methoxy-2-methyl-phenyl)-1H-indole

Was prepared in a manner identical to Example 23. MS (M+H)=350.

### Example 29

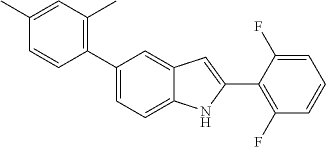

### 2-(2,6-Difluoro-phenyl)-5-(2,4-dimethyl-phenyl)-1H-indole

Was prepared in a manner identical to Example 23. MS (M+H)=334.

### Example 30

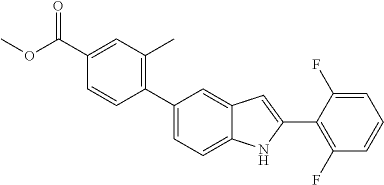

Was prepared in a manner identical to Example 23. MS (M+H)=378.

### Example 31

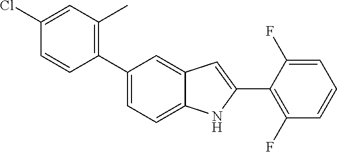

### 5-(4-Chloro-2-methyl-phenyl)-2-(2,6-difluoro-phenyl)-1H-indole

Was prepared in a manner identical to Example 23. MS (M+H)=353.

### Example 32

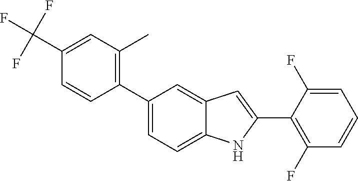

### 2-(2,6-Difluoro-phenyl)-5-(2-methyl-4-trifluoromethyl-phenyl)-1H-indole

Was prepared in a manner identical to Example 23. MS (M+H)=388.

### Example 33

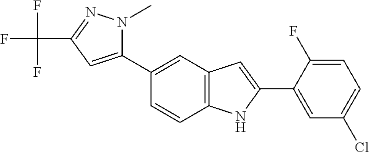

### 2-(5-Chloro-2-fluoro-phenyl)-5-(2-methyl-5-trifluoromethyl-2H-pyrazol-3-yl)-1H-indole

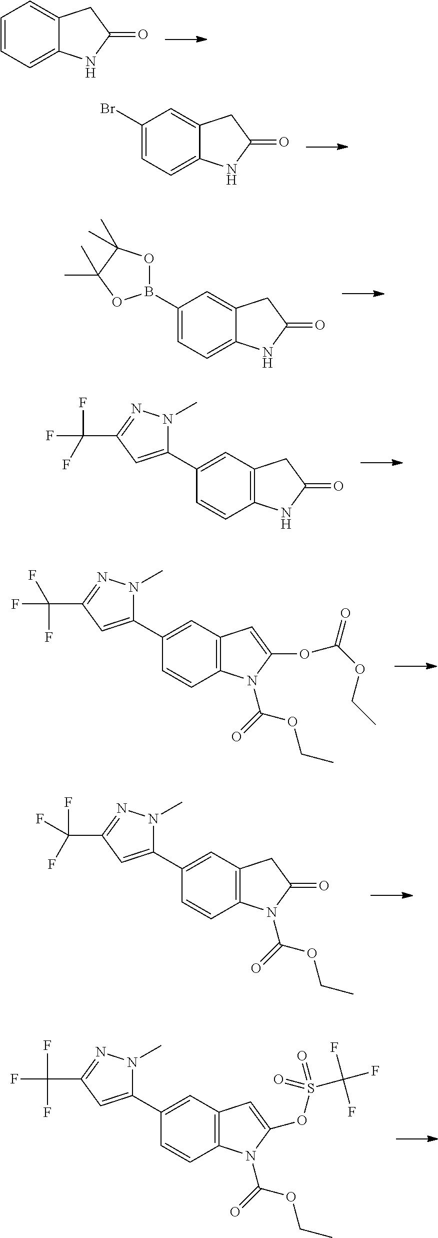

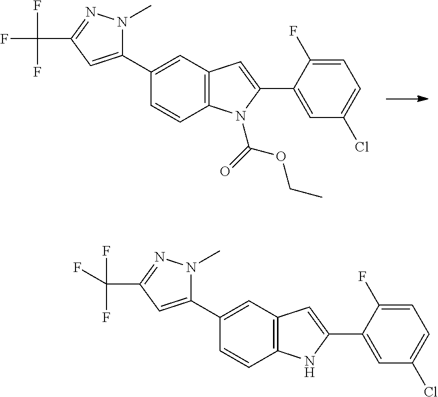

Bromo-1,3-dihydro-indol-2-one: To a solution of 1,3-Dihydro-indol-2-one (20 g, 133.15 mmol) in acetonitrile (300 ml) at 0° C. was added NBS (30.76 gm, 173.8 mmol) in several portions and the solution stirred at this temperature for 3 h. Water was added to the reaction mixture, upon which a white solid precipitated. The solid was collected by filtration, washed with hot water and dried under vacuum to obtain compound 5-Bromo-1,3-dihydro-indol-2-one (28 g, 88%).

5-(4,4,5,5-Tetramethyl-[1,3,2]dioxaborolan-2-yl)-1,3-dihydro-indol-2-one: To a solution of 5-Bromo-1,3-dihydro-indol-2-one (10 g, 47.1 mmol) in dioxane (120 ml) was added bispinacolato diborane (26.25 gm, 103.7 mmol); the reaction was purged with nitrogen for 30 min followed by addition of potassium acetate (13.86 g, 141 mmol) and Pd(dppf)Cl2 (1.92 g 2.3 mmol). The reaction mixture was warmed to 100° C. and stirred at this temperature for 16 h. After the completion of the reaction it was filtered through Celite and the filtrate was diluted with water, extracted with EtOAc. The combine organic layer ware washed with brine, dried over Na₂SO₄, concentrated and purified by column chromatography to obtain 5-(4,4,5,5-Tetramethyl-[1,3,2]dioxaborolan-2-yl)-1,3-dihydro-indol-2-one, (7.8 g, 64%).

5-(2,5-Dimethyl-2H-pyrazol-3-yl)-1,3-dihydro-indol-2-one: A solution of Trifluoromethanesulfonic acid 2-methyl-5-trifluoromethyl-2H-pyrazol-3-yl ester (Intermediate 11, 1.5 g, 5.03 mmol) and 5-(4,4,5,5-Tetramethyl-[1,3,2]dioxaborolan-2-yl)-1,3-dihydro-indol-2-one (3.45 g, 11.54 mmol) in 1,4-dioxane (50 mL) was degassed by purging with nitrogen (20 min) and then aqueous K₂CO₃ (2 M in water, 7.14 mL) was added and purged with nitrogen (30 min). Pd(dppf)Cl₂ (10 mol %, 472 mg) was then added to the above reaction mixture and stirred at 100° C. for 4 h. After the completion the reaction was filtered through Celite and the filtrate was diluted with water and extracted with EtOAc. The organic phase was washed with brine, dried over Na₂SO₄ and concentrated. The Crude compound was purified by column chromatography to obtain 5-(2,5-Dimethyl-2H-pyrazol-3-yl)-1,3-dihydro-indol-2-one (810 mg, 49%).

Ethoxycarbonyloxy-5-(2-methyl-5-trifluoromethyl-2H-pyrazol-3-yl)-indole-1 carboxylic acid ethyl ester: Ethylchloroformate (1.36 mL, 14.23 mmol) was added to a solution of 5-(2,5-Dimethyl-2H-pyrazol-3-yl)-1,3-dihydro-indol-2-one (800 mg, 2.84 mmol) in THF (16 mL) and triethylamine (2.39 mL, 17.07 mmol) at 0° C. The reaction was warmed to room temperature and stirred at this temperature for 20 h. The solvent was then removed and re-dissolved in DCM, washed with water and brine. The organic layer separated, dried over Na₂SO₄ and concentrated to obtain 2-Ethoxycarbonyloxy-5-(2-methyl-5-trifluoromethyl-2H-pyrazol-3-yl)-indole-1 carboxylic acid ethyl ester: Ethylchloroformate (1.2 g, 95%).

5-(2-Methyl-5-trifluoromethyl-2H-pyrazol-3-yl)-2-oxo-2,3-dihydro-indole-1-carboxylic acid ethyl ester

5-(2-Methyl-5-trifluoromethyl-2H-pyrazol-3-yl)-2-oxo-2,3-dihydro-indole-1-carboxylic acid ethyl ester: 2-Ethoxycarbonyloxy-5-(2-methyl-5-trifluoromethyl-2H-pyrazol-3-yl)-indole-1 carboxylic acid ethyl ester (1.2 g, 2.82 mmol) was dissolved in DMF (10 mL) at 0° C. and (NH₄)₂CO₃ (0.57 g, 5.64 mmol) was added and stirred from 0° C. to 25° C. for 1 h. The entire mixture was then poured into water and extracted with DCM. The organic phase was washed with brine, dried over Na₂SO₄ and concentrated. The Crude compound was purified by column chromatography to give 5-(2-Methyl-5-trifluoromethyl-2H-pyrazol-3-yl)-2-oxo-2,3-dihydro-indole-1-carboxylic acid ethyl ester (570 mg, 52.5%).

5-(2-Methyl-5-trifluoromethyl-2H-pyrazol-3-yl)-2-trifluoromethanesulfonyloxy-indole-1-carboxylic acid ethyl ester: To a dichloromethane (20 ml) solution of 5-(2-Methyl-5-trifluoromethyl-2H-pyrazol-3-yl)-2-oxo-2,3-dihydro-indole-1-carboxylic acid ethyl ester (540 mg, 1.52 mmol) was added DIPEA (1.01 mL, 6.116 mmol) at 0° C. followed by Tf₂O (0.76 mL, 4.58 mmol) and stirred at this temperature for 1 h. The reaction mixture was then quenched with ice water and extracted with DCM. The combined organic layer was washed with brine, dried over Na₂SO₄ and concentrated. The crude compound was then purified by column chromatography to obtain 5-(2-Methyl-5-trifluoromethyl-2H-pyrazol-3-yl)-2-trifluoromethanesulfonyloxy-indole-1-carboxylic acid ethyl ester (220 mg, 29%).

2-(5-Chloro-2-fluoro-phenyl)-5-(2-methyl-5-trifluoromethyl-2H-pyrazol-3-yl)-indole-1-carboxylic acid ethyl ester: To a solution of 5-(2-Methyl-5-trifluoromethyl-2H-pyrazol-3-yl)-2-trifluoromethanesulfonyloxy-indole-1-carboxylic acid ethyl ester (135 mg, 0.312 mmol) and 2-fluoro-5-chloro-boronic acid (82 mg, 0.468 mmol) in 1,4-dioxane (4 mL) was degassed and purged with nitrogen (10 min) and then aqueous K₂CO₃ (2 M, 0.2 mL) was added and purged with nitrogen again (20 min). Pd (dppf)Cl₂ (10 mol %, 23 mg) was added to the above reaction mixture and stirred at 100° C. for 4 h. After the completion of the reaction it was filtered through Celite and concentrated. The crude material was purified by CombiFlash column chromatography to obtain 2-(5-Chloro-2-fluoro-phenyl)-5-(2-methyl-5-trifluoromethyl-2H-pyrazol-3-yl)-indole-1-carboxylic acid ethyl ester (62 mg, 48%).

2-(5-Chloro-2-fluoro-phenyl)-5-(2-methyl-5-trifluoromethyl-2H-pyrazol-3-yl)-1H-indole: 2-(5-Chloro-2-fluoro-phenyl)-5-(2-methyl-5-trifluoromethyl-2H-pyrazol-3-yl)-indole-1-carboxylic acid ethyl ester (62 mg, 0.150 mmol) was dissolved in EtOH (5 ml) and NaOH (3 M, 0.1 mL) was added at 0° C. This was then allowed to warm to 25° C. and stirred at this temperature for 3 h. After the completion of the reaction the solvents were removed and water was added to the residue, which was extracted with EtOAc. The organic phase was washed with brine, dried over Na₂SO₄ and concentrated. The crude compound was purified by column chromatography to obtain 2-(5-Chloro-2-fluoro-phenyl)-5-(2-methyl-5-trifluoromethyl-2H-pyrazol-3-yl)-1H-indole (32 mg, 63%). MS (M+H)=394.

### Example 34

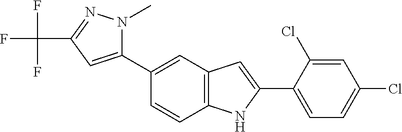

### 2-(2,4-Dichloro-phenyl)-5-(2-methyl-5-trifluoromethyl-2H-pyrazol-3-yl)-1H-indole

Prepared in a manner identical to that described for example 33. MS (M+H)=410.

### Example 35

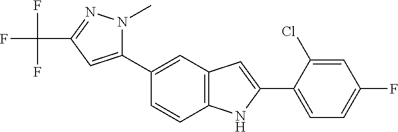

### 2-(2-Chloro-4-fluoro-phenyl)-5-(2-methyl-5-trifluoromethyl-2H-pyrazol-3-yl)-1H-indole

Prepared in a manner identical to that described for example 33. MS (M+H)=393.

### Example 36

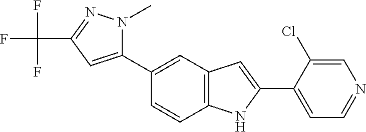

### 2-(3-Chloro-pyridin-4-yl)-5-(2-methyl-5-trifluoromethyl-2H-pyrazol-3-yl)-1H-indole

Prepared in a manner identical to that described for example 33. MS (M+H)=377.

### Example 37

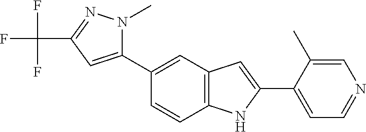

### 2-(3-Methyl-pyridin-4-yl)-5-(2-methyl-5-trifluoromethyl-2H-pyrazol-3-yl)-1H-indole

Prepared in a manner identical to that described for example 33. MS (M+H)=375.

### Example 38

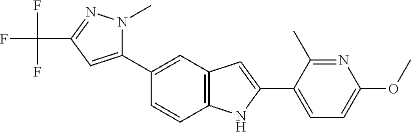

### 2-(6-Methoxy-2-methyl-pyridin-3-yl)-5-(2-methyl-5-trifluoromethyl-2H-pyrazol-3-yl)-1H-indole

Prepared in a manner identical to that described for example 33. MS (M+H)=387.

### Example 39

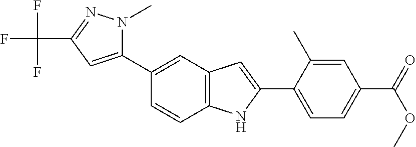

### Methyl-4-[5-(2-methyl-5-trifluoromethyl-2H-pyrazol-3-yl)-1H-indol-2-yl]-benzoic acid methyl ester

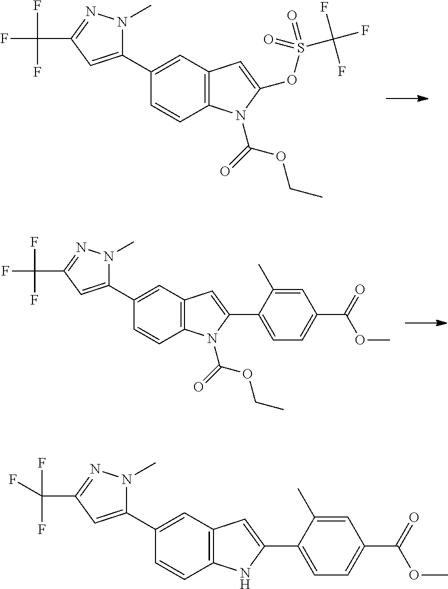

2-(4-Methoxycarbonyl-2-methyl-phenyl)-5-(2-methyl-5-trifluoromethyl-2H-pyrazol-3-yl)-indole-1-carboxylic acid ethyl ester: To a solution of 5-(2-Methyl-5-trifluoromethyl-2H-pyrazol-3-yl)-2-trifluoromethanesulfonyloxy-indole-1-carboxylic acid ethyl ester (60 mg, 0.124 mmol) and 4-(Methoxycarbonyl)-2-methylbenzeneboronic acid (68 mg, 0.247 mmol) in 1,4-dioxane (4 mL) was degassed and purged with nitrogen (10 min) and then aqueous K₂CO₃ (2 M, 0.15 mL) was added and purged with nitrogen again (20 min). Pd (dppf)Cl₂ (10 mol %, 12 mg) was added to the above reaction mixture and stirred at 100° C. for 4 h. After the completion of the reaction it was filtered through Celite and concentrated. The crude material was purified by column chromatography to obtain 2-(4-Methoxycarbonyl-2-methyl-phenyl)-5-(2-methyl-5-trifluoromethyl-2H-pyrazol-3-yl)-indole-1-carboxylic acid ethyl ester (25 mg, 41%).

Methyl-4-[5-(2-methyl-5-trifluoromethyl-2H-pyrazol-3-yl)-1H-indol-2-yl]-benzoic acid methyl ester: To a solution of 2-(4-Methoxycarbonyl-2-methyl-phenyl)-5-(2-methyl-5-trifluoromethyl-2H-pyrazol-3-yl)-indole-1-carboxylic acid ethyl ester (40 mg, 0.08 mmol) was dissolved in MeOH (4 ml) and NaOH (3 M, 0.027 mL) was added at 0° C. This was then stirred at 0° C. for 3 h. After the completion of the reaction the solvents were removed and neutralize by aq HCl (1N) extracted with EtOAc. The organic phase was washed with brine, dried over Na₂SO₄ and concentrated. The crude compound was purified by column chromatography to obtain 3-Methyl-4-[5-(2-methyl-5-trifluoromethyl-2H-pyrazol-3-yl)-1H-indol-2-yl]-benzoic acid methyl ester (20 mg, 58%), MS (M+H)=414.

### Example 40

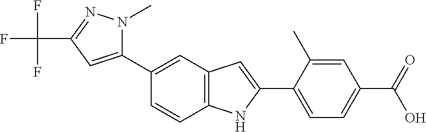

### Methyl-4-[5-(2-methyl-5-trifluoromethyl-2H-pyrazol-3-yl)-1H-indol-2-yl]-benzoic acid methyl ester

Methyl-4-[5-(2-methyl-5-trifluoromethyl-2H-pyrazol-3-yl)-1H-indol-2-yl]-benzoic acid methyl ester: To a solution of 2-(4-Methoxycarbonyl-2-methyl-phenyl)-5-(2-methyl-5-trifluoromethyl-2H-pyrazol-3-yl)-indole-1-carboxylic acid ethyl ester (40 mg, 0.08 mmol) was dissolved in MeOH (4 ml) and NaOH (3 M, 0.054 mL) was added at 0° C. This was then allowed to warm to 25° C. and stirred at this temperature for 3 h (TLC). After the completion of the reaction the solvents were removed and neutralize by aq HCl (1N) extracted with EtOAc. The organic phase was washed with brine, dried over Na₂SO₄ and concentrated. The crude compound was purified by column chromatography to obtain 3-Methyl-4-[5-(2-methyl-5-trifluoromethyl-2H-pyrazol-3-yl)-1H-indol-2-yl]-benzoic acid methyl ester (12 mg, 36%), MS (M+H)=400.

### Example 41

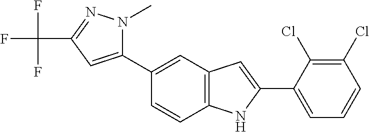

### 2-(2,3-Dichloro-phenyl)-5-(2-methyl-5-trifluoromethyl-2H-pyrazol-3-yl)-1H-indole

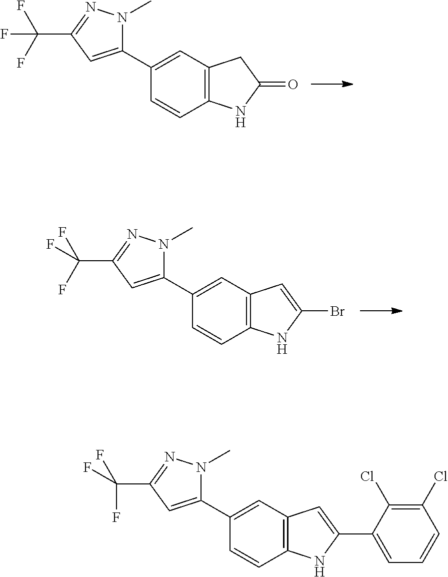

Bromo-5-(2-methyl-5-trifluoromethyl-2H-pyrazol-3-yl)-1H-indole: To solution of compound 5-(2-Methyl-5-trifluoromethyl-2H-pyrazol-3-yl)-1,3-dihydro-indol-2-one (0.8 g, 2.85 mmol) in ethylene dichloride was added POBr₃ (1.63 g, 5.7 mmol) and imidazole (0.232 g, 3.42 mmol) and the reaction was heated at 90° C. for 2 h. After the completion of the reaction it was cooled to 25° C. and saturated NaHCO₃ was added and the mixture was extracted with EtOAc (2×20 mL). The organic phase (EtOAc layer) was washed with brine, dried over Na₂SO₄ and concentrated to obtain 2-Bromo-5-(2-methyl-5-trifluoromethyl-2H-pyrazol-3-yl)-1H-indole (100 mg, 10%).

2-(2,3-Dichloro-phenyl)-5-(2-methyl-5-trifluoromethyl-2H-pyrazol-3-yl)-1H-indole: To a solution of 2-Bromo-5-(2-methyl-5-trifluoromethyl-2H-pyrazol-3-yl)-1H-indole (80 mg, 0.28 mmol) and 2,3-Dichlorobenzeneboronic acid (53 mg, 0.28 mmol) in 1,4-dioxane (2 mL) was degassed and purged with nitrogen (10 min) and then aqueous K₂CO₃ (2 M, 0.2 mL) was added and purged with nitrogen again (20 min). Pd(dppf)₂Cl₂ (10 mol %, 21 mg) was added to the above reaction mixture and stirred at 100° C. After 18 h the reaction mixture was filtered through Celite and the filtrate extracted with EtOAc (3×20 mL). The organic phase (EtOAc layer) was washed with brine, dried over Na₂SO₄ and concentrated to a residue which was purified by column chromatography (10-30% EtOAc-Hexane) to obtain 2-(2,3-Dichloro-phenyl)-5-(2-methyl-5-trifluoromethyl-2H-pyrazol-3-yl)-1H-indole (25 mg, 26%), MS (M+H)=410.

### Example 42

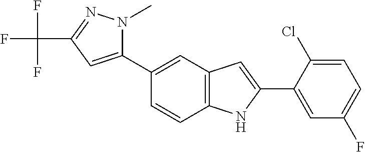

### 2-(2-Chloro-5-fluoro-phenyl)-5-(2-methyl-5-trifluoromethyl-2H-pyrazol-3-yl)-1H-indole

Prepared in a manner identical to that described for example 41. MS (M+H)=394.

### Example 43

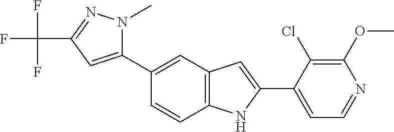

### 2-(3-Chloro-2-methoxy-pyridin-4-yl)-5-(2-methyl-5-trifluoromethyl-2H-pyrazol-3-yl)-1H-indole

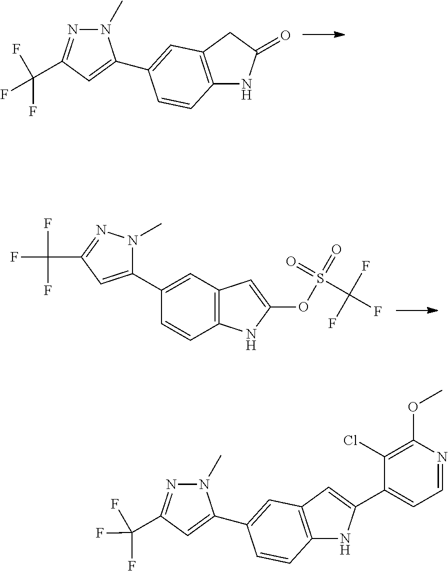

Trifluoromethanesulfonic acid 5-(2-methyl-5-trifluoromethyl-2H-pyrazol-3-yl)-1H-indol-2-yl ester: To a solution of 5-(2-Methyl-5-trifluoromethyl-2H-pyrazol-3-yl)-1,3-dihydro-indol-2-one (100 mg, 0.36 mmol) in DCM (8 ml) at 0° C. was added 2,6-Di-tert-butyl-4-methylpyridine (109.5 mg, 0.54 mmol) and it was stirred at this temperature for 10 min followed by addition of trifluoromethanesulfonic anhydride (109.5 mg, 0.54 mmol). The reaction mixture was stirred at 0° C. for 1 h, quenched with water and extracted with DCM. The combine organic layers were washed with brine, dried over Na₂SO₄, concentrated and purified by column chromatography to obtain Trifluoromethanesulfonic acid 5-(2-methyl-5-trifluoromethyl-2H-pyrazol-3-yl)-1H-indol-2-yl ester (120 mg, 82%).

2-(3-Chloro-2-methoxy-pyridin-4-yl)-5-(2-methyl-5-trifluoromethyl-2H-pyrazol-3-yl)-1H-indole: A solution of Trifluoromethanesulfonic acid 5-(2-methyl-5-trifluoromethyl-2H-pyrazol-3-yl)-1H-indol-2-yl ester (100 mg, 0.2427 mmol) and (3-Chloro-2-methoxypyridin-4-yl)boronic acid (101 mg mg, 0.4720 mmol) in 1,4-dioxane (2 mL) was degassed and purged with nitrogen (10 min) and then aqueous K₂CO₃ (2 M, 0.4 mL) was added and purged with nitrogen again (20 min). Pd(dppf)Cl₂ (20 mol %, 40 mg) was added to the above reaction mixture and stirred at 100° C. for 2 h. After the completion of the reaction it was filtered through Celite and the filtrate was diluted with water and extracted with EtOAc. The organic phase was washed with brine, dried over Na₂SO₄ and concentrated. The Crude material was purified by column chromatography to obtain 2-(3-Chloro-2-methoxy-pyridin-4-yl)-5-(2-methyl-5-trifluoromethyl-2H-pyrazol-3-yl)-1H-indole (20 mg, 20%), MS (M+H)=407.

### Example 44

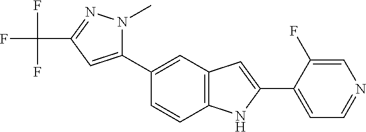

### 2-(3-Fluoro-pyridin-4-yl)-5-(2-methyl-5-trifluoromethyl-2H-pyrazol-3-yl)-1H-indole

Prepared in a manner identical to that described for example 43. MS (M+H)=361.

### Example 45

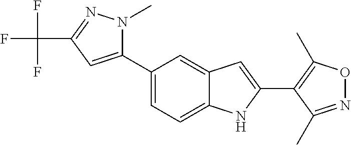

### 2-(3,5-Dimethyl-isoxazol-4-yl)-5-(2-methyl-5-trifluoromethyl-2H-pyrazol-3-yl)-1H-indole

Prepared in a manner identical to that described for example 43. MS (M+H)=362.

### Example 46

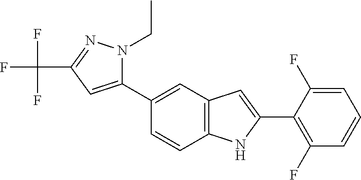

### 2-(2,6-Difluoro-phenyl)-5-(2-ethyl-5-trifluoromethyl-2H-pyrazol-3-yl)-1H-indole

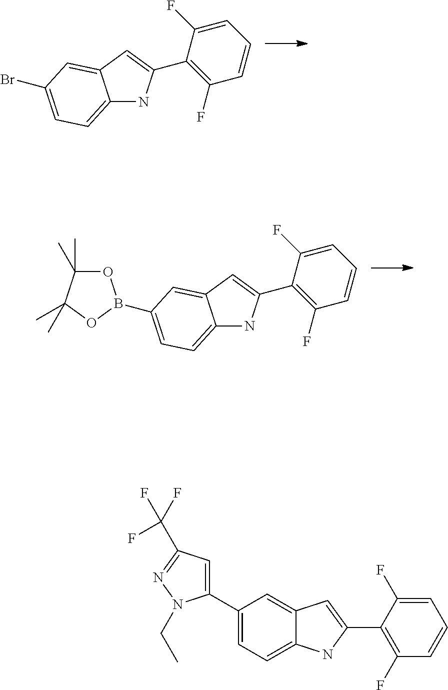

2-(2,6-Difluoro-phenyl)-5-(4,4,5,5-tetramethyl-[1,3,2]dioxaborolan-2-yl)-1H-indole: To a solution of 5-Bromo-2-(2,6-difluoro-phenyl)-1H-indole (200 mg, 0.64 mmol) in acetonitrile (7 ml) was added bispinacolatodiborane (328 mg, 1.29 mmol) and potassium acetate (191 mg, 1.94 mmol). The above reaction mass was purged with nitrogen for 20 min then Pd(dppf)Cl₂ (30 mol %, 47 mg) was added and stirred at 100° C. for 14 h. After the completion of the reaction it was filtered through Celite and the filtrate was diluted with water and extracted with EtOAc. The organic phase. was washed with brine, dried over Na₂SO₄ and concentrated. The crude compound was purified by column chromatography to obtain 2-(2,6-Difluoro-phenyl)-5-(4,4,5,5-tetramethyl-[1,3,2]dioxaborolan-2-yl)-1H-indole (130 mg, 60%).

2-(2,6-Difluoro-phenyl)-5-(2-ethyl-5-trifluoromethyl-2H-pyrazol-3-yl)-1H-indole: To a solution of obtain 2-(2,6-Difluoro-phenyl)-5-(4,4,5,5-tetramethyl-[1,3,2]dioxaborolan-2-yl)-1H-indole (100 mg, 0.28 mmol) and Trifluoro-methanesulfonic acid 2-ethyl-5-trifluoromethyl-2H-pyrazol-3-yl ester (Intermediate 12, 131.83 mg, 0.422 mmol) in 1,4-dioxane (4 mL) was degassed and purged with nitrogen (10 min) and then aqueous K₂CO₃ (2 M, 0.6 mL) was added and purged with nitrogen again (20 min). Pd(dppf)Cl₂ (10 mol %, 23 mg) was added to the above reaction mixture and stirred at 100° C. for 4 h. After the completion of the reaction it was filtered through Celite and the filtrate extracted with EtOAc. The organic phase was washed with brine, dried over Na₂SO₄ and concentrated. The Crude material was purified by column chromatography to obtain 2-(2,6-Difluoro-phenyl)-5-(2-ethyl-5-trifluoromethyl-2H-pyrazol-3-yl)-1H-indole (15 mg, 13%), MS (M+H)=392.

### Example 47

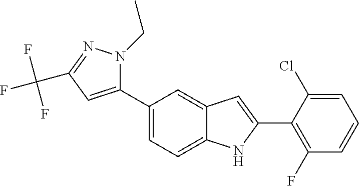

### 2-(2-Chloro-6-fluoro-phenyl)-5-(2-ethyl-5-trifluoromethyl-2H-pyrazol-3-yl)-1H-indole

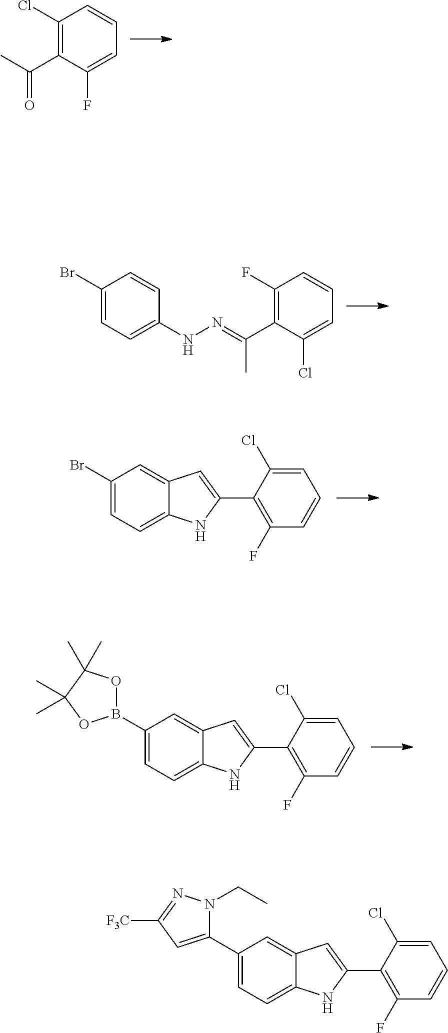

N-(4-Bromo-phenyl)-N′-[1-(2-chloro-6-fluoro-phenyl)-eth-(E)-ylidene]-hydrazine: To a solution of 1-(2-chloro-6-fluoro-phenyl)-ethanone (3 g, 17 mmol) and (4-bromo-phenyl)-hydrazine (4.66 g, 20.9 mmol) in EtOH (15 ml) was added aq. KOAc (5.12 g, 52.1 mmol in 10 ml water) and stirred at 25° C. for 16 hrs., diluted with water and extracted with hexanes. The organic phase was washed with brine, dried over Na₂SO₄ and concentrated to give N-(4-bromo-phenyl)-N′-[1-(2-chloro-6-fluoro-phenyl)-eth-(E)-ylidene]-hydrazine (2.5 g, 42%).

Bromo-2-(2-chloro-6-fluoro-phenyl)-1H-indole: N-(4-Bromo-phenyl)-N′-[1-(2-chloro-6-fluoro-phenyl)-eth-(E)-ylidene]-hydrazine (400 mg, 1.17 mmol) was treated with polyphosphoric acid (1 g), heated to 110° C. and stirred for 1 h. The temperature was decreased to 70° C. then a (1:5) mixture of water and EtOAc were added. The organic layer was washed with brine, dried over Na₂SO₄ and concentrated. The crude material was purified by column chromatography to give 5-bromo-2-(2-chloro-6-fluoro-phenyl)-1H-indole (350 mg, 92%).

2-(2-Chloro-6-fluoro-phenyl)-5-(4,4,5,5-tetramethyl-[1,3,2]dioxaborolan-2-yl)-1H-indole: To a solution of 5-bromo-2-(2-chloro-6-fluoro-phenyl)-1H-indole (1.5 g, 4.6 mmol) in acetonitrile (28 ml) was added bis-pinacolato diborane (2.34 g, 9.25 mmol) and potassium acetate (1.35 g, 13.8 mmol). The reaction mixture was purged with nitrogen for 20 min then Pd(dppf)Cl₂ (30 mol %, 1.13 g) was added and stirred at 100° C. for 14 h. The reaction mixture was filtered through Celite and the filtrate was diluted with water and extracted with EtOAc. The organic phase was washed with brine, dried over Na₂SO₄ and concentrated. The crude compound was purified by column chromatography to give 2-(2-chloro-6-fluoro-phenyl)-5-(4,4,5,5-tetramethyl-[1,3,2]dioxaborolan-2-yl)-1H-indole (900 mg, 52%).

2-(2-Chloro-6-fluoro-phenyl)-5-(2-ethyl-5-trifluoromethyl-2H-pyrazol-3-yl)-1H-indole: Prepared in a similar manner to the final step of Example 46 replacing 2-(2,6-di-fluorophenyl)-5-(4,4,5,5-tetramethyl-[1,3,2]dioxaborolan-2-yl)-1H-indole with 2-(2-chloro-6-fluoro-phenyl)-5-(4,4,5,5-tetramethyl-[1,3,2]dioxaborolan-2-yl)-1H-indole. MS (M+H)=408.

### Example 48

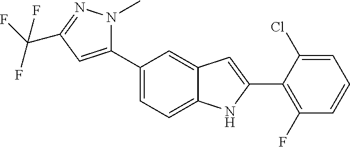

### 2-(2-Chloro-6-fluoro-phenyl)-5-(2-methyl-5-trifluoromethyl-2H-pyrazol-3-yl)-1H-indole

Prepared in a manner identical to Example 47, using the material prepared in Example 47 and Intermediate 11. MS (M+H)=394.

### Example 49

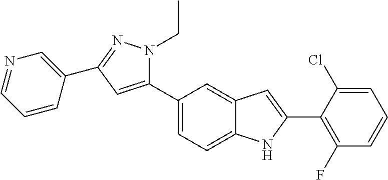

### 2-(2-Chloro-6-fluoro-phenyl)-5-(2-ethyl-5-pyridin-3-yl-2H-pyrazol-3-yl)-1H-indole

Prepared in a manner identical to Example 47, using the material prepared in Example 47 and Intermdiate 6. MS (M+H)=417.

### Example 50

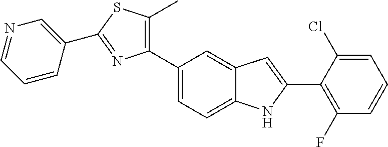

### 2-(2-Chloro-6-fluoro-phenyl)-5-(5-methyl-2-pyridin-3-yl-thiazol-4-yl)-1H-indole

Prepared in a manner identical to Example 47, using the material prepared in Example 47 and Intermediate 13. MS (M+H)=420.

### Example 51

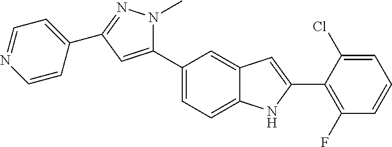

### 2-(2-Chloro-6-fluoro-phenyl)-5-(2-methyl-5-pyridin-4-yl-2H-pyrazol-3-yl)-1H-indole

Prepared in a manner identical to Example 47, using the material prepared in Example 47 and Intermediate 4. MS (M+H)=403.

### Example 52

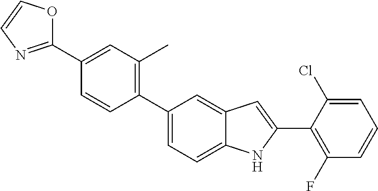

### 2-(2-Chloro-6-fluoro-phenyl)-5-(2-methyl-4-oxazol-2-yl-phenyl)-1H-indole

Prepared in a manner identical to Example 47, using the material prepared in Example 47 and Intermediate 9. MS (M+H)=403.

### Example 53

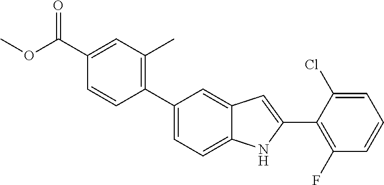

### 4-[2-(2-Chloro-6-fluoro-phenyl)-1H-indol-5-yl]-3-methyl-benzoic acid methyl ester

Prepared in a manner identical to Example 47, using the material prepared in Example 47 and the commercially available 4-iodo-3-methyl-benzoic acid methyl ester. MS (M+H)=394.

### Example 54

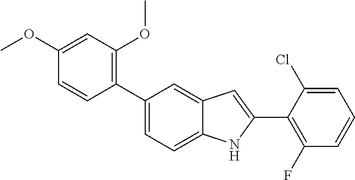

### 2-(2-chloro-6-fluoro-phenyl)-5-(2,4-dimethoxy-phenyl)-1H-indole

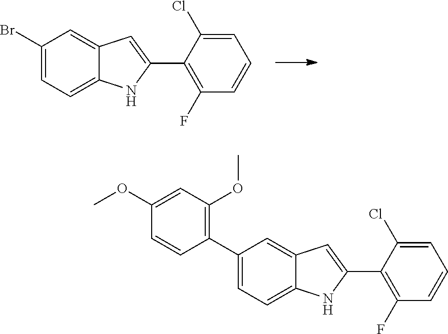

A solution of 5-bromo-2-(2-chloro-6-fluoro-phenyl)-1H-indole (100 mg, 0.308 mmol) and 2,4-dimethoxy-phenyl-boronic acid (56 mg, 0.31 mmol) in 1,4-dioxane (2 mL) was degassed and purged with nitrogen (10 min), then aqueous K₂CO₃ (2 M, 0.2 mL) was added and purged with nitrogen again (20 min). Pd(dppf)Cl₂ (10 mol %, 25 mg) was added to the above reaction mixture and stirred at 100° C. for 4 hrs. The cooled reaction mixture was filtered through Celite and the filtrate was diluted with water, extracted with EtOAc. The organic phase was washed with brine, dried over Na₂SO₄ and concentrated. The crude material was purified by column chromatography (10-30% EtOAc/hexanes) to give 2-(2-chloro-6-fluoro-phenyl)-5-(2,4-dimethoxy-phenyl)-1H-indole (20 mg, 18%), MS (M+H)=382.

### Example 55

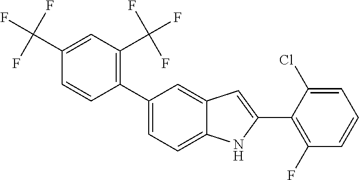

### 5-(2,4-Bis-trifluoromethyl-phenyl)-2-(2-chloro-6-fluoro-phenyl)-1H-indole

Prepared in a manner identical to Example 54, using the commercially available 2,4-bis-trifluoromethyl-phenyl-boronic acid. MS (M+H)=458.

### Example 56

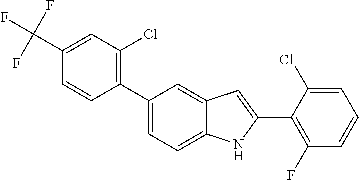

### 2-(2-Chloro-6-fluoro-phenyl)-5-(2-chloro-4-trifluoromethyl-phenyl)-1H-indole

Prepared in a manner identical to Example 54, using the commercially available 2-chloro-4-trifluoromethyl-phenyl-boronic acid. MS (M+H)=424.

### Example 57

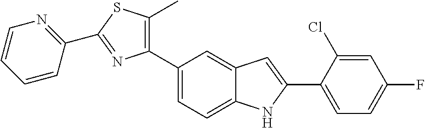

### 2-(2-Chloro-4-fluoro-phenyl)-5-(5-methyl-2-pyridin-2-yl-thiazol-4-yl)-1H-indole

Prepared in a manner identical to that described above in Example 9 substituting commercially available 2-chloro-4-fluoro-phenyl-boronic acid in the penultimate step. MS (M+H)=420.

### Example 58

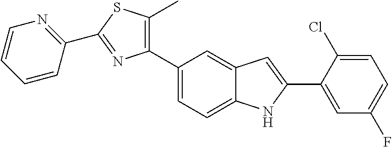

### 2-(2-Chloro-5-fluoro-phenyl)-5-(5-methyl-2-pyridin-2-yl-thiazol-4-yl)-1H-indole

Prepared in a manner identical to that described above in Example 9 substituting commercially available 2-chloro-5-fluoro-phenylboronic acid in the penultimate step. MS (M+H)=420.

### Example 59

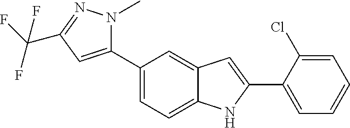

### 2-(2-Chloro-phenyl)-5-(2-methyl-5-trifluoromethyl-2H-pyrazol-3-yl)-1H-indole

Prepared in a manner identical to that described in Example 5 substituting Intermediate 15 in the Suzuki step. MS (M+H)=376.

### Example 60

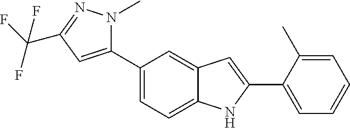

### 5-(2-Methyl-5-trifluoromethyl-2H-pyrazol-3-yl)-2-o-tolyl-1H-indole

Prepared in a manner identical to that described in Example 6 substituting Intermediate 15 in the Suzuki step. MS (M+H)=356.

### Example 61

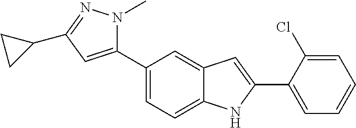

### 2-(2-Chloro-phenyl)-5-(5-cyclopropyl-2-methyl-2H-pyrazol-3-yl)-1H-indole

Prepared in a manner identical to that described in Example 5 substituting Intermediate 16 in the Suzuki step. MS (M+H)=349.

### Example 62

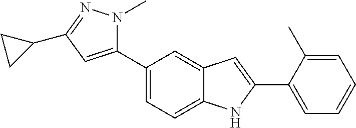

### 5-(5-Cyclopropyl-2-methyl-2H-pyrazol-3-yl)-2-o-tolyl-1H-indole

Prepared in a manner identical to that described in Example 6 substituting intermediate 16 in the Suzuki step. MS (M+H)=328.

### Example 63

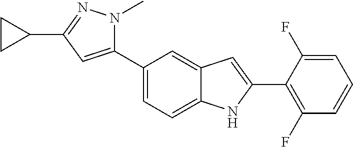

### 5-(5-Cyclopropyl-2-methyl-2H-pyrazol-3-yl)-2-(2,6-difluoro-phenyl)-1H-indole

Prepared in a manner identical to that described in Example 15 substituting intermediate 16 in the Suzuki step. MS (M+H)=350.

### Example 64

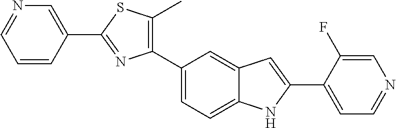

### 2-(3-Fluoro-pyridin-4-yl)-5-(5-methyl-2-pyridin-3-yl-thiazol-4-yl)-1H-indole

Prepared in a manner identical to that described in Example 9 substituting thionicotimamide in the thiazole formation. MS (M+H)=387.

### Example 65

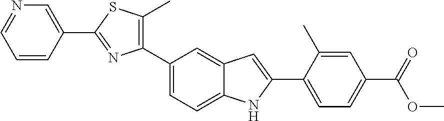

### Methyl-4-[5-(5-methyl-2-pyridin-3-yl-thiazol-4-yl)-1H-indol-2-yl]-benzoic acid methyl ester

Prepared in a manner identical to that described in Example 9 substituting thionicotimamide in the thiazole formation and using the commercially available 4-(methoxycarbonyl)-2-methylphenyl boronic acid in the Suzuki step. MS (M+H)=440.

### Example 66

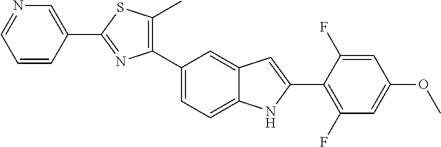

### 2-(2,6-Difluoro-4-methoxy-phenyl)-5-(5-methyl-2-pyridin-3-yl-thiazol-4-yl)-1H-indole

Prepared in a manner identical to that described in Example 9 substituting thionicotimamide in the thiazole formation and using the commercially available 2,6-difluoro-4-methoxyphenyl boronic acid in the Suzuki step. MS (M+H)=434.

### Example 67

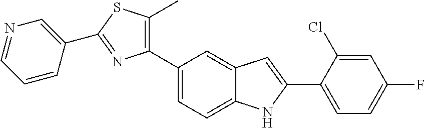

### 2-(2-Chloro-4-fluoro-phenyl)-5-(5-methyl-2-pyridin-3-yl-thiazol-4-yl)-1H-indole

Prepared in a manner identical to that described in Example 9 substituting thionicotimamide in the thiazole formation and using the commercially available 2-chloro-4-fluoro-phenyl-boronic acid in the Suzuki step. MS (M+H)=420.

### Example 68

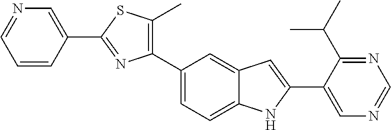

### 2-(4-Isopropyl-pyrimidin-5-yl)-5-(5-methyl-2-pyridin-3-yl-thiazol-4-yl)-1H-indole

Prepared in a manner identical to that described in Example 9 substituting thionicotimamide in the thiazole formation and using the commercially available 4-isopropylpyrimidine-5-boronic acid in the Suzuki step. MS (M+H)=412.

### Example 69

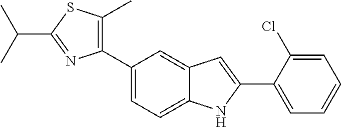

### 2-(2-Chloro-phenyl)-5-(2-isopropyl-5-methyl-thiazol-4-yl)-1H-indole

Prepared in a manner identical to that described in Example 9 substituting thioisobutyramide in the thiazole formation and using the commercially available 2-chloro-phenylboronic acid in the Suzuki step. MS (M+H)=367.

### Example 70

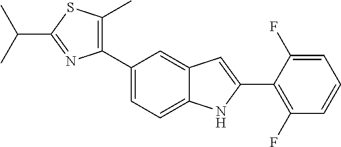

### 2-(2,6-Difluoro-phenyl)-5-(2-isopropyl-5-methyl-thiazol-4-yl)-1H-indole

Prepared in a manner identical to that described in Example 9 substituting thioisobutyramide in the thiazole formation and using the commercially available 2,6-difluoro-phenylboronic acid in the Suzuki step. MS (M+H)=369.

### Example 71

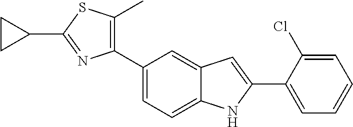

### 2-(2,6-Difluoro-phenyl)-5-(2-isopropyl-5-methyl-thiazol-4-yl)-1H-indole

Prepared in a manner identical to that described in Example 9 substituting cyclopropanecarbothioamide in the thiazole formation and using the commercially available 2-chloro-phenylboronic acid in the Suzuki step. MS (M+H)=365.

### Example 72

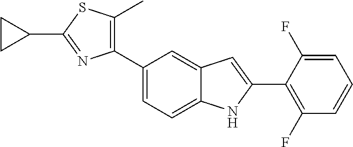

### 5-(2-Cyclopropyl-5-methyl-thiazol-4-yl)-2-(2,6-difluoro-phenyl)-1H-indole

Prepared in a manner identical to that described in Example 9 substituting cyclopropanecarbothioamide in the thiazole formation and using the commercially available 2,6-difluorophenylboronic acid in the Suzuki step. MS (M+H)=367.

### Example 73

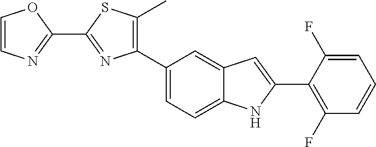

### 2-(2,6-Difluoro-phenyl)-5-(5-methyl-2-oxazol-2-yl-thiazol-4-yl)-1H-indole

Prepared in a manner identical to that described in Example 9 substituting oxazole-2-carbothioamide in the thiazole formation and using the commercially available 2,6-difluorophenylboronic acid in the Suzuki step. MS (M+H)=394.

### Example 74

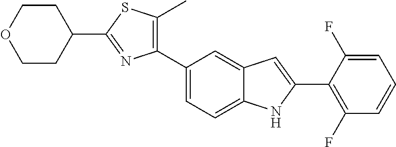

### 2-(2,6-Difluoro-phenyl)-5-[5-methyl-2-(tetrahydro-pyran-4-yl)-thiazol-4-yl]-1H-indole

Prepared in a manner identical to that described in Example 9 substituting tetrahydropyran-4-carbothioamide in the thiazole formation and using the commercially available 2,6-difluorophenylboronic acid in the Suzuki step. MS (M+H)=411.

### Example 75

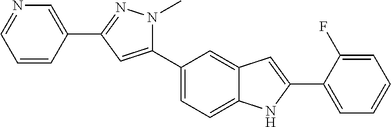

### 2-(2-Fluoro-phenyl)-5-(2-methyl-5-pyridin-3-yl-2H-pyrazol-3-yl)-1H-indole

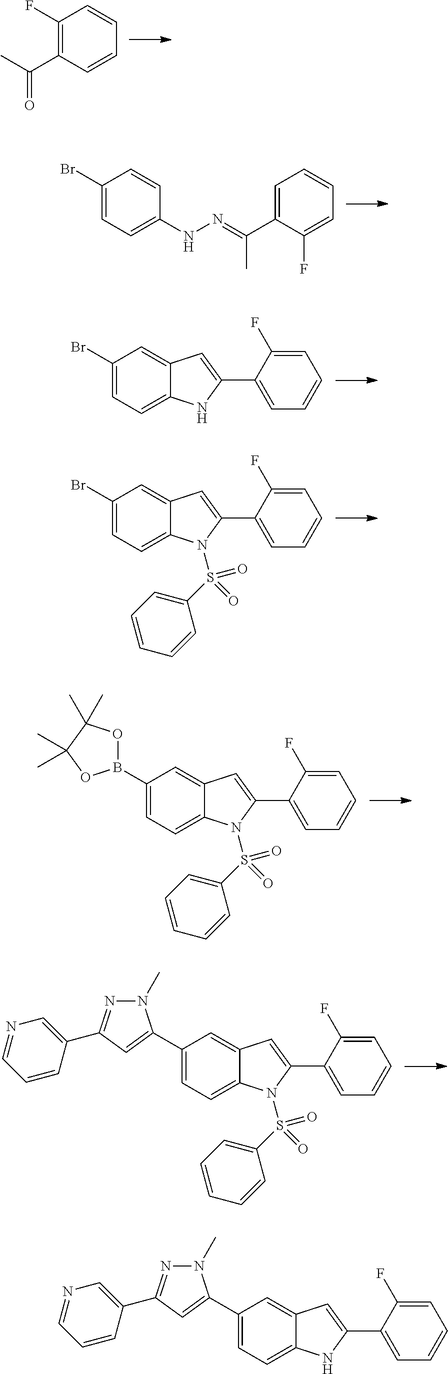

N-(4-Bromo-phenyl)-N′-[1-(2-fluoro-phenyl)-eth-(E)-ylidene]-hydrazine: To a solution of 1-(2-fluoro-phenyl)-ethanone (3.1 g, 22 mmol) and (4-bromophenyl)-hydrazine (5.0 g, 22 mmol) in EtOH was added KOAc (2.2 g, 22 mmol) and stirred at 25° C. for 16 hrs. The reaction mixture was extracted with hexanes (4×50 mL) and the organic phase was washed with brine, dried over Na₂SO₄ and concentrated to give N-(4-bromo-phenyl)-N′-[1-(2-fluoro-phenyl)-eth-(E)-ylidene]-hydrazine (5.5 g, 80%).

Bromo-2-(2-fluoro-phenyl)-1H-indole (3): To a 70° C. solution of polyphosphoric acid was added N-(4-Bromo-phenyl)-N′-[1-(2-fluoro-phenyl)-eth-(E)-ylidene]-hydrazine (5.5 g, 18 mmol). The reaction mixture was then heated to 110° C. for 2 h. The temperature was decreased to 25° C. and ice-water was added and extracted with EtOAc (3×50 mL). The organic layer was washed with brine, dried over Na₂SO₄ and concentrated. This was purified by column chromatography (Hexane) to obtain 5-bromo-2-(2-fluoro-phenyl)-1H-indole (2 g, 39%).

Benzenesulfonyl-5-bromo-2-(2-fluoro-phenyl)-1H-indole: To a 0° C. solution of 5-bromo-2-(2-fluoro-phenyl)-1H-indole (1.8 g, 6.2 mmol) in DMF was added NaH (0.22 g, 9.3 mmol), stirred for 30 min., then benzenesulfonylchloride (1.31 g, 7.44 mmol) was added dropwise at 0° C. and warmed the reaction mixture to 25° C. and stirred for 2 hrs., The reaction mixture was extracted with EtOAc (3×50 mL). The organic phase was washed with brine, dried over Na₂SO₄ and purified by combiflash chromatography to give 1-benzenesulfonyl-5-bromo-2-(2-fluoro-phenyl)-1H-indole (2.0 g, 74%).

Benzenesulfonyl-2-(2-fluoro-phenyl)-5-(4,4,5,5-tetramethyl-[1,3,2]dioxaborolan-2-yl)-1H-indole: To a solution of 1-benzenesulfonyl-5-bromo-2-(2-fluoro-phenyl)-1H-indole (1.6 g, 3.7 mmol) in 1,4-dioxane was added bis-pinacolatodiborane (1.88 g, 7.44 mmol) and KOAc (0.73 g, 7.4 mmol). The reaction mixture was stirred at 110° C. for 14 hrs., then the cooled reaction mixture was extracted with EtOAc (3×50 mL). The organic phase was washed with brine, dried over Na₂SO₄ and concentrated. The crude compound was purified by combiflash column chromatography (2% EtOAc-Hexane) to give 1-benzenesulfonyl-2-(2-fluoro-phenyl)-5-(4,4,5,5-tetramethyl-[1,3,2]dioxaborolan-2-yl)-1H-indole (0.80 g, 44%).

Benzenesulfonyl-2-(2-fluoro-phenyl)-5-(2-methyl-5-pyridin-3-yl-2H-pyrazol-3-yl)-1H-indole: A solution of 1-benzenesulfonyl-2-(2-fluoro-phenyl)-5-(4,4,5,5-tetramethyl-[1,3,2]dioxaborolan-2-yl)-1H-indole (150 mg, 0.31 mmol) and Intermediate 6 (144 mg, 0.47 mmol) in 1,4-dioxane (2 mL) was degassed and purged with nitrogen (10 min), then aqueous K₂CO₃ (2 M, 0.31 mL) was added and purged with nitrogen again (20 min). Pd(dppf)₂Cl₂ (10 mol %, 25 mg) was added to the above reaction mixture and stirred at 100° C. for 18 hrs. The cooled reaction mixture was filtered through Celite and the filtrate extracted with EtOAc (3×20 mL). The organic phase (EtOAc layer) was washed with brine, dried over Na₂SO₄ and concentrated to give a crude material that was purified by column chromatography (1% MeOH/CH₂Cl₂) to give 1-benzenesulfonyl-2-(2-fluoro-phenyl)-5-(2-methyl-5-pyridin-3-yl-2H-pyrazol-3-yl)-1H-indole (100 mg, 63%).

2-(2-Fluoro-phenyl)-5-(2-methyl-5-pyridin-3-yl-2H-pyrazol-3-yl)-1H-indole: To a solution of 1-benzenesulfonyl-2-(2-fluoro-phenyl)-5-(2-methyl-5-pyridin-3-yl-2H-pyrazol-3-yl)-1H-indole (90 mg, 0.18 mmol) in THF/MeOH (2:1), was added Cs₂CO₃ (175 mg, 0.535 mmol) and stirred at 25° C. for 24 h. The reaction mixture was concentrated and extracted with EtOAc (3×10 mL). The organic phase was washed with brine, dried over Na₂SO₄ and concentrated. The crude compound was purified by combiflash column chromatography (1:99 MeOH/CH₂Cl₂) to give 2-(2-fluoro-phenyl)-5-(2-methyl-5-pyridin-3-yl-2H-pyrazol-3-yl)-1H-indole (26 mg, 39%), MS (M+H)=369.

### Example 76

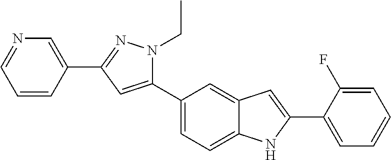

### 2-(2-Fluoro-phenyl)-5-(2-ethyl-5-pyridin-3-yl-2H-pyrazol-3-yl)-1H-indole

Prepared in a manner identical to that described in example 75, substituting intermediate 7 in the Suzuki step. MS (M+H)=383.

### Example 77

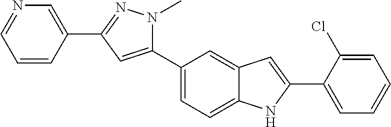

### 2-(2-Chloro-phenyl)-5-(2-methyl-5-pyridin-3-yl-2H-pyrazol-3-yl)-1H-indole

Prepared in a manner identical to that described in example 75, starting from the commercially available 1-(2-chloro-phenyl)-ethanone and using Intermediate 6 in the Suzuki step. MS (M+H)=385.

### Example 78

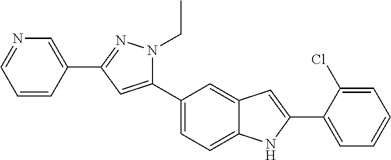

### 2-(2-Chloro-phenyl)-5-(2-ethyl-5-pyridin-3-yl-2H-pyrazol-3-yl)-1H-indole

Prepared in a manner identical to that described in example 75, starting from the commercially available 1-(2-chloro-phenyl)-ethanone and using Intermediate 7 in the Suzuki step. MS (M+H)=399.

### Example 79

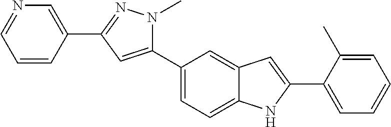

### 5-(2-Methyl-5-pyridin-3-yl-2H-pyrazol-3-yl)-2-o-tolyl-1H-indole

Prepared in a manner identical to that described in example 75, starting from the commercially available 2′-methylacetophenone and using Intermediate 6 in the Suzuki step. MS (M+H)=365.

### Example 80

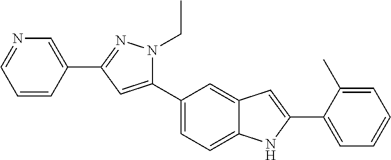

### 5-(2-Ethyl-5-pyridin-3-yl-2H-pyrazol-3-yl)-2-o-tolyl-1H-indole

Prepared in a manner identical to that described in example 75, starting from the commercially available 2′-methylacetophenone and using Intermediate 7 in the Suzuki step. MS (M+H)=379.

### Example 81

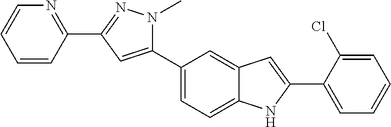

### 2-(2-Chloro-phenyl)-5-(2-methyl-5-pyridin-2-yl-2H-pyrazol-3-yl)-1H-indole

Prepared in a manner identical to that described in example 75, starting from the commercially available 1-(2-chloro-phenyl)-ethanone and using Intermediate 17 in the Suzuki step. MS (M+H)=385.

### Example 82

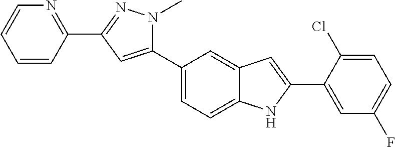

### 2-(2-Chloro-5-fluoro-phenyl)-5-(2-methyl-5-pyridin-2-yl-2H-pyrazol-3-yl)-1H-indole

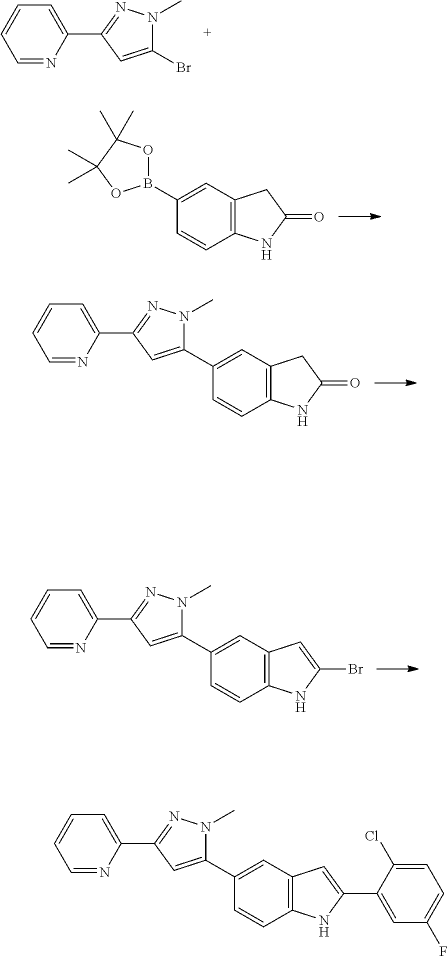

5-(2-Methyl-5-pyridin-2-yl-2H-pyrazol-3-yl)-1,3-dihydro-indol-2-one: A solution of Intermediate 17 (760 mg, 3.16 mmol) and 5-(4,4,5,5-tetramethyl-[1,3,2]dioxaborolan-2-yl)-1,3-dihydro-indol-2-one (820 mg, 3.16 mmol) in 1,4-dioxane (25 mL) was purged with nitrogen (20 min) and then aqueous K₂CO₃ (2 M, 1.2 mL) was added and purged with nitrogen again (30 min). Pd(dppf)Cl₂ (10 mol %, 258 mg, 0.316 mmol) was then added to the above reaction mixture and stirred at 100° C. for 4 h. The reaction mixture was filtered through Celite and the filtrate was diluted with water and extracted with EtOAc. The organic phase was washed with brine, dried over Na₂SO₄ and concentrated. The crude compound was purified by column chromatography to give 5-(2-methyl-5-pyridin-2-yl-2H-pyrazol-3-yl)-1,3-dihydro-indol-2-one (340 mg, 37%).

Bromo-5-(2-methyl-5-pyridin-2-yl-2H-pyrazol-3-yl)-1H-indole: To a solution of 5-(2-methyl-5-pyridin-2-yl-2H-pyrazol-3-yl)-1,3-dihydro-indol-2-one (500 mg, 1.72 mmol) in dry dichloroethane (35 ml) was added a POBr₃ solution (1M in dichloroethane, 3.4 ml, 3.4 mmol). The reaction mixture was reflux for 30 min., then cooled to 70° C. and imidazole (140 mg, 2.06 mmol) was added and refluxed for 90 min. To the cooled reaction mixture was added ice-water, then neutralized using aq. NaHCO₃ and extracted with dichloromethane. The organic phase was washed with brine, dried over Na₂SO₄ and concentrated under vacuum. The crude compound was purified by column chromatography to give 2-bromo-5-(2-methyl-5-pyridin-2-yl-2H-pyrazol-3-yl)-1H-indole (300 mg, 49%).

2-(2-Chloro-5-fluoro-phenyl)-5-(2-methyl-5-pyridin-2-yl-2H-pyrazol-3-yl)-1H-indole: A solution of 2-bromo-5-(2-methyl-5-pyridin-2-yl-2H-pyrazol-3-yl)-1H-indole (65 mg, 0.19 mmol) and 2-chloro-5-fluorophenylboronic acid (38 mg, 0.22 mmol) in acetonitrile (1.5 mL) was purged with nitrogen (10 min), then aqueous K₂CO₃ (2 M, 0.16 mL) was added and purged with nitrogen again (20 min). Pd (dppf)Cl₂ (10 mol %, 14 mg) was added to the above reaction mixture and stirred at 100° C. for 4 h. The cooled reaction mixture was filtered through Celite and concentrated. The crude material was purified by column chromatography to give 2-(2-Chloro-5-fluoro-phenyl)-5-(2-methyl-5-pyridin-2-yl-2H-pyrazol-3-yl)-1H-indole (27 mg, 37%), MS (M+H)=403.

### Example 83

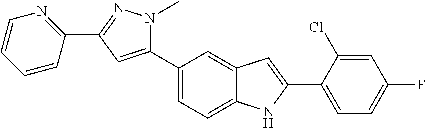

### 2-(2-Chloro-4-fluoro-phenyl)-5-(2-methyl-5-pyridin-2-yl-2H-pyrazol-3-yl)-1H-indole

Prepared as described in Example 82 using 2-chloro-4-fluoroboronic acid in the final step. MS (M+H)=403.

### Example 84

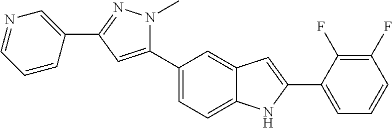

### 2-(2,3-Difluoro-phenyl)-5-(2-methyl-5-pyridin-3-yl-2H-pyrazol-3-yl)-1H-indole

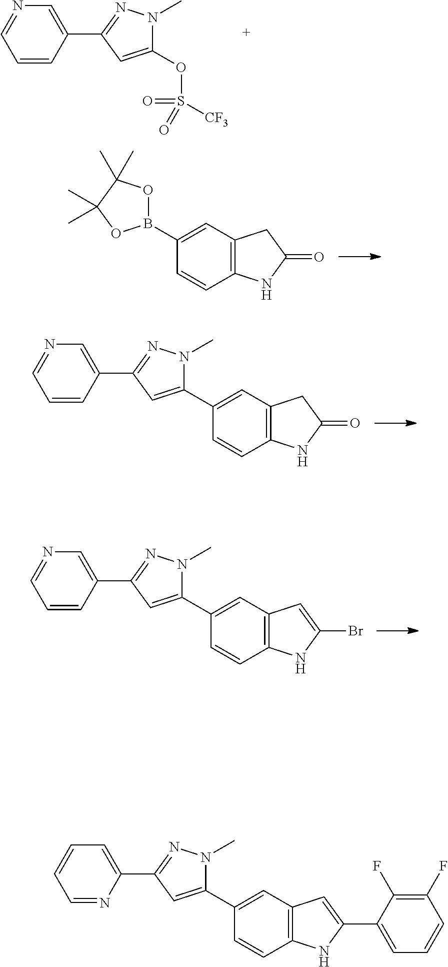

5-(2-Methyl-5-pyridin-3-yl-2H-pyrazol-3-yl)-1,3-dihydro-indol-2-one: A solution of Intermediate 6 (2.0 g, 6.4 mmol) and 5-(4,4,5,5-tetramethyl-[1,3,2]dioxaborolan-2-yl)-1,3-dihydro-indol-2-one (1.68 g, 6.4 mmol) in 1,4-dioxane (60 mL) was degassed and purged with nitrogen (20 min) and then aqueous K₂CO₃ (2 M, 4 mL) was added and purged with nitrogen again (30 min). Pd(dppf)Cl₂ (10 mol %, 562 mg) was then added to the above reaction mixture and stirred at 100° C. for 4 h. The cooled reaction mixture was filtered through Celite and the filtrate was diluted with water and extracted with EtOAc. The organic phase was washed with brine, dried over Na₂SO₄ and concentrated. The crude compound was purified by column chromatography to give 5-(2-methyl-5-pyridin-3-yl-2H-pyrazol-3-yl)-1,3-dihydro-indol-2-one (1.2 g, 64%).

Bromo-5-(2-methyl-5-pyridin-3-yl-2H-pyrazol-3-yl)-1H-indole: To a solution of 5-(2-methyl-5-pyridin-3-yl-2H-pyrazol-3-yl)-1,3-dihydro-indol-2-one (290 mg, 0.68 mmol) in dry dichloroethane (10 ml) was added POBr₃ solution (1M in dichloroethane, 1.3 ml, 1.3 mmol) and it was then reflux for 30 min. Then the reaction mixture was cooled to 70° C. and imidazole (60 mg, 0.75 mmol) was added and reflux for 90 min. After completion of reaction it was cooled to RT. Ice-water was then added to quench the reaction, neutralized using aq. NaHCO₃ and extracted with DCM. The organic phase was washed with brine, dried over Na₂SO₄ and concentrated under vacuum. The crude compound was purified by column chromatography followed by prep-HPLC to give 2-bromo-5-(2-methyl-5-pyridin-3-yl-2H-pyrazol-3-yl)-1H-indole (100 mg, 28%).

2-(2,3-Difluoro-phenyl)-5-(2-methyl-5-pyridin-3-yl-2H-pyrazol-3-yl)-1H-indole: A solution of 2-bromo-5-(2-methyl-5-pyridin-3-yl-2H-pyrazol-3-yl)-1H-indole (80 mg, 0.23 mmol) and 2,3-difluorophenyl boronic acid (43 mg, 0.27 mmol) in dioxane (4 mL) was purged with nitrogen (10 min) and then aqueous K₂CO₃ (2 M, 0.2 mL) was added and purged with nitrogen again (20 min), Pd(dppf)Cl₂ (10 mol %, 18 mg) was added, then stirred at 100° C. for 4 h. The cooled reaction mixture was filtered through Celite and concentrated. The crude material was purified by column chromatography to give 2-(2,3-Difluoro-phenyl)-5-(2-methyl-5-pyridin-3-yl-2H-pyrazol-3-yl)-1H-indole (25 mg, 29%), MS (M+H)=387.

### Example 85

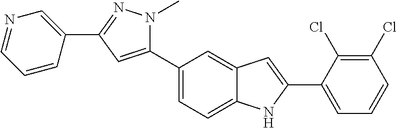

### 2-(2,3-Dichloro-phenyl)-5-(2-methyl-5-pyridin-3-yl-2H-pyrazol-3-yl)-1H-indole

Prepared as described in Example 84 using commercially available 2,3-dichloro-phenylboronic acid in the final step. MS (M+H)=419.

### Example 86

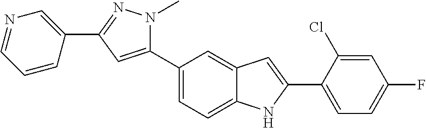

### 2-(2-Chloro-4-fluoro-phenyl)-5-(2-methyl-5-pyridin-3-yl-2H-pyrazol-3-yl)-1H-indole

Prepared as described in Example 84 using commercially available 2-chloro-4-fluoroboronic acid in the final step. MS (M+H)=403.

### Example 87

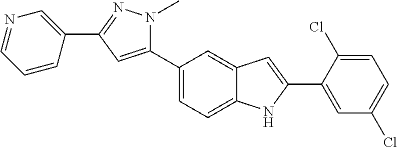

### 2-(2,5-Dichloro-phenyl)-5-(2-methyl-5-pyridin-3-yl-2H-pyrazol-3-yl)-1H-indole

Prepared as described in Example 84 using commercially available 2,5-dichlorophenylboronic acid in the final step. MS (M+H)=419.

### Example 88

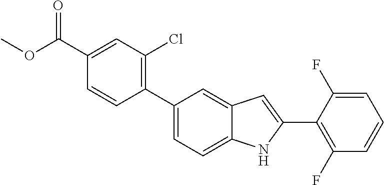

### 4-[2-(2,6-Difluoro-phenyl)-1H-indol-5-yl]-3-chloro-benzoic acid methyl ester

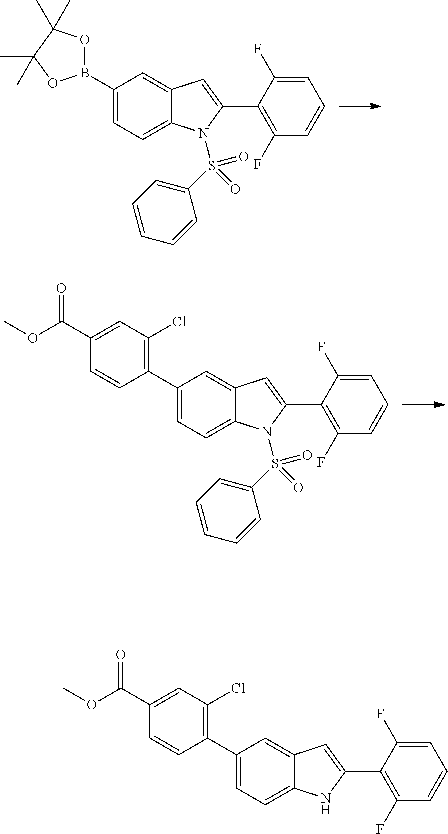

4-[1-Benzenesulfonyl-2-(2,6-difluoro-phenyl)-1H-indol-5-yl]-3-chloro-benzoic acid

methyl ester: A solution of 1-benzenesulfonyl-2-(2,6-difluoro-phenyl)-5-(4,4,5,5-tetramethyl-[1,3,2]dioxaborolan-2-yl)-1H-indole (200 mg, 0.40 mmol) and 4-bromo-3-chloro-benzoic acid methyl ester (82 mg, 0.60 mmol) in 1,4-dioxane (5 mL) was purged with nitrogen (10 min), then Cs₂CO₃ (263 mg, 0.80 mmol) and Pd(dppf)Cl₂ (33 mg, 0.040 mmol) were added, and purged with nitrogen again (5 min). The reaction mixture was stirred at 100° C. for 4 h. After the completion of reaction it was filtered through Celite and the filtrate was diluted with water and extracted with EtOAc. The organic phase was washed with brine, dried over Na₂SO₄ and concentrated. The crude compound was purified by CombiFlash column chromatography to give 4-[1-benzenesulfonyl-2-(2,6-difluoro-phenyl)-1H-indol-5-yl]-3-chloro-benzoic acid methyl ester (90 mg, 41%).

4-[2-(2,6-Difluoro-phenyl)-1H-indol-5-yl]-3-chloro-benzoic acid methyl ester: To a solution of 4-[1-benzenesulfonyl-2-(2,6-difluoro-phenyl)-1H-indol-5-yl]-3-methyl-benzoic acid methyl ester (90 mg, 0.167 mmol) in THF/MeOH (2:1, 3 ml) was added Cs₂CO₃ (148 mg, 0.45 mmol) and stirred at 25° C. for 24 h. The reaction mixture was concentrated, then added water and extracted with EtOAc. The organic phase was washed with brine, dried over Na₂SO₄ and concentrated. The crude compound was purified by combiflash column chromatography to give 4-[2-(2,6-difluoro-phenyl)-1H-indol-5-yl]-3-methyl-benzoic acid methyl ester (7 mg, 11%), MS (M+H)=398.

### Example 89

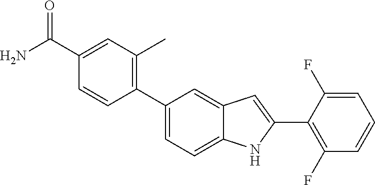

### 4-[2-(2,6-Difluoro-phenyl)-1H-indol-5-yl]-3-methyl-benzamide

Prepared in a manner identical to Example 88 using commercially available 4-bromo-3-methyl-benzamide in the Suzuki step. MS (M+H)=363.

### Example 90

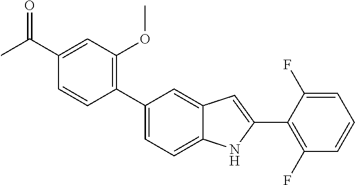

### 2-(2,6-Difluoro-phenyl)-5-(2,4-dimethoxy-phenyl)-1H-indole

Prepared in a manner identical to Example 88 using commercially available 1-bromo-2,4-dimethoxy-benzene in the Suzuki step. MS (M+H)=366.

### Example 91

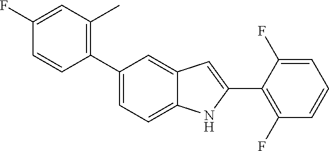

### 2-(2,6-Difluoro-phenyl)-5-(4-fluoro-2-methyl-phenyl)-1H-indole

Prepared in a manner identical to Example 88 using commercially available 1-bromo-4-fluoro-2-methyl-benzene in the Suzuki step. MS (M+H)=338.

### Example 92

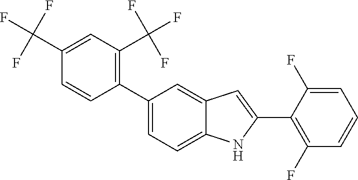

### 5-(2,4-Bis-trifluoromethyl-phenyl)-2-(2,6-difluoro-phenyl)-1H-indole

Prepared in a manner identical to Example 88 using commercially available 1-bromo-2,4-bis-trifluoromethyl-benzene in the Suzuki step. MS (M+H)=442.

### Example 93

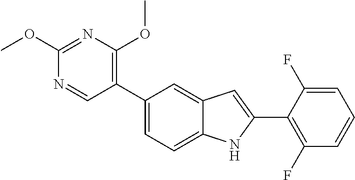

### 2-(2,6-Difluoro-phenyl)-5-(2,4-dimethoxy-pyrimidin-5-yl)-1H-indole

Prepared in a manner identical to Example 88 using commercially available 1-bromo-2,4-dimethoxy-benzene in the Suzuki step. MS (M+H)=368.

### Example 94

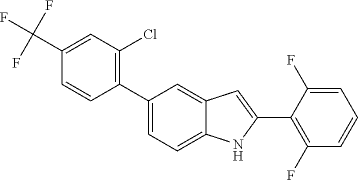

### 5-(2-Chloro-4-trifluoromethyl-phenyl)-2-(2,6-difluoro-phenyl)-1H-indole

Prepared in a manner identical to Example 88 using commercially available 1-bromo-2-chloro-4-trifluoromethyl-benzene in the Suzuki step. MS (M+H)=408.

### Example 95

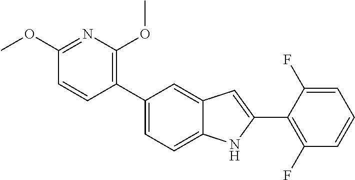

### 2-(2,6-Difluoro-phenyl)-5-(2,6-dimethoxy-pyridin-3-yl)-1H-indole

Prepared in a manner identical to Example 88 using commercially available 3-bromo-2,6-dimethoxy-pyridine in the Suzuki step. MS (M+H)=367.

### Example 96

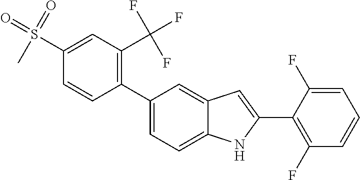

### 2-(2,6-Difluoro-phenyl)-5-(4-methanesulfonyl-2-trifluoromethyl-phenyl)-1H-indole

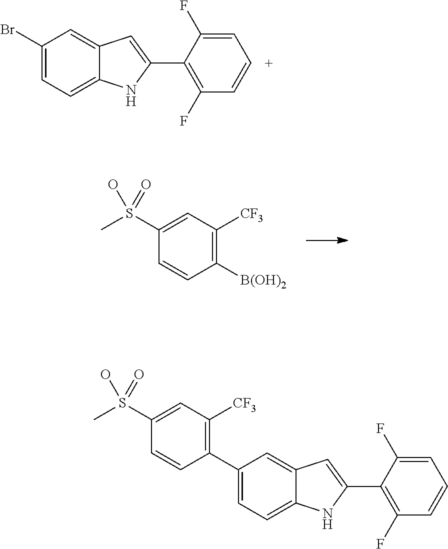

Prepared using the identical Suzuki reaction conditions described in Example 88 using 5-bromo-2-(2,6-difluoro-phenyl)-1H-indole (described in Example 1) and 4-(methylsulfonyl)-2-(trifluoromethyl)phenyl boronic acid as the coupling partners. MS (M+H)=452.

### Example 97

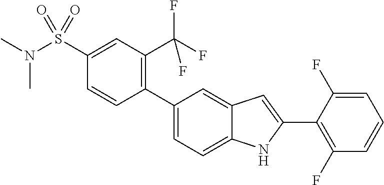

### 4-[2-(2,6-Difluoro-phenyl)-1H-indol-5-yl]-N,N-dimethyl-3-trifluoromethyl-benzenesulfonamide

Prepared in a manner identical to Example 96 using commercially available 4-(N,N-dimethylsulfamoyl)-2-trifluoromethyl-phenylboronic acid. MS (M+H)=427.

### Example 98

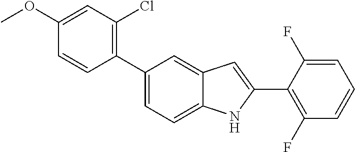

### 5-(2-Chloro-4-methoxy-phenyl)-2-(2,6-difluoro-phenyl)-1H-indole

Prepared in a manner identical to Example 96 using commercially available 2-chloro-4-methoxy-phenylboronic acid. MS (M+H)=370.

### Example 99

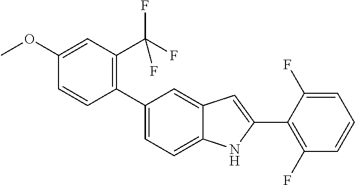

### 2-(2,6-Difluoro-phenyl)-5-(4-methoxy-2-trifluoromethyl-phenyl)-1H-indole

Prepared in a manner identical to Example 96 using commercially available 4-methoxy-2-trifluoromethyl-phenylboronic acid. MS (M+H)=404.

### Example 100

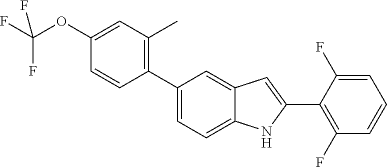

### 2-(2,6-Difluoro-phenyl)-5-(2-methyl-4-trifluoromethoxy-phenyl)-1H-indole

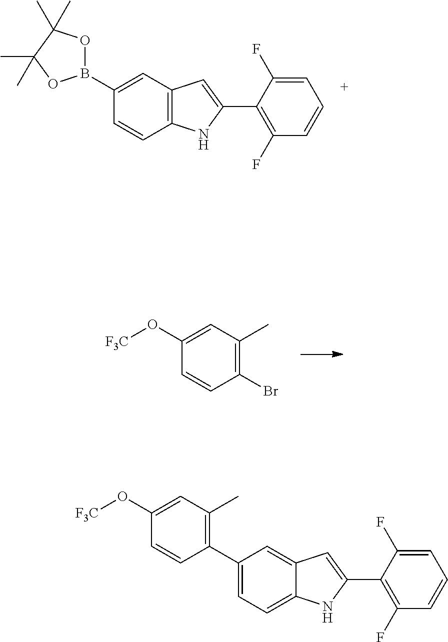

Prepared using the identical Suzuki reaction conditions to that described in Example 88 using 2-(2,6-Difluoro-phenyl)-5-(4,4,5,5-tetramethyl-[1,3,2]dioxaborolan-2-yl)-1H-indole and commercially available 1-bromo-2-methyl-4-trifluoromethoxy-benzene as the coupling partners. MS (M+H)=404.

### Example 101

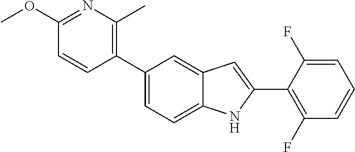

### 2-(2,6-Difluoro-phenyl)-5-(6-methoxy-2-methyl-pyridin-3-yl)-1H-indole

Prepared in a manner identical to Example 100 using 3-bromo-6-methoxy-2-methyl-pyridine. MS (M+H)=351.

### Example 102

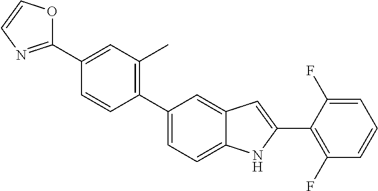

### 2-(2,6-Difluoro-phenyl)-5-(2-methyl-4-oxazol-2-yl-phenyl)-1H-indole

Prepared in a manner identical to Example 100 using Intermediate 14. MS (M+H)=387.

### Example 103

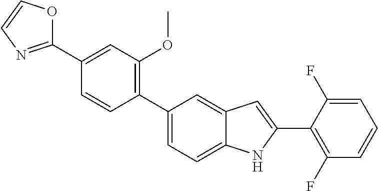

### 2-(2,6-Difluoro-phenyl)-5-(2-methoxy-4-oxazol-2-yl-phenyl)-1H-indole

Prepared in a manner identical to Example 100 using Intermediate 18. MS (M+H)=403.

### Example 104

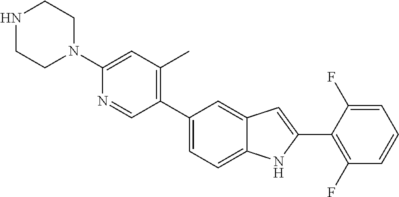

### 2-(2,6-Difluoro-phenyl)-5-(4-methyl-6-piperazin-1-yl-pyridin-3-yl)-1H-indole

Prepared in a manner identical to Example 100 using 1-(5-bromo-4-methyl-pyridin-2-yl)-piperazine. MS (M+H)=405.

### Example 105

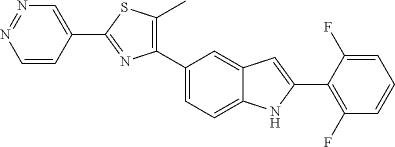

### 2-(2,6-Difluoro-phenyl)-5-(5-methyl-2-pyridazin-4-yl-thiazol-4-yl)-1H-indole

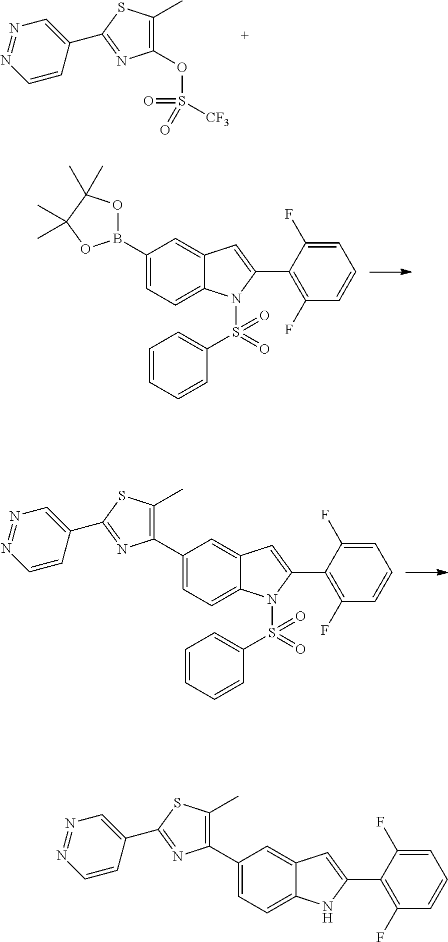

Benzenesulfonyl-2-(2,6-difluoro-phenyl)-5-(5-methyl-2-pyridazin-4-yl-thiazol-4-yl)-1H-indole: A solution of Intermediate 19 (72 mg, 0.22 mmol) and 1-benzenesulfonyl-2-(2,6-difluoro-phenyl)-5-(2-ethyl-5-phenyl-2H-pyrazol-3-yl)-1H-indole (100 mg, 0.20 mmol) in 1,4-dioxane (5 mL) was purged with nitrogen (10 min), then aqueous K₂CO₃ (2 M, 0.2 mL) was added and purged with nitrogen again (5 min). Pd(dppf)Cl₂ (10 mol %, 17 mg, 0.02 mmol) was then added to the above reaction mixture and stirred at 100° C. for 4 h. The reaction mixture was filtered through Celite and the filtrate was diluted with water then extracted with EtOAc. The organic phase was washed with brine, dried, concentrated under vacuum and purified by column chromatography to give 1-benzenesulfonyl-2-(2,6-difluoro-phenyl)-5-(5-methyl-2-pyridazin-4-yl-thiazol-4-yl)-1H-indole (65 mg, 59%).

2-(2,6-difluoro-phenyl)-5-(5-methyl-2-pyridazin-4-yl-thiazol-4-yl)-1H-indole: To a solution of 1-benzenesulfonyl-2-(2,6-difluoro-phenyl)-5-(5-methyl-2-pyridazin-4-yl-thiazol-4-yl)-1H-indole (65 mg, 0.12 mmol) in THF/MeOH (2:1, 6 ml) was added Cs₂CO₃ (116 mg, 0.358 mmol) and stirred at 25° C. for 24 h. The reaction mixture was concentrated, then added water and extracted with EtOAc. The organic phase was washed with brine, dried, concentrated under vacuum and purified by column chromatography to give 2-(2,6-difluoro-phenyl)-5-(5-methyl-2-pyridazin-4-yl-thiazol-4-yl)-1H-indole (30 mg, 62%), MS (M+H)=405.

### Example 106

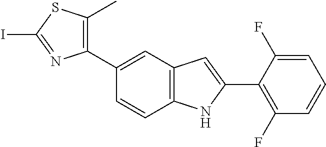

### 2-(2,6-Difluoro-phenyl)-5-(2-iodo-5-methyl-thiazol-4-yl)-1H-indole

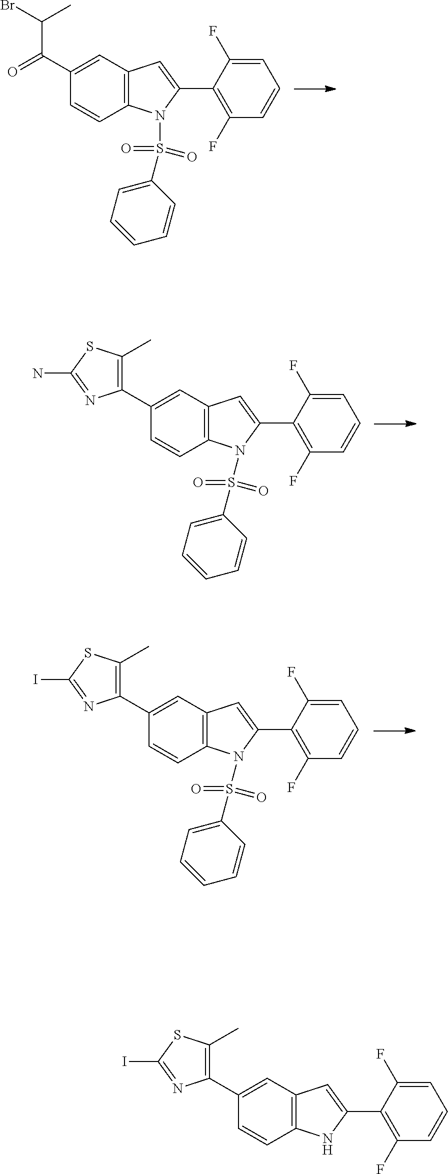

4-[1-Benzenesulfonyl-2-(2,6-difluoro-phenyl)-1H-indol-5-yl]-5-methyl-thiazol-2-ylamine: To a solution of 1-[1-Benzenesulfonyl-2-(2,6-difluoro-phenyl)-1H-indol-5-yl]-2-bromo-propan-1-one (1.5 g, 2.98 mmol) in Ethanol (50 ml) was added thiourea (452 mg, 5.95 mmol). The reaction was refluxed for 12 h, after which the solvent was removed and the crude material purified by column chromatography to give 4-[1-Benzenesulfonyl-2-(2,6-difluoro-phenyl)-1H-indol-5-yl]-5-methyl-thiazol-2-ylamine (1.2 g, 84%).

Benzenesulfonyl-2-(2,6-difluoro-phenyl)-5-(2-iodo-5-methyl-thiazol-4-yl)-1H-indole: To a solution of 4-[1-Benzenesulfonyl-2-(2,6-difluoro-phenyl)-1H-indol-5-yl]-5-methyl-thiazol-2-ylamine (200 mg, 0.415 mmol) in dicholomethane/diiodomethane (10/0.5 ml) and CH2I2 (0.5 ml) was added t-BuONO (0.15 ml, 1.24 mmol). The reaction was stirred at room temperature for 30 min, after which the solvent was removed and crude was purified by column chromatography to give 1-Benzenesulfonyl-2-(2,6-difluoro-phenyl)-5-(2-iodo-5-methyl-thiazol-4-yl)-1H-indole (160 mg, 65%).

2-(2,6-Difluoro-phenyl)-5-(2-iodo-5-methyl-thiazol-4-yl)-1H-indole: To a solution of 1-Benzenesulfonyl-2-(2,6-difluoro-phenyl)-5-(2-iodo-5-methyl-thiazol-4-yl)-1H-indole (100 mg, 0.167 mmol) in THF/MeOH (2:1) (3 ml) and was added Cs₂CO₃ (108 mg, 0.334 mmol). The mixture was stirred at 25° C. for 24 h, after which the solvent was removed and replaced with EtOAc, and this was washed with brine, dried over Na₂SO₄, concentrated and the crude material purified by column chromatography to give 2-(2,6-Difluoro-phenyl)-5-(2-iodo-5-methyl-thiazol-4-yl)-1H-indole (22 mg, 29%), MS (M+H)=453.

### Example 107

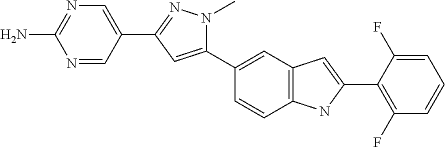

### 5-{5-[2-(2,6-Difluoro-phenyl)-1H-indol-5-yl]-1-methyl-1H-pyrazol-3-yl}-pyrimidin-2-ylamine

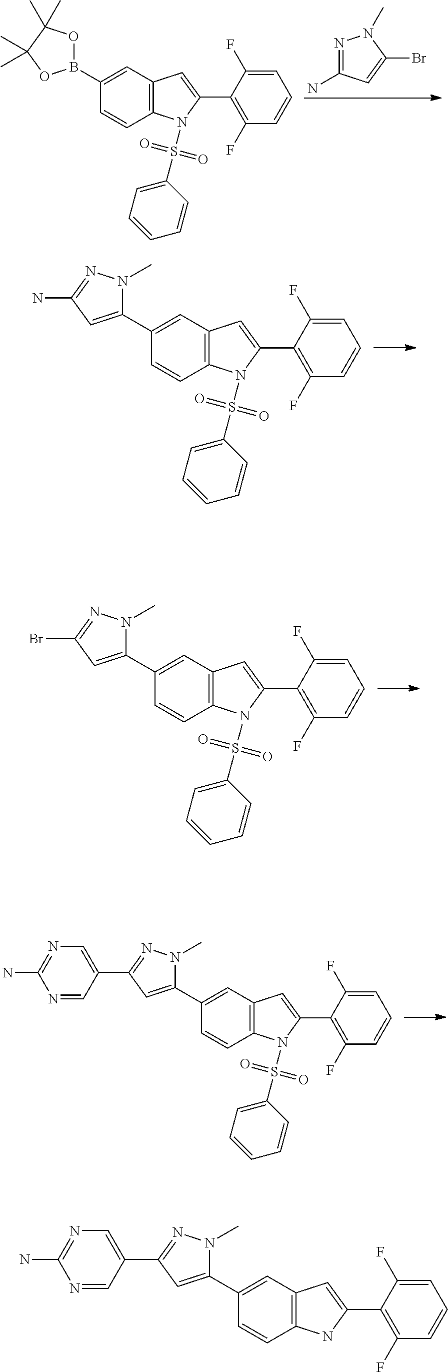

5-[1 Benzenesulfonyl-2-(2,6-difluoro-phenyl)-1H-indol-5-yl]-1-methyl-1H-pyrazol-3-ylamine: To a solution of 5-Bromo-1-methyl-1H-pyrazol-3-ylamine (1.28 g, 7.27 mmol) and 1-Benzenesulfonyl-2-(2,6-difluoro-phenyl)-5-(4,4,5,5-tetramethyl [1,3,2]dioxaborolan-2-yl)-1H-indole (4 g, 8.08 mmol) in 1,4-dioxane (40 mL) was degassed with nitrogen (10 min), then aqueous K₂CO₃ (2 M, 8.1 ml, 16.16 mmol) was added and the mixture purged again with nitrogen (10 min). Pd(dppf)Cl₂ (660 mg, 0.808 mmol) was then added to the above reaction mixture and stirred at 100° C. for 4 h. Upon cooling the mixture was filtered through Celite, and the filtrate extracted with EtOAc (3×20 mL). The organic phase (EtOAc layer) was washed with brine, dried over Na₂SO₄, concentrated, and purified by column chromatography to give 5-[1 Benzenesulfonyl-2-(2,6-difluoro-phenyl)-1H-indol-5-yl]-1-methyl-1H-pyrazol-3-ylamine (2.05 g, 55%).

Benzenesulfonyl-5-(5-bromo-2-methyl-2H-pyrazol-3-yl)-2-(2,6-difluoro-phenyl)-1H-indole: To an acidic solution (catalytic sulphuric acid) of 5-[1 Benzenesulfonyl-2-(2,6-difluoro-phenyl)-1H-indol-5-yl]-1-methyl-1H-pyrazol-3-ylamine (50 mg, 0.107 mmol) in acetonitrile (2 ml) was added dropwise an aqueous solution of NaNO2 (8 mg, 0.107 mmol) under ice-cooling, and the mixture was stirred for 30 min. Copper (I) bromide (24 mg, 0.161 mmol) in HBr (0.05 ml) was added to the reaction mixture. He reaction mixture was stirred at 0° C. for 30 min. The reaction mixture was basified by aq NaHCO3 and extracted with EtOAc. The organic phase was washed with brine, dried over Na₂SO₄, concentrated and purified by column chromatography to give 1-Benzenesulfonyl-5-(5-bromo-2-methyl-2H-pyrazol-3-yl)-2-(2,6-difluoro-phenyl)-1H-indole (20 mg, 35%).

5-{5-[1-Benzenesulfonyl-2-(2,6-difluoro-phenyl)-1H-indol-5-yl]-1-methy 1-1H-pyrazol-3-yl}-pyrimidin-2-ylamine: To a solution of 1-Benzenesulfonyl-5-(5-bromo-2-methyl-2H-pyrazol-3-yl)-2-(2,6-difluoro-phenyl)-1H-indole (50 mg, 0.09 mmol) and Pyrimidin-2-ylamine-4-boronic acid (21 mg, 0.09 mmol) in 1,4-dioxane (2 mL) was degassed with nitrogen (10 min), then aqueous K₂CO₃ (2 M, 0.09 mL) was added and the mixture purged again (5 min). Pd(dppf)Cl₂ (8 mg, 0.009 mmol) was then added to the above reaction mixture and stirred at 100° C. for 4 h. Upon cooling the mixture was filtered through Celite, and the filtrate extracted with EtOAc (3×20 mL). The organic phase (EtOAc layer) was washed with brine, dried over Na₂SO₄, concentrated, and purified by column chromatography to give 5-{5-[1-Benzenesulfonyl-2-(2,6-difluoro-phenyl)-1H-indol-5-yl]-1-methyl-1H-pyrazol-3-yl}-pyrimidin-2-ylamine (18 mg, 35%).

5-{5-[2-(2,6-Difluoro-phenyl)-1H-indol-5-yl]-1-methyl-1H-pyrazol-3-yl}-pyrimidin-2-ylamine: To a solution of 5-{5-[1-Benzenesulfonyl-2-(2,6-difluoro-phenyl)-1H-indol-5-yl]-1-methyl-1H-pyrazol-3-yl}-pyrimidin-2-ylamine (18 mg, 0.033 mmol) in THF/MeOH (2:1) (6 ml) and Cs₂CO₃ (32 mg, 0.099 mmol) was added. The mixture was stirred at 25° C. for 24 h, after which the solvent was removed and replaced with EtOAc, and this was washed with brine, dried over Na₂SO₄, concentrated, and the crude material purified by column chromatography to give 5-{5-[2-(2,6-Difluoro-phenyl)-1H-indol-5-yl]-1-methyl-1H-pyrazol-3-yl}-pyrimidin-2-ylamine (10 mg, 75%), MS (M+H)=403.

### Example 108

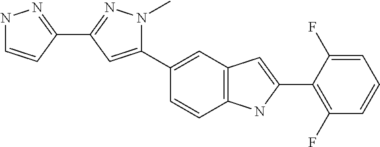

### 2-(2,6-Difluoro-phenyl)-5-(1-methyl-1H,1′H-[3,3]bipyrazolyl-5-yl)-1H-indole

2-(2,6-Difluoro-phenyl)-5-(1-methyl-1H,1′H-[3,3′]bipyrazolyl-5-yl)-1H-indole was prepared in a manner identical to 5-{5-[2-(2,6-Difluoro-phenyl)-1H-indol-5-yl]-1-methyl-1H-pyrazol-3-yl}-pyrimidin-2-ylamine with the following materials 1-Benzenesulfonyl-5-(5-bromo-2-methyl-2H-pyrazol-3-yl)-2-(2,6-difluoro-phenyl)-1H-indole and 5-pyrazole boronic acid. MS (M+H)=376.

### Example 109

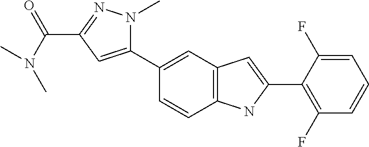

### 5-[2-(2-Fluoro-6-methyl-phenyl)-1H-indol-5-yl]-1-methyl-1H-pyrazole-3-carboxylic acid dimethylamide

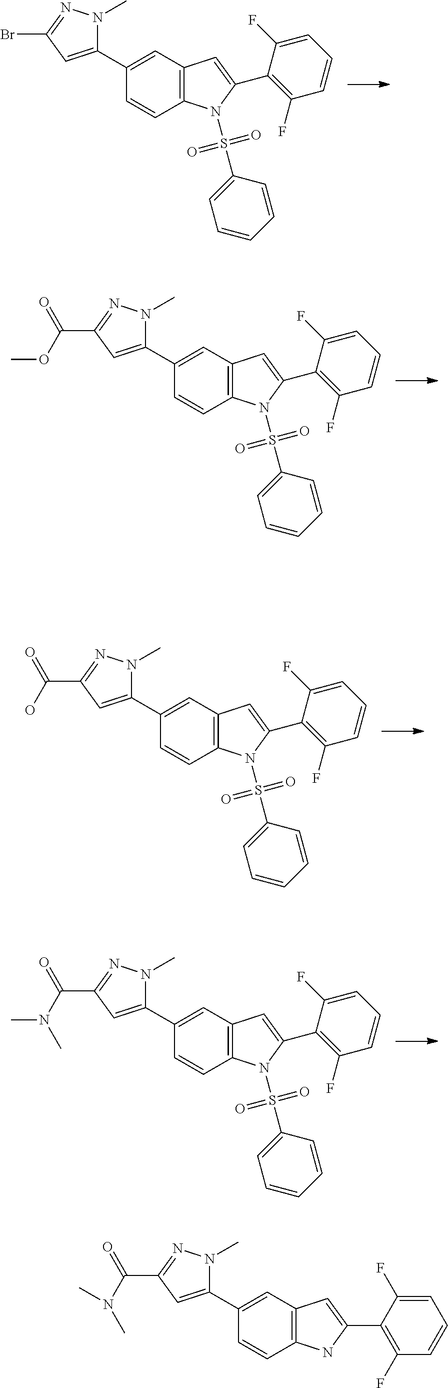

5-[1 Benzenesulfonyl-2-(2,6-difluoro-phenyl)-1H-indol-5-yl]-1-methyl-1H-pyrazole-3-carboxylic acid methyl ester: To a solution of 1-Benzenesulfonyl-5-(5-bromo-2-methyl-2H-pyrazol-3-yl)-2-(2,6-difluoro-phenyl)-1H-indole (100 mg, 0.189 mmol) in MeOH (10 ml) was degassed with nitrogen (10 min), then TEA (0.5 ml) was added. 1,3 bis(diphenylphosphino)propane (9 mg, 0.0189 mmol) and Pd(OAc)₂ (3 mg, 0.009 mmol) was then added to the above mixture and stirred under autoclave at 220 psi (CO pressure) and at 80° C. for 18 h. Upon cooling the mixture was filtered through Celite, and the filtrate extracted with EtOAc. The organic phase (EtOAc layer) was washed with brine, dried over Na₂SO₄, concentrated, and purified by column chromatography to give 5-[1 Benzenesulfonyl-2-(2,6-difluoro-phenyl)-1H-indol-5-yl]-1-methyl-1H-pyrazole-3-carboxylic acid methyl ester (30 mg, 31%).

5-[1-Benzenesulfonyl-2-(2,6-difluoro-phenyl)-1H-indol-5-yl]-1-methyl-1H-pyrazole-3-carboxylic acid: To a solution of 5-[1 Benzenesulfonyl-2-(2,6-difluoro-phenyl)-1H-indol-5-yl]-1-methyl-1H-pyrazole-3-carboxylic acid methyl ester (85 mg, 0.16 mmol) in THF/MeOH/H₂O (6:3:2) (11 ml) was added Lithium hydroxide (11 mg, 0.25 mmol). The mixture was stirred at 25° C. for 6 h, after which the solvent was removed and acidified with HCl (1 M) up to pH-1 and extracted with dichlromethane. The organic layer was washed with brine, dried over Na₂SO₄, concentrated, and the crude material purified by column chromatography to give 5-[1-Benzenesulfonyl-2-(2,6-difluoro-phenyl)-1H-indol-5-yl]-1-methyl-1H-pyrazole-3-carboxylic acid (41 mg, 50%).

5-[1-Benzenesulfonyl-2-(2,6-difluoro-phenyl)-1H-indol-5-yl]-1-methyl-1H-pyrazole-3-carboxylic acid dimethylamide: To a solution of 5-[1-Benzenesulfonyl-2-(2,6-difluoro-phenyl)-1H-indol-5-yl]-1-methyl-1H-pyrazole-3-carboxylic acid (40 mg, 0.08 mmol) in DMF (3 ml) was added EDCI (23 mg, 0.122 mmol), HOBt (15 mg, 0.097 mmol), DIPEA (0.034 ml, 0.249 mmol) and dimethyl amine (2M, 0.1 ml, 0.2 mmol) at room temperature. Stirring was continued for 12 h after which the solvent was removed and replaced with dicholoromethane, and this was washed with brine, dried over Na₂SO₄, concentrated, and the crude material purified by column chromatography to give 5-[1-Benzenesulfonyl-2-(2,6-difluoro-phenyl)-1H-indol-5-yl]-1-methyl-1H-pyrazole-3-carboxylic acid dimethylamide (25 mg, 59%).

5-[2-(2-Fluoro-6-methyl-phenyl)-1H-indol-5-yl]-1-methyl-1H-pyrazole-3-carboxylic acid dimethylamide was prepared in a manner identical to 5-{5-[2-(2,6-Difluoro-phenyl)-1H-indol-5-yl]-1-methyl-1H-pyrazol-3-yl}-pyrimidin-2-ylamine with the following materials 5-[1-Benzenesulfonyl-2-(2,6-difluoro-phenyl)-1H-indol-5-yl]-1-methyl-1H-pyrazole-3-carboxylic acid dimethylamide. MS (M+H)=382.

### Example 110

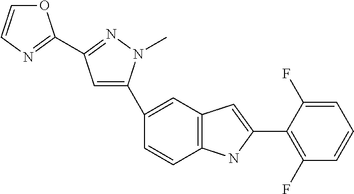

### 2-(2,6-Difluoro-phenyl)-5-(2-methyl-5-oxazol-2-yl-2H-pyrazol-3-yl)-1H-indole

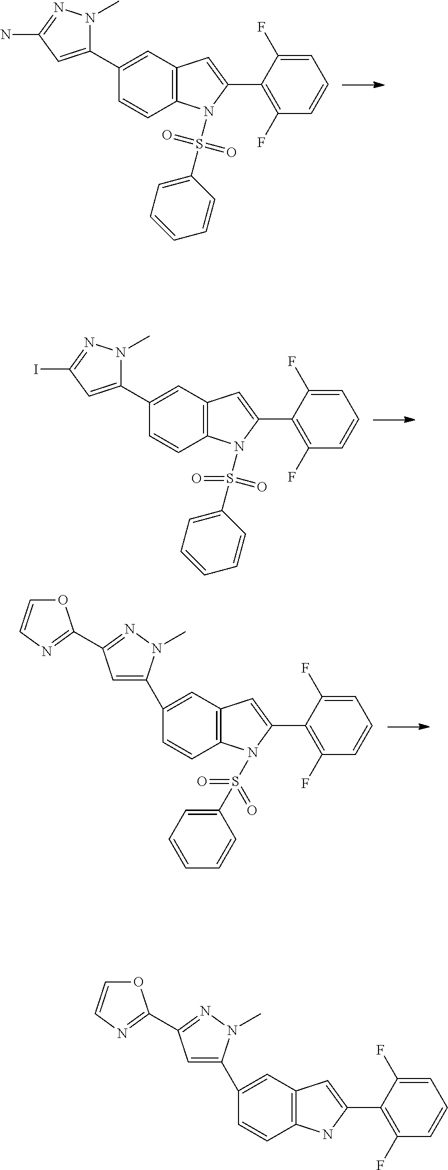

2-(2,6-Difluoro-phenyl)-1-[((E)-hexa-1,3,5-triene)-3-sulfonyl]-5-(5-iodo-2-methyl-2H-pyrazol-3-yl)-1H-indole: To a solution of 5-[1-Benzenesulfonyl-2-(2,6-difluor phenyl)-1H-indol-5-yl]-1-methyl-1H-pyrazol-3-ylamine (100 mg, 0.215 mmol) in Dichloromethane (5 ml) and Diiodomethane (0.5 ml) was added t-BuONO (0.04 ml, 0.323 mmol). The mixture was stirred at 25° C. for 30 min, after which dichloromethane was evaporated and the crude material purified by column chromatography to give to give 2-(2,6-Difluoro-phenyl)-1-[((E)-hexa-1,3,5-triene)-3-sulfonyl]-5-(5-iodo-2-methyl-2H-pyrazol-3-yl)-1H-indol (50 mg, 40%).

Benzenesulfonyl-2-(2,6-difluoro-phenyl)-5-(2-methyl-5-oxazol-2-yl-2H-pyrazol-3-yl)-1H-indole: To a solution of 2-(2,6-Difluoro-phenyl)-1-[((E)-hexa-1,3,5-triene)-3-sulfonyl]-5-(5-iodo-2-methyl-2H-pyrazol-3-yl)-1H-indol (100 mg, 0.173 mmol) and 2-Tributylstannanyl-oxazole (124 mg, 0.347 mmol) in 1,4-dioxane (3 mL) was degassed with nitrogen (10 min). Pd(dppf)Cl₂ (10 mol %, 15 mg, 0.0173 mmol) was then added to the above reaction mixture and stirred at 100° C. for 4 h. Upon cooling the mixture was filtered through Celite, and the filtrate extracted with EtOAc. The organic phase (EtOAc layer) was washed with brine, dried over Na₂SO₄, concentrated, and purified by column chromatography to give 1-Benzenesulfonyl-2-(2,6-difluoro-phenyl)-5-(2-methyl-5-oxazol-2-yl-2H-pyrazol-3-yl)-1H-indole (40 mg, 44.57%).

2-(2,6-Difluoro-phenyl)-5-(2-methyl-5-oxazol-2-yl-2H-pyrazol-3-yl)-1H-indole: 2-(2,6-Difluoro-phenyl)-5-(2-methyl-5-oxazol-2-yl-2H-pyrazol-3-yl)-1H-indole was prepared in a manner identical to 5-{5-[2-(2,6-Difluoro-phenyl)-1H-indol-5-yl]-1-methyl-1H-pyrazol-3-yl}-pyrimidin-2-ylamine with the following material 1-Benzenesulfonyl-2-(2,6-difluoro-phenyl)-5-(2-methyl-5-oxazol-2-yl-2H-pyrazol-3-yl)-1H-indole. MS (M+H)=377.

### Example 111

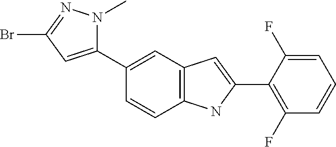

### 5-(5-Bromo-2-methyl-2H-pyrazol-3-yl)-2-(2,6-difluoro-phenyl)-1H-indole

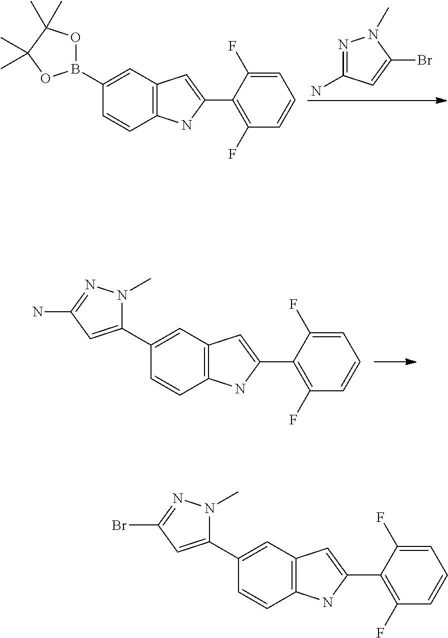

5-[2-(2,6-Difluoro-phenyl)-1H-indol-5-yl]-1-methyl-1H-pyrazol-3-ylamine: To a solution of 5-Bromo-1-methyl-1H-pyrazol-3-ylamine (2.057 g, 11.68 mmol) and 2-(2,6-Difluoro-phenyl)-5-(4,4,5,5-tetramethyl-[1,3,2]dioxaborolan-2-yl)-1H-indole (4 g, 12.98 mmol) in 1,4-dioxane (60 mL) was degassed with nitrogen (20 min), then aqueous K₂CO₃ (2 M, 13 mL) was added and the mixture purged again (10 min). Pd(dppf)Cl₂ (10 mol %, 1.0597 g, 1.298 mmol) was then added to the above reaction mixture and stirred at 100° C. for 4 h. Upon cooling the mixture was filtered through Celite, and the filtrate extracted with EtOAc. The organic phase (EtOAc layer) was washed with brine, dried over Na₂SO₄, concentrated, and purified by column chromatography to give 5-[2-(2,6-Difluoro-phenyl)-1H-indol-5-yl]-1-methyl-1H-pyrazol-3-ylamine (2 g, 53%).

5-(5-Bromo-2-methyl-2H-pyrazol-3-yl)-2-(2,6-difluoro-phenyl)-1H-indole: 5-(5-Bromo-2-methyl-2H-pyrazol-3-yl)-2-(2,6-difluoro-phenyl)-1H-indole was prepared in a manner identical to 1-Benzenesulfonyl-5-(5-bromo-2-methyl-2H-pyrazol-3-yl)-2-(2,6-difluoro-phenyl)-1H-indole with the following material 5-[2-(2,6-Difluoro-phenyl)-1H-indol-5-yl]-1-methyl-1H-pyrazol-3-ylamine. MS (M+H)=388.

### Example 112

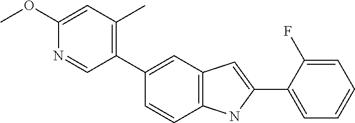

### 2-(2-Fluoro-phenyl)-5-(6-methoxy-4-methyl-pyridin-3-yl)-1H-indole

2-(2-Fluoro-phenyl)-5-(6-methoxy-4-methyl-pyridin-3-yl)-1H-indole: 2-(2-Fluoro-phenyl)-5-(6-methoxy-4-methyl-pyridin-3-yl)-1H-indole was prepared in a manner identical to 2-(2,6-Difluoro-phenyl)-5-(2-methyl-5-pyridin-3-yl-2H-pyrazol-3-yl)-1H-indole with the following materials 1-Benzenesulfonyl-5-bromo-2-(2-fluoro-phenyl)-1H-indole and 2-Methoxy-4-methylpyridine-5-boronic acid. MS (M+H)=333.

### Example 113

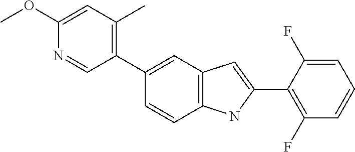

### 2-(2,6-Difluoro-phenyl)-5-(6-methoxy-4-methyl-pyridin-3-yl)-1H-indole

2-(2,6-Difluoro-phenyl)-5-(6-methoxy-4-methyl-pyridin-3-yl)-1H-indole was prepared in a manner identical to 2-(2,6-Difluoro-phenyl)-5-(2-methyl-4-trifluoromethoxy-phenyl)-1H-indole with the following materials 2-(2,6-Difluoro-phenyl)-5-(4,4,5,5-tetramethyl[1,3,2]dioxaborolan-2-yl)-1H-indole and 5-Bromo-2-methoxy-4-methyl-pyridine. MS (M+H)=351.

### Example 114

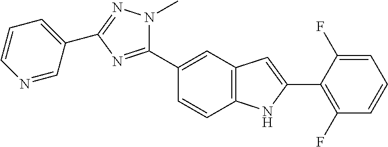

### 2-(2,6-Difluoro-phenyl)-5-(2-methyl-5-pyridin-3-yl-2H-[1,2,4]triazol-3-yl)-1H-indole

2-(2,6-Difluoro-phenyl)-5-(2-methyl-5-pyridin-3-yl-2H-[1,2,4]triazol-3-yl)-1H-indole was prepared in a manner identical to 2-(2,6-Difluoro-phenyl)-5-(2-methyl-4-trifluoromethoxy-phenyl)-1H-indole with the following materials 2-(2,6-Difluoro-phenyl)-5-(4,4,5,5-tetramethyl[1,3,2]dioxaborolan-2-yl)-1H-indole and 3-(5-bromo-1-methyl-1H-[1,2,4]triazol-3-yl)-pyridine (Intermediate 20), MS (M+H)=388.

### Example 115

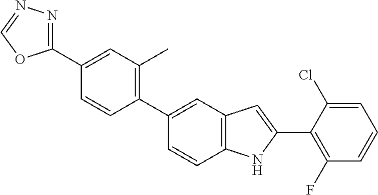

### 2-(2-Chloro-6-fluoro-phenyl)-5-(2-methyl-4-[1,3,4]oxadiazol-2-yl-phenyl)-1H-indole

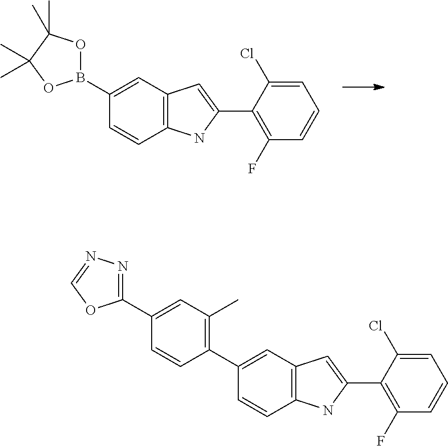

2-(2-Chloro-6-fluoro-phenyl)-5-(2-methyl-4-[1,3,4]oxadiazol-2-yl-phenyl)-1H-indole: To a solution of 2-(2-Chloro-6-fluoro-phenyl)-5-(4,4,5,5-tetramethyl-[1,3,2]dioxaborolan-2-yl)-1H-indole (100 mg, 0.269 mmol) and 2-(4-bromo-3-methyl-phenyl)-[1,3,4]oxadiazole (64 mg, 0.27 mmol) in 1,4-dioxane (3 mL) was degassed with nitrogen (10 min), then aqueous K₂CO₃ (74 mg, 0.54 mmol) was added and purged with nitrogen again (10 min). Pd(dppf)Cl₂ (21 mg, 0.027 mmol) was then added to the above reaction mixture and stirred at 100° C. for 4 h. Upon cooling the mixture was filtered through Celite, and the filtrate extracted with EtOAc. The organic phase (EtOAc layer) was washed with brine, dried over Na₂SO₄, concentrated, and purified by column chromatography to give 2-(2-Chloro-6-fluoro-phenyl)-5-(2-methyl-[1,3,4]oxadiazol-2-yl-phenyl)-1H-indole (20 mg, 20%) as light yellow solid, MS (M+H)=404.

### Example 116

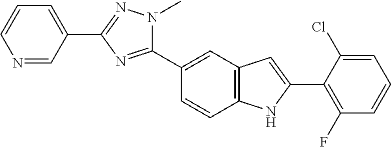

### 2-(2-Chloro-6-fluoro-phenyl)-5-(2-methyl-5-pyridin-3-yl-2H-[1,2,4]-triazol-3-yl)-1H-indole

Prepared in a manner identical to example 115. Substituting Intermediate 20 in the Suzuki coupling step. MS (M+H)=404.

### Example 117

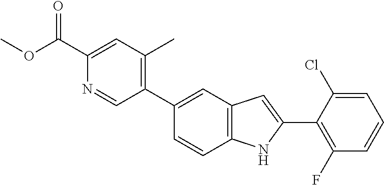

### 5-[2-(2-Chloro-6-fluoro-phenyl)-1H-indol-5-yl]-4-methyl-pyridine-2-carboxylic acid methyl ester

Prepared in a manner identical to example 115. Substituting intermediate 22 in the Suzuki coupling step. MS (M+H)=395.

### Example 118

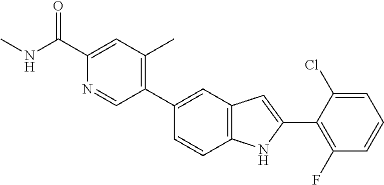

### 5-[2-(2-Chloro-6-fluoro-phenyl)-1H-indol-5-yl]-4-methyl-pyridine-2-carboxylic acid methylamide

Prepared in a manner identical to example 115. Substituting intermediate 23 in the Suzuki coupling step. MS (M+H)=394.

### Example 119

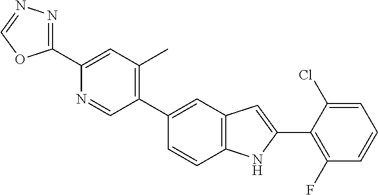

### 2-(2-Chloro-6-fluoro-phenyl)-5-(4-methyl-6-[1,3,4]oxadiazol-2-yl-pyridin-3-yl)-1H-indole

Prepared in a manner identical to example 115. Substituting intermediate 24 in the Suzuki coupling step. MS (M+H)=405.

### Example 120

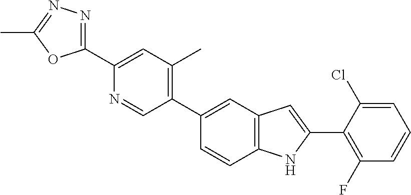

### 2-(2-Chloro-6-fluoro-phenyl)-5-[4-methyl-6-(5-methyl-[1,3,4]oxadiazol-2-yl)-pyridin-3-yl]-1H-indole

Prepared in a manner identical to example 115. Substituting intermediate 26 in the Suzuki coupling step. MS (M+H)=419.

### Example 121

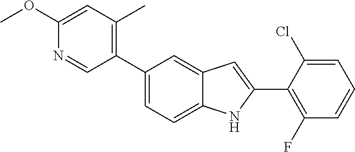

### 2-(2-Chloro-6-fluoro-phenyl)-5-(6-methoxy-4-methyl-pyridin-3-yl)-1H-indole

Prepared in a manner identical to Example 100 using the appropriate aryl halide. MS (M+H)=367.

### Example 122

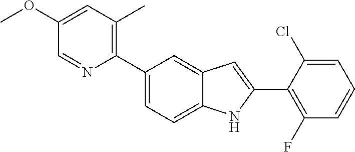

### 2-(2-Chloro-6-fluoro-phenyl)-5-(5-methoxy-3-methyl-pyridin-2-yl)-1H-indole

Prepared in a manner identical to Example 100 using the appropriate aryl halide. MS (M+H)=367.

### Example 123

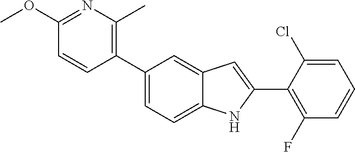

### 2-(2-Chloro-6-fluoro-phenyl)-5-(6-methoxy-2-methyl-pyridin-3-yl)-1H-indole

Prepared in a manner identical to Example 100 using the appropriate aryl halide. MS (M+H)=367.

### Example 124

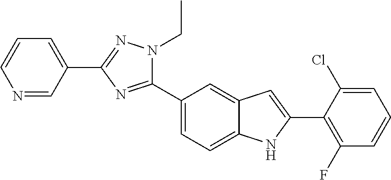

### 2-(2-Chloro-6-fluoro-phenyl)-5-(2-ethyl-5-pyridin-3-yl-2H-[1,2,4]triazol-3-yl)-1H-indole

Prepared in a manner identical to example 115. Substituting Intermediate 27 in the Suzuki coupling step. MS (M+H)=418.

### Example 125

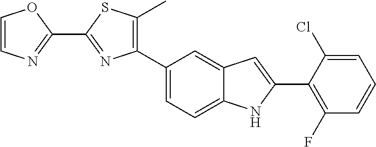

### 2-(2-Chloro-6-fluoro-phenyl)-5-(5-methyl-2-oxazol-2-yl-thiazol-4-yl)-1H-indole

Prepared in a manner identical to example 115. Substituting Intermediate 28 in the Suzuki coupling step. MS (M+H)=410.

### Example 126

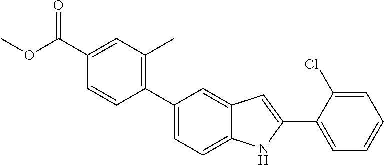

### 4-[2-(2-Chloro-phenyl-1H-indol-5-yl]-3-methyl-benzoic acid methyl ester

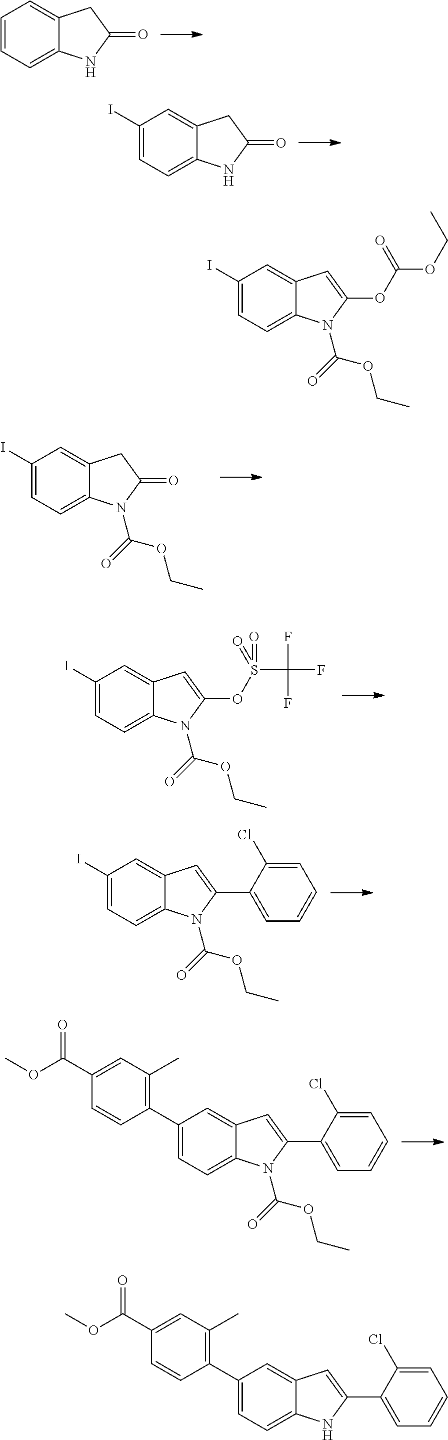

iodo-oxindole: To solution of oxindole (4.43 g, 33.3 mmol) in AcOH (35 mL) was added NIS (9 g, 40.0 mmol) at rt. The mixture was stirred for 1.5 hours at which point water (60 mL) was slowly added dropwise, followed by about 5 mL of EtOAc to solubilize impurities. The solid was filtered, washed with a small amount of EtOAc followed by diethyl ether, to give 5-iodo-oxindole (5.4 g, 62%) as a light pink solid clean by proton NMR in DMSO.

Ethyl 2-(ethoxycarbonyloxy)-5-iodo-1H-indole-1-carboxylate: To a 0° C. solution of 5-iodoindolin-2-one (4.67 g, 18.0 mmol) in anhydrous THF (75 mL), and Et₃N (7.53 mL, 54.0 mmol), was added ethyl chloroformate (5.14 mL, 54.0 mmol) dropwise, the reaction mixture was stirred at rt for 1 hr, then partitioned between EtOAc and water, the organic layer was dried over Na₂SO₄, filtered and concentrated to give Ethyl 2-(ethoxycarbonyloxy)-5-iodo-1H-indole-1-carboxylate (−7 g) that was sufficiently pure to be carried on to the next step.

Ethyl 5-iodo-2-oxoindoline-1-carboxylate: To a 0° C. solution of ethyl 2-(ethoxycarbonyloxy)-5-iodo-1H-indole-1-carboxylate (5.95 g, 14.8 mmol) in 50 mL of DMF was added ammonium carbonate (1.42 g, 14.8 mmol). The reaction mixture was stirred at 0° C. for 20 min., then stirred for 3 hrs., while maintaining the temp. between 0° C. and 15° C., partitioned between EtOAc and water, the organic layer was washed with brine, dried over sodium sulfate, filtered, and concentrated to give Ethyl 5-iodo-2-oxoindoline-1-carboxylate (4.92 g, 100%) as a light-brown solid.

Ethyl 5-iodo-2-(trifluoromethylsulfonyloxy)-1H-indole-1-carboxylate: To a 0° C. solution of ethyl 5-iodo-2-oxoindoline-1-carboxylate (4.90 g, 14.8 mmol), and DIPEA (5 mL, 29 mmol) in 200 mL of CH₂Cl₂ was added trifluoromethanesulfonic anhydride (3.80 mL, 22.6 mmol) dropwise, keeping the reaction temp between 0 and 4° C., the reaction mixture was stirred for 3 hrs., slowly warming to RT, ice water and CH₂Cl₂ were added, partitioned, washed organic layer with a 5% aq. sodium carbonate solution, brine, dried over sodium sulfate, filtered, concentrated and purified by flash chromatography (4:96 EtOAc/hexanes) to give Ethyl 5-iodo-2-(trifluoromethylsulfonyloxy)-1H-indole-1-carboxylate (4.19 g, 61%) as a light-brown solid.

Ethyl 2-(2-chlorophenyl)-5-iodo-1H-indole-1-carboxylate: To a flask was added ethyl 5-iodo-2-(trifluoromethylsulfonyloxy)-1H-indole-1-carboxylate (4.19 g, 9.05 mmol), 2-chlorophenylboronic acid (1.84 g, 11.8 mmol), a 2M solution of NaHCO₃ (36 mL, 72 mmol), toluene (90 mL), and EtOH (54 mL), then degassed the reaction mixture with N₂ and added Pd(PPh₃)₄ (523 mg, 5 mol %). The reaction mixture was heated to 60° C. for 6 h, stirred overnight at RT, then partitioned between EtOAc and water, the organic layer was washed with brine, dried over sodium sulfate, filtered, concentrated and purified by flash chromatography (5:95 EtOAc/hexanes) to give Ethyl 2-(2-chlorophenyl)-5-iodo-1H-indole-1-carboxylate (2.4 g, 62%) as a pale-yellow solid.

Ethyl 2-(2-chlorophenyl)-5-(4-(methoxycarbonyl)-2-methylphenyl)-1H-indole-1-carboxylate: To a flask was added; ethyl 2-(2-chlorophenyl)-5-iodo-1H-indole-1-carboxylate (1.02 g, 2.4 mmol), 3-methyl-4-(4,4,5,5-tetramethyl-[1,3,2]dioxaborolan-2-yl)-benzoic acid methyl ester (0.86 g, 3.1 mmol), K₂CO₃ (0.38 g, 3.6 mmol), dioxane (15 mL), and water (3 mL). The reaction mixture was degassed with N₂ and added Pd(dppf)Cl₂*CH₂Cl₂ (98 mg, 5 mol %). The reaction mixture was heated to 60° C. for 6 hrs., stirred overnight at RT, then partitioned between EtOAc and water, the aqueous layer was extracted twice more with EtOAc, and the combined organic layers were washed with brine, dried over sodium sulfate, filtered, concentrated and twice purified by flash chromatography (3:97 and 5:95 EtOAc/hexanes) to give Ethyl 2-(2-chlorophenyl)-5-(4-(methoxycarbonyl)-2-methylphenyl)-1H-indole-1-carboxylate (0.67 g, 63%) as a colorless liquid.

4-[2-(2-chloro-phenyl)-1H-indol-5-yl]-3-methyl-benzoic acid methyl ester: To a solution of carbamate (0.297 g, 0.663 mmol) in 6.6 mL of MeOH and 3 mL of THF, was added K₂CO₃ (101 mg, 0.729 mmol). The reaction mixture was stirred at RT for 5 hrs., partitioned between EtOAc and water, the organic layer was washed with brine, dried over sodium sulfate, filtered and concentrated to give 4-[2-(2-chloro-phenyl)-1H-indol-5-yl]-3-methyl-benzoic acid methyl ester (0.230 g, 92%), MS (M+H)=376.

### Example 127

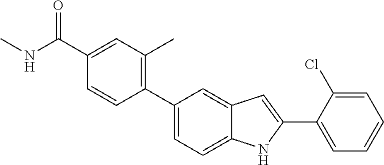

### 4-[2-(2-Chloro-phenyl)-1H-indol-5-yl]-3,N-dimethyl-benzamide

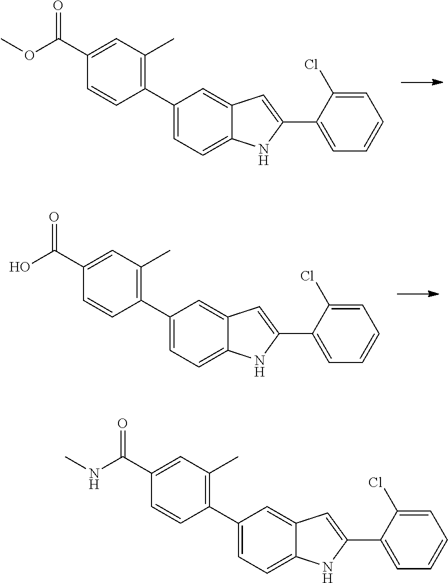

4-[2-(2-Chloro-phenyl)-1H-indol-5-yl]-3-methyl-benzoic acid: To a solution of 4-[2-(2-chloro-phenyl)-1H-indol-5-yl]-3-methyl-benzoic acid methyl ester (0.12 g, 0.32 mmol) in EtOH (5 ml) was added a solution of KOH in water (5 ml). The reaction mixture was heated to 100° C. for 4 hours; most of the EtOH was evaporated, the aqueous solution was adjusted to pH<2, the solid was collected and washed with water 3 times, after drying in a vacuum oven gave 4-[2-(2-chloro-phenyl)-1H-indol-5-yl]-3-methyl-benzoic acid as a pale yellow solid (115 mg, 99%).

4-[2-(2-Chloro-phenyl)-1H-indol-5-yl]-3,N-dimethyl-benzamide: To a solution of 4-[2-(2-chloro-phenyl)-1H-indol-5-yl]-3-methyl-benzoic acid (34 mg, 0.94 mmol), methyl amine hydrochloride (9 mg, 0.13 mmol) and HBTU (43 mg, 0.11 mmol) in DMF (2 ml) was added DIPEA (18 mg, 0.14 mmol). The reaction mixture was stirred for 4 hours at room temperature, water was added, the resulting solid was collected by filtration, and washed with water 3 times, the crude compound was purified by flash chromatography (5% MeOH/CH₂Cl₂) to give 4-[2-(2-chloro-phenyl)-1H-indol-5-yl]-3,N-dimethyl-benzamide, white solid (0.030 g, 85%). MS (M+H)=375.

### Example 128

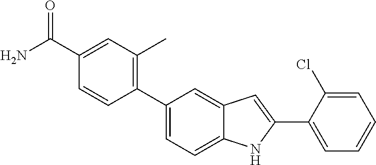

### 4-[2-(2-Chloro-phenyl)-1H-indol-5-yl]-3-methyl-benzamide

4-[2-(2-Chloro-phenyl)-1H-indol-5-yl]-3-methyl-benzamide: To a solution of 4-[2-(2-chloro-phenyl)-1H-indol-5-yl]-3-methyl-benzoic acid (0.043 g, 0.12 mmol) was added ammonium bicarbonate (28 mg, 0.36 mmol) and 2-ethoxy-1-ethoxycarbonyl-1,2-dihydroquinoline (35 mg, 0.14 mmol). The reaction mixture was stirred at room temperature for one day, partitioned between EtOAc and water. The organic phase was washed with brine, dried over Na₂SO₄, filtered and concentrated under reduced pressure. The crude material was purified by flash chromatography (using 30:70 then a 70:30 ratio of EtOAc/hexanes) to give 4-[2-(2-chloro-phenyl)-1H-indol-5-yl]-3-methyl-benzamide as a white solid (28 mg, 65%), MS (M+H)=361.

### Example 129

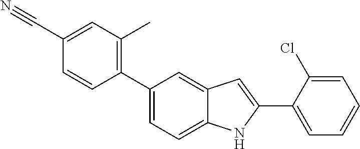

### 4-[2-(2-Chloro-phenyl)-1H-indol-5-yl]-3-methyl-benzonitrile

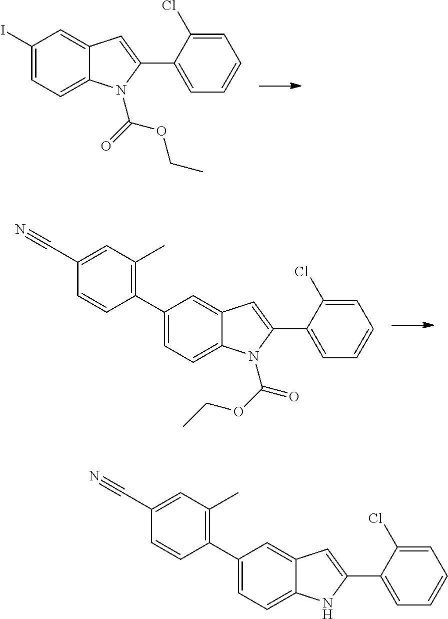

### Step 1: 2-(2-Chloro-phenyl)-5-(4-cyano-2-methyl-phenyl)-indole-1-carboxylic acid ethyl ester

A suspension of ethyl 2-(2-chlorophenyl)-5-iodo-1H-indole-1-carboxylate (100 mg, 235 mmol, Eq: 1.00), 4-cyano-2-methylphenylboronic acid (49.2 mg, 305 μmmol, Eq: 1.3), Potassium carbonate (97.4 mg, 705 μmmol, Eq: 3) in Dioxane (3.00 ml) and Water (0.6 ml) was purged with nitrogen (10 min) and then 1,1′-bis(diphenylphosphino)ferrocenedichloro palladium(II) dichloromethane complex (19.2 mg, 23.5 μmmol, Eq: 0.1) was added and r×n. mixture was heated at 100 C for 4 hr. Filtered through a pad of Celite, washed with DCM, solvent removed in vacuo, the residue redissolved in DCM, washed with water, brine, dried (Magnesium sulfate). Concentrated, chromatographed (silica gel, 10% EtOAc-Hexane) to obtain ethyl 2-(2-chlorophenyl)-5-(4-cyano-2-methylphenyl)-1H-indole-1-carboxylate (60 mg, 145 μmmol, 61.6% yield) as a off-white powder. LC/MS: (M+H)=415.

### Step 2: 4-[2-(2-Chloro-phenyl)-1H-indol-5-yl]-3-methyl-benzonitrile

A room temperature suspension of ethyl 2-(2-chlorophenyl)-5-(4-cyano-2-methylphenyl)-1H-indole-1-carboxylate (60.0 mg, 145 μmmol, Eq: 1.00) and Potassium carbonate (22.0 mg, 159 μmol, Eq: 1.1) in a mixture of THF (2 ml) andMeOH (1.00 ml) was stirred for 10 hr. The r×n. mixture was partitioned between saturated aqueous NH4Cl and EtOAc. The organiclayer was separated, dried (Magnesium sulfate). Concentrated, chromatographed (silica gel, 5% EtOAc-Hexane) to obtain 4-(2-(2-chlorophenyl)-1H-indol-5-yl)-3-methylbenzonitrile (45 mg, 131 μmmol, 90.8% yield) as a off-white powder. LC/MS: (M+H)=343.

### Example 130

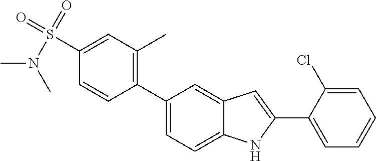

### 4-[2-(2-Chloro-phenyl)-1H-indol-5-yl]-3,N,N-trimethyl-benzenesulfonamide

Similarly prepared, using 4-(N,N-dimethylsulfamoyl)-2-methylphenylboronic acid in step 1.

4-[2-(2-Chloro-phenyl)-1H-indol-5-yl]-3,N,N-trimethyl-benzenesulfonamide, LC/MS (M+H)=426

### Example 131

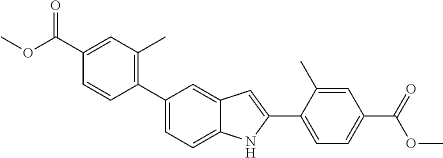

### 4-[5-(4-carbomethoxy-2-methyl-phenyl)-1H-indol-2-yl]-3-methyl-benzoic acid methyl ester

Similarly prepared, substituting 3-methyl-4-(4,4,5,5-tetramethyl-[1,3,2]dioxaborolan-2-yl)-benzoic acid methyl ester in the first Suzuki coupling. MS (M+H)=414.

### Example 132

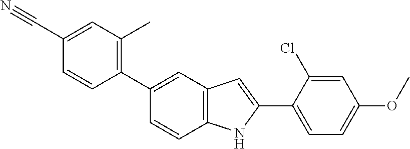

### 4-[2-(2-Chloro-4-methoxy-phenyl)-1H-indol-5-yl]-3-methyl-benzonitrile

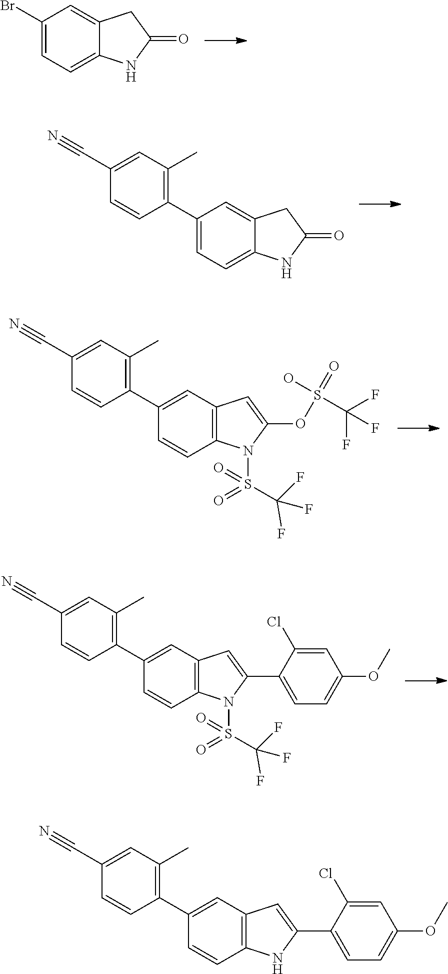

### Step 1 3-Methyl-4-(2-oxo-2,3-dihydro-1H-indol-5-yl)-benzonitrile

To a pressure flask was added; 5-bromoindolin-2-one (10.0 g, 47.2 mmol), 4-cyano-2-methylphenylboronic acid (9.11 g, 56.6 mmol) were combined with DMF (370 ml) to give a light brown solution, a solution of sodium carbonate in water (37 ml) was added, while the mixture was degassed with nitrogen, a catalytic amount of Pd(dppf)Cl₂*CH₂Cl₂ was added, and the flask sealed. The reaction mixture was heated to 90° C. for 15 h., water was added, the dark solid was collected, the solid was washed with water, MeOH and 20% EtOAc/hexanes to give 3-methyl-4-(2-oxo-2,3-dihydro-1H-indol-5-yl)-benzonitrile as a dark purple solid (12.1 g, 103%).

### Step 2 Trifluoro-methanesulfonic acid 5-(4-cyano-2-methyl-phenyl)-1-trifluoromethanesulfonyl-1-indol-2-yl ester

To a solution of 3-methyl-4-(2-oxo-2,3-dihydro-1H-indol-5-yl)-benzonitrile (11 g, 44.3 mmol) and DIPEA (22.9 g, 177 mmol) in CH₂Cl₂ (660 ml), was added (CF₃SO₂)₂O dropwise at 0° C., stirred at 0° C. about 2 hours, ice water was added, partitioned between CH₂Cl₂ and 0.5N HCl aq. solution, the organic phase was dried over Na₂SO₄, filtered and concentrated under reduced pressure. The crude material was purified by filtering through a pad of silica gel (using 5:95, 8:92 and 20:80 ratios of EtOAc/hexanes) to give a crude yellow solid, which was re-crystallized from EtOAc/hexanes to give trifluoro-methanesulfonic acid 5-(4-cyano-2-methyl-phenyl)-1-trifluoromethanesulfonyl-1-indol-2-yl ester as a off-white crystals, 6.74 g. Another crop of material was obtained by purification of the filtrate by flash chromatography (5%-8% EtOAc/hexanes) to give a second crop of trifluoro-methanesulfonic acid 5-(4-cyano-2-methyl-phenyl)-1-trifluoromethanesulfonyl-1-indol-2-yl ester as a pale yellow foam, 3.90 g. (total yield=10.64 g, 47%).

### Step 3 4-[2-(2-Chloro-4-methoxy-phenyl)-1-trifluoromethanesulfonyl-1H-indol-5-yl]-3-methyl-benzonitrile

Trifluoro-methanesulfonic acid 5-(4-cyano-2-methyl-phenyl)-1-trifluoromethanesulfonyl-1-indol-2-yl ester (31 mg, 0.06 mmol) and 2-Chloro-4-Methoxyphenylboronic acid (13.5 mg, 0.073 mmol) were mixed with toluene (0.5 ml), EtOH (0.3 ml) and NaHCO₃ (19.2 mg, 0.018 mmol) aqueous solution (0.2 ml), while the mixture was degassed with N₂, a catalytic amount of Pd(Ph₃P)₄ was added, the reaction mixture was heated to 80° C. for 3 hours, stirred overnight at room temperature; partitioned between EtOAc and water, the organic phase was washed with brine, dried over Na₂SO₄, filtered and concentrated under reduced pressure. The crude material was purified by the filtering through a pad of silica gel (5% EtOAc/hexanes) to give 4-[2-(2-chloro-4-methoxy-phenyl)-1-trifluoromethanesulfonyl-1H-indol-5-yl]-3-methyl-benzonitrile as a white solid (30 mg, 98%).

### Step 4 4-[2-(2-Chloro-4-methoxy-phenyl)-1H-indol-5-yl]-3-methyl-benzonitrile

To a solution of 4-[2-(2-chloro-4-methoxy-phenyl)-1-trifluoromethanesulfonyl-1H-indol-5-yl]-3-methyl-benzonitrile (30 mg) in THF (1 ml) and MeOH (1 ml), K₂CO₃ (50 mg) was added, the mixture was stirred at room temperature for one day, partitioned between EtOAc and water (3×), washed with brine, the organic phase was dried over Na₂SO₄, filtered and concentrated under reduced pressure, and the residue was purified by flash chromatography (10-30% EtOAc/hexanes) to give a crude pale-yellow solid, re-purified on preparative TLC plate (20% EtOAc/hexanes) to give 4-[2-(2-chloro-4-methoxy-phenyl)-1H-indol-5-yl]-3-methyl-benzonitrile as an off-white foam (19 mg, 86%). MS (M+H)=373.

### Example 133

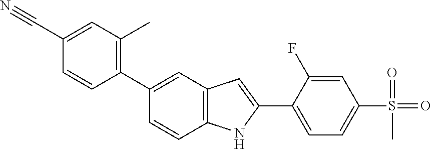

### 4-[2-(2-Fluoro-4-methanesulfonyl-phenyl)-1H-indol-5-yl]-3-methyl-benzonitrile

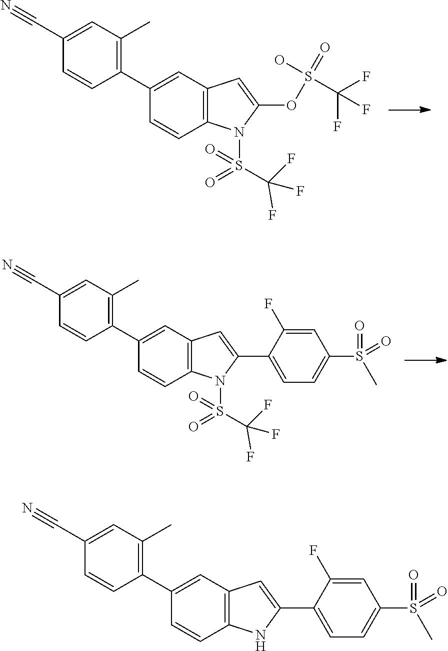

### Step 1 4-[2-(2-Fluoro-4-methanesulfonyl-phenyl)-1-trifluoromethanesulfonyl-1H-indol-5-yl]-3-methyl-benzonitrile

Similarly prepared as in Example 132, but replacing 2-chloro-4-methoxyphenylboronic acid with 2-fluoro-4-(methylsulfonyl)phenylboronic acid to give 4-[2-(2-fluoro-4-methanesulfonyl-phenyl)-1-trifluoromethanesulfonyl-1H-indol-5-yl]-3-methyl-benzonitrile.

### Step 2 4-[2-(2-Fluoro-4-methanesulfonyl-phenyl)-1H-indol-5-yl]-3-methyl-benzonitrile

To a solution of 4-[2-(2-fluoro-4-methanesulfonyl-phenyl)-1-trifluoromethanesulfonyl-1H-indol-5-yl]-3-methyl-benzonitrile (0.157 g, 0.293 mmol) in THF (4 ml), was added 3N NaOH aqueous solution (4 ml), the mixture was stirred for one day at room temperature; partitioned between EtOAc and water, the organic phase was washed with brine, dried over Na₂SO₄, filtered and concentrated under reduced pressure, the residue was purified by filtering through a pad of silica gel (20:80 to 35:65 EtOAc/Hexane) to give a yellow solid, which was re-crystallized from EtOAc/hexanes to give 4-[2-(2-fluoro-4-methanesulfonyl-phenyl)-1H-indol-5-yl]-3-methyl-benzonitrile as a yellow solid (59 mg, 49% in 2 steps). MS (M+H)=405.

### Example 134

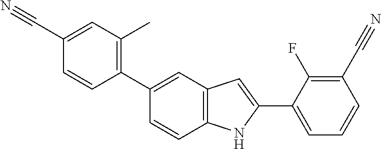

### 4-[2-(2-Fluoro-3-cyano-phenyl)-1H-indol-5-yl]-3-methyl-benzonitrile

Prepared in a similar fashion as the previous example replacing 2-fluoro-4-(methylsulfonyl)phenylboronic acid in Step 1 of Example 133 with 3-borono-2-fluorobenzonitrile to give 4-[2-(2-Fluoro-3-cyano-phenyl)-1H-indol-5-yl]-3-methyl-benzonitrile. MS (M+H)=352.

### Example 135

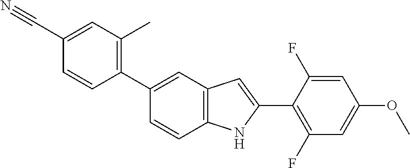

### 4-(2-(2,6-difluoro-4-methoxyphenyl)-1H-indol-5-yl)-3-methylbenzonitrile

Prepared in a similar fashion as the previous example replacing 2-fluoro-4-(methylsulfonyl)phenylboronic acid in Step 1 of Example 133 with 2,6-difluoro-4-methoxyphenylboronic acid to give 4-(2-(2,6-difluoro-4-methoxyphenyl)-1H-indol-5-yl)-3-methylbenzonitrile. MS (M+H)=375.

### Example 136

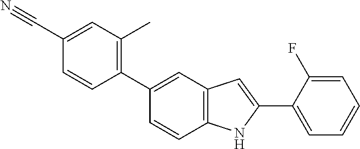

### 4-(2-(2-fluorophenyl)-1H-indol-5-yl)-3-methylbenzonitrile

Prepared in a similar fashion as the previous example replacing 2-fluoro-4-(methylsulfonyl)phenylboronic acid in Step 1 of Example 133 with 2-fluorophenylboronic acid to give 4-(2-(2-fluorophenyl)-1H-indol-5-yl)-3-methylbenzonitrile. MS (M+H)=327.

### Example 137

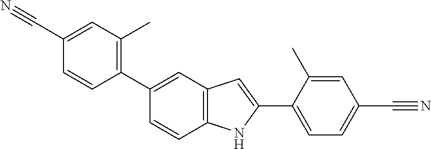

### 4-(2-(4-Cyano-2-methylphenyl)-1H-indol-5-yl)-3-methylbenzonitrile

Prepared in a similar fashion as the previous example replacing 2-fluoro-4-(methylsulfonyl)phenylboronic acid in Step 1 of Example 133 with 4-cyano-2-methylphenylboronic to give 4-(2-(4-Cyano-2-methylphenyl)-1H-indol-5-yl)-3-methylbenzonitrile. MS (M+H)=348.

### Example 138

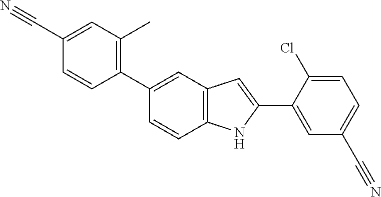

### 4-(2-(2-Chloro-5-cyanophenyl)-1H-indol-5-yl)-3-methylbenzonitrile

Prepared in a similar fashion as the previous example replacing 2-fluoro-4-(methylsulfonyl)phenylboronic acid in Step 1 of Example 133 with 2-chloro-5-cyanophenylboronic acid to give 4-(2-(2-chloro-5-cyanophenyl)-1H-indol-5-yl)-3-methylbenzonitrile. MS (M+H)=368.

### Example 139

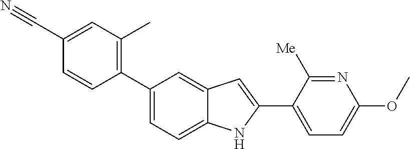

### 4-(2-(6-methoxy-2-methylpyridin-3-yl)-1H-indol-5-yl)-3-methylbenzonitrile

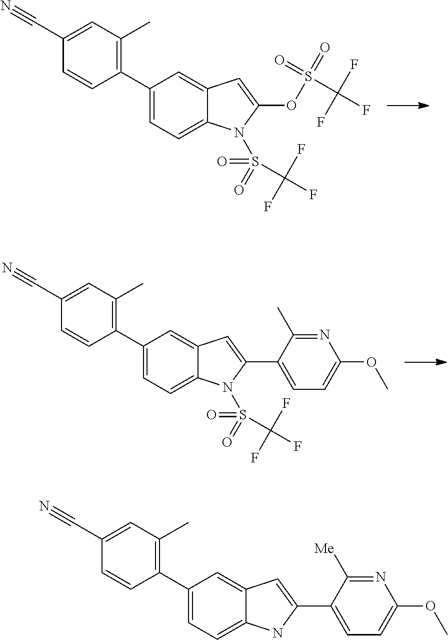

### 4-(2-(6-methoxy-2-methylpyridin-3-yl)-1-(trifluoromethylsulfonyl)-1-1H-indol-5-yl)-3-methylbenzonitrile

To a reaction vial was added: 5-(4-cyano-2-methylphenyl)-1-(trifluoromethylsulfonyl)-1H-indol-2-yl trifluoromethanesulfonate (250 mg, 0.49 mmol), 2-methyl-6-metoxypyridine-3-boronic acid (98 mg, 0.59 mmol), tetrakis(triphenylphosphine)palladium(0) (28 mg, 0.24 mmol), toluene (2.5 ml), Ethanol (1.5 ml) and water (1.00 ml). The reaction mixture was degassed with nitrogen, the vial sealed and stirred while heating to 80° C. for 3 hrs. The cooled reaction mixture was partitioned between ethyl acetate and water, the organic layer was washed with water and brine, dried over sodium sulfate, filtered and concentrated under reduced pressure. The crude 4-(2-(6-methoxy-2-methylpyridin-3-yl)-1-(trifluoromethylsulfonyl)-1H-indol-5-yl)-3-methylbenzonitrile was used in the next step without further purification.

### 4-(2-(6-methoxy-2-methylpyridin-3-yl)-1H-indol-5-yl)-3-methylbenzonitrile

To a solution of 4-(2-(6-methoxy-2-methylpyridin-3-yl)-1-(trifluoromethylsulfonyl)-1H-indol-5-yl)-3-methylbenzonitrile (237 mg, 0.49 mmol) in THF (3 ml), was added a 3N NaOH aqueous solution (3 ml) and the mixture was stirred overnight at room temperature. The reaction mixture was partitioned between EtOAc and water, the organic phase was washed with water and brine, dried over Na₂SO₄, filtered and concentrated under reduced pressure, and the residue was purified by filtering through a pad of silica gel (0% to 35% EtOAc/hexanes) to give 4-(2-(6-methoxy-2-methylpyridin-3-yl)-1H-indol-5-yl)-3-methylbenzonitrile as a pink solid (83 mg, 48% in 2 steps). MS (M+H)=354.

### Example 140

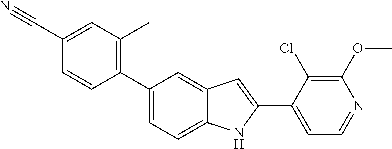

### 4-(2-(3-chloro-2-methoxypyridin-4-yl)-1H-indol-5-yl)-3-methylbenzonitrile

Prepared in a manner identical to Example 139, but replacing 2-methyl-6-methoxypyridine-3-boronic acid with 3-chloro-2-methoxypyridine-4-boronic acid. MS (M+H)=374.

### Example 141

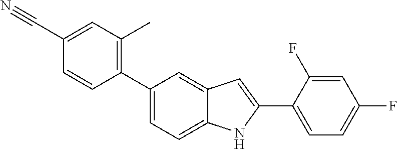

### 4-(2-(2,4-difluorophenyl)-1H-indol-5-yl)-3-methylbenzonitrile

Prepared in a manner identical to Example 139, but replacing 2-methyl-6-methoxypyridine-3-boronic acid with difluorophenylboronic acid. MS (M+H)=345.

### Example 142

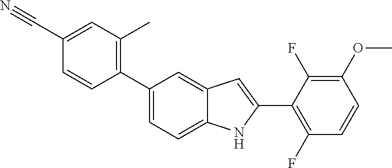

### 4-(2-(2,6-difluoro-3-methoxyphenyl)-1H-indol-5-yl)-3-methylbenzonitrile

Prepared in a manner identical to Example 139, but replacing 2-methyl-6-methoxypyridine-3-boronic acid with 2,6-difluoro-3-methoxyphenylboronic acid. MS (M+H)=375.

### Example 143

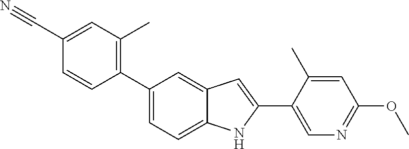

### 4-(2-(6-methoxy-4-methylpyridin-3-yl)-1H-indol-5-yl)-3-methylbenzonitrile

Prepared in a manner identical to Example 139, but replacing 2-methyl-6-methoxypyridine-3-boronic acid with 2-methoxy-4-picoline-5-boronic acid. MS (M+H)=354.

### Example 144

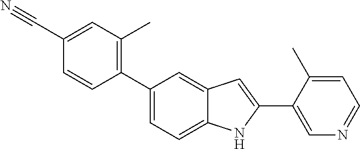

### methyl-4-(2-(4-methylpyridin-3-yl)-1H-indol-5-yl)benzonitrile

Prepared in a manner identical to Example 139, but replacing 2-methyl-6-methoxypyridine-3-boronic acid with 4-methylpyridine-3-boronic acid. MS (M+H)=324.

### Example 145

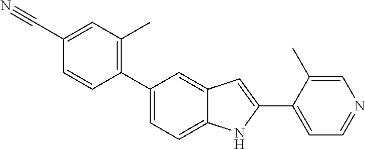

### methyl-4-(2-(3-methylpyridin-4-yl)-1H-indol-5-yl)benzonitrile

Prepared in a manner identical to Example 139, but replacing 2-methyl-6-methoxypyridine-3-boronic acid with 3-methylpyridine-4-boronic acid. MS (M+H)=324.

### Example 146

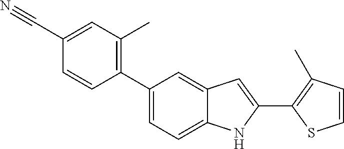

### methyl-4-(2-(3-methylthiophen-2-yl)-1H-indol-5-yl)benzonitrile

Prepared in a manner identical to Example 139, but replacing 2-methyl-6-methoxypyridine-3-boronic acid with 3-methylthiophene-2-boronic acid. MS (M+H)=329.

### Example 147

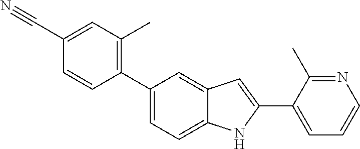

### methyl-4-(2-(2-methylpyridin-3-yl)-1H-indol-5-yl)benzonitrile

Prepared in a manner identical to Example 139, but replacing 2-methyl-6-methoxypyridine-3-boronic acid with 2-methylpyridine-3-boronic acid pinacol ester. MS (M+H)=324.

### Example 148

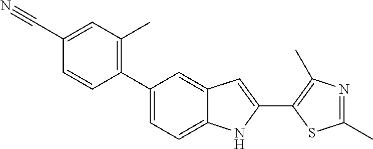

### 4-(2-(2,4-dimethylthiazol-5-yl)-1H-indol-5-yl)-3-methylbenzonitrile

Prepared in a manner identical to Example 139, but replacing 2-methyl-6-methoxypyridine-3-boronic acid with 2,4-dimethyl-5-(4,4,5,5-tetramethyl-1,3,2-dioxaborolan-2-yl)-1,3-thiazole. MS (M+H)=344.

### Example 149

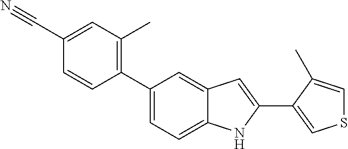

### methyl-4-(2-(4-methylthiophen-3-yl)-1H-indol-5-yl)benzonitrile

Prepared in a manner identical to Example 139, but replacing 2-methyl-6-methoxypyridine-3-boronic acid with 4-methyl-3-thiopheneboronic acid. MS (M+H)=329.

### Example 150

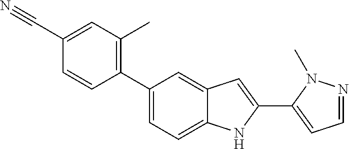

### methyl-4-(2-(1-methyl-1H-pyrazol-5-yl)-1H-indol-5-yl)benzonitrile

Prepared in a manner identical to Example 139, but replacing 2-methyl-6-methoxypyridine-3-boronic acid with 1-methyl-1H-pyrazole-5-boronic acid pinacol ester. MS (M+H)=313.

### Example 151

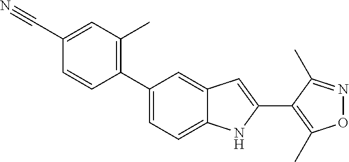

### 4-(2-(3,5-dimethylisoxazol-4-yl)-1H-indol-5-yl)-3-methylbenzonitrile

Prepared in a manner identical to Example 139, but replacing 2-methyl-6-methoxypyridine-3-boronic acid with 3,5-dimethylisoxazole-4-boronic acid. MS (M+H)=328.

### Example 152

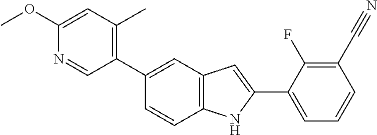

### fluoro-3-(5-(6-methoxy-4-methylpyridin-3-yl)-1H-indol-2-yl)benzonitrile

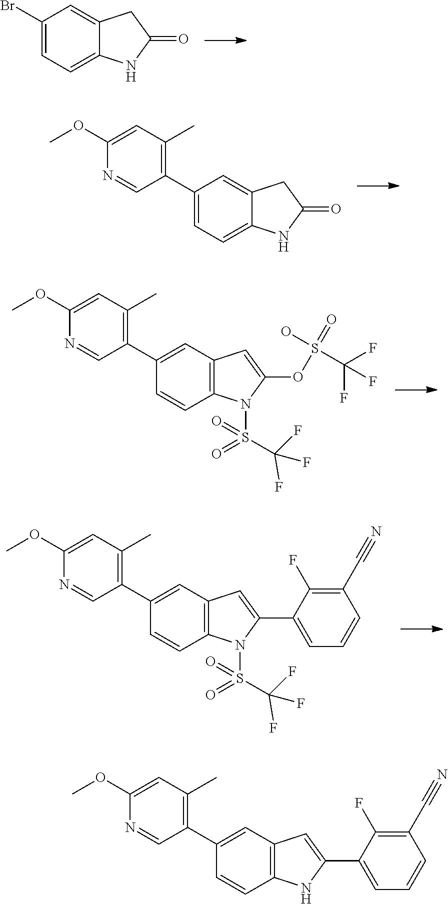

### Step 1 5-(6-methoxy-4-methylpyridin-3-yl)indolin-2-one

To a pressure flask was added a mixture of 5-bromoindolin-2-one (1.1 g, 5.19 mmol) and 6-methoxy-4-methylpyridin-3-ylboronic acid (996 mg, 5.97 mmol) in DMF (30 ml) to give a light brown solution, a solution of sodium carbonate in water (3 ml) was added, while the mixture was degassed with nitrogen, a catalytic amount of [1,1′-bis(diphenylphosphino)ferrocene]dichloropalladium(II) complex with dichloromethane was added and the flask was sealed. The reaction mixture was heated to 90° C. and stirred for 7 h. After the reaction mixture was cooled, water was added, the dark solid was collected, and was washed with the following; twice with water, a solution of 20% EtOAc/hexanes (3 times), a small amount of ethyl acetate (twice), and MeOH (twice), to give 5-(6-methoxy-4-methylpyridin-3-yl)indolin-2-one as a dark solid (0.94 g, 71%).

### Step 2 5-(6-methoxy-4-methylpyridin-3-yl)-1-(trifluoromethylsulfonyl)-1H-indol-2-yl trifluoromethanesulfonate

To a solution of 5-(6-methoxy-4-methylpyridin-3-yl)indolin-2-one (0.2 g, 0.079 mmol) in DMF (5 ml), was added NaH (60% dispersion in mineral oil, 0.094 g, 2.36 mmol) at 0° C., stirred at room temperature for about 15 minutes, N-phenyl-bis(trifluoromethanesulfonimide) (0.843 g, 2.36 mmol) was added, the mixture was stirred at room temperature for one hour, then partitioned between EtOAc and water, the organic phase was washed with brine, dried over Na₂SO₄, filtered and concentrated under reduced pressure. The crude material was purified by flash chromatography (4-8% EtOAc/hexanes) to give 5-(6-methoxy-4-methylpyridin-3-yl)-1-(trifluoromethylsulfonyl)-1H-indol-2-yl trifluoromethanesulfonate (0.050 g, 12%).

### Step 3 2-fluoro-3-(5-(6-methoxy-4-methylpyridin-3-yl)-1-(trifluoromethylsulfonyl)-1H-indol-2-yl)benzonitrile

Similarly prepared as Step 1 in Example 133, but replacing 2-fluoro-4-(methylsulfonyl)phenylboronic acid with 3-borono-2-fluorobenzonitrile to give 2-fluoro-3-(5-(6-methoxy-4-methylpyridin-3-yl)-1-(trifluoromethylsulfonyl)-1H-indol-2-yl)benzonitrile.

### Step 4 2-fluoro-3-(5-(6-methoxy-4-methylpyridin-3-yl)-1H-indol-2-yl)benzonitrile

Similarly prepared as Step 2 in Example 133 to give 2-fluoro-3-(5-(6-methoxy-4-methylpyridin-3-yl)-1H-indol-2-yl)benzonitrile. MS (M+H)=358.

### Example 153

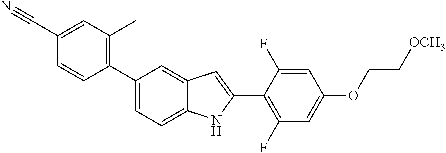

### 4-(2-(2,6-difluoro-4-(2-methoxyethoxy)phenyl)-1H-indol-5-yl)-3-methylbenzonitrile

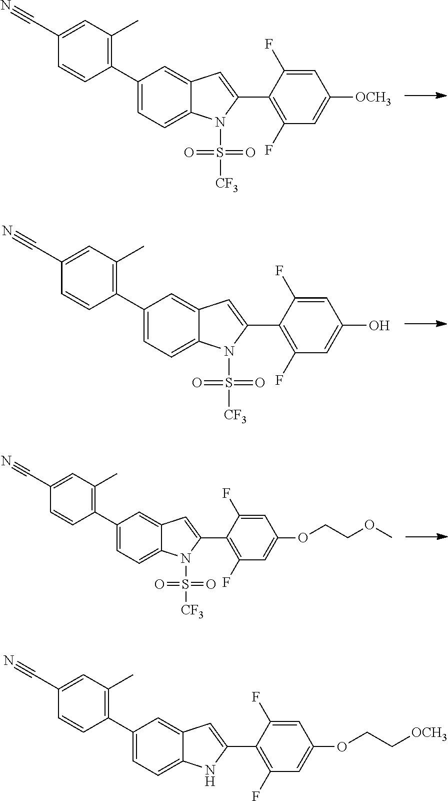

### Step_{—}1 4-(2-(2,6-difluoro-4-hydroxyphenyl)-1-(trifluoromethylsulfonyl)-1H-indol-5-yl)-3-methylbenzonitrile

A solution of 4-(2-(2,6-difluoro-4-methoxyphenyl)-1-(trifluoromethylsulfonyl)-1H-indol-5-yl)-3-methylbenzonitrile (1.27 g, 2.51 mmol) and LiI (1.01 g, 7.53 mmol) in collidine was stirred at 180° C. for 2 h. The reaction mixture was cooled to room temperature and a 10% HCl solution was added, extracted with CH₂Cl₂, washed with water and brine, dried over Na₂SO₄, filtered and concentrated under reduced pressure. The residue was purified by flash chromatography (0% to 35% EtOAc/hexanes) to give 4-(2-(2,6-difluoro-4-hydroxyphenyl)-1-(trifluoromethylsulfonyl)-1H-indol-5-yl)-3-methylbenzonitrile as a white solid (1.13 g, 92%).

### Step_{—}2 4-(2-(2,6-difluoro-4-(2-methoxyethoxy)phenyl)-1-(trifluoromethylsulfonyl)-1H-indol-5-yl)-3-methylbenzonitrile

To a solution of 4-(2-(2,6-difluoro-4-hydroxyphenyl)-1-(trifluoromethylsulfonyl)-1H-indol-5-yl)-3-methylbenzonitrile (80 mg, 0.16 mmol) in DMF, potassium carbonate (90 mg, 0.65 mmol) and 2-bromoethyl methyl ether (34 mg, 23 μl, 0.22 mmol) were added. The reaction mixture was stirred at 70° C. overnight, then raised to 120° C. for 2 hrs., to the cooled reaction mixture was added and the resulting precipitated was filtrated, washed with water and dried under reduced pressure to give 4-(2-(2,6-difluoro-4-(2-methoxyethoxy)phenyl)-1-(trifluoromethylsulfonyl)-1H-indol-5-yl)-3-methylbenzonitrile, which was used directly in the next step without further purification.

### Step_{—}3 4-(2-(2,6-difluoro-4-(2-methoxyethoxy)phenyl)-1H-indol-5-yl)-3-methylbenzonitrile

To a solution of 4-(2-(2,6-difluoro-4-(2-methoxyethoxy)phenyl)-1-(trifluoromethylsulfonyl)-1H-indol-5-yl)-3-methylbenzonitrile (89.2 mg, 162 μmmol, Eq: 1.00) in THF (3 ml), was added 3N NaOH aqueous solution (3 ml). Mixture stirred overnight at room temperature; partitioned between EtOAc and water, EtOAc phase was washed by brine, dried over Na₂SO₄, filtered and concentrated under reduced pressure. The residue was purified by flash chromatography (0% to 35% EtOAc/Hexane) to give 4-(2-(2,6-difluoro-4-(2-methoxyethoxy)phenyl)-1H-indol-5-yl)-3-methylbenzonitrile as a white solid (54 mg, 80% in 2 steps). MS (M+H)=419.

### Example 154

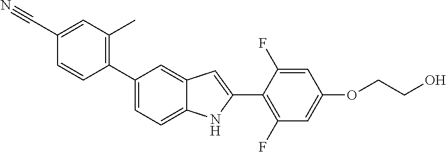

### 4-(2-(2,6-difluoro-4-(2-hydroxyethoxy)phenyl)-1H-indol-5-yl)-3-methylbenzonitrile

Similarly prepared as described for the previous example, but replacing 2-bromoethyl methyl ether with 2-bromoethanol for Step 2 in Example 153 to give 4-(2-(2,6-difluoro-4-(2-hydroxyethoxy)phenyl)-1H-indol-5-yl)-3-methylbenzonitrile as a white solid. MS (M+H)=405.

### Example 155

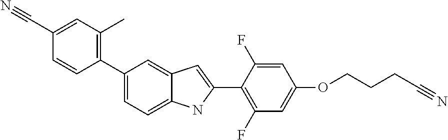

### 4-(2-(4-(3-cyanopropoxy)-2,6-difluorophenyl)-1H-indol-5-yl)-3-methylbenzonitrile

Similarly prepared as described for the previous example, but replacing 2-bromoethyl methyl ether with 4-bromobutanenitrile for Step 2 in Example 153, and the reaction was heated to 120° C. for greater than 2 hrs., until de-protection was complete giving the product, 4-(2-(4-(3-cyanopropoxy)-2,6-difluorophenyl)-1H-indol-5-yl)-3-methylbenzonitrile as a white solid, directly thus avoiding step 3. MS (M+H)=428.

### Example 156

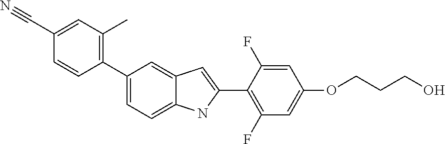

### 4-(2-(2,6-difluoro-4-(3-hydroxypropoxy)phenyl)-1H-indol-5-yl)-3-methylbenzonitrile

Similarly prepared as described for the previous example, but replacing 2-bromoethyl methyl ether with 3-bromopropan-1-ol for Step 2 in Example 153, and the reaction was heated to 120° C. for greater than 2 hrs., until de-protection was complete giving the product, 4-(2-(2,6-difluoro-4-(3-hydroxypropoxy)phenyl)-1H-indol-5-yl)-3-methylbenzonitrile as a white solid, directly thus avoiding step 3. MS (M+H)=419.

### Example 157

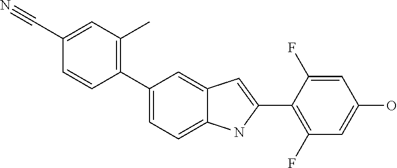

### 4-(2-(2,6-difluoro-4-hydroxyphenyl)-1H-indol-5-yl)-3-methylbenzonitrile

This compound was isolated as the sole by-product in a reaction described in a similar manner to the previous example, but replacing 2-bromoethyl methyl ether with bromoacetonitrile for Step 2 in Example 153, and the reaction was heated to 120° C. for greater than 2 hrs., until de-protection was complete giving the product, 4-(2-(2,6-difluoro-4-hydroxyphenyl)-1H-indol-5-yl)-3-methylbenzonitrile as an off-white solid, directly thus avoiding step 3. MS (M+H)=361.

### Example 158

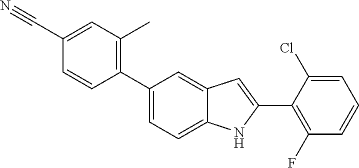

### 4-[2-(2-chloro-6-fluorophenyl)-1H-indol-5-yl]-3-methylbenzonitrile

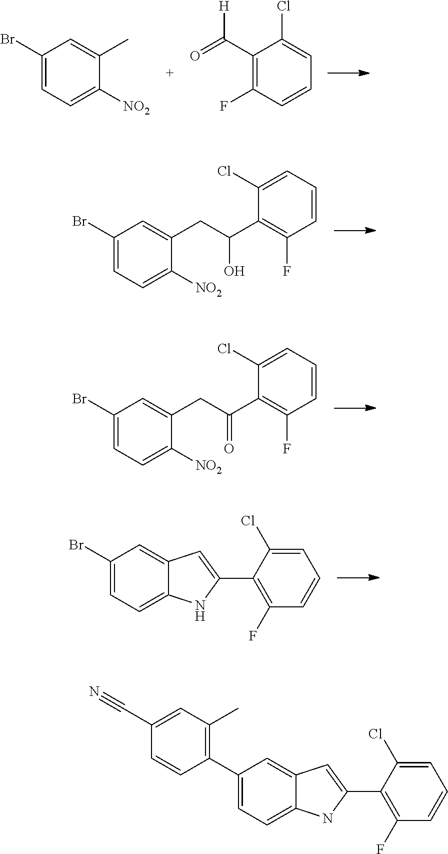

### Step_{—}1 2-(5-bromo-2-nitrophenyl)-1-(2-chloro-6-fluorophenyl)ethanol

To a solution of 4-bromo-2-methyl-1-nitrobenzene (6.54 g, 30 mmol) and 2-chloro-6-fluorobenzaldehyde (4.78 g, 30 mmol) in DMSO (10 ml), was added DBU (4.5 ml, 30 mmol) dropwise. The reaction mixture was stirred at room temperature for 4 hours, then partitioned between EtOAc and water, the organic phase was washed with water and brine, dried over Na₂SO₄, filtered and concentrated under reduced pressure. The crude material was purified by filtering through a pad of silica gel (0% to 20% EtOAc/Hexane) to give the product (7.2 g, 64%). MS (M−H)=374.

### Step_{—}2 2-(5-bromo-2-nitrophenyl)-1-(2-chloro-6-fluorophenyl)ethanone

To a 0° C. solution of 2-(5-bromo-2-nitrophenyl)-1-(2-chloro-6-fluorophenyl)ethanol (7.2 g, 19 mmol) in dichloromethane (90 ml), was added Dess-Martin periodinane (8.97 g, 21.1 mmol) and the reaction mixture was stirred for 2 hours allowing it to warm up to room temperature, partitioned between EtOAc and water, the organic phase was washed with water, aqueous sodium bicarbonate (3 times) and brine, dried over Na₂SO₄, filtered and concentrated under reduced pressure to give the product (7.15 g, 100%). MS (M−H)=372.

### Step_{—}3 5-bromo-2-(2-chloro-6-fluorophenyl)-1H-indole

To a solution of 2-(5-bromo-2-nitrophenyl)-1-(2-chloro-6-fluorophenyl)ethanone (7.15 g, 19.2 mmol) in acetic acid (200 ml) and methanol (200 ml), was added iron powder (8.58 g, 154 mmol). The reaction mixture was stirred at room temperature for 3 hours, filtered through a paper filter, concentrated under reduce pressure, added water and extracted with EtOAc. The organic phase was washed with water, aqueous sodium bicarbonate (2 times) and brine, dried over Na₂SO₄, filtered and concentrated under reduced pressure. The crude product was purified by flash chromatography (0% to 20% EtOAc/Hexanes) to give the product as a crystalline solid (5.68 g, 91%). MS (M+H)=326.

4-[2-(2-chloro-6-fluorophenyl)-1H-indol-5-yl]-3-methylbenzonitrile: A suspension of 5-bromo-2-(2-chloro-6-fluorophenyl)-1H-indole (100 mg, 308 μmmol, Eq: 1.00), 4-cyano-2-methylphenylboronic acid (64.5 mg, 401 μmmol, Eq: 1.3) and Potassium carbonate (128 mg, 924 mmol, Eq: 3) in Dioxane (3.00 ml) and Water (0.6 ml) was purged with nitrogen (10 min) and then 1,1′-bis(diphenylphosphino)ferrocenedichloro palladium(II) (22.5 mg, 30.8 μmmol, Eq: 0.1) was added and r×n. mixture was heated at 100 C for 3 hr. Rxn. mixture diluted with water, extracted with DCM, washed with brine, dried (Magnesium sulfate). Strip to obtain an oil (0.13 g), chromatographed (silica gel, 10% EtOAc-Hexane to obtain 4-(2-(2-chloro-6-fluorophenyl)-1H-indol-5-yl)-3-methylbenzonitrile (90.4 mg, 251 μmmol, 81% yield) as a white foam. LC/MS: (M+H)=361.

### Example 159

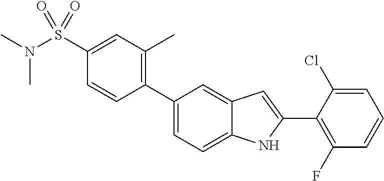

### 4-[2-(2-Chloro-6-fluoro-phenyl)-1H-indol-5-yl]-3,N,N-trimethyl-benzenesulfonamide

Prepared as described in Example 158, using 4-(N,N-dimethylsulfamoyl)-2-methylphenylboronic acid and 5-bromo-3-methyl-2-phenyl-1H-indole.

4-[2-(2-Chloro-6-fluoro-phenyl)-1H-indol-5-yl]-3,N,N-trimethyl-benzenesulfonamide, LC/MS (M+H)=443

### Example 160

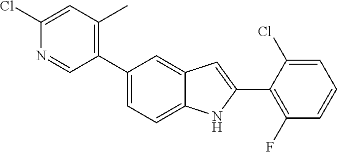

### 2-(2-Chloro-6-fluoro-phenyl)-5-(6-chloro-4-methyl-pyridin-3-yl)-1H-indole

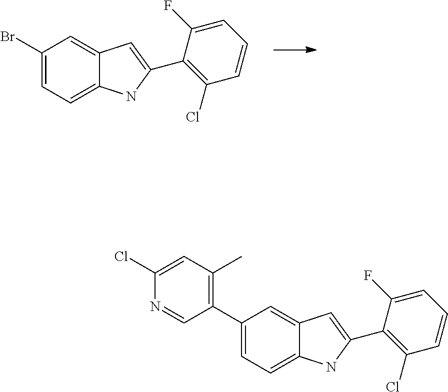

### 2-(2-Chloro-6-fluoro-phenyl)-5-(6-chloro-4-methyl-pyridin-3-yl)-1H-indole

Bromo-2-(2-chloro-6-fluorophenyl)-1H-indole (73 mg, 225 μmmol), 6-chloro-4-methylpyridine-3-boronic acid (50 mg, 292 μmmol), and [1,1′-bis(diphenylphosphono)ferrocene]dichloropalladium(II) (33 mg, 45.1 μmmol) were combined with Dioxane (4 mL) and flushed with nitrogen. A solution of potassium carbonate (94 mg, 680 mmol) in water (1 mL) was added and mixture was heated in a sealed tube at 80° C. for 1 h. The mixture was cooled, diluted with ethyl acetate, washed with brine, dried over Na₂SO₄, filtered and concentrated under reduced pressure. The resulting crude compound was purified by flash column chromatography (silica gel, 25 g, 10% to 20% ethyl acetate in hexanes) to give 2-(2-chloro-6-fluoro-phenyl)-5-(6-chloro-4-methyl-pyridin-3-yl)-1H-indole. MS (M+H)=371.

### Example 161

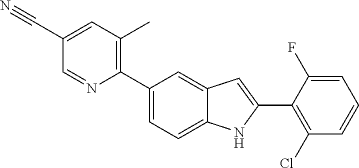

### 6-(2-(2-chloro-6-fluorophenyl)-1H-indol-5-yl)-5-methylnicotinonitrile

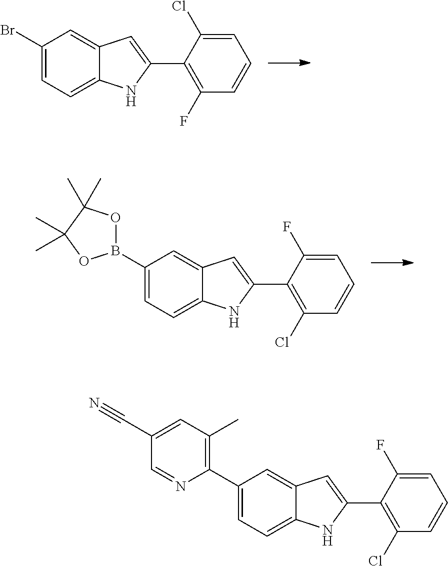

### 2-(2-chloro-6-fluorophenyl)-5-(4,4,5,5-tetramethyl-1,3,2-dioxaborolan-2-yl)-1H-indole

To a reaction vial was added: 5-bromo-2-(2-chloro-6-fluorophenyl)-1H-indole (5.68 g, 18 mmol), bis(pinacolato)diboron (5.78 g, 22.8 mmol), 1,1′-bis(diphenylphosphino)ferrocene-palladium(II) dichloride dichloromethane complex (1.43 g, 9.7 mol %), potassium acetate (6.87 g, 70.0 mmol), dioxane (20 ml). The reaction mixture was degassed with nitrogen, the vial sealed and stirred while heating to 110° C. for 3 hrs. The cooled reaction mixture was filtered through celite, eluted with EtOH and EtOAc and concentrated under reduced pressure. The residue was redissolved in EtOAc and washed with water and brine, dried (Na₂SO₄), filtered and concentrated under reduced pressure then purified by flash chromatography (10:90 EtOAc/hexanes to 100% EtOAc) to give the product as a light brown solid (4.46 g, 69%).

### 6-(2-(2-chloro-6-fluorophenyl)-1H-indol-5-yl)-5-methylnicotinonitrile

To a reaction vial was added: 2-(2-Chloro-6-fluoro-phenyl)-5-(4,4,5,5-tetramethyl-[1,3,2]dioxaborolan-2-yl)-1H-indole (125 mg, 0.34 mmol) 6-bromo-5-methylnicotinonitrile (80 mg, 0.40 mmol), tetrakis(triphenylphosphine)palladium (0) (19 mg, 0.17 mmol, 5 mol %), sodium bicarbonate (85 mg, 1.0 mmol), toluene (2.5 ml), ethanol (1.5 ml) and water (1.00 ml). The reaction mixture was degassed with nitrogen, the vial sealed and heated to 80° C. for 3 hrs. The cooled reaction mixture was partitioned between EtOAc and water, washed with water and brine, dried (Na₂SO₄), filtered and concentrated under reduced pressure. The residue was purified by filtering through a pad of silica gel (0% to 35% EtOAc/hexanes) to give 6-[2-(2-chloro-6-fluorophenyl)-1H-indol-5-yl]-5-methylnicotinonitrile as a yellow solid (74 mg, 61%). MS (M+H)=362.

### Example 162

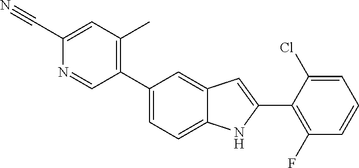

### 5-(2-(2-chloro-6-fluorophenyl)-1H-indol-5-yl)-4-methylpicolinonitrile

Similarly prepared using the above procedure outlined in Example 161, but replacing 6-bromo-5-methylnicotinonitrile with 5-bromo-4-methylpicolinonitrile. MS (M+H)=362.

### Example 163

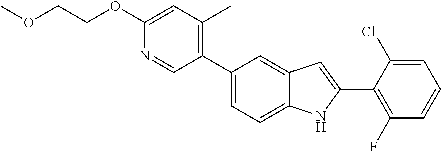

### 2-(2-chloro-6-fluorophenyl)-5-(6-(2-methoxyethoxy)-4-methylpyridin-3-yl)-1H-indole

Similarly prepared using the above procedure outlined in Example 161, but replacing 6-bromo-5-methylnicotinonitrile with 5-bromo-2-(2-methoxyethoxy)-4-methylpyridine. MS (M+H)=411.

### Example 164

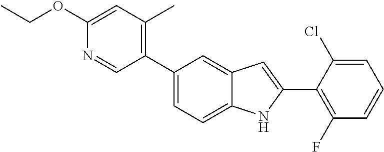

### 2-(2-chloro-6-fluorophenyl)-5-(6-ethoxy-4-methylpyridin-3-yl)-1H-indole

Similarly prepared using the above procedure outlined in Example 161, but replacing 6-bromo-5-methylnicotinonitrile with 5-bromo-2-ethoxy-4-methylpyridine. MS (M+H)=381

### Example 165

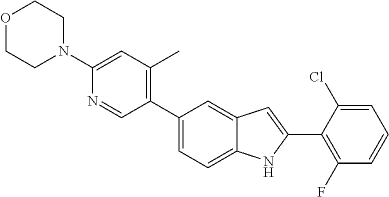

### 4-(5-(2-(2-chloro-6-fluorophenyl)-1H-indol-5-yl)-4-methylpyridin-2-yl)morpholine

Similarly prepared using the above procedure outlined in Example 161, but replacing 6-bromo-5-methylnicotinonitrile with 4-(5-bromo-4-methylpyridin-2-yl)morpholine. MS (M+H)=422.

### Example 166

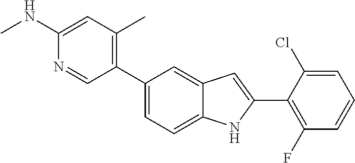

### 5-(2-(2-chloro-6-fluorophenyl)-1H-indol-5-yl)-N,4-dimethylpyridin-2-amine

Similarly prepared using the above procedure outlined in Example 161, but replacing 6-bromo-5-methylnicotinonitrile with 5-bromo-N,4-dimethylpyridin-2-amine. MS (M+H)=366.

### Example 167

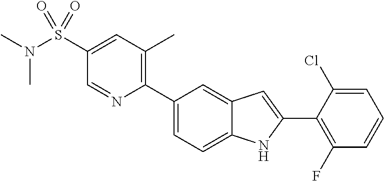

### 6-(2-(2-chloro-6-fluorophenyl)-1H-indol-5-yl)-N,N,5-trimethylpyridine-3-sulfonamide

Similarly prepared using the above procedure outlined in Example 161, but replacing 6-bromo-5-methylnicotinonitrile with 6-chloro-5-methyl-pyridine-3-sulfonic acid dimethylamide. MS (M+H)=444.

### Example 168

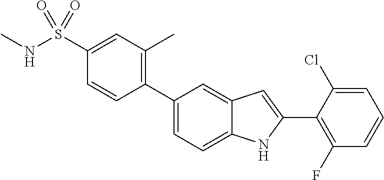

### 4-(2-(2-chloro-6-fluorophenyl)-1H-indol-5-yl)-N,3-dimethylbenzenesulfonamide

Similarly prepared using the above procedure outlined in Example 161, but replacing 6-bromo-5-methylnicotinonitrile with 4-bromo-N,3-dimethylbenzenesulfonamide. MS (M+H)=429.

### Example 169

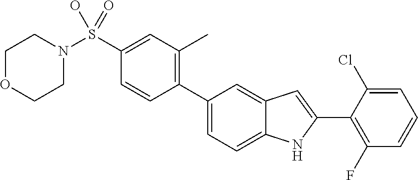

### 4-(4-(2-(2-chloro-6-fluorophenyl)-1H-indol-5-yl)-3-methylphenylsulfonyl)morpholine

Similarly prepared using the above procedure outlined in Example 161, but replacing 6-bromo-5-methylnicotinonitrile with 4-(4-chloro-3-methyl-benzenesulfonyl)-morpholine. MS (M+H)=485.

### Example 170

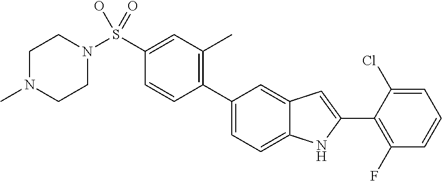

### 2-(2-chloro-6-fluorophenyl)-5-(2-methyl-4-(4-methylpiperazin-1-ylsulfonyl)phenyl)-1H-indole

Similarly prepared using the above procedure outlined in Example 161, but replacing 6-bromo-5-methylnicotinonitrile with 1-(4-Chloro-3-methyl-benzenesulfonyl)-4-methyl-piperazine. MS (M+H)=499.

### Example 171

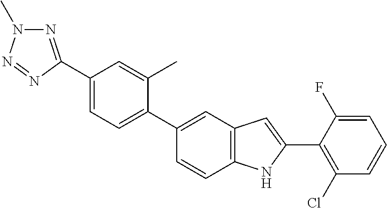

### 2-(2-chloro-6-fluorophenyl)-5-(2-methyl-4-(2-methyl-2H-tetrazol-5-yl)phenyl)-1H-indole

Similarly prepared using the above procedure outlined in Example 161, but replacing 6-bromo-5-methylnicotinonitrile with 5-(4-bromo-3-methylphenyl)-2-methyl-2H-tetrazole. MS (M+H)=418.

### Example 172

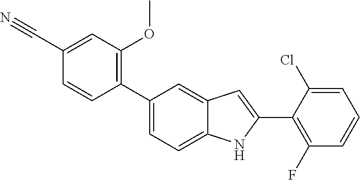

### 4-[2-(2-Chloro-6-fluoro-phenyl)-1H-indol-5-yl]-3-methoxy-benzonitrile

Similarly prepared using the above procedure outlined in Example 161, but replacing 6-bromo-5-methylnicotinonitrile with 4-bromo-3-methoxybenzonitrile. MS (M+H)=377.

### Example 173

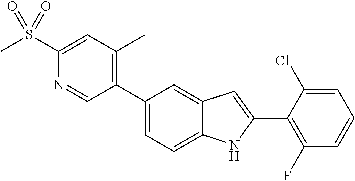

### 2-(2-Chloro-6-fluoro-phenyl)-5-(6-methanesulfonyl-4-methyl-pyridin-3-yl)-1H-indole

Similarly prepared using the above procedure outlined in Example 161, but replacing 6-bromo-5-methylnicotinonitrile with 5-bromo-4-methyl-2-(methylsulfonyl)pyridine. MS (M+H)=415.

### Example 174

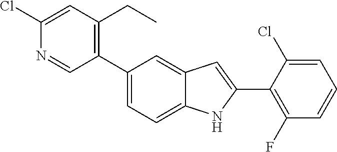

### 5-(6-Chloro-4-ethyl-pyridin-3-yl)-2-(2-chloro-6-fluoro-phenyl)-1H-indole

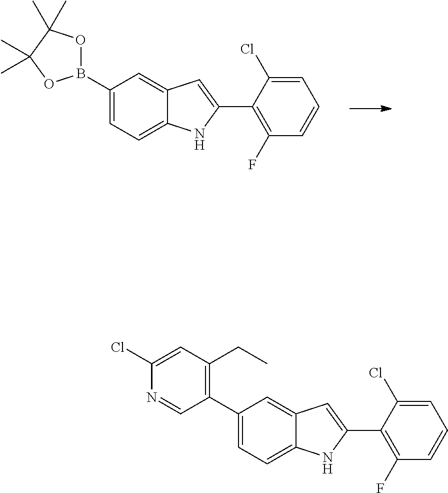

### Step 3 5-(6-Chloro-4-ethyl-pyridin-3-yl)-2-(2-chloro-6-fluoro-phenyl)-1H-indole

2-(2-chloro-6-fluorophenyl)-5-(4,4,5,5-tetramethyl-1,3,2-dioxaborolan-2-yl)-1H-indole (80 mg, 215 μmmol, Eq: 1.00), 2-chloro-4-ethyl-5-iodopyridine (57.6 mg 215 μmmol, Eq: 1.00), tetrakis(triphenylphosphine)palladium (0) (24.9 mg, 21.5 μmmol, Eq: 0.1) and potassium carbonate (89.3 mg, 646 μmmol, Eq: 3) in dioxane (3.83 ml)/Water (957 μl) was heated to 93° C. for 3 hrs. Dried onto silica gel for purification using a 5-15% EtOAc/Hex gradient. Obtained 5-(6-Chloro-4-ethyl-pyridin-3-yl)-2-(2-chloro-6-fluoro-phenyl)-1H-indole (63 mg, 76% yield) as a white solid; MS (M+H)=386.

### Example 175

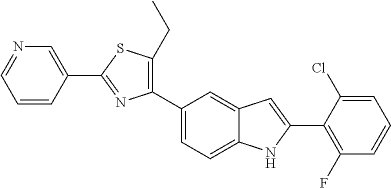

### 4-[2-(2-chloro-6-fluorophenyl)-1H-indol-5-yl]-5-ethyl-2-(pyridin-3-yl)thiazole

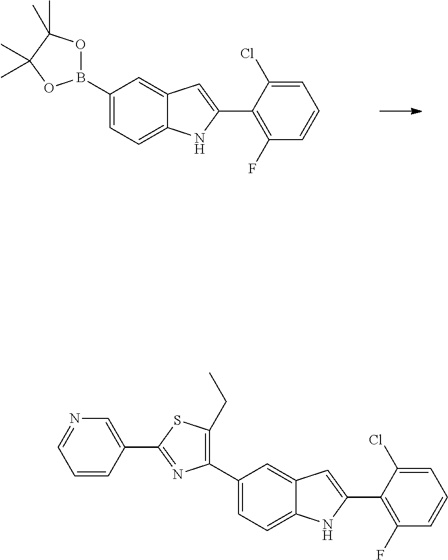

4-(2-(2-chloro-6-fluorophenyl)-1H-indol-5-yl)-5-ethyl-2-(pyridin-3-yl)thiazole: A suspension of 2-(2-chloro-6-fluorophenyl)-5-(4,4,5,5-tetramethyl-1,3,2-dioxaborolan-2-yl)-1H-indole (200 mg, 538 μmmol, Eq: 1.00), Triffluoro-methanesulfonic acid 5-ethyl-2-pyridin-3-yl-thiazole-4-yl ester (Intermediate 37, 218 mg, 646 μmmol, Eq: 1.2), 1,1′-bis(diphenylphosphino)ferrocenedichloro palladium(II) (39.4 mg, 53.8 μmmol, Eq: 0.1) and Potassium carbonate (223 mg, 1.61 mmol, Eq: 3) in Dioxane (4.00 ml) and Water (1.0 ml) was purged with nitrogen (10 min) and heated at 100 C for 3 hrs. Diluted with water, extracted with DCM, organic layer washed with brine, dried (Magnesium sulfate). Strip, chromatographed (silica gel, 30% EtOAc-Hexane) to obtain 4-(2-(2-chloro-6-fluorophenyl)-1H-indol-5-yl)-5-ethyl-2-(pyridin-3-yl)thiazole (112 mg, 258 μmmol, 48.0% yield) as a light yellow powder. LC/MS: (M+H)=434

### Example 176

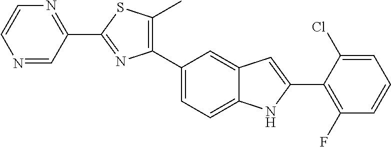

### 2-(2-Chloro-6-fluoro-phenyl)-5-(5-methyl-2-pyrazin-2-yl-thiazol-4-yl)-1H-indole

Prepared as described in Example 175 substituting Intermediate 38 as the triflate coupling partner.

2-(2-Chloro-6-fluoro-phenyl)-5-(5-methyl-2-pyrazin-2-yl-thiazol-4-yl)-1H-indole, LC/MS (M+H)=421

### Example 177

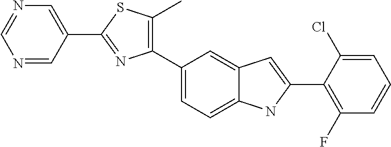

### 2-(2-Chloro-6-fluoro-phenyl)-5-(5-methyl-2-pyrimidin-5-yl-thiazol-4-yl)-1H-indole

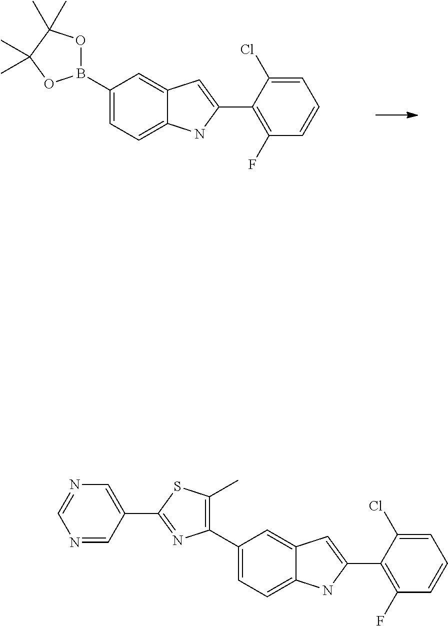

In a 50 mL round-bottomed flask, 2-(2-chloro-6-fluorophenyl)-5-(4,4,5,5-tetramethyl-1,3,2-dioxaborolan-2-yl)-1H-indole (54 mg, 145 μmmol), 5-methyl-2-(pyrimidin-5-yl)thiazol-4-yl trifluoromethanesulfonate (47.3 mg, 145 μmmol), [1,1′-bis(diphenyl phosphino)ferrocenedichloropalladium (II) (21.3 mg, 29.1 μmmol, Eq: 0.2) and potassium carbonate (60.2 mg, 436 μmmol) were combined with Dioxane (6.67 ml) to give a red suspension. The resultant reaction was heated to 80° C. and stirred for 1 h. The reaction mixture was poured into 50 mL H₂O and extracted with ethyl acetate (3×20 mL). The organic layers were dried over MgSO₄ and concentrated in vacuo. The crude material was purified by flash column chromatography (silica gel, 12 g, 15% to 25% ethyl acetate in hexanes). Fraction 21-26 were combined to give 33 mgs of 2-(2-Chloro-6-fluoro-phenyl)-5-(5-methyl-2-pyrimidin-5-yl-thiazol-4-yl)-1H-indole as light yellow solid. Second purification by preparative reverse phase HPLC (Supercosil™ Cat# 59174, 25 cm×21.2 mm×12 micron, 20 to 95% acetonitrile/water with 0.05% TFA) gave 2-(2-Chloro-6-fluoro-phenyl)-5-(5-methyl-2-pyrimidin-5-yl-thiazol-4-yl)-1H-indole as a TFA salt (13 mg, 9.02%) of as a lyophilized solid. MS (M+H)=421.

### Example 178

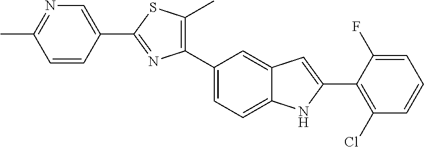

### 2-(2-Chloro-6-fluoro-phenyl)-5-[5-methyl-2-(6-methyl-pyridin-3-yl)-thiazol-4-yl]-1H-indole

2-(2-Chloro-6-fluoro-phenyl)-5-[5-methyl-2-(6-methyl-pyridin-3-yl)-thiazol-4-yl]-1H-indole was prepared in a manner identical Example 177 with the following materials 2-(2,6-difluorophenyl)-5-(4,4,5,5-tetramethyl-1,3,2-dioxaborolan-2-yl)-1H-indole and Trifluoro-methanesulfonic acid 5-methyl-2-(6-methyl-pyridin-3-yl)-thiazol-4-yl ester. MS (M+H)=434.

### Example 179

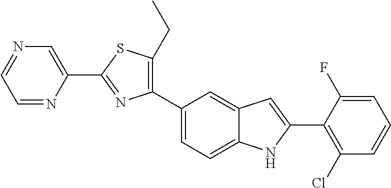

### 2-(2-Chloro-6-fluoro-phenyl)-5-(5-ethyl-2-pyrazin-2-yl-thiazol-4-yl)-1H-indole

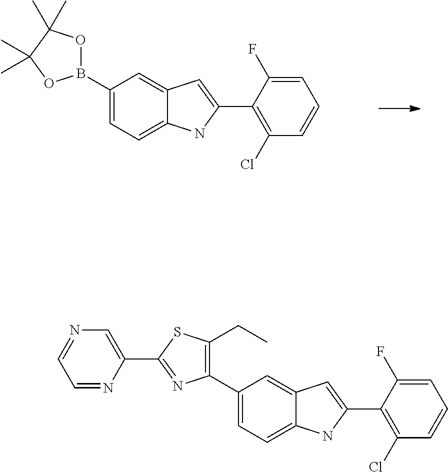

2-(2-Chloro-6-fluoro-phenyl)-5-(5-ethyl-2-pyrazin-2-yl-thiazol-4-yl)-1H-indole: In a 10 mL round-bottomed flask, 2-(2-Chloro-6-fluoro-phenyl)-5-(4,4,5,5-tetramethyl-1,3,2-dioxaborolan-2-yl)-1H-indole (80 mg, 215 μmmol), Trifluoro-methanesulfonic acid 5-ethyl-2-pyrazin-2-yl-thiazol-4-yl ester (73 mg, 215 μmmol) and [1,1′-bis(diphenylphosphino)ferrocenedichloropalladium (II) (32 mg, 43 μmmol) and potassium carbonate (89 mg, 646 μmmol) were combined with dioxane (7 ml) to give a red suspension and the resultant reaction was heated to 80° C. and stirred for 1 h. The reaction mixture was poured into 50 mL H₂O and extracted with EtOAc (3×20 mL). The organic layers were dried over MgSO₄ and concentrated in vacuo. The crude material was purified by flash column chromatography (silica gel, 12 g, 20% to 25% ethyl acetate in hexanes) to give 2-(2-Chloro-6-fluoro-phenyl)-5-(5-ethyl-2-pyrazin-2-yl-thiazol-4-yl)-1H-indole (33 mg, 35.2%) as light yellow solid. MS (M+H)=435.

### Example 180

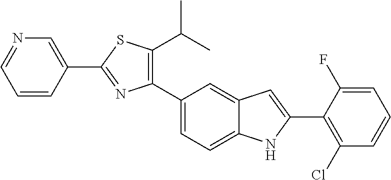

### 2-(2-Chloro-6-fluoro-phenyl)-5-(5-isopropyl-2-pyridin-3-yl-thiazol-4-yl)-1H-indole

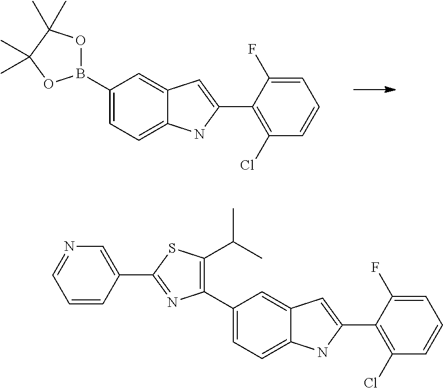

2-(2-Chloro-6-fluoro-phenyl)-5-(5-isopropyl-2-pyridin-3-yl-thiazol-4-yl)-1H-indole: In a 10 mL round-bottomed flask, 2-(2-Chloro-6-fluoro-phenyl)-5-(4,4,5,5-tetramethyl-1,3,2-dioxaborolan-2-yl)-1H-indole (150 mg, 404 μmmol), trifluoro-methanesulfonic acid 5-isopropyl-2-pyridin-3-yl-thiazol-4-yl ester (171 mg, 485 μmmol) and [1,1′-bis(diphenylphosphino) ferrocenedichloropalladium (II) (59 mg, 80.7 μmmol) and potassium carbonate (167 mg, 1.21 mmol) were combined with dioxane (10 ml) to give a red suspension and the resultant reaction was heated to 80° C. and stirred for 12 h. The reaction mixture was poured into 50 mL H₂O and extracted with EtOAc (3×20 mL). The organic layers were dried over MgSO₄ and concentrated in vacuo. The crude material was purified by flash column chromatography (silica gel, 40 g, 20% to 25% ethyl acetate in hexanes). to give 2-(2-Chloro-6-fluoro-phenyl)-5-(5-isopropyl-2-pyridin-3-yl-thiazol-4-yl)-1H-indole (28 mg) as light yellow solid. Second purification by flash column chromatography (silica gel, 12 g, 20% to 25% EtOAc in hexanes). to give 2-(2-Chloro-6-fluoro-phenyl)-5-(5-isopropyl-2-pyridin-3-yl-thiazol-4-yl)-1H-indole (16 mg, 8.85%). MS (M+H)=448.

### Example 181

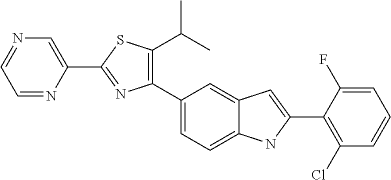

### 2-(2-Chloro-6-fluoro-phenyl)-5-(5-isopropyl-2-pyrazin-2-yl-thiazol-4-yl)-1H-indole

2-(2-Chloro-6-fluoro-phenyl)-5-(5-isopropyl-2-pyrazin-2-yl-thiazol-4-yl)-1H-indole was prepared in a manner identical to 2-(2,6-Difluoro-phenyl)-5-(5-methyl-2-pyrazin-2-yl-thiazol-4-yl)-1H-indole with the following materials 2-(2,6-difluorophenyl)-5-(4,4,5,5-tetramethyl-1,3,2-dioxaborolan-2-yl)-1H-indole and trifluoro-methanesulfonic acid 5-isopropyl-2-pyrazin-2-yl-thiazol-4-yl ester. MS (M+H)=449.

### Example 182

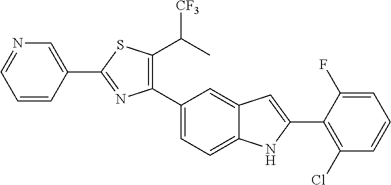

### 2-(2-chloro-6-fluoro-phenyl)-5-[2-pyridin-3-yl-5-(2,2,2-trifluoro-1-methyl-ethyl)-thiazol-4-yl]-1H-indole

2-(2-chloro-6-fluoro-phenyl)-5-[2-pyridin-3-yl-5-(2,2,2-trifluoro-1-methyl-ethyl)-thiazol-4-yl]-1H-indole was prepared in a manner identical to 2-(2,6-Difluoro-phenyl)-5-(5-methyl-2-pyrazin-2-yl-thiazol-4-yl)-1H-indole with the following materials 2-(2,6-difluorophenyl)-5-(4,4,5,5-tetramethyl-1,3,2-dioxaborolan-2-yl)-1H-indole and Trifluoro-methanesulfonic acid 2-pyridin-3-yl-5-(2,2,2-trifluoro-1-methyl-ethyl)-thiazol-4-yl ester. MS (M+H)=502.

### Example 183

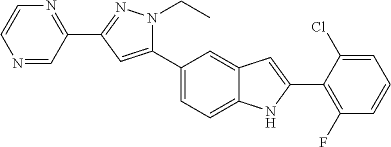

### 2-(2-chloro-6-fluorophenyl)-5-(1-ethyl-3-(pyrazin-2-yl)-1H-pyrazol-5-yl)-1H-indole

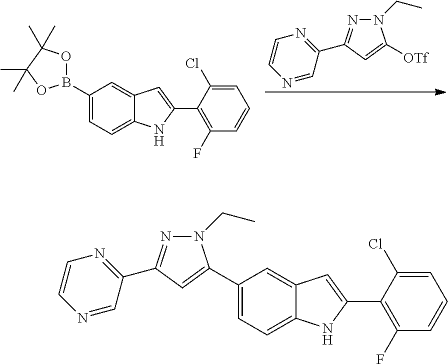

2-(2-chloro-6-fluorophenyl)-5-(4,4,5,5-tetramethyl-1,3,2-dioxaborolan-2-yl)-1H-indole (75 mg, 202 μmmol, Eq: 1.00), 1-ethyl-3-(pyrazin-2-yl)-1H-pyrazol-5-yl trifluoromethanesulfonate (78.0 mg, 242 μmmol, Eq: 1.2), potassium carbonate (83.7 mg, 605 mmol, Eq: 3) and tetrakis(triphenylphosphine)palladium (0) (23.3 mg, 20.2 μmmol, Eq: 0.1) in Dioxane (3.59 ml)/Water (897 μl) was heated at 90° C. for 3 hrs. Dried onto silica gel for purification using a 15-60% EtOAc/Hex gradient. Obtained 2-(2-chloro-6-fluorophenyl)-5-(1-ethyl-3-(pyrazin-2-yl)-1H-pyrazol-5-yl)-1H-indole (11 mg, 26.3 μmmol, 13.0% yield) as a brown solid; MS (M+H)=419.

### Example 184

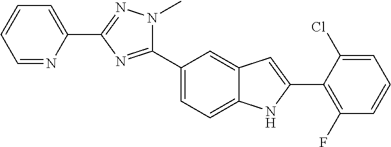

### 2-(2-Chloro-6-fluoro-phenyl)-5-(2-methyl-5-pyridin-2-yl-2H-[1,2,4]-triazol-3-yl)-1H-indole

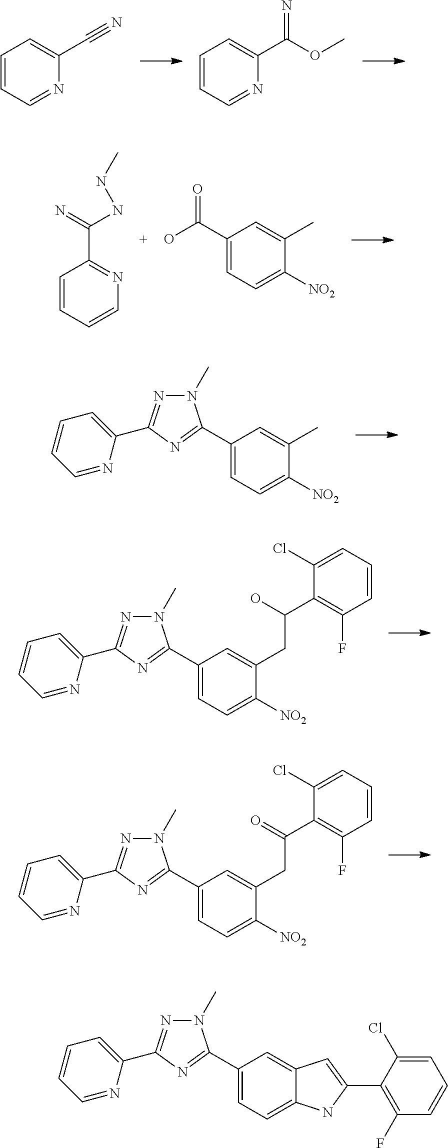

Methyl-2-picolinimidate: Stirred 2-picolinonitrile (3 g, 28.8 mmol, Eq: 1.00) in methanol (25 ml), added Sodium Methoxide as a 4.6 M solution in methanol (Aldrich) (12.5 ml, 57.6 mmol, Eq: 2) dropwise. Stirred at room temperature 24 hours. Removed majority of methanol with rotary evaporation, diluted ethyl acetate, washed water, brine, dried over magnesium sulfate. Evaporated solvent under vacuum, pumped down to give an oil (3.4 g, 87%) methyl-2-picolinimidate, which was used without purification.

### Step 2 N′-methyl-2-picolinimidohydrazide

Stirred methyl picolinimidate (1.65 g, 12.1 mmol, Eq: 1.00) in Pyridine (10 ml), added methylhydrazine (558 mg, 12.1 mmol, Eq: 1), stirred at room temperature 1.5 hours. Removed solvent under vacuum pyridine to a thick oil Product slowly crystallizes under vacuum pump, triturated with ether 4× to give a yellow solid white solid (365 mg., 20%), N′-methyl-2-picolinimidohydrazide, used as is with no purification.

### Step 3 2-(1-methyl-5-(3-methyl-4-nitrophenyl)-1H-1,2,4-triazol-3-yl)pyridine

Stirred 3-methyl-4-nitrobenzoic acid (120 mg, 662 μmmol, Eq: 1.00) in a tube in THF (3 ml) under nitrogen. Added carbonyl diimidazole (118 mg, 729 μmmol, Eq: 1.1), stirred at room temperature 1 hour. Added N′-methyl-2-picolinimidohydrazide (99.5 mg, 662 μmmol, Eq: 1.00), heated to 80 C. Heated a total of 8 hours. Cooled, stirred at room temperature overnight. Diluted methylene chloride, washed water 2×, brine, dried over magnesium sulfate, chromatographed using Analogix system (20% to 100% ethyl acetate in hexanes) to give 2-(1-methyl-5-(3-methyl-4-nitrophenyl)-1H-1,2,4-triazol-3-yl)pyridine (90 mgs, 46%) as a white solid.

### Step 4 1-(2-chloro-6-fluorophenyl)-2-(5-(1-methyl-3-(pyridin-2-yl)-1H-1,2,4-triazol-5-yl)-2-nitrophenyl)ethanol

Stirred 2-(1-methyl-5-(3-methyl-4-nitrophenyl)-1H-1,2,4-triazol-3-yl)pyridine (85 mg, 288 μmmol, Eq: 1.00) in DMSO (2 ml) under N2, added 2-chloro-6-fluorobenzaldehyde (45.6 mg, 288 μmmol, Eq: 1.00), then DBU (43.8 mg, 43.4 μl, 288 μmmol, Eq: 1.00). Stirred at room temperature 24 hours, diluted ethyl acetate, washed water 3×, brine, dried magnesium sulfate. Removed solvent under vacuum to give a foam, crude 1-(2-chloro-6-fluorophenyl)-2-(5-(1-methyl-3-(pyridin-2-yl)-1H-1,2,4-triazol-5-yl)-2-nitrophenyl)ethanol (115 mg, 88%) took on with no further purification.

### Step 5 1-(2-Chloro-6-fluoro-phenyl)-2-[5-(2-methyl-5-pyridin-2-yl-2H-[1,2,4]triazol-3-yl)-2-nitro-phenyl]-ethanone

Stirred 1-(2-chloro-6-fluorophenyl)-2-(5-(1-methyl-3-(pyridin-2-yl)-1H-1,2,4-triazol-5-yl)-2-nitrophenyl)ethanol (115 mg, 253 μmmol, Eq: 1.00) in dichloromethane (5 ml), added Dess-Martin Periodinane (107 mg, 253 μmmol, Eq: 1.00), stirred at room temperature 18 hours. Diluted methylene chloride, washed water, saturated aqueous sodium bicarbonate (2×), brine, dried magnesium sulfate. Removed solvent under vacuum, chromatographed (80% to 100% ethyl acetate/hexanes) to give an oil, 1-(2-Chloro-6-fluoro-phenyl)-2-[5-(2-methyl-5-pyridin-2-yl-2H-[1,2,4]triazol-3-yl)-2-nitro-phenyl]-ethanone (36 mg, 31%).

### Step 6 2-(2-Chloro-6-fluoro-phenyl)-5-(2-methyl-5-pyridin-2-yl-2H-[1,2,4]triazol-3-yl)-1H-indole

Stirred 1-(2-chloro-6-fluorophenyl)-2-(5-(1-methyl-3-(pyridin-2-yl)-1H-1,2,4-triazol-5-yl)-2-nitrophenyl)ethanone (35 mg, 77.5 μmmol, Eq: 1.00) in Acetic Acid (2 ml), added Iron (34.6 mg, 620 μmmol, Eq: 8), stirred at room temperature, for 16 hours, then heated to 80 C for 8 hours, added iron=35 mg, heated at 80 C for 4 hours, cooled to room temperature, Filtered through a paper filter, diluted methylene chloride, washed water, bicarb (2×), brine, dried magnesium sulfate. Removed solvent under vacuum, chromatographed (50% to 80% ethyl acetate in hexanes) to give a solid, 35 mg. This material was purified on prep-TLC, on two plates, eluting with 5% Methanol in methylene chloride and 0.1% ammonium hydroxide. Collected second band from top, stirred in 5% methanol/methylene chloride for 3 hours, filtered, Removed solvent under vacuum to give 2-(2-Chloro-6-fluoro-phenyl)-5-(2-methyl-5-pyridin-2-yl-2H-[1,2,4]triazol-3-yl)-1H-indole (10 mg, 32%): MS (M+H)=405.

### Example 185

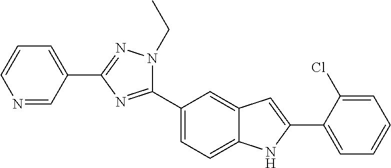

### 2-(2-Chloro-phenyl)-5-(2-ethyl-5-pyridin-3-yl-2H-[1,2,4]-triazol-3-yl)-1H-indole

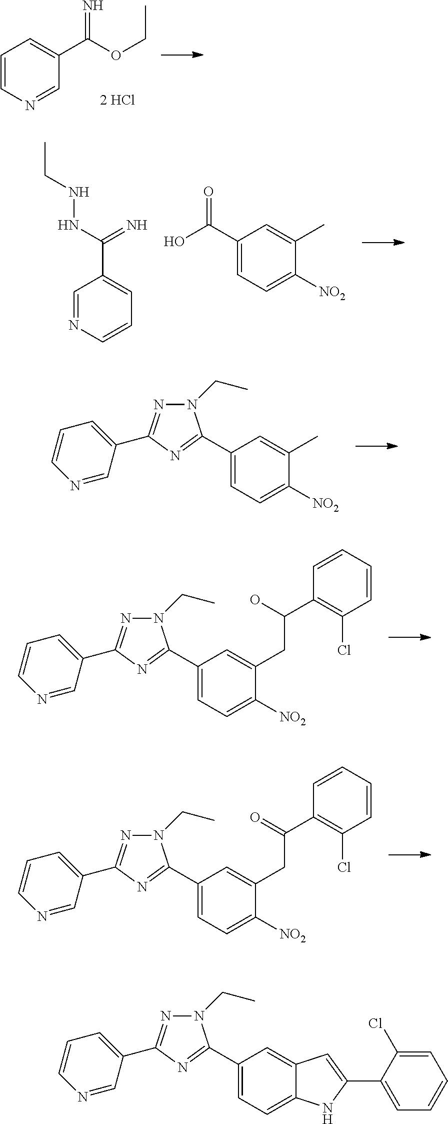

### Step 1 N′-ethylnicotinimidohydrazide

Added ethyl nicotinimidate dihydrochloride (Prepared as reported in J. Am. Chem. Soc. 1986, 108, 1989-1996, 4 g, 17.9 mmol, Eq: 1.00) to Pyridine (20 ml), stirred 5 minutes, then added ethylhydrazine oxalate (2.96 g, 19.7 mmol, Eq: 1.1). Stirred at room temperature overnight. Added ether, filtered through a sintered glass funnel, washed precipitate with ether 3×, pumped down to give N′-ethylnicotinimidohydrazide as a yellow solid (2.9 g, 100%). Took on without further purification.

### Step 2 3-(1-ethyl-5-(3-methyl-4-nitrophenyl)-1H-1,2,4-triazol-3-yl)pyridine

Stirred 3-methyl-4-nitrobenzoic acid (3.52 g, 19.4 mmol, Eq: 1.1) in THF (50 ml), added Carbonyl Diimidazole (3.15 g, 19.4 mmol, Eq: 1.1), heated to 50 C for 20 min. Cooled slightly, and added N′-ethylnicotinimidohydrazide (2.9 g, 17.7 mmol, Eq: 1.00), then pyridine (2.79 g, 2.86 ml, 35.3 mmol, Eq: 2), heated to 80 C for 45 min. Cooled, stirred at room temperature overnight. Continued heated to 90 C for 9 hours. Cooled, stirred at room temperature overnight. Diluted ethyl acetate, washed saturated aqueous sodium bicarbonate (3×), brine, dried magnesium sulfate. Removed solvent under vacuum, chromatographed (30% to 100% ea/hex over 20 minutes, then 5 minutes of elution at 100% ea). Collected last eluting spot to give 2.3 g, solid. Chromatographed this material under the same conditions to give 2.1 g 3-(1-ethyl-5-(3-methyl-4-nitrophenyl)-1H-1,2,4-triazol-3-yl)pyridine, app. 66% pure, used as is.

### Step 3 1-(2-chlorophenyl)-2-(5-(1-ethyl-3-(pyridin-3-yl)-1H-1,2,4-triazol-5-yl)-2-nitrophenyl)ethanol

Stirred 3-(1-ethyl-5-(3-methyl-4-nitrophenyl)-1H-1,2,4-triazol-3-yl)pyridine (530 mg, 1.71 mmol, Eq: 1.00) and 2-chlorobenzaldehyde (241 mg, 1.71 mmol, Eq: 1.00) in DMSO, added DBU (287 mg, 284 μl, 1.88 mmol, Eq: 1.1) dropwise. Stirred at room temperature overnight. Diluted ethyl acetate, washed water 3×, brine, dried over magnesium sulfate, chromatographed (20% to 100% ea/hex) to give 1-(2-chlorophenyl)-2-(5-(1-ethyl-3-(pyridin-3-yl)-1H-1,2,4-triazol-5-yl)-2-nitrophenyl)ethanol as an impure oil, 299 mg (about 80% pure), took on as is.

### Step 4 1-(2-Chloro-phenyl)-2-[5-(2-ethyl-5-pyridin-3-yl-2H-[1,2,4]triazol-3-yl)-2-nitro-phenyl]-ethanone

Stirred 1-(2-chlorophenyl)-2-(5-(1-ethyl-3-(pyridin-3-yl)-1H-1,2,4-triazol-5-yl)-2-nitrophenyl)ethanol (300 mg, 667 μmmol, Eq: 1.00) in methylene chloride (5 ml) at room temperature, added Dess-Martin periodinane (283 mg, 667 μmmol, Eq: 1.00) all at once. Stirred at room temperature overnight. Added Dess-Martin periodinane (283 mg, 667 μmmol, Eq: 1.00), stirred at room temperature 4 hours. Diluted methylene chloride, washed water 2×, sat. sodium bicarbonate solution (aqueous) 2×, brine, dried magnesium sulfate. Back extracted aqueous 2× methylene chloride, combined organic layers, dried, Removed solvent under vacuum, chromatographed (50% to 100% ea/hex) to give 1-(2-Chloro-phenyl)-2-[5-(2-ethyl-5-pyridin-3-yl-2H-[1,2,4]triazol-3-yl)-2-nitro-phenyl]-ethanone as an oil (105 mg, 35%).

### Step 5 2-(2-Chloro-phenyl)-5-(2-ethyl-5-pyridin-3-yl-2H-[1,2,4]triazol-3-yl)-1H-indole

Stirred 1-(2-chlorophenyl)-2-(5-(1-ethyl-3-(pyridin-3-yl)-1H-1,2,4-triazol-5-yl)-2-nitrophenyl)ethanone (101 mg, 226 μmmol) at room temperature for 4 hours. Diluted dichloromethane, washed water 2×, saturated aqueous sodium bicarbonate solution 2×, brine, added sodium bicarbonate to aqueous layers until pH ca 9, extracted aqueous 2× dichloromethane, combined organic layers, dried magnesium sulfate. Removed solvent under vacuum, chromatographed (45% to 100% ea/hex), recovered 67 mg oil. Chromatographed (0% to 5% methanol in dichloromethane over 20 minutes), two peaks elute with the major peak having the longer retention time. Collected this peak, placed in drying pistol under vacuum overnight to give 2-(2-Chloro-phenyl)-5-(2-ethyl-5-pyridin-3-yl-2H-[1,2,4]triazol-3-yl)-1H-indole (14 mg, 14%) MS (M+H)=401.

### Example 186

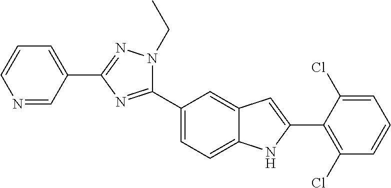

2-(2,6-Dichloro-phenyl)-5-(2-ethyl-5-pyridin-3-yl-2H-[1,2,4]triazol-3-yl)-1H-indole

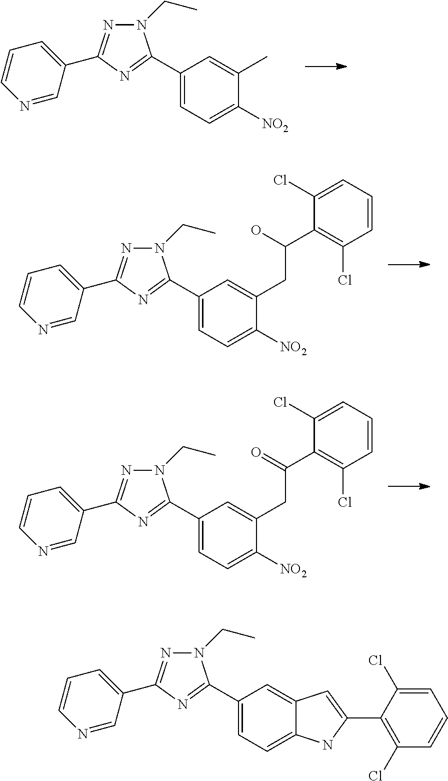

### Step 1 1-(2,6-dichlorophenyl)-2-(5-(1-ethyl-3-(pyridin-3-yl)-1H-1,2,4-triazol-5-yl)-2-nitrophenyl)ethanol

Stirred 3-(1-ethyl-5-(3-methyl-4-nitrophenyl)-1H-1,2,4-triazol-3-yl)pyridine (550 mg, 1.78 mmol, Eq: 1.00) in DMSO (10 ml), added 2,6-dichlorobenzaldehyde (467 mg, 2.67 mmol, Eq: 1.5) and then DBU (271 mg, 268 μl, 1.78 mmol, Eq: 1.00), stirred at room temperature overnight. Diluted water, extracted ethyl acetate 3×, washed water 2×, brine, dried over magnesium sulfate. Removed solvent under vacuum, chromatographed (0 to 5% methanol in dichloromethane over 20 min on analogix 40 g column) to give 1-(2,6-dichlorophenyl)-2-(5-(1-ethyl-3-(pyridin-3-yl)-1H-1,2,4-triazol-5-yl)-2-nitrophenyl)ethanol (144 mg, 17%) as a solid.

### Step 2 1-(2,6-dichlorophenyl)-2-(5-(1-ethyl-3-(pyridin-3-yl)-1H-1,2,4-triazol-5-yl)-2-nitrophenyl)ethanone

Stirred 1-(2,6-dichlorophenyl)-2-(5-(1-ethyl-3-(pyridin-3-yl)-1H-1,2,4-triazol-5-yl)-2-nitrophenyl)ethanol (144 mg, 297 μmmol, Eq: 1.00) in dichloromethane (10 ml), added Dess-Martin Periodinane (139 mg, 327 μmmol, Eq: 1.1), stirred 4 hours. Diluted dichloromethane, washed water, bicarb (2×), brine, dried magnesium sulfate. Removed solvent under vacuum to give crude 1-(2,6-dichlorophenyl)-2-(5-(1-ethyl-3-(pyridin-3-yl)-1H-1,2,4-triazol-5-yl)-2-nitrophenyl)ethanone (135 mg, 94%), used without purification in the next reaction.

### Step 3 2-(2,6-Dichloro-phenyl)-5-(2-ethyl-5-pyridin-3-yl-2H-[1,2,4]triazol-3-yl)-1H-indole

Added acetic acid to 1-(2,6-dichlorophenyl)-2-(5-(1-ethyl-3-(pyridin-3-yl)-1H-1,2,4-triazol-5-yl)-2-nitrophenyl)ethanone (135 mg, 280 μmmol, Eq: 1.00), then Iron filings (125 mg, 2.24 mmol, Eq: 8), stirred at room temperature 6 hours, washed water, saturated aqueous sodium bicarbonate (2×), added solid sodium bicarbonate to aqueous layers until pH ca 9, back extracted aqueous layers with methylene chloride 1×, combined dichloromethane layers, washed brine, dried over magnesium sulfate. Chromatographed (0 to 6% Methanol/dichloromethane) on 12 g analogix column over 20 min. to give 2-(2,6-Dichloro-phenyl)-5-(2-ethyl-5-pyridin-3-yl-2H-[1,2,4]triazol-3-yl)-1H-indole (13 mg, 11%), MS (M+H)=435.

### Example 187

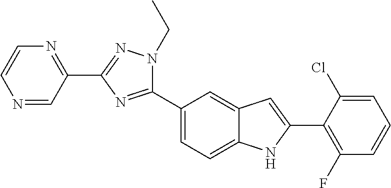

### 2-(2-Chloro-6-fluoro-phenyl)-5-(2-ethyl-5-pyrazin-2-yl-2H-[1,2,4]triazol-3-yl)-1H-indole

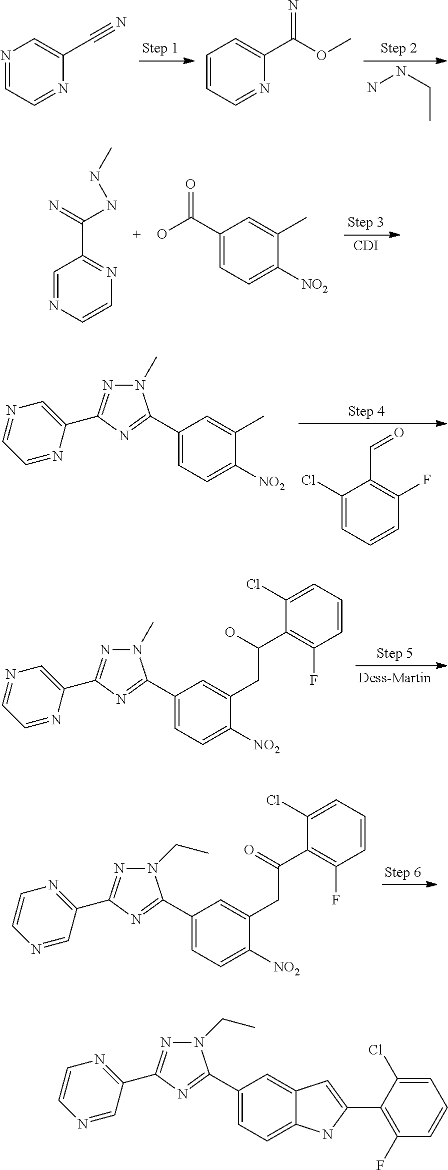

### Step 1 Methyl pyrazine-2-carbimidate

Stirred pyrazine-2-carbonitrile (5 g, 47.6 mmol, Eq: 1.00) in methanol (50 ml) at room temperature, added Sodium Methoxide as a 4.6 M solution in methanol (Aldrich) (15.5 ml, 71.4 mmol, Eq: 1.5) slowly. Stirred at room temp; a ppt forms after 5 minutes. Stirred 2 hours, evaporated most Methanol under vacuum, filtered, washed white solid with methanol 3×, placed in flask and pumped down to give methylpyrazine-2-carbimidate (5.1 g, 78%)

### Step 2 N′-ethylpyrazine-2-carboximidhydrazide oxalate

Stirred methylpyrazine-2-carbimidate (5.1 g, 37.2 mmol, Eq: 1.00) in Pyridine (75 ml) at room temperature, added ethylhydrazine oxalate (6.7 g, 44.6 mmol, Eq: 1.2), stirred at room temp. overnight. Diluted ether, filtered solid that forms, washed solid with ether 3×, placed in flask under vacuum to give N′-ethylpyrazine-2-carboximidhydrazide oxalate (8.4 g, 88%) as a solid.

### Step 3 2-(1-ethyl-5-(3-methyl-4-nitrophenyl)-1H-1,2,4-triazol-3-yl)pyrazine

Stirred 3-methyl-4-nitrobenzoic acid (4.47 g, 24.7 mmol, Eq: 1.5) in THF (50 ml) at room temp., added CDI (4.00 g, 24.7 mmol, Eq: 1.5), heated to 60 C for 1 hour. Cooled to room temp., added pyridine (2.6 g, 2.66 ml, 32.9 mmol, Eq: 2), then N′-ethylpyrazine-2-carboximidhydrazide oxalate (4.2 g, 16.5 mmol) all at once. Heated at 60 C overnight, then raised temperature to 85 C for 5 hours. Cooled to room temp., diluted ethyl acetate, washed water, saturated aqueous sodium bicarbonate solution (2×), brine, dried magnesium sulfate. Removed solvent under vacuum, chromatographed (0 to 6% Methanol in dichloromethane on a 150 g Analogix column, then chromatographed major product 50% to 100% ethyl acetate in hexanes), to give 2-(1-ethyl-5-(3-methyl-4-nitrophenyl)-1H-1,2,4-triazol-3-yl)pyrazine as an oil that slowly crystallizes (1.2 g, 23%).

### Step 4 1-(2-chloro-6-fluorophenyl)-2-(5-(1-ethyl-3-(pyrazin-2-yl)-1H-1,2,4-triazol-5-yl)-2-nitrophenyl)ethanol

Stirred 2-(1-ethyl-5-(3-methyl-4-nitrophenyl)-1H-1,2,4-triazol-3-yl)pyrazine (0.600 g, 1.93 mmol, Eq: 1.00) in DMSO (5 ml), added 2-chloro-6-fluorobenzaldehyde (460 mg, 2.9 mmol, Eq: 1.5), then DBU (324 mg, 321 μl, 2.13 mmol, Eq: 1.1) via syringe. Stirred at room temperature overnight. Diluted water, extracted ethyl acetate 2×, washed water 2×, brine, dried magnesium sulfate. Removed solvent under vacuum, chromatographed (65% to 100% ea in hex over 20 minutes, 40 g analogix column) to give 1-(2-chloro-6-fluorophenyl)-2-(5-(1-ethyl-3-(pyrazin-2-yl)-1H-1,2,4-triazol-5-yl)-2-nitrophenyl)ethanol (401 mg, 44%).

### Step 5 1-(2-chloro-6-fluorophenyl)-2-(5-(1-ethyl-3-(pyrazin-2-yl)-1H-1,2,4-triazol-5-yl)-2-nitrophenyl)ethanone

Stirred 1-(2-chloro-6-fluorophenyl)-2-(5-(1-ethyl-3-(pyrazin-2-yl)-1H-1,2,4-triazol-5-yl)-2-nitrophenyl)ethanol (401 mg, 855 μmmol, Eq: 1.00) in dichloromethane (5 ml), added Dess-Martin periodinane (399 mg, 94 μmol, Eq: 1.1) all at once at room temperature. Stirred 1.5 hours, diluted dichloromethane, washed saturated aqueous sodium bicarbonate solution 2×, brine, dried magnesium sulfate. Removed solvent under vacuum to give 1-(2-chloro-6-fluorophenyl)-2-(5-(1-ethyl-3-(pyrazin-2-yl)-1H-1,2,4-triazol-5-yl)-2-nitrophenyl)ethanone (385 mg, 96%). Took on without further purification.

### Step 6 2-(2-Chloro-6-fluoro-phenyl)-5-(2-ethyl-5-pyrazin-2-yl-2H-[1,2,4]triazol-3-yl)-1H-indole

Stirred 1-(2-chloro-6-fluorophenyl)-2-(5-(1-ethyl-3-(pyrazin-2-yl)-1H-1,2,4-triazol-5-yl)-2-nitrophenyl)ethanone (385 mg, 825 μmmol, Eq: 1.00) in Acetic Acid (10 ml), added Iron filings (368 mg, 6.6 mmol, Eq: 8). Stirred at room temperature overnight. Filtered through a paper filter, washed filter paper with dichloromethane, washed organic layers with water (2×), saturated aqueous sodium bicarbonate solution (2×), brine, dried magnesium sulfate. Removed solvent under vacuum, chromatographed (0 to 5% Methanol in dichloromethane, then rechromatographed major product collected with 60 to 100% Ethyl acetate/Hexanes), to give 2-(2-Chloro-6-fluoro-phenyl)-5-(2-ethyl-5-pyrazin-2-yl-2H-[1,2,4]triazol-3-yl)-1H-indole as a solid (129 mg, 37%), MS (M+H)=420.

### Example 188

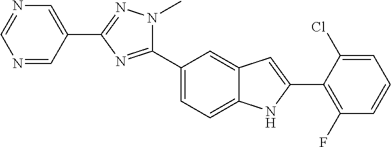

### 2-(2-Chloro-6-fluoro-phenyl)-5-(2-methyl-5-pyrimidin-5-yl-2H-[1,2,4]triazol-3-yl)-1H-indole

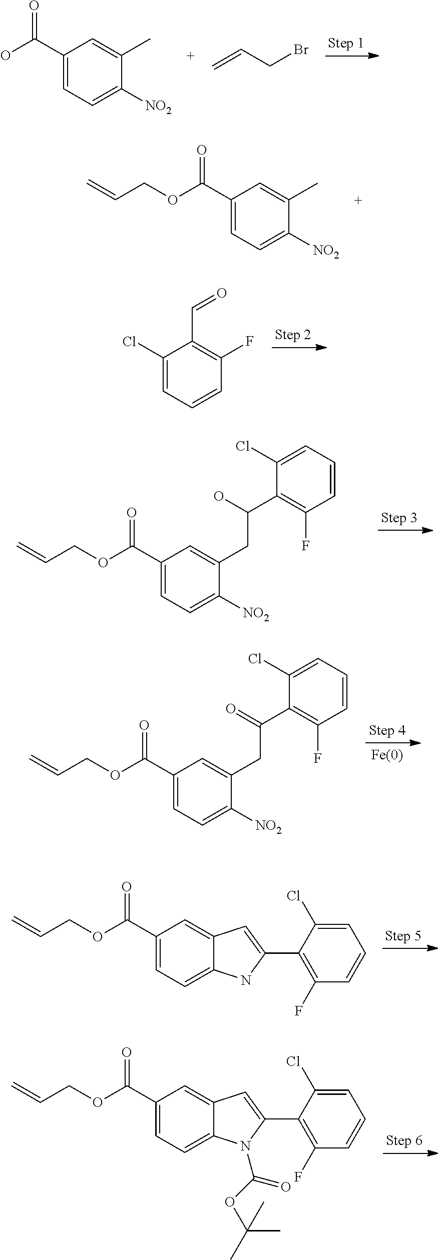

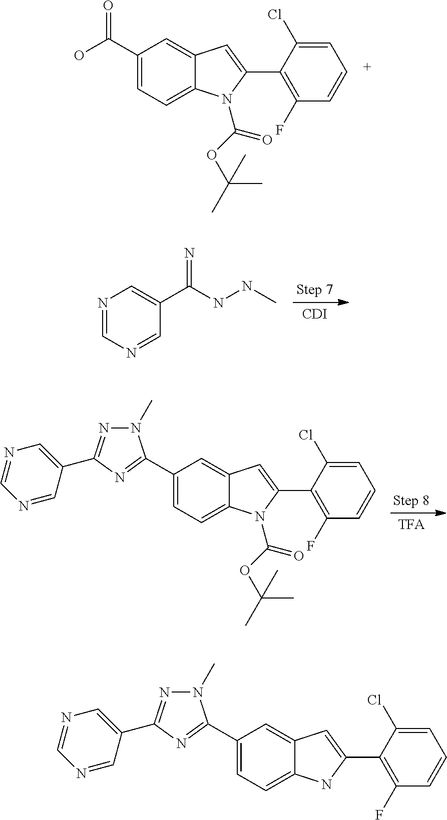

### N′-methylpyrimidine-5-carboximidhydrazide

Stirred pyrimidine-5-carbonitrile (2 g, 19.0 mmol, Eq: 1.00) in Methanol (16.0 ml) at room temperature, added sodium methoxide as a 4.6 M solution in methanol (Aldrich) (8.27 ml, 38.1 mmol, Eq: 2) slowly, stirred at room temperature overnight. Removed most solvent under vacuum, diluted ethyl acetate, washed water, brine, dried magnesium sulfate. Rotovaped to give methylpyrimidine-5-carbimidate as oil (1.6 g, 61%). This material was used without purification in the next reaction.

Stirred methylpyrimidine-5-carbimidate (400 mg, 2.92 mmol, Eq: 1.00) at room temperature in Pyridine (5 ml), added methylhydrazine (148 mg, 3.21 mmol, Eq: 1.1), stirred 4 hours. Rotovaped, pumped down to give N′-methylpyrimidine-5-carboximidhydrazide as an orange solid (452 mg, >100%) of about 90% purity, which was used as is in Step 7 of the following preparation.

### Step 1 Allyl 3-methyl-4-nitrobenzoate

Stirred 3-methyl-4-nitrobenzoic acid (5 g, 27.6 mmol, Eq: 1.00) in DMF (50 ml), added 3-bromoprop-1-ene (3.67 g, 30.4 mmol, Eq: 1.1) and Potassium Carbonate (4.58 g, 33.1 mmol, Eq: 1.2), stirred at room temperature overnight. Diluted ether, washed water 1×, saturated aqueous sodium bicarbonate solution. 2×, brine, dried over magnesium sulfate. Rotovaped to give allyl 3-methyl-4-nitrobenzoate (6.0 g, 98%) as an oil.

### Step 2 alkyl 3-(2-(2-chloro-6-fluorophenyl)-2-hydroxyethyl)-4-nitrobenzoate

Stirred allyl 3-methyl-4-nitrobenzoate (2 g, 9.04 mmol, Eq: 1.00) in DMSO (20 ml), added 2-chloro-6-fluorobenzaldehyde (2.15 g, 13.6 mmol, Eq: 1.5), then DBU (1.51 g, 1.5 ml, 9.95 mmol, Eq: 1.1) via syringe. Stirred at room temperature overnight. Diluted water, about 250 ml, extracted ethyl ether/ethyl acetate (1:1) 2×, combined organic layers, washed water 2×, brine, dried magnesium sulfate. Rotovaped to give an oil. Chromatographed (2% to 15% ea/hex, 120 g Analogix column over 22 minutes) to give allyl 3-(2-(2-chloro-6-fluorophenyl)-2-hydroxyethyl)-4-nitrobenzoate (1.1 g, 32%).

### Step 3 Allyl 3-(2-(2-chloro-6-fluorophenyl)-2-oxoethyl)-4-nitrobenzoate

Stirred allyl 3-(2-(2-chloro-6-fluorophenyl)-2-hydroxyethyl)-4-nitrobenzoate (1.1 g, 2.9 mmol, Eq: 1.00), in methylene chloride (15 ml), added Dess-Martin Periodinane (1.35 g, 3.19 mmol, Eq: 1.1), stirred at room temperature overnight. Diluted methylene chloride, washed water, bicarb (3×), brine, dried MgSO4. Rotovaped, chromatographed (5% to 50% ethyl acetate in hexanes) to give allyl 3-(2-(2-chloro-6-fluorophenyl)-2-oxoethyl)-4-nitrobenzoate (855 mg, 78%) as an oil.

### Step 4 Allyl 2-(2-chloro-6-fluorophenyl)-1H-indole-5-carboxylate

Stirred allyl 3-(2-(2-chloro-6-fluorophenyl)-2-oxoethyl)-4-nitrobenzoate (855 mg, 2.26 mmol, Eq: 1.00) in Acetic Acid (10 ml), added Iron (758 mg, 13.6 mmol, Eq: 6), stirred at room temperature overnight. Filtered through a paper filter, washed with methylene chloride 3× times, washed methylene chloride with water, bicarb (2×), brine, dried magnesium sulfate. Rotovaped to give allyl 2-(2-chloro-6-fluorophenyl)-1H-indole-5-carboxylate as an oil which slowly solidifies (550 mg, 74%).

### Step 5 5-allyl 1-tert-butyl 2-(2-chloro-6-fluorophenyl)-1H-indole-1,5-dicarboxylate

Stirred allyl 2-(2-chloro-6-fluorophenyl)-1H-indole-5-carboxylate (550 mg, 1.67 mmol, Eq: 1.00) in dichloromethane, added di-tert-butyl dicarbonate (400 mg, 426 μl, 1.83 mmol, Eq: 1.1), then DMAP (20 mg, 167 μmmol, Eq: 0.1), stirred 3 hours. Diluted methylene chloride, washed water 2×, brine, dried magnesium sulfate. Chromatographed (3% to 15% ethyl acetate in hexanes) to give 5-allyl 1-tert-butyl 2-(2-chloro-6-fluorophenyl)-1H-indole-1,5-dicarboxylate as an oil (385 mg, 54%).

### Step 6 1-(tert-butoxycarbonyl)-2-(2-chloro-6-fluorophenyl)-1H-indole-5-carboxylic acid

Stirred 5-allyl 1-tert-butyl 2-(2-chloro-6-fluorophenyl)-1H-indole-1,5-dicarboxylate (357 mg, 830 μmmol, Eq: 1.00) in THF (5 ml), added tetrakis(triphenylphosphine)palladium(0) (96.0 mg, 83.0 μmmol, Eq: 0.1), then Morpholine (362 mg, 362 μl, 4.15 mmol, Eq: 5), stirred at room temperature 30 min. Diluted water, added 500 ul Acetic acid (glacial), extracted ethyl acetate 3× (emulsion forms. Added ca. 100 ul AcOH), washed organic layers with brine, dried MgSO4. Rotovaped to give a foam, 1-(tert-butoxycarbonyl)-2-(2-chloro-6-fluorophenyl)-1H-indole-5-carboxylic acid (425 mg., >100%). Took on as is.

### Step 7 tert-Butyl 2-(2-chloro-6-fluorophenyl)-5-(1-methyl-3-(pyrimidin-5-yl)-1H-1,2,4-triazol-5-yl)-1H-indole-1-carboxylate

Stirred 1-(tert-butoxycarbonyl)-2-(2-chloro-6-fluorophenyl)-1H-indole-5-carboxylic acid (100 mg, 257 μmmol, Eq: 1.00) in THF (3 ml), added carbonyl diimidazole (45.8 mg, 282 μmmol, Eq: 1.1). Stirred 1.5 hours at room temperature. Added N′-methylpyrimidine-5-carboximidhydrazide (38.8 mg, 257 μmmol, Eq: 1.00, prepared as described below), heated to 50 C for 1 hour, added 80 mg of N′-methylpyrimidine-5-carboximidhydrazide. Heated to 60 C for 2 hours, then cooled to 45 C and heated for 72 hours. Reaction goes dry. Dissolved residue in ethyl acetate, washed water, brine, dried magnesium sulfate. Rotovaped, chromatographed (5% to 50% ethyl acetate in hexanes) to give tert-butyl 2-(2-chloro-6-fluorophenyl)-5-(1-methyl-3-(pyrimidin-5-yl)-1H-1,2,4-triazol-5-yl)-1H-indole-1-carboxylate (35 mg, 27%).

### Step 8 2-(2-Chloro-6-fluoro-phenyl)-5-(2-methyl-5-pyrimidin-5-yl-2H-[1,2,4]triazol-3-yl)-1H-indole

Stirred tert-butyl 2-(2-chloro-6-fluorophenyl)-5-(1-methyl-3-(pyrimidin-5-yl)-1H-1,2,4-triazol-5-yl)-1H-indole-1-carboxylate (35 mg, 69.3 μmmol, Eq: 1.00) in dichloromethane, added TFA (474 mg, 320 μl, 4.16 mmol, Eq: 60) and stirred at room temperature overnight. Added TFA=100 ul. Stirred 5 hours, added 5 drops aqueous ammonium hydroxide solution (until ppt stops forming), filtered on micro paper filter, collected solid, washed solids into separatory funnel with methylene chloride and aqueous saturated sodium bicarbonate solution, separated layers, extracted aqueous saturated sodium bicarbonate solution 1× methylene chloride, combined organic layers, washed aqueous saturated sodium bicarbonate solution, water, brine, dried magnesium sulfate, rotovaped to give 2-(2-Chloro-6-fluoro-phenyl)-5-(2-methyl-5-pyrimidin-5-yl-2H-[1,2,4]triazol-3-yl)-1H-indole (3 mg, 11%), MS (M+H)=406.

### Example 189

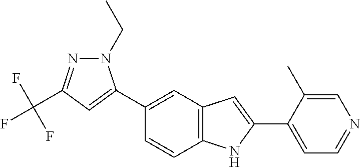

### 5-(1-ethyl-3-(trifluoromethyl)-1H-pyrazol-5-yl)-2-(3-methylpyridin-4-yl)-1H-indole

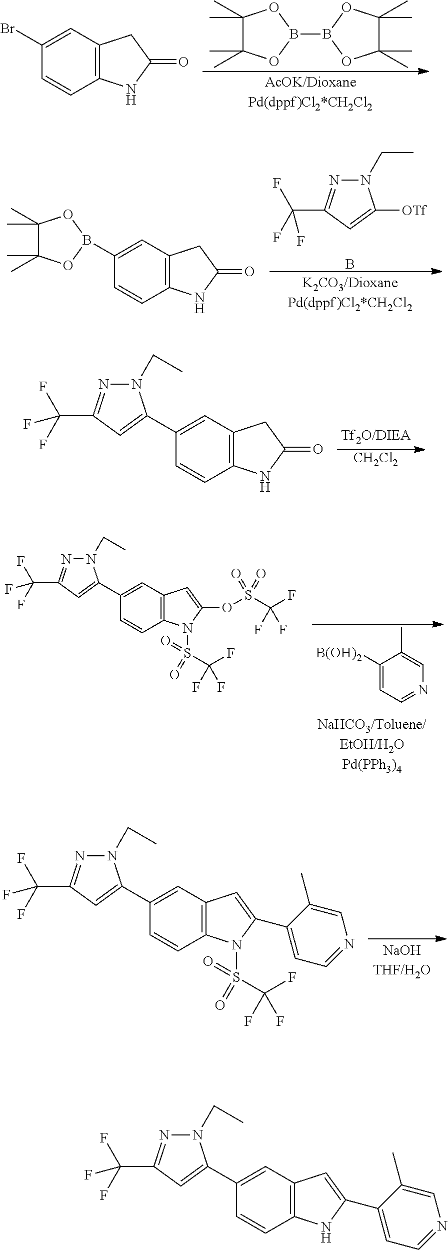

### Step 1 5-(4,4,5,5-tetramethyl-1,3,2-dioxaborolan-2-yl)indolin-2-one

To a solution of 5-bromoindolin-2-one (5.00 g, 23.6 mmol) in dry dioxane (60 ml) was added bis(pinacolato)diboron (7.78 g, 30.7 mmol) and KOAc (4.63 g, 47.2 mmol), while the mixture was degassed with the nitrogen, Pd(dppf)Cl₂*CH₂Cl₂ (0.96 g, 1.18 mmol) was added, the mixture was heated to 80° C. overnight, then the cooled reaction mixture was partitioned between EtOAc and water, the organic phase was washed with brine, dried over Na₂SO₄, filtered and concentrated under reduced pressure, the residual solid was washed withMeOH, EtOAc and hexanes to give 5-(4,4,5,5-tetramethyl-1,3,2-dioxaborolan-2-yl)indolin-2-one as a light brown solid (2.89 g). A second crop of product was obtained by combining the washing solutions and purified by filtration through a pad of silica gel (using 2:8, 4:6, 6:4, and 8:2 mixtures of EtOAc/Hexanes solutions) to give more 5-(4,4,5,5-tetramethyl-1,3,2-dioxaborolan-2-yl)indolin-2-one as an orange solid (2.63 g, for a total yield=5.53 g, 90%).

### Step 2 5-(1-ethyl-3-(trifluoromethyl)-1H-pyrazol-5-yl)indolin-2-one

To a solution of 5-(4,4,5,5-tetramethyl-1,3,2-dioxaborolan-2-yl)indolin-2-one (1.2 g, 4.63 mmol) and 1-ethyl-3-(trifluoromethyl)-1H-pyrazol-5-yl trifluoromethanesulfonate (Intermediate 12, 2.17 g, 6.95 mmol) in 1,4-dioxane (35 ml) was added 7 mL of an aqueous K₂CO₃ solution (1.92, 13.9 mmol), while the mixture was degassed by nitrogen, [1,1′-Bis(diphenylphosphino)ferrocene]dichloropalladium(II) complex with dichloromethane (0.378 g, 0.046 mmol) was added, the mixture was heated to 80° C. overnight, the cooled reaction mixture was partitioned between EtOAc and water, the organic phase was washed with brine, dried over Na₂SO₄, filtered and concentrated under reduced pressure, the crude material was purified by flash chromatography (5-25% EtOAc/hexanes) to give 5-(1-ethyl-3-(trifluoromethyl)-1H-pyrazol-5-yl)indolin-2-one as a pale yellow solid (0.55 g, 40%).

### Step 3 5-(1-ethyl-3-(trifluoromethyl)-1H-pyrazol-5-yl)-1-(trifluoromethylsulfonyl)-1H-indol-2-yl trifluoromethanesulfonate

To a 0° C. solution of 5-(1-ethyl-3-(trifluoromethyl)-1H-pyrazol-5-yl)indolin-2-one (0.97 g, 3.29 mmol) and DIPEA (1.27 g, 9.86 mmol) in CH₂Cl₂ (50 ml) was added (CF₃SO₂)₂O (2.32 g, 8.21 mmol) dropwise, stirred in an ice bath for 40 minutes, then a saturated aqueous NH₄Cl solution was added, partitioned between CH₂Cl₂ and water, the organic phase was washed with brine, dried over Na₂SO₄, filtered and concentrated under reduced pressure, the crude material was purified by filtering through a pad of silica gel (5-8% EtOAc/Hexane) to give a light yellow oil which solidified under high vacuum to give 5-(1-ethyl-3-(trifluoromethyl)-1H-pyrazol-5-yl)-1-(trifluoromethylsulfonyl)-1H-indol-2-yltrifluoromethanesulfonate as an off-white solid (1.47 g, 80%).

### 5-(1-ethyl-3-(trifluoromethyl)-1H-pyrazol-5-yl)-2-(3-methylpyridin-4-yl)-1-(trifluoromethylsulfonyl)-1H-indole

To a flask was added 5-(1-ethyl-3-(trifluoromethyl)-1H-pyrazol-5-yl)-1-(trifluoromethylsulfonyl)-1H-indol-2-yl (96 mgs, 0.17 mmole), 3-methylpyridine-4-boronic acid (25 mg, 0.18 mmole), toluene (2.5 ml), EtOH (1.5 ml), a solution of NaHCO₃ (43 mgs, 0.52 mmol) in water (1 mL), while the mixture was degassed with N₂, Pd(Ph₃P)₄ (10 mgs, 0.009 mmole) was added. The reaction mixture was heated to 80° C. overnight, then the cooled reaction mixture was partitioned between EtOAc and water, the organic layer was washed with brine, dried over Na₂SO₄, filtered and concentrated under reduced pressure to give 5-(1-ethyl-3-[trifluoromethyl]-1H-pyrazol-5-yl)-2-(3-methylpyridin-4-yl)-1-(trifluoromethylsulfonyl)-1H-indole, which was used directly in the next step without purification.

### 5-(1-ethyl-3-(trifluoromethyl)-1H-pyrazol-5-yl)-2-(3-methylpyridin-4-yl)-1H-indole

A mixture of 5-(1-ethyl-3-(trifluoromethyl)-1H-pyrazol-5-yl)-2-(3-methylpyridin-4-yl)-1-(trifluoromethylsulfonyl)-1H-indole in THF (3 ml) and a 3N NaOH aqueous solution (3 ml) was stirred at room temperature for one day, partitioned between EtOAc and water, the organic layer was washed with brine, dried over Na₂SO₄, filtered and concentrated under reduced pressure, the crude material was purified twice by preparative TLC using (8:2 EtOAc/hexanes), and further purified by preparative TLC using (5:95 MeOH/CH₂Cl₂ and 0.1% NH₄OH) to give 5-(1-ethyl-3-(trifluoromethyl)-1H-pyrazol-5-yl)-2-(3-methylpyridin-4-yl)-1H-indole (7 mg, 10% in 2 steps). MS (M+H)=371.

### Example 190

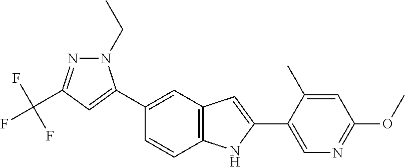

### 5-(1-ethyl-3-(trifluoromethyl)-1H-pyrazol-5-yl)-2-(6-methoxy-4-methylpyridin-3-yl)-1H-indole

Prepared in a manner identical to Example 189 to give 5-(1-ethyl-3-(trifluoromethyl)-1H-pyrazol-5-yl)-2-(6-methoxy-4-methylpyridin-3-yl)-1H-indole. MS (M+H)=401.

### Example 191

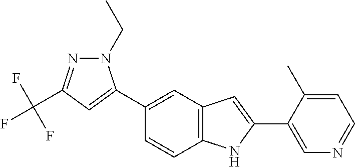

### 5-(1-ethyl-3-(trifluoromethyl)-1H-pyrazol-5-yl)-2-(4-methylpyridin-3-yl)-1H-indole

Prepared in a manner identical to Example 189 to give 5-(1-ethyl-3-(trifluoromethyl)-1H-pyrazol-5-yl)-2-(4-methylpyridin-3-yl)-1H-indole. MS (M+H)=371.

### Example 192

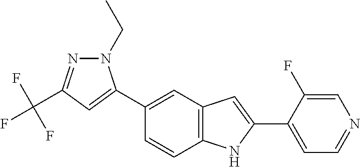

### 5-(1-ethyl-3-(trifluoromethyl)-1H-pyrazol-5-yl)-2-(3-fluoropyridin-4-yl)-1H-indole

Prepared in a manner identical to Example 189 to give 5-(1-ethyl-3-(trifluoromethyl)-1H-pyrazol-5-yl)-2-(3-fluoropyridin-4-yl)-1H-indole. MS (M+H)=375.

### Example 193

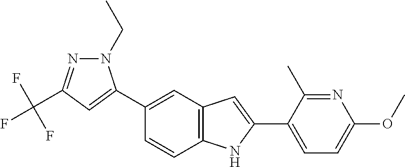

### 5-(1-ethyl-3-(trifluoromethyl)-1H-pyrazol-5-yl)-2-(6-methoxy-2-methylpyridin-3-yl)-1H-indole

Prepared in a manner identical to Example 189 to give 5-(1-ethyl-3-(trifluoromethyl)-1H-pyrazol-5-yl)-2-(6-methoxy-2-methylpyridin-3-yl)-1H-indole. MS (M+H)=401.

### Example 194

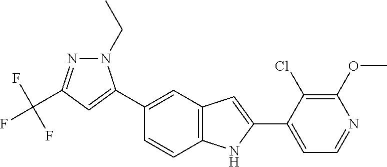

### 2-(3-chloro-2-methoxypyridin-4-yl)-5-(1-ethyl-3-(trifluoromethyl)-1H-pyrazol-5-yl)-1H-indole

Prepared in a manner identical to Example 189 to give 2-(3-chloro-2-methoxypyridin-4-yl)-5-(1-ethyl-3-(trifluoromethyl)-1H-pyrazol-5-yl)-1H-indole. MS (M+H)=421.

### Example 195

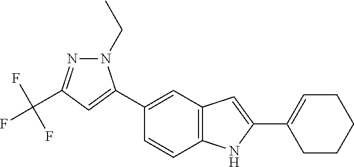

### cyclohexyl-5-(1-ethyl-3-(trifluoromethyl)-1H-pyrazol-5-yl)-1H-indole

and

### cyclohexenyl-5-(1-ethyl-3-(trifluoromethyl)-1H-pyrazol-5-1-1-trifluoromethylsulfonyl)-1H-indole

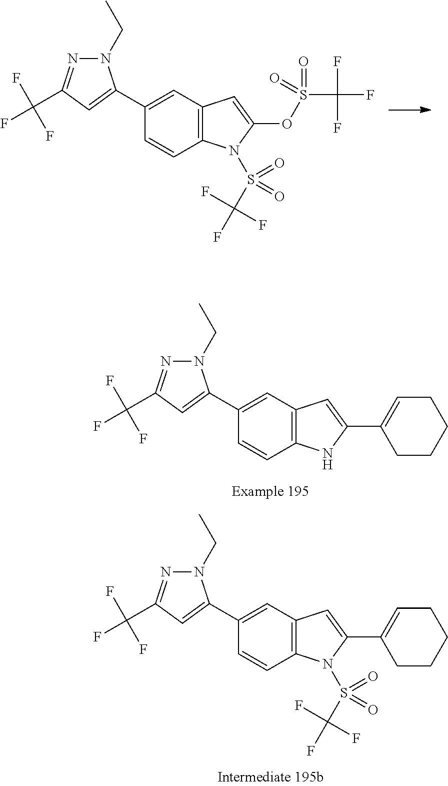

Prepared in a manner identical to Example 189 but replacing 3-methylpyridine-4-boronic acid with 1-cyclohexen-1-yl-boronic acid to give a mixture of products which were separated and purified by preparative TLC (20:80 EtOAc/Hexanes) to give (2-cyclohexenyl-5-[1-ethyl-3-{trifluoromethyl}-1H-pyrazol-5-yl]-1H-indole) as a pale-yellow solid, MS (M+H)=360, and Intermediate 195b (2-cyclohexenyl-5-(1-ethyl-3-(trifluoromethyl)-1H-pyrazol-5-yl)-1-(trifluoromethylsulfonyl)-1H-indole) as a colorless gum.

### Example 196

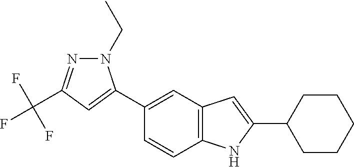

### Cyclohexyl-5-(1-ethyl-3-(trifluoromethyl)-1H-pyrazol-5-yl)-1H-indole and [2-(2-Cyclohexyl-ethyl)-4-(2-ethyl-5-trifluoromethyl-2H-pyrazol-3-yl)-phenyl]-methyl-amine

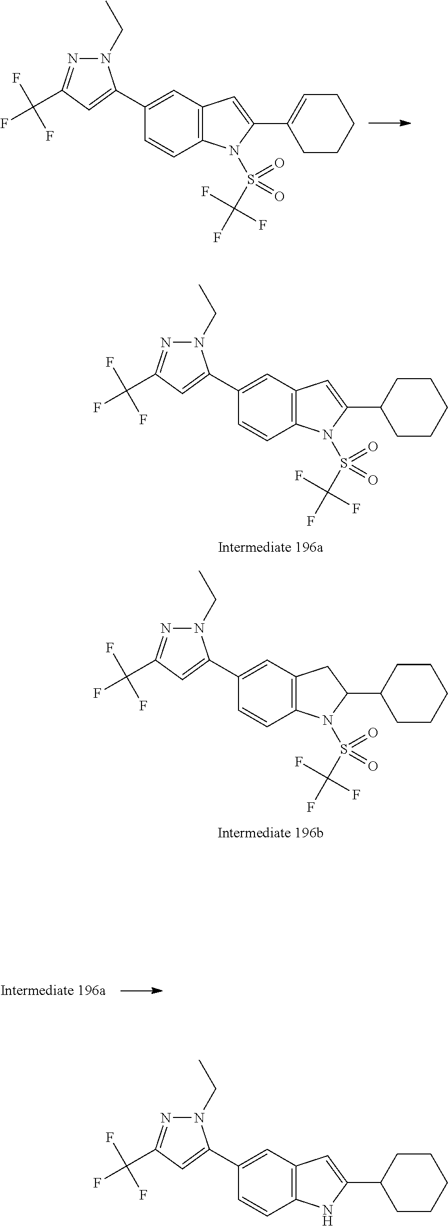

### Step 1 2-cyclohexyl-5-(1-ethyl-3-(trifluoromethyl)-1H-pyrazol-5-yl)-1-(trifluoromethylsulfonyl)-1H-indole

To a solution of 2-cyclohexenyl-5-(1-ethyl-3-(trifluoromethyl)-1H-pyrazol-5-yl)-1-(trifluoromethylsulfonyl)-1H-indole (80 mg, 0.16 mmol) in EtOAc (5 ml), was added 10% Pd/C (80 mg) under nitrogen, the reaction mixture was stirred at room temperature under an H₂ balloon for 10 days; the reaction mixture was filtered through celite, washed with EtOAc, the organic phase was washed with brine, dried over Na₂SO₄, filtered and concentrated under reduced pressure, the crude material was purified by flash chromatography (5-10% EtOAc/Hexanes) to give 2-cyclohexyl-5-(1-ethyl-3-(trifluoromethyl)-1H-pyrazol-5-yl)-1-(trifluoromethylsulfonyl)-1H-indole (Intermediate 196a), as a colorless gum (33 mg, 41%) and 2-cyclohexyl-5-(1-ethyl-3-(trifluoromethyl)-1H-pyrazol-5-yl)-1-(trifluoromethylsulfonyl)indoline (Intermediate 196b) as a colorless gum (30 mg, 37%).

### Step 2 2-cyclohexyl-5-(1-ethyl-3-(trifluoromethyl)-1H-pyrazol-5-yl)-1H-indole

Deprotected in a manner identical to Example 189 to give 2-cyclohexyl-5-(1-ethyl-3-(trifluoromethyl)-1H-pyrazol-5-yl)-1-(trifluoromethylsulfonyl)-1H-indole. MS (M+H)=362.

### Example 197

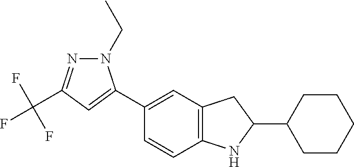

[2-(2-Cyclohexyl-ethyl)-4-(2-ethyl-5-trifluoromethyl-2H-pyrazol-3-yl)-phenyl]-methyl-amine

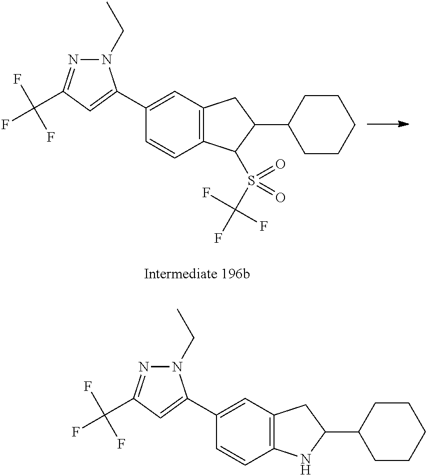

To a solution of 2-cyclohexyl-5-(1-ethyl-3-(trifluoromethyl)-1H-pyrazol-5-yl)-1-(trifluoromethylsulfonyl)indoline (Intermediate 196b, 30 mg, 0.61 mmol) in 5 mL of diethyl ether was added lithium aluminum hydride (14 g, 0.36 mmol) and refluxed for 4 hrs., then stirred at 45° C. overnight. The cooled reaction mixture was partitioned between water and EtOAc, the organic layer was washed with brine, dried over sodium sulfate, filtered and concentrated. The crude material was loaded onto silica gel and purified by flash chromatography (5:95-13:87 EtOAc/hexanes), then dried under high vacuum for 1 day to give [2-(2-cyclohexyl-ethyl)-4-(2-ethyl-5-trifluoromethyl-2H-pyrazol-3-yl)-phenyl]-methyl-amine as a light-yellow gum (16 mg, 65%). MS (M+H)=364.

### Example 198

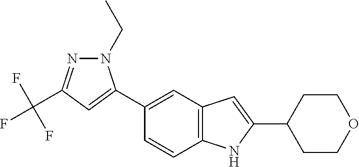

### 5-(1-ethyl-3-(trifluoromethyl)-1H-pyrazol-5-yl)-2-(tetrahydro-2H-pyran-4-yl)-1H-indole

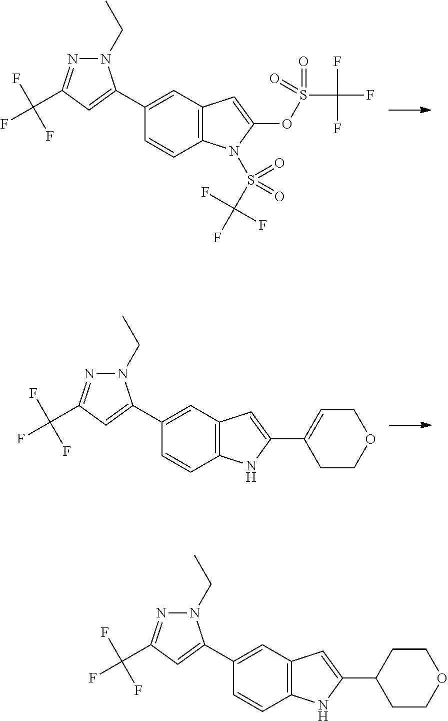

### Step 1 2-(3,6-dihydro-2H-pyran-4-yl)-5-(1-ethyl-3-(trifluoromethyl)-1H-pyrazol-5-yl)-1H-indole

Prepared in a manner identical to Example 189 replacing 3-methylpyridine-4-boronic acid with 3,6-Dihydro-2H-pyran-4-boronic acid pinacol ester to give 2-(3,6-dihydro-2H-pyran-4-yl)-5-(1-ethyl-3-(trifluoromethyl)-1H-pyrazol-5-yl)-1H-indole.

### Step 2 5-(1-ethyl-3-(trifluoromethyl)-1H-pyrazol-5-yl)-2-(tetrahydro-2H-pyran-4-yl)-1H-indole

To a mixture of 2-(3,6-dihydro-2H-pyran-4-yl)-5-(1-ethyl-3-(trifluoromethyl)-1H-pyrazol-5-yl)-1H-indole (38 mg, 105 μmmol) and ammonium formate (66.3 mg, 1.05 mmol) in MeOH (5 ml), 10% palladium on carbon (38 mg, 35.7 μmmol) was added under nitrogen. The reaction mixture was refluxed for 30 minutes, catalyst was filtered off, washed withMeOH, the combined filtrate was evaporated, the residue was partitioned between CH₂Cl₂ and brine, the organic phase was washed with brine, dried over Na₂SO₄, filtered and concentrated under reduced pressure, the crude material was purified by flash chromatography (15-50% EtOAc/Hexane) to give 5-(1-ethyl-3-(trifluoromethyl)-1H-pyrazol-5-yl)-2-(tetrahydro-2H-pyran-4-yl)-1H-indole, white solid (36 mg, 94%). MS (M+H)=364.

### Example 199

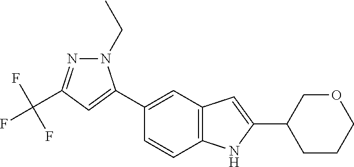

### 5-(1-ethyl-3-(trifluoromethyl)-1H-pyrazol-5-yl)-2-(tetrahydro-2H-pyran-3-yl)-1H-indole

Prepared in a manner identical to Example 198 replacing 3,6-Dihydro-2H-pyran-4-boronic acid pinacol with 2-(3,4-Dihydro-2H-pyran-5-yl)-4,4,5,5-tetramethyl-1,3,2-dioxaborolane to give 2-(3,4-dihydro-2H-pyran-5-yl)-5-(1-ethyl-3-(trifluoromethyl)-1H-pyrazol-5-yl)-1H-indole. MS (M+H)=364.

### Example 200

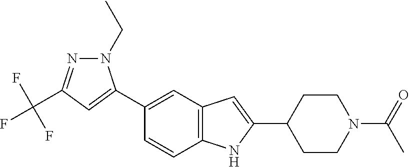

### 1-(4-(5-(1-ethyl-3-(trifluoromethyl)-1H-pyrazol-5-yl)-1H-indol-2-yl)piperidin-1-yl)ethanone

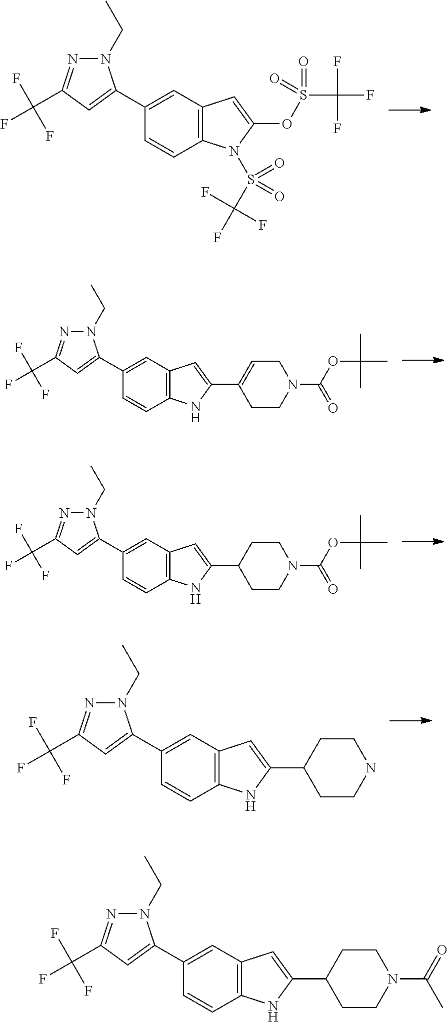

### Step 1 tert-butyl 4-(5-(1-ethyl-3-(trifluoromethyl)-1H-pyrazol-5-yl)-1H-indol-2-yl)-5,6-dihydropyridine-1(2H)-carboxylate

The Suuzki coupling was carried out in a manner identical to Example 189 but replacing 3-methylpyridine-4-boronic acid with [1-(tert-butoxycarbonyl)-1,2,3,6-tetrahydropyridin-4-yl]boronic acid to give tert-butyl-4-(5-(1-ethyl-3-(trifluoromethyl)-1H-pyrazol-5-yl)-1H-indol-2-yl)-5,6-dihydropyridine-1-carboxylate.

### Step 2 tert-butyl 4-(5-(1-ethyl-3-(trifluoromethyl)-1H-pyrazol-5-yl)-1H-indol-2-yl)piperidine-1-carboxylate

The hydrogenation was carried out in a manner identical to Example 198, but replacing 2-(3,6-dihydro-2H-pyran-4-yl)-5-(1-ethyl-3-(trifluoromethyl)-1H-pyrazol-5-yl)-1H-indole with tert-butyl 4-(5-(1-ethyl-3-(trifluoromethyl)-1H-pyrazol-5-yl)-1H-indol-2-yl)-5,6-dihydropyridine-1(2H)-carboxylate to give tert-butyl 4-(5-(1-ethyl-3-(trifluoromethyl)-1H-pyrazol-5-yl)-1H-indol-2-yl)piperidine-1-carboxylate.

### Step 3 5-(1-ethyl-3-(trifluoromethyl)-1H-pyrazol-5-yl)-2-(piperidin-4-yl)-1H-indole

A mixture of tent-butyl 4-(5-(1-ethyl-3-(trifluoromethyl)-1H-pyrazol-5-yl)-1H-indol-2-yl)piperidine-1-carboxylate (80 mg, 173 μmmol) and trifluoroacetic acid (1.48 g, 1 ml, 13.0 mmol) in CH₂CL₂ (5 ml) was stirred at room temperature for 3 hours, the mixture was poured into a slurry of ice and an aqueous NaHCO₃ solution (pH=7-8), partitioned between CH₂Cl₂ and water, the organic phase was washed with brine, dried over Na₂SO₄, filtered and concentrated under reduced pressure, the crude material was used directly in the next step.

### Step 4 1-(4-(5-(1-ethyl-3-(trifluoromethyl)-1H-pyrazol-5-yl)-1H-indol-2-yl)piperidin-1-yl)ethanone

To a suspension of 5-(1-ethyl-3-(trifluoromethyl)-1H-pyrazol-5-yl)-2-(piperidin-4-yl)-1H-indole (30 mg, 0.83 mmol) and TEA (17 mg, 23 μL, 0.17 mmol) in CH₂Cl₂ (5 ml) was added acetic anhydride (13 mg, 12 μL, 0.12 mmol) dropwise. The reaction mixture was stirred at room temperature overnight, partitioned between EtOAc and brine, the organic phase was dried over Na₂SO₄, filtered and concentrated under reduced pressure, the crude material was purified by flash chromatography (4:96 MeOH/EtOAc and 0.1% NH₄OH) to give 1-(4-(5-(1-ethyl-3-(trifluoromethyl)-1H-pyrazol-5-yl)-1H-indol-2-yl)piperidin-1-yl)ethanone, light yellow solid (22 mg, 65%). MS (M+H)=405.

### Example 201

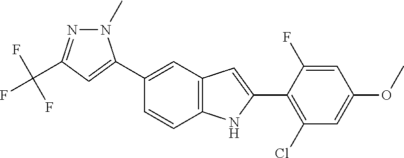

### 2-(2-chloro-6-fluoro-4-methoxyphenyl)-5-(1-methyl-3-(trifluoromethyl)-1H-pyrazol-5-yl)-1H-indole

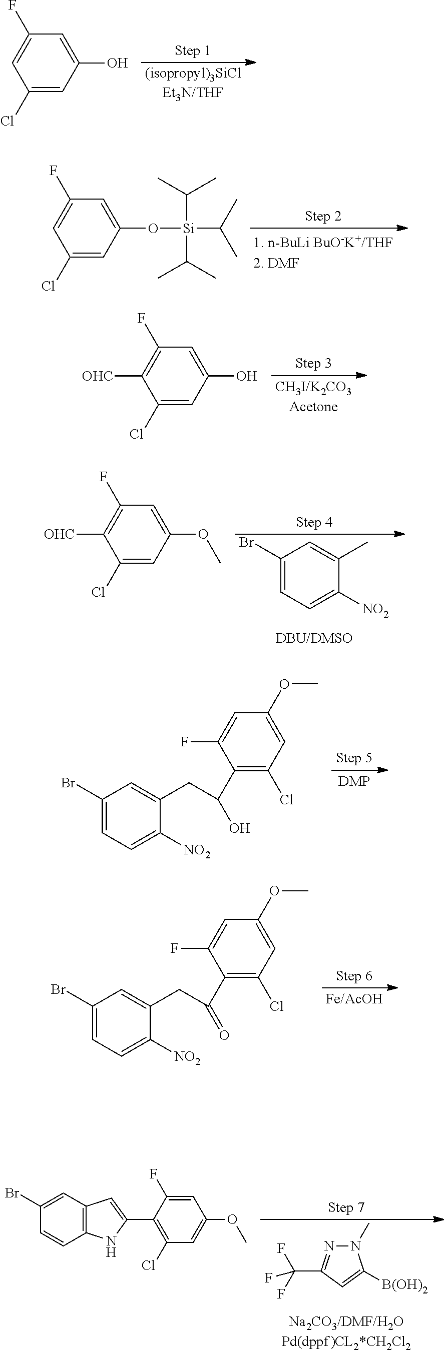

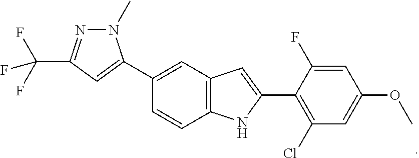

### Step 1 (3-chloro-5-fluorophenoxy)triisopropylsilane

To a solution of 3-chloro-5-fluorophenol (5 g, 34.1 mmol) in THF (70 ml), was added Et₃N (5.18 g, 51.2 mmol), followed by triisopropylsilyl chloride (7.24 g, 37.5 mmol) at room temperature, the mixture was stirred at room temperature overnight, the reaction mixture was concentrated, the resulting solid was filtered off, washed with EtOAc, the combined filtrate was washed with brine, dried over Na₂SO₄, filtered and concentrated under reduced pressure. The crude material was purified by flash chromatography (5% EtOAc/hexanes) to give (3-chloro-5-fluorophenoxy)triisopropylsilane as a colorless oil (10.4 g, 101%).

### Step 2 2-chloro-6-fluoro-4-hydroxybenzaldehyde

To a pre-cooled (−78° C.) solution of potassium tert-butoxide (1M, 36.9 ml, 36.9 mmol) in dry THF (100 ml) mixed with n-BuLi (1.6 M in hexane, 23.1 ml, 36.9 mmol) was added a solution of (3-chloro-5-fluorophenoxy)triisopropylsilane in THF (20 ml) dropwise, between −75 to −72° C., the mixture was stirred at −75° C. for 45 min., then DMF (2.7 g, 36.9 mmol) was added at −75° C., and stirred at the same temperature for 2 hours, water was added, then the mixture was partitioned between EtOAc and water, the organic phase was washed with brine, dried over Na₂SO₄, filtered and concentrated under reduced pressure. The crude material was purified by filtering through a pad of silica gel (10%, 20% EtOAc/hexanes) to give 2-chloro-6-fluoro-4-hydroxybenzaldehyde as a yellow solid (3.5 g, 65%).

### Step 3 2-chloro-6-fluoro-4-methoxybenzaldehyde

To a mixture of 2-chloro-6-fluoro-4-hydroxybenzaldehyde (3.42 g, 19.6 mmol) with K₂CO₃ (10.8 g, 78.4 mmol) in dry DMF (80 ml), was added iodomethane (9.08 g, 64 mmol), the mixture was stirred at room temperature overnight, partitioned between EtOAc and water, the organic phase was washed with brine, dried over Na₂SO₄, filtered and concentrated under reduced pressure. The crude material was purified by filtering through a pad of silica gel (20% EtOAc/hexanes) to give 2-chloro-6-fluoro-4-methoxybenzaldehyde as a yellow solid (3.67 g, 99%).

### Preparation of Compound 4-bromo-2-methyl-1-nitrobenzene

To a 0° C. solution of 3-methyl-4-nitroaniline in acetone (200 ml), was added 48% aq. HBr (22 ml), followed by a solution of NaNO₂ (4.76 g, 69 mmol) in water (20 ml) dropwise, between −10 to −6° C., the mixture was stirred between −6° C. to 1° C. for 20 minutes, solid CuBr (1.89 g, 133.1 mmol) was added in portions, (keeping the temperature below 15° C.), the mixture was stirred below 14° C. until nitrogen bubbling ceased. Most of the acetone was evaporated, the solid was filtered and washed with more water, the solid was dissolved in methylene chloride, dried over Na₂SO₄, filtered and concentrated under reduced pressure, the crude material was purified by flash chromatography (0-2% EtOAc/hexanes) to give a crude yellow solid, which was crystallized from very minimal amount of hexanes to give compound 4-bromo-2-methyl-1-nitrobenzene as a light yellow solid (6.66 g, 47%).

### Step 4 2-(5-bromo-2-nitrophenyl)-1-(2-chloro-6-fluoro-4-methoxyphenyl)ethanol

To a mixture of 4-bromo-2-methyl-1-nitrobenzene (4.17 g, 19.3 mmol) and 2-chloro-6-fluoro-4-methoxybenzaldehyde (3.64 g, 19.3 mmol) in DMSO (50 ml) was added 2,3,4,6,7,8,9,10-octahydropyrimido[1,2-a]azepine or DBU (3.53 g, 3.49 ml, 23.2 mmol) dropwise at room temperature, the mixture was stirred at room temperature for 4 hours, TLC showed there were still both of SM left, so an extra 1 ml of DBU was added, continued stirring overnight. The reaction mixture was poured into ice water, extracted with EtOAc, and the organic phase was washed with brine, dried over Na₂SO₄, filtered and concentrated under reduced pressure, the crude material was purified by flash chromatography (2%-40% EtOAc/hexanes) to give 2-(5-bromo-2-nitrophenyl)-1-(2-chloro-6-fluoro-4-methoxyphenyl)ethanol as a yellow solid (5.4 g, 69%).

### Step 5 2-(5-bromo-2-nitrophenyl)-1-(2-chloro-6-fluoro-4-methoxyphenyl)ethanone

To a solution of 2-(5-bromo-2-nitrophenyl)-1-(2-chloro-6-fluoro-4-methoxyphenyl)ethanol (5.4 g, 13.3 mmol) in CH₂Cl₂ (100 ml) was added Dess-martin periodinane (6.79 g, 16.0 mmol). The mixture was stirred at room temperature for 4 hours, partitioned between NaHCO₃ aqueous solution and CH₂Cl₂, the aqueous solution was twice extracted with EtOAc, and the combined organic phase was washed with brine, dried over Na₂SO₄, filtered and concentrated under reduced pressure to give 2-(5-bromo-2-nitrophenyl)-1-(2-chloro-6-fluoro-4-methoxyphenyl)ethanone as a light brown oil (8.74 g), which was used without purification in the next step.

### Step 6 5-bromo-2-(2-chloro-6-fluoro-4-methoxyphenyl)-1H-indole

To a round-bottomed flask was added 2-(5-bromo-2-nitrophenyl)-1-(2-chloro-6-fluoro-4-methoxyphenyl)ethanone (5.37 g, 13.3 mmol) and glacial AcOH (300 ml) to give a suspension. To the suspension were added; 100 ml of EtOH (to increase the solubility), and Iron (10.96 g, 196.3 mmol). The mixture was stirred at room temperature for one day (all SM were dissolved), the solid was filtered, washed with more EtOAc, then most of the EtOH and EtOAc were evaporated, the HOAc solution was poured into ice, and the resulting solid was collected by filtration, washed with water, the solid was dissolved into EtOAc, and the organic solution was washed with brine, dried over Na₂SO₄, filtered and concentrated under reduced pressure, the crude material was purified by flash chromatography (10-20% EtOAc/hexanes) to give 5-bromo-2-(2-chloro-6-fluoro-4-methoxyphenyl)-1H-indole as a yellow solid (4.45 g, 94%).

### Step 7 2-(2-chloro-6-fluoro-4-methoxyphenyl)-5-(1-methyl-3-(trifluoromethyl)-1H-pyrazol-5-yl)-1H-indole

To a vial was added 5-bromo-2-(2-chloro-6-fluoro-4-methoxyphenyl)-1H-indole (50 mg, 0.14 mmol), 1-methyl-5-(4,4,5,5-tetramethyl-1,3,2-dioxaborolan-2-yl)-3-trifluoromethyl)-1H-pyrazole (0.047 g, 0.169 mmol), and DMF (2 ml) to give a light brown solution, a solution of sodium carbonate (0.022 g, 0.212 mmol) in water (0.2 ml) was added, while the mixture was degassed with nitrogen, 1,1′-Bis(diphenylphosphino)-ferrocene-palladium(II) dichloride dichloromethane complex (5.8 mg, 4 mol %) was added and the vial sealed. The reaction mixture was heated to 80° C. and stirred overnight, water was added, partitioned between EtOAc and water, and the organic phase was washed with brine, dried over Na₂SO₄, filtered and concentrated under reduced pressure, the crude material was twice purified by preparative TLC plates (20% EtOAc/hexanes) to give a yellow gum, which was dissolved in EtOAc, washed with water 3 times and brine once, dried over Na₂SO₄, filtered and concentrated under reduced pressure to give a yellow solid, washed with hexanes twice to give 2-(2-chloro-6-fluoro-4-methoxyphenyl)-5-(1-methyl-3-(trifluoromethyl)-1H-pyrazol-5-yl)-1H-indole as an off-white solid (11 mg, 18%). MS (M+H)=424.

### Example 202

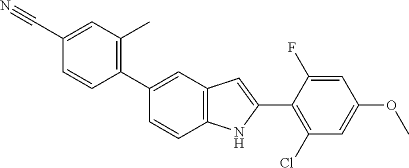

### 4-(2-(2-chloro-6-fluoro-4-methoxyphenyl)-1H-indol-5-yl)-3-methylbenzonitrile

Prepared in a manner identical to Example 201 replacing 1-methyl-5-(4,4,5,5-tetramethyl-1,3,2-dioxaborolan-2-yl)-3-trifluoromethyl)-1H-pyrazole with 4-cyano-2-methylphenylboronic acid in the Suzuki step to give 4-(2-(2-chloro-6-fluoro-4-methoxyphenyl)-1H-indol-5-yl)-3-methylbenzonitrile. MS (M+H)=391.

### Example 203

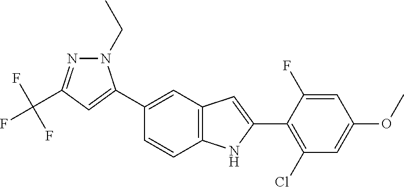

### 2-(2-chloro-6-fluoro-4-methoxyphenyl)-5-(1-ethyl-3-(trifluoromethyl)-1H-pyrazol-5-yl)-1H-indole

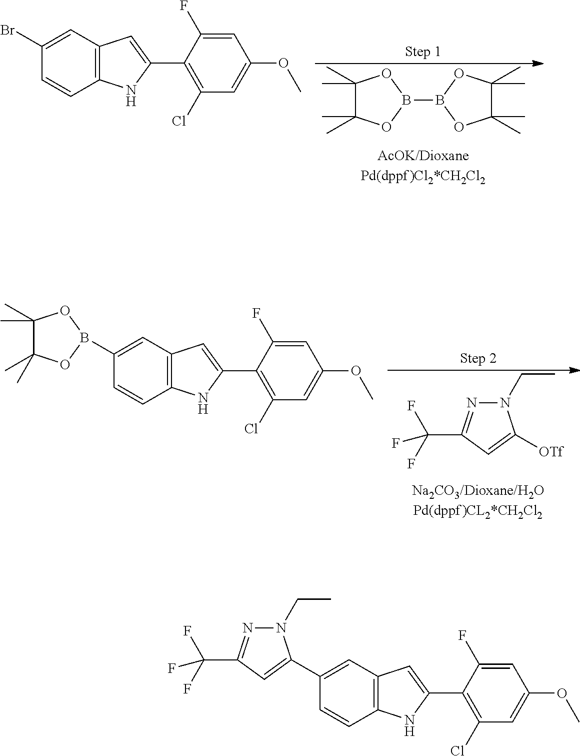

### Step 1 2-(2-chloro-6-fluoro-4-methoxyphenyl)-5-(4,4,5,5-tetramethyl-1,3,2-dioxaborolan-2-yl)-1H-indole

Prepared in a manner identical to Example 189 replacing 5-bromoindolin-2-one with 5-bromo-2-(2-chloro-6-fluoro-4-methoxyphenyl)-1H-indole to give 2-(2-chloro-6-fluoro-4-methoxyphenyl)-5-(4,4,5,5-tetramethyl-1,3,2-dioxaborolan-2-yl)-1H-indole.

### Step 2 2-(2-chloro-6-fluoro-4-methoxyphenyl)-5-(1-ethyl-3-(trifluoromethyl)-1H-indole

Prepared in a manner identical to Example 189 using 2-(2-chloro-6-fluoro-4-methoxyphenyl)-5-(4,4,5,5-tetramethyl-1,3,2-dioxaborolan-2-yl)-1H-indole and Trifluoro-methanesulfonic acid 2-ethyl-5-trifluoromethyl-2H-pyrazol-3-yl ester (Intermediate 12) to give 2-(2-chloro-6-fluoro-4-methoxyphenyl)-5-(1-ethyl-3-(trifluoromethyl)-1H-pyrazol-5-yl)-1H-indole. MS (M+H)=438.

### Example 204

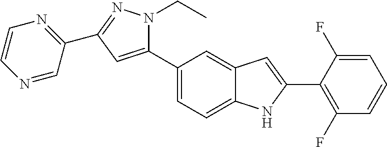

### 2-(2,6-difluorophenyl)-5-(1-ethyl-3-(pyrazin-2-yl)-1H-pyrazol-5-yl)-1H-indole

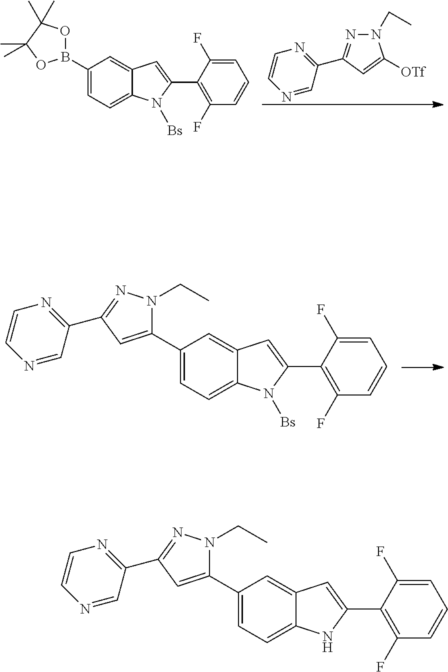

### Step 1 2-(2,6-difluorophenyl)-5-(1-ethyl-3-(pyrazin-2-yl)-1H-pyrazol-5-yl)-1-(phenylsulfonyl)-1H-indole

2-(2,6-difluorophenyl)-1-(phenylsulfonyl)-5-(4,4,5,5-tetramethyl-1,3,2-dioxaborolan-2-yl)-1H-indole (94 mg, 190 μmmol, Eq: 1.00), 1-ethyl-3-(pyrazin-2-yl)-1H-pyrazol-5-yl trifluoromethanesulfonate (79.5 mg, 247 μmmol, Eq: 1.3), tetrakis(triphenylphosphine)palladium (0) (21.9 mg, 19.0 μmmol, Eq: 0.1) and potassium carbonate (78.7 mg, 569 μmmol, Eq: 3) in Dioxane (3.37 ml)/Water (843 μl) was heated to 90° C. under N₂ for 2 hrs. Dried reaction onto silica gel for purification using a 30-60% EtOAc/Hex gradient. Obtained 2-(2,6-difluorophenyl)-5-(1-ethyl-3-(pyrazin-2-yl)-1H-pyrazol-5-yl)-1-(phenylsulfonyl)-1H-indole (80 mg, 148 μmmol, 78% yield) as a white powder.

### Step 2 2-(2,6-difluorophenyl)-5-(1-ethyl-3-(pyrazin-2-yl)-1H-pyrazol-5-yl)-1H-indole

2-(2,6-difluorophenyl)-5-(1-ethyl-3-(pyrazin-2-yl)-1H-pyrazol-5-yl)-1-(phenylsulfonyl)-1H-indole (80 mg, 148 μmmol, Eq: 1.00) and cesium carbonate (120 mg, 369 μmmol, Eq: 2.5) in THF (1.97 ml)/MeOH (985 μl) were stirred overnight at r.t. Removed solvents in vacuo. Residue was diluted with ether and water. Washed with water and brine. Water was back-washed with DCM. Organics were combined and dried over MgSO4. Filtered off MgSO₄ and removed solvents. Obtained 2-(2,6-difluorophenyl)-5-(1-ethyl-3-(pyrazin-2-yl)-1H-pyrazol-5-yl)-1H-indole (54 mg, 135 μmmol, 91% yield) as an off-white solid; MS (M+H)=402

### Example 205

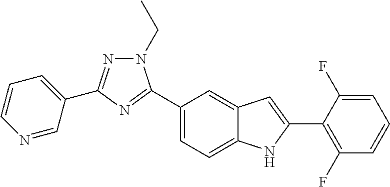

### 2-(2,6-Difluoro-phenyl)-5-(2-ethyl-5-pyridin-3-yl-2H-[1,2,4]triazol-3-yl)-1H-indole

Prepared in a manner identical to Example 46 substituting Intermediate 27 in the Suzuki coupling step. MS (M+H)=402.

### Example 206

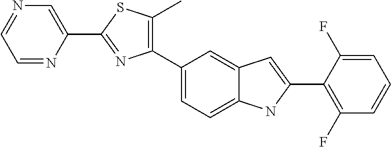

### 2-(2,6-Difluoro-phenyl)-5-(5-methyl-2-pyrazin-2-yl-thiazol-4-yl)-1H-indole

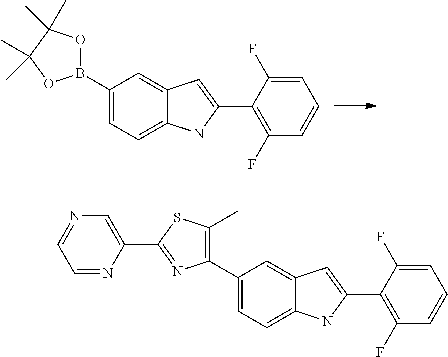

In a 10 mL, round-bottomed flask, 2-(2,6-difluorophenyl)-5-(4,4,5,5-tetramethyl-1,3,2-dioxaborolan-2-yl)-1H-indole (75 mg, 211 μmmol), Trifluoro-methanesulfonic acid 5-methyl-2-pyrazin-2-yl-thiazol-4-yl ester (82.4 mg, 253 μmmol) and [1,1′-bis(diphenylphosphino)ferrocenedichloropalladium (II) (30.9 mg, 42.2 μmmol, Eq: 0.2) and potassium carbonate (87.5 mg, 633 μmmol) were combined with dioxane (5 ml) to give a red suspension and the resultant reaction was heated to 80° C. and stirred for 1 h. The reaction mixture was poured into 50 mL H₂O and extracted with ethyl acetate (3×20 mL). The organic layers were dried over MgSO₄ and concentrated in vacuo. The crude material was purified by flash column chromatography (silica gel, 12 g, 15% to 25% ethyl acetate in hexanes). to give 2-(2,6-Difluoro-phenyl)-5-(5-methyl-2-pyrazin-2-yl-thiazol-4-yl)-1H-indole (33 mg, 38.6%) as light yellow solid. MS (M+H)=405.

### Example 207

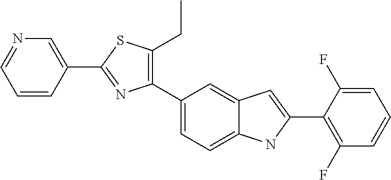

### 2-(2,6-Difluoro-phenyl)-5-(5-ethyl-2-pyridin-3-yl-thiazol-4-yl)-1H-indole

2-(2,6-Difluoro-phenyl)-5-(5-ethyl-2-pyridin-3-yl-thiazol-4-yl)-1H-indole was prepared in a manner identical to 2-(2,6-Difluoro-phenyl)-5-(5-methyl-2-pyrazin-2-yl-thiazol-4-yl)-1H-indole with the following materials 2-(2,6-difluorophenyl)-5-(4,4,5,5-tetramethyl-1,3,2-dioxaborolan-2-yl)-1H-indole and 5-ethyl-2-(pyrazin-2-yl)thiazol-4-yl trifluoromethanesulfonate. MS (M+H)=418.

### Example 208

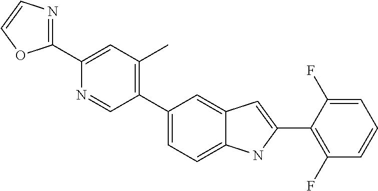

### 2-(2,6-Difluoro-phenyl)-5-(4-methyl-6-oxazol-2-yl-pyridin-3-yl)-1H-indole

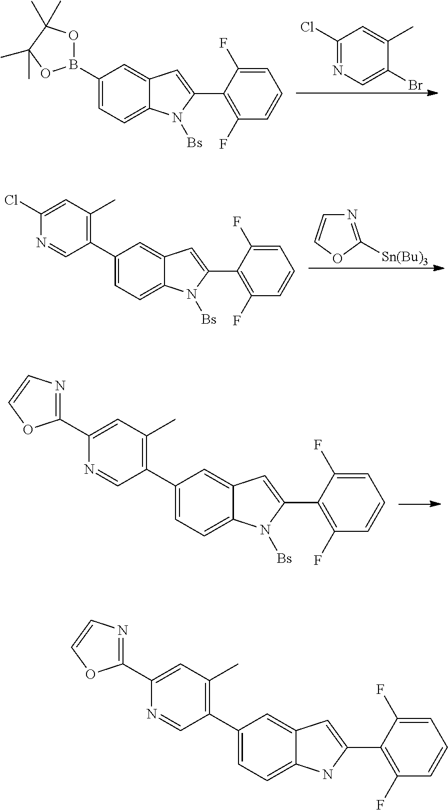

### Step 1 5-(6-chloro-4-methylpyridin-3-yl)-2-(2,6-difluorophenyl)-1-(phenylsulfonyl)-1H-indole

2-(2,6-difluorophenyl)-1-(phenylsulfonyl)-5-(4,4,5,5-tetramethyl-1,3,2-dioxaborolan-2-yl)-1H-indole (500 mg, 1.01 mmol, Eq: 1.00), 5-bromo-2-chloro-4-methylpyridine (188 mg, 908 mmol, Eq: 0.9), 1,1′-bis(diphenylphosphino)ferrocene-palladium(II) dichloride dichloromethane complex (165 mg, 202 μmmol, Eq: 0.2) and potassium carbonate (419 mg, 3.03 mmol, Eq: 3) in Dioxane (17.9 ml) and Water (4.49 ml) were heated to 80° C. under N₂ for 2 hrs. Diluted with EtOAc and washed with brine (1×) and water (1×). Dried organic layer onto silica gel for purification using a 10-40% EtOAc/Hex gradient. Obtained 5-(6-chloro-4-methylpyridin-3-yl)-2-(2,6-difluorophenyl)-1-(phenylsulfonyl)-1H-indole (370 mg, 748 μmmol, 74.1% yield) as a white solid.

### Step 2 5-(6-chloro-4-methylpyridin-3-yl)-2-(2,6-difluorophenyl)-1-(phenylsulfonyl)-1H-indole

To a solution of 5-(6-chloro-4-methylpyridin-3-yl)-2-(2,6-difluorophenyl)-1-(phenylsulfonyl)-1H-indole (500 mg, 1.01 mmol, Eq: 1.00) in Dioxane (5.05 ml) was added 2-(tributylstannyl)oxazole (470 mg, 1.31 mmol, Eq: 1.3) followed by 1,1′-bis(diphenylphosphino)ferrocene-palladium(II) dichloride dichloromethane complex (165 mg, 202 μmmol, Eq: 0.2) and heated to 90° over night. Dried reaction mixture onto silica gel for purification using a 30-60% EtOAc/Hex gradient. Obtained 5-(6-chloro-4-methylpyridin-3-yl)-2-(2,6-difluorophenyl)-1-(phenylsulfonyl)-1H-indole (56 mg, 11% yield) as a solid.

### Step 3 2-(5-(2-(2,6-difluorophenyl)-1H-indol-5-yl)-4-methylpyridin-2-yl)oxazole

2-(5-(2-(2,6-difluorophenyl)-1-(phenylsulfonyl)-1H-indol-5-yl)-4-methylpyridin-2-yl)oxazole (56 mg, 106 μmmol, Eq: 1.00) and cesium carbonate (69.2 mg, 212 μmmol, Eq: 2) in THF (1.42 ml)/Methanol (708 μl) was stirred at r.t. over weekend. Diluted with Et₂O and washed with water (1×). Dried organic layer onto silica gel for purification using a 30-40% EtOAc/Hex gradient. Obtained 2-(5-(2-(2,6-difluorophenyl)-1H-indol-5-yl)-4-methylpyridin-2-yl)oxazole (39 mg, 101 μmmol, 94.8% yield) as a white waxy solid; MS (M+H)=388

### Example 209

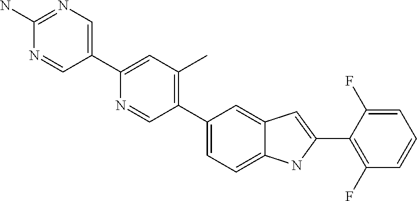

### 5-{5-[2-(2,6-Difluoro-phenyl)-1H-indol-5-yl]-4-methyl-pyridin-2-yl}-pyrimidin-2-ylamine

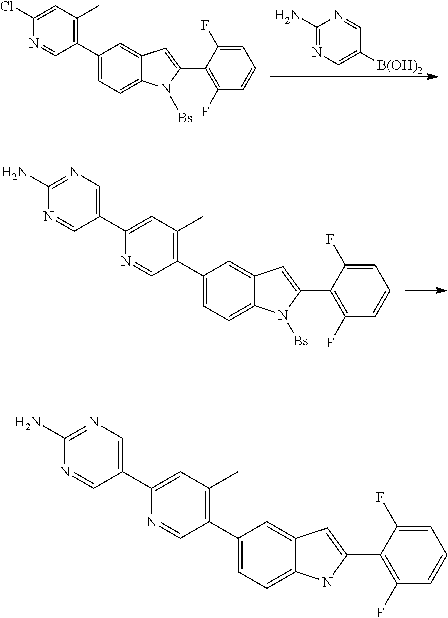

### Step 1 5-{5-[1-Benzenesulfonyl-2-(2,6-difluoro-phenyl)-1H-indol-5-yl]-4-methyl-pyridin-2-yl}-pyrimidin-2-ylamine

5-(6-chloro-4-methylpyridin-3-yl)-2-(2,6-difluorophenyl)-1-(phenylsulfonyl)-1H-indole (150 mg, 303 μmmol, Eq: 1.00), 2-aminopyrimidin-5-ylboronic acid (63.2 mg, 455 μmmol, Eq: 1.5), cesium carbonate (296 mg, 909 μmmol, Eq: 3), tetrakis(triphenylphosphine)palladium (0) (17.5 mg, 15.2 μmmol, Eq: 0.05) in Dioxane (6.25 ml)/Water (1.25 ml) was heated to 90° C. under N₂ for 3 hrs. Dried unto silica for purification using a 60-100% EtOAc/Hex gradient. Obtained 5-{5-[1-Benzenesulfonyl-2-(2,6-difluoro-phenyl)-1H-indol-5-yl]-4-methyl-pyridin-2-yl}-pyrimidin-2-ylamine (168 mg, 95.4% yield) as a white solid.

### Step 2 5-{5-[2-(2,6-Difluoro-phenyl)-1H-indol-5-yl]-4-methyl-pyridin-2-yl}-pyrimidin-2-ylamine

5-(5-(2-(2,6-difluorophenyl)-1-(phenylsulfonyl)-1H-indol-5-yl)-4-methylpyridin-2-yl)pyrimidin-2-amine (160 mg, 289 μmmol, Eq: 1.00) and cesium carbonate (235 mg, 723 μmmol, Eq: 2.5) in THF (7.71 ml)/Methanol (3.85 ml) was stirred at r.t. over night. Increased temperature to 60° C. for 8 hrs. Dried onto silica gel for purification using a 5-30% DCM/(20% DCM/MeOH) gradient. Further purified using HPLC. Obtained 5-{5-[2-(2,6-Difluoro-phenyl)-1H-indol-5-yl]-4-methyl-pyridin-2-yl}-pyrimidin-2-ylamine (32 mg, 26.8% yield) as an off-white solid; MS (M+H)=414

### Example 210

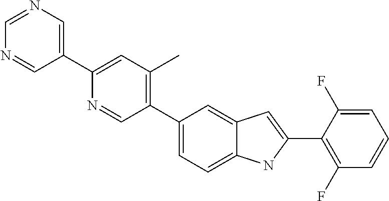

### 2-(2,6-Difluoro-phenyl)-5-(4-methyl-6-pyrimidin-5-yl-pyridin-3-yl)-1H-indole

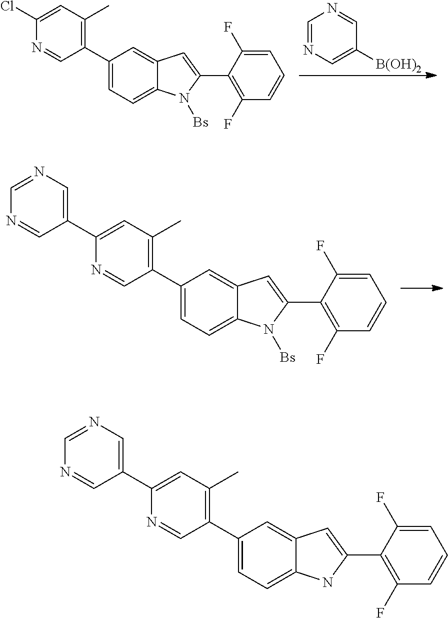

### Step 1 1-Benzenesulfonyl-2-(2,6-difluoro-phenyl)-5-(4-methyl-6-pyrimidin-5-yl-pyridin-3-yl)-1H-indole

5-(6-chloro-4-methylpyridin-3-yl)-2-(2,6-difluorophenyl)-1-(phenylsulfonyl)-1H-indole (150 mg, 303 μmmol, Eq: 1.00), pyrimidin-5-ylboronic acid (56.3 mg, 455 μmmol, Eq: 1.5), cesium carbonate (296 mg, 909 μmmol, Eq: 3), tetrakis(triphenylphosphine)palladium (0) (17.5 mg, 15.2 μmol, Eq: 0.05) in Dioxane (6.25 ml)/Water (1.25 ml) was heated to 90° C. under N₂ for 2 hrs. Dried onto silica gel for purification using a 30-70% EtOAc/Hex gradient. Obtained 1-Benzenesulfonyl-2-(2,6-difluoro-phenyl)-5-(4-methyl-6-pyrimidin-5-yl-pyridin-3-yl)-1H-indole (160 mg, 98% yield) as a white solid.

### Step 2 2-(2,6-Difluoro-phenyl)-5-(4-methyl-6-pyrimidin-5-yl-pyridin-3-yl)-1H-indole

2-(2,6-difluorophenyl)-5-(4-methyl-6-(pyrimidin-5-yl)pyridin-3-yl)-1-(phenylsulfonyl)-1H-indole (160 mg, 297 μmmol, Eq: 1.00) and cesium carbonate (242 mg, 743 μmmol, Eq: 2.5) in THF (7.92 ml)/MeOH (3.96 ml) were stirred over night at r.t. Increased temperature to 60° C. for 8 hrs. Dried onto silica gel for purification using a 5-10% DCM/(20% DCM/MeOH) gradient. Further purified by HPLC. Obtained 2-(2,6-Difluoro-phenyl)-5-(4-methyl-6-pyrimidin-5-yl-pyridin-3-yl)-1H-indole (16 mg, 13.5% yield) as a white solid; MS (M+H)=399

### Example 211

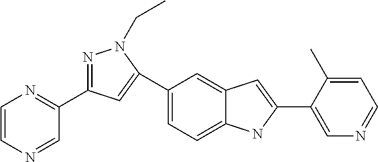

### 2-(4-Methyl-pyridin-3-yl)-5-(4,4,5,5-tetramethyl-1,3,2-dioxaborolan-2yl)-1H-indole

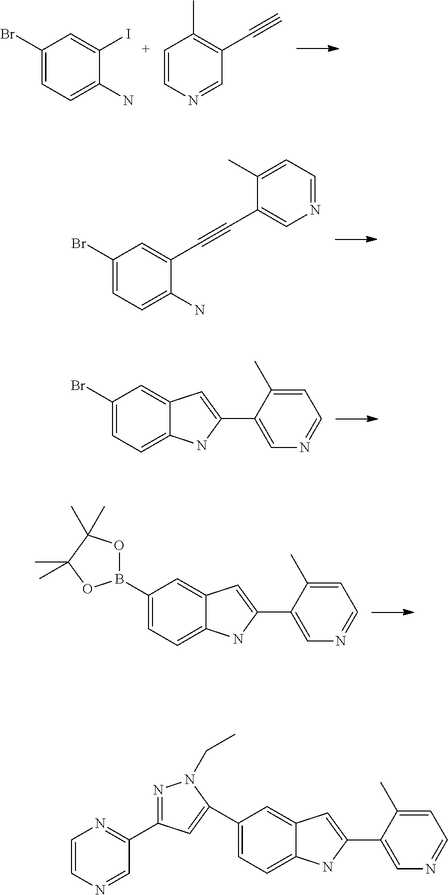

### Step 1 4-Bromo-2-(4-methyl-pyridin-3-ylethynyl)-phenylamine

Bromo-2-iodoaniline (2.07 g, 6.95 mmol), 3-ethynyl-4-methylpyridine (Intermediate 46, 915 mg, 7.81 mmol), tetrakis(triphenylphosphine)palladium(0) (401 mg, 347 μmmol) and copper (I) iodide (66.2 mg, 347 μmmol) were combined with DMF (28.3 mL) and triethylamine (13.8 mL), flushed with nitrogen and heated at 55° C. for 4 h. The reaction mixture was cooled, diluted with water and extracted with ethyl acetate. The organic layers were combined, washed with brine, dried over Na₂SO₄, filtered and concentrated under reduced pressure. The resulting crude compound was purified by flash column chromatography (silica gel, 120 g, 50% to 80% ethyl acetate in hexanes) to give 4-bromo-2-(4-methyl-pyridin-3-ylethynyl)-phenylamine (1.86 g, 93%) which was used directly without further purification. MS (M+H)=287.

### Step 2 5-Bromo-2-(4-methyl-pyridin-3-yl)-1H-indole

Bromo-2-((4-methylpyridin-3-yl)ethynyl)aniline (1.86 g, 6.48 mmol) and gold (III) chloride (118 mg, 389 μmmol) were combined with ethanol (85 mL) and heated at 67° C. for 5 h. Ethyl acetate was added (60 mL), filtered through celite, concentrated under reduced pressure, triturated from hot ethyl acetate, cooled and filtered to give 5-bromo-2-(4-methyl-pyridin-3-yl)-1H-indole (1.38 g, 74%) which was used directly without further purification. MS (M+H)=287.

### Step 3 2-(4-Methyl-pyridin-3-yl)-5-(4,4,5,5-tetramethyl-1,3,2-dioxaborolan-2-yl)-1H-indole

Bromo-2-(4-methylpyridin-3-yl)-1H-indole (0.45 g, 1.57 mmol), bis(pina colato)diboron (517 mg, 2.04 mmol) and potassium acetate (308 mg, 3.13 mmol) were combined with dioxane (8 mL) and flushed with nitrogen. 1,1′-bis(diphenyl phosphino)ferrocene-palladium (II) dichloride dichloromethane complex (128 mg, 157 μmmol) was added. The mixture was heated at 100° C. for 2 h. The mixture was cooled, diluted with ethyl acetate, washed with water and brine, dried over Na₂SO₄, filtered and concentrated under reduced pressure. The resulting crude compound was purified by flash column chromatography (silica gel, 40 g, 50% to 80% ethyl acetate in hexanes) to give 2-(4-methyl-pyridin-3-yl)-5-(4,4,5,5-tetramethyl-1,3,2-dioxaborolan-2-yl)-1H-indole (0.325 g, 62%). MS (M+H)=335.

### 5-(2-Ethyl-5-pyrazin-2-yl-2H-pyrazo-1-3-yl)-2-(4-methyl-pyridin-3-yl)-1H-indole

2-(4-Methylpyridin-3-yl)-5-(4,4,5,5-tetramethyl-1,3,2-dioxaborolan-2-yl)-1H-indole (115 mg, 344 μmmol), 1-ethyl-3-(pyrazin-2-yl)-1H-pyrazol-5-yl trifluoromethanesulfonate (133 mg, 413 μmmol) and potassium carbonate (142 mg, 1.03 mmol) were combined with dioxane (6 mL) and water (1.5 mL). Tetrakis(triphenylphosphine)palladium(0) (40 mg, 34.6 μmmol) was added. The mixture was flushed with nitrogen and heated at 90° C. for 4 h. The mixture was cooled, diluted with ethyl acetate, washed with brine, dried over Na₂SO₄, filtered and concentrated under reduced pressure. The resulting crude compound was purified by flash column chromatography (silica gel, 40 g, 80% to 100% ethyl acetate in hexanes) followed by second purification with preparative reverse phase HPLC (Supercosil™ Cat# 59174, 25 cm×21.2 mm×12 micron, 20 to 95% acetonitrile/water with 0.05% TFA) and removal of the TFA through an ethyl acetate/aqueous sodium bicarbonate workup gave 5-(2-ethyl-5-pyrazin-2-yl-2H-pyrazo-1-3-yl)-2-(4-methyl-pyridin-3-yl)-1H-indole (6 mg, 5%). MS (M+H)=381.

### Example 212

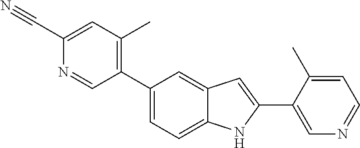

### Methyl-5-[2-(4-methyl-pyridin-3-yl)-1H-indol-5-yl]-pyridine-2-carbonitrile

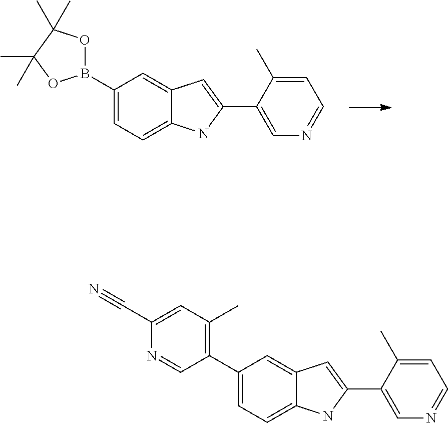

To a reaction vial was added 2-(4-methylpyridin-3-yl)-5-(4,4,5,5-tetramethyl-1,3,2-dioxaborolan-2-yl)-1H-indole (80 mg, 239 μmmol), 5-bromo-4-methylpyridine-2-carbonitrile (47.2 mg, 239 μmmol), Tetrakis(triphenylphosphine)palladium(0) (27.8 mg, 24.1 μmmol), and sodium bicarbonate (60.3 mg, 718 μmmol), in toluene (3 mL), ethanol (2 mL) and water (1 mL). The reaction mixture was degassed with nitrogen, sealed and heated to 80° C. while stirring for 2 hrs. The reaction mixture was cooled, filtered through celite, partitioned, dried over MgSO₄, filtered and then purified by flash column chromatography (silica gel, 25 g, 20% to 80% ethyl acetate in hexanes), to give 4-Methyl-5-[2-(4-methyl-pyridin-3-yl)-1H-indol-5-yl]-pyridine-2-carbonitrile as a white solid (26 mg). Second purification by preparative reverse phase HPLC (Supercosil™ Cat# 59174, 25 cm×21.2 mm×12 micron, 20 to 95% acetonitrile/water with 0.05% TFA) and removal of the TFA through an ethyl acetate/aqueous sodium bicarbonate workup gave 4-Methyl-5-[2-(4-methyl-pyridin-3-yl)-1H-indol-5-yl]-pyridine-2-carbonitrile (7 mg, 9.02%) of as a lyophilized solid. MS (M+H)=325.

### Example 213

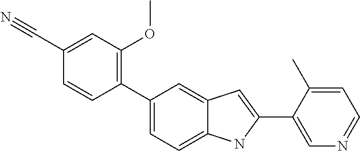

### Methoxy-5-[2-(4-methyl-pyridin-3-yl)-1H-indol-5-yl]-pyridine-2-carbonitrile

Methoxy-5-[2-(4-methyl-pyridin-3-yl)-1H-indol-5-yl]-pyridine-2-carbonitrile was prepared in a manner identical to 4-Methyl-5-[2-(4-methyl-pyridin-3-yl)-1H-indol-5-yl]-pyridine-2-carbonitrile with the following materials (2-(4-methylpyridin-3-yl)-5-(4,4,5,5-tetramethyl-1,3,2-dioxaborolan-2-yl)-1H-indole and 4-Bromo-3-methoxybenzo nitrile MS (M+H)=340.

### Example 214

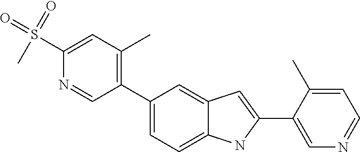

### 5-(6-Methanesulfonyl-4-methyl-pyridin-3-yl)-2-(4-methyl-pyridin-3-yl)1indole

5-(6-Methanesulfonyl-4-methyl-pyridin-3-yl)-2-(4-methyl-pyridin-3-yl)1indole was prepared in a manner identical to 4-Methyl-5-[2-(4-methyl-pyridin-3-yl)-1H-indol-5-yl]-pyridine-2-carbonitrile with the following materials (2-(4-methylpyridin-3-yl)-5-(4,4,5,5-tetramethyl-1,3,2-dioxaborolan-2-yl)-1H-indole and 5-bromo-4-methyl-2-(methylsulfonyl)pyridine. MS (M+H)=378.

### Example 215

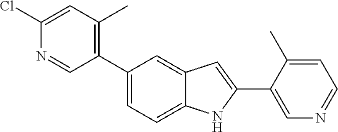

### 5-(6-Chloro-4-methyl-pyridin-3-yl)-2-(4-methyl-pyridin-3-yl)-1H-indole

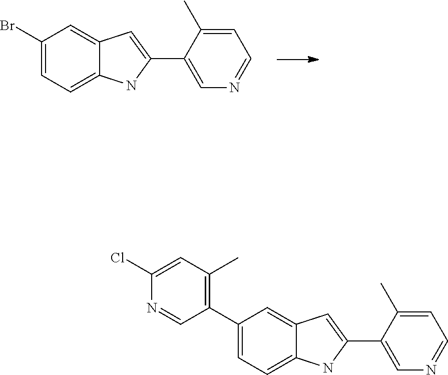

Bromo-2-(4-methylpyridin-3-yl)-1H-indole (500 mg, 1.74 mmol), 2-chloro-4-methylpyridine-5-boronic acid (518 mg, 3.02 mmol) and potassium carbonate (722 mg, 5.22 mmol) were combined with dioxane (20 mL) and water (2 mL).

Tetrakis(triphenylphosphine)palladium(0) (161 mg, 139 μmmol) was added. The mixture was flushed with nitrogen and heated at 80° C. for 23 h. The mixture was cooled, diluted with ethyl acetate, washed with brine, dried over Na₂SO₄, filtered and concentrated under reduced pressure. The residue was treated with acetone and methanol, filtered warm through celite and concentrated under reduced pressure. The resulting crude compound was purified by flash column chromatography (silica gel, 120 g, 1% to 5% methanol in dichloromethane) to give 5-(6-chloro-4-methyl-pyridin-3-yl)-2-(4-methyl-pyridin-3-yl)-1H-indole (133.4 mg, 23%). MS (M+H)=334.

### Example 216

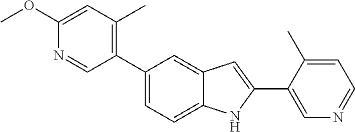

### 5-(6-Methoxy-4-methyl-pyridin-3-yl)-2-(4-methyl-pyridin-3-yl)-1H-indole

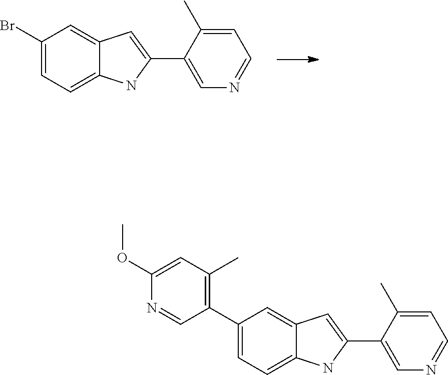

To a reaction vial was added 5-bromo-2-(4-methylpyridin-3-yl)-1H-indole (100 mg, 348 mmol), 2-Methoxy-4-methylpyridine-5-boronic acid (75.6 mg, 453 μmmol, Tetrakis(triphenylphosphine)palladium(0) (34.8 mg, 30.1 μmmol), sodium bicarbonate (87.8 mg, 1.04 mmol) in toluene (3 mL), ethanol (2 mL) and water (1 mL). The reaction mixture was degassed with nitrogen, sealed and heated to 80° C. while stirring for 2 hrs. The reaction mixture was cooled, filtered through celite, partitioned, dried over MgSO₄, filtered and purified by flash column chromatography (silica gel, 25 g, 20% to 80% ethyl acetate in hexanes), and lyophilized it to give 5-(6-Methoxy-4-methyl-pyridin-3-yl)-2-(4-methyl-pyridin-3-yl)-1H-indol (89 mg, 77.6%). MS (M+H)=330.

### Example 217

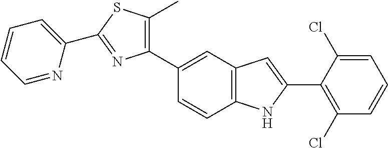

### 2-(2,6-Dichloro-phenyl)-5-(5-methyl-2-pyridin-2-yl-thiazol-4-yl)-1H-indole

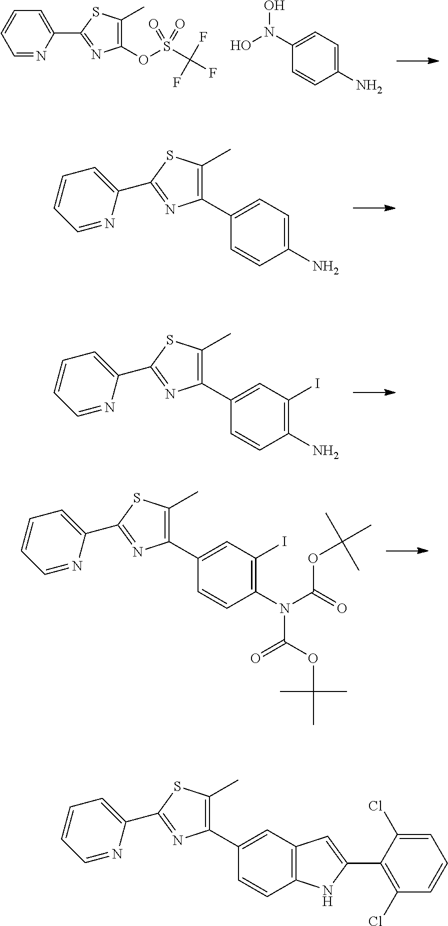

4-(5-Methyl-2-pyridin-2-yl-thiazol-4-yl)-phenylamine: To a solution of Trifluoro-methanesulfonic acid 5-methyl-2-pyridin-2-yl-thiazol-4-yl ester (Intermediate 1, 500 mg, 1.26 mmol) and 4-aminophenylboronic acid (417 mg, 1.9 mmol) in DMF (8 mL) was added aq. K₂CO₃ (2M, 1.26 ml, 2.52 mmol). The mixture was then purged with nitrogen (10 min), after which Pd(PPh₃)₄ (88 mg, 0.076 mmol) was added and the mixture heated at 100° C. for 12 h. Upon cooling, the mixture was filtered through Celite and the filtrate was diluted with water and extracted with EtOAc. The organic phase was washed with brine, dried, concentrated, and the crude mass was purified by column chromatography (25-30% EtOAC-Hexane) to give 445-Methyl-2-pyridin-2-yl-thiazol-4-yl)-phenylamine (700 mg, 94.8%) as a white solid.

Iodo-4-(5-methyl-2-pyridin-2-yl-thiazol-4-yl)-phenylamine: To a stirred solution of 445-Methyl-2-pyridin-2-yl-thiazol-4-yl)-phenylamine (3 gm, 11.85 mmol) in DCM-AcOH (2:1, 90 ml) was added benzyl trimethyl ammonium dichloroiodate (4.95 gm, 14.22 mmol). The reaction mixture was heated to 55° C. for 1.5 hr, after which it was evaporated under reduced pressure and crude material purified by column chromatography (40% EtOAc-Hexanes) to give 2-Iodo-4-(5-methyl-2-pyridin-2-yl-thiazol-4-yl)-phenylamine (1.9 gm, 40.1%) as a yellow solid.

N,N-bis-tert-butyl carbamate-2-Iodo-4-(5-methyl-2-pyridin-2-yl-thiazol-4-yl)-phenylamine: 2-Iodo-4-(5-methyl-2-pyridin-2-yl-thiazol-4-yl)-phenylamine (1.8 gm, 4.57 mmol) was dissolved in THF (9 ml) and catalytic amount of DMAP was added followed by BOC-anhydride (1.8 ml, 9.15 mmol). The reaction mixture was then heated to reflux for 1 h, evaporated under reduced pressure, and the crude material was purified by column chromatography (25% EtOAc-Hexanes) to give N,N-bis-tert-butyl carbamate-2-Iodo-4-(5-methyl-2-pyridin-2-yl-thiazol-4-yl)-phenylamine (1.5 gm, 55.2%) as a yellow solid.

2-(2,6-Dichloro-phenyl)-5-(5-methyl-2-pyridin-2-yl-thiazol-4-yl)-1H-indole: To a mixture of N,N-bis-tert-butyl carbamate-2-Iodo-4-(5-methyl-2-pyridin-2-yl-thiazol-4-yl)-phenylamine (150 mg, 0.253 mmol), 1,3-Dichloro-2-ethynyl-benzene (Intermediate 47, 64.5 mg, 0.3794 mmol) and i-Pr₂NH (0.5 ml, 0.35 mmol) in DMAC-Water (1:1, 1 ml) (28 ml) was added Pd(PPh₃)₄ (18 mg, 0.015 mmol) and CuI (5 mg, 0.025 mmol). The mixture was stirred at 100° C. for 10 min under microwave conditions. After which the reaction was cooled to RT, diluted with water, and extracted with DCM. The organic phase was washed with brine, dried, concentrated, and the crude material was purified by column chromatography (15% EtOAC-Hexane) to give 2-(2,6-Dichloro-phenyl)-5-(5-methyl-2-pyridin-2-yl-thiazol-4-yl)-1H-indole (20 mg, 18%) as an off white solid, MS (M+H)=436.

### Example 218

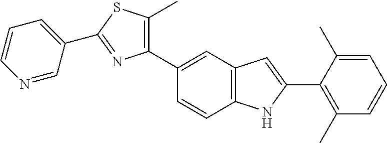

### 2-(2,6-Dimethyl-phenyl)-5-(5-methyl-2-pyridin-3-yl-thiazol-4-yl)-1H-indole

Prepared in a manner identical to Example 217. Substituting Intermediate 1 in the initial Suzuki coupling step and intermediate 48 in the Sonagashira coupling step. MS (M+H)=396.

### Example 219

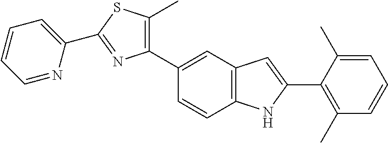

### 2-(2,6-Dimethyl-phenyl)-5-(5-methyl-2-pyridin-2-yl-thiazol-4-yl)-1H-indole

Prepared in a manner identical to Example 217. Substituting Intermediate 48 in the Sonagashira coupling step. MS (M+H)=396.

### Example 220

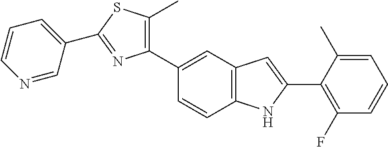

### 2-(2-Fluoro-6-methyl-phenyl)-5-(5-methyl-2-pyridin-3-yl-thiazol-4-yl)-1H-indole

Prepared in a manner identical to Example 217. Substituting Intermediate 49 in the Sonagashira coupling step. MS (M+H)=400.

### Example 221

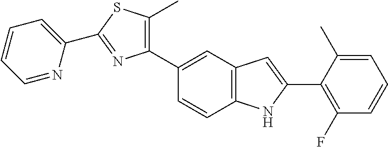

### 2-(2-Fluoro-6-methyl-phenyl)-5-(5-methyl-2-pyridin-2-yl-thiazol-4-yl)-1H-indole

Prepared in a manner identical to Example 217. Substituting Intermediate 13 in the initial Suzuki step and Intermediate 49 in the Sonagashira coupling step. MS (M+H)=400.

### Example 222

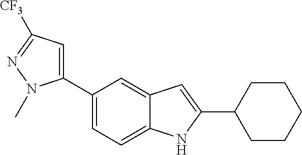

Cyclohexyl-5-(2,5-dimethyl-2H-pyrazol-3-yl)-1H-indole

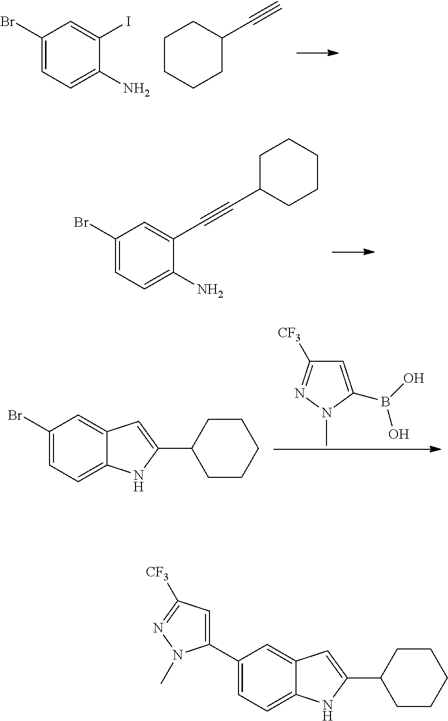

### Step 1 4-bromo-2-(cyclohexylethynyl)aniline

Bromo-2-iodoaniline (2 g, 6.71 mmol, Eq: 1.00), ethynylcyclohexane (799 mg, 7.38 mmol, Eq: 1.1), tetrakis(triphenylphosphine)palladium (0) (388 mg, 336 μmmol, Eq: 0.05) and copper(I) iodide (63.9 mg, 336 μmmol, Eq: 0.05) in triethylamine (13.4 ml, 6.71 mmol, Eq: 1.00) and DMF (26.9 ml) were heated to 120° C. overnight. Diluted with EtOAc and washed with water (2×) and brine (1×). The organic layer was dried onto silica gel for purification using a 10-22% EtOAc/Hex gradient. Obtained 4-bromo-2-(cyclohexylethynyl)aniline (585 mg, 2.1 mmol, 31.3% yield) as a brown oily semi solid.

### Step 2 5-bromo-2-cyclohexyl-1H-indole

bromo-2-(cyclohexylethynyl)aniline (585 mg, 2.1 mmol, Eq: 1.00) and gold(III) chloride (38.3 mg, 126 μmmol, Eq: 0.06) were heated at 67° C. in EtOH (42.1 ml) overnight. Dried reaction onto silica gel for purification using a 7-17% EtOAc/Hex gradient. Obtained 5-bromo-2-cyclohexyl-1H-indole (370 mg, 1.33 mmol, 63.2% yield) as a white solid.

### Step 3 2-Cyclohexyl-5-(2,5-dimethyl-2H-pyrazol-3-yl)-1H-indole

bromo-2-cyclohexyl-1H-indole (45 mg, 162 μmmol, Eq: 1.00), 1-methyl-3-(trifluoromethyl)-1H-pyrazol-5-ylboronic acid (31.4 mg, 162 μmmol, Eq: 1.00), 1,1′-bis(diphenylphosphino)ferrocene-palladium(II) dichloride dichloromethane complex (26.4 mg, 32.4 μmmol, Eq: 0.2) and potassium carbonate (67.1 mg, 485 μmmol, Eq: 3) in Dioxane (2.88 ml)/Water (719 μl) was heated to 80° C. for 4 hrs. Dried reaction mixture onto silica gel for purification using an 8-18% EtOAc/Hex gradient. Obtained 2-Cyclohexyl-5-(2,5-dimethyl-2H-pyrazol-3-yl)-1H-indole (14 mg, 24.9% yield) as white solid; MS (M+H)=348.

### Example 223

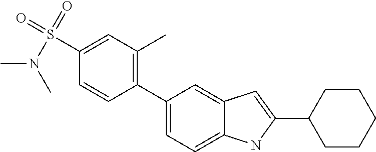

### 4-(2-cyclohexyl-1H-indol-5-yl)-N,N,3-trimethylbenzenesulfonamide

bromo-2-cyclohexyl-1H-indole (100 mg, 359 μmmol, Eq: 1.00), 4-(N,N-dimethylsulfamoyl)-2-methylphenylboronic acid (114 mg, 467 μmmol, Eq: 1.3), tetrakis(triphenylphosphine)palladium (0) (41.5 mg, 35.9 μmmol, Eq: 0.1) and potassium carbonate (149 mg, 1.08 mmol, Eq: 3) in Dioxane (6.39 ml)/Water (1.6 ml) was heated to 93° C. under N₂ for 1.5 hr. Reaction was dried onto silica gel and purified using an EtOAc/Hex gradient. Obtained 4-(2-cyclohexyl-1H-indol-5-yl)-N,N,3-trimethylbenzenesulfonamide (90 mg, 227 μmmol, 63% yield) as a white solid; MS (M+H)=398

### Example 224

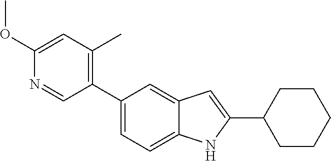

### cyclohexyl-5-(6-methoxy-4-methylpyridin-3-yl)-1H-indole

bromo-2-cyclohexyl-1H-indole (80 mg, 288 μmmol, Eq: 1.00), 6-methoxy-4-methylpyridin-3-ylboronic acid (62.4 mg, 374 μmmol, Eq: 1.3), potassium carbonate (119 mg, 863 mmol, Eq: 3) and tetrakis(triphenylphosphine)palladium (0) (33.2 mg, 28.8 μmmol, Eq: 0.1) in dioxane (5.11 ml)/Water (1.28 ml) was heated to 93° C. for 2 hrs. Dried onto silica gel for purification using a 10-30% EtOAc/Hex gradient. Obtained 2-cyclohexyl-5-(6-methoxy-4-methylpyridin-3-yl)-1H-indole (67 mg, 209 μmmol, 73% yield) as a yellow solid; MS (M+H)=321.

### Example 225

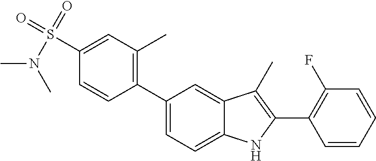

### 4-(2-(2-fluorophenyl)-3-methyl-1H-indol-5-yl)-N,N,3-trimethylbenzenesulfonamide

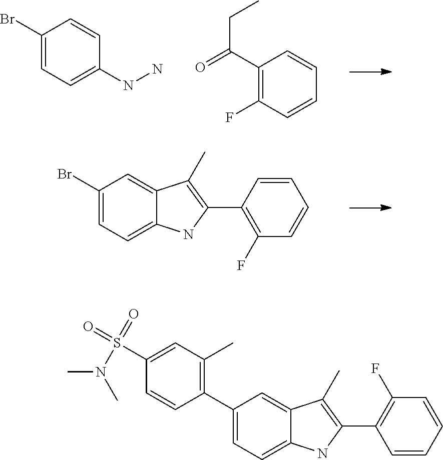

### Step 1: 5-Bromo-2-(2-fluoro-phenyl)-3-methyl-1H-indole

A mixture of (4-bromophenyl)hydrazine hydrochloride (1 g, 4.47 mmol, Eq: 1) and 1-(2-fluorophenyl)propan-1-one (681 mg, 4.47 mmol, Eq: 1) in acetic acid (11.2 mL) was refluxed for 2 hr. Cooled to room temperature and removed acetic acid in vacuo. Extracted with EtOAc, water, brine. Organic layer was collected and purified using a 5% to 30% EtOAc/Hex gradient. Obtained 5-Bromo-2-(2-fluoro-phenyl)-3-methyl-1H-indole (950 mg, 70% yield) as a light orange solid.

### Step 2: 4-(2-(2-fluorophenyl)-3-methyl-1H-indol-5-yl)-N,N,3-trimethylbenzenesulfonamide

bromo-2-(2-fluorophenyl)-3-methyl-1H-indole (100 mg, 329 μmmol, Eq: 1.00), 4-(N,N-dimethylsulfamoyl)-2-methylphenylboronic acid (79.9 mg, 329 μmmol, Eq: 1.00), potassium carbonate (136 mg, 986 μmmol, Eq: 3) tetrakis(triphenylphosphine)palladium (0) (38.0 mg, 32.9 mmol, Eq: 0.1) was heated at 90° C. for 4 hrs. Dried onto silica gel and purified using an EtOAc/Hex gradient. Obtained 4-(2-(2-fluorophenyl)-3-methyl-1H-indol-5-yl)-N,N,3-trimethylbenzenesulfonamide (50 mg, 118 μmmol, 36% yield) as an off-white solid; MS (M+H)=

### Example 226

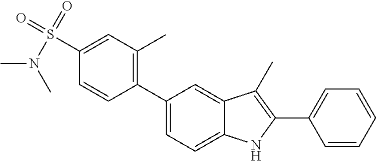

### N,N,3-trimethyl-4-(3-methyl-2-phenyl-1H-indol-5-yl)benzenesulfonamide

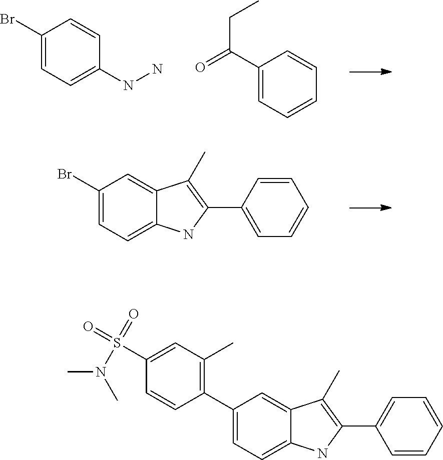

### Step 1

A mixture of (4-bromophenyl)hydrazine hydrochloride (1 g, 4.47 mmol, Eq: 1) and propiophenone (600 mg, 4.47 mmol, Eq: 1) in acetic acid (11.2 mL) was refluxed for 2 hr. Cooled to room temperature and removed acetic acid in vacuo. Extracted with EtOAc, water, brine. Organic layer was collected and purified using a 5% to 30% EtOAc/Hex gradient. Obtained 5-Bromo-3-methyl-2-phenyl-1H-indole (750 mg, 59% yield) as a light brown solid.

### Step 2

A solution of 5-bromo-3-methyl-2-phenyl-1H-indole (77 mg, 269 μmmol, Eq: 1.00), 4-(N, N-dimethylsulphamoyl)-2-methylbenzeneboronic acid (78.5 mg, 323 μmmol, Eq: 1.20) and potassium carbonate (112 mg, 807 μmmol, Eq: 3.0) in Dioxane (3.00 ml) and Water (0.8 ml) was purged with nitrogen (10 min) then 1,1′-bis(diphenylphosphino)ferrocenedichloro palladium(II) (19.7 mg, 26.9 μmmol, Eq: 0.1) was added to the reaction mixture and heated at 110 C for 1 hr. Filtered through a pad of Celite, washed with DCM, solvent removed in vacuo, the residue redissolved in DCM, washed with water, dried (MgSO4). Concentrated, chromatographed (silica gel, 20% EtOAc-Hexane) to give N,N,3-trimethyl-4-(3-methyl-2-phenyl-1H-indol-5-yl)benzenesulfonamide (61 mg, 151 μmmol, 56% yield) as a white powder. LC/MS (M+H)=405

### Example 227

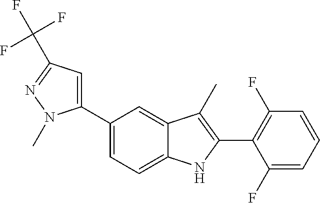

### 2-(2,6-Difluoro-phenyl)-5-(2,5-dimethyl-2H-pyrazol-3-yl)-3-methyl-1H-indole

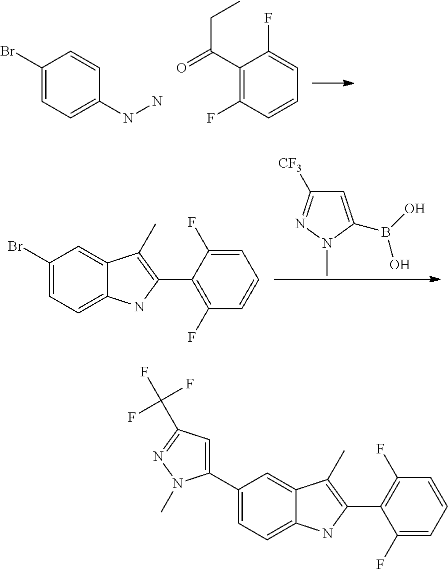

### Step 1: 5-bromo-2-(2,6-difluorophenyl)-3-methyl-1H-indole

A mixture of (4-bromophenyl)hydrazine hydrochloride (1 g, 4.47 mmol, Eq: 1) and 1-(2,6-difluorophenyl)propan-1-one (7611 mg, 4.47 mmol, Eq: 1) in acetic acid (11.2 mL) was refluxed for 2 hr. Cooled to room temperature and precipitated formed. Triturated with both EtOAc and Et2O and filtered off solids. The mother liquor was chromatographed using a 15-50% EtOAc/Hex gradient. Obtained 5-bromo-2-(2,6-difluorophenyl)-3-methyl-1H-indole (1.0 g, 69.8% yield) as a crystalline solid.

### Step 2: 2-(2,6-Difluoro-phenyl)-5-(2,5-dimethyl-2H-pyrazol-3-yl)-3-methyl-1H-indole

bromo-2-(2,6-difluorophenyl)-3-methyl-1H-indole (100 mg, 310 μmmol, Eq: 1.00), 1-methyl-3-(trifluoromethyl)-1H-pyrazol-5-ylboronic acid (78.3 mg, 404 μmmol, Eq: 3), potassium carbonate (129 mg, 931 μmmol, Eq: 3) tetrakis(triphenylphosphine)palladium (0) (31.0 mg, 35.9 mmol, Eq: 0.1) was heated at 93° C. for 2 hrs. Dried onto silica gel and purified using a 10-25% EtOAc/Hex gradient. Obtained 2-(2,6-Difluoro-phenyl)-5-(2,5-dimethyl-2H-pyrazol-3-yl)-3-methyl-1H-indole (56 mg, 46.1% yield) as an off-white solid; MS (M+H)=392

### Example 228

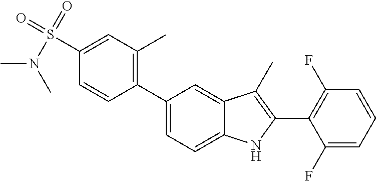

### 4-[2-(2,6-Difluoro-phenyl)-3-methyl-1H-indol-5-yl]-3,N,N-trimethyl-benzenesulfonamide

bromo-2-(2,6-difluorophenyl)-3-methyl-1H-indole (90 mg, 279 μmmol, Eq: 1.00), 4-(N,N-dimethylsulfamoyl)-2-methylphenylboronic acid (88.3 mg, 363 μmmol, Eq: 1.3), tetrakis(triphenylphosphine)palladium (0)) (32.3 mg, 27.9 μmmol, Eq: 0.1) and potassium carbonate (116 mg, 838 μmmol, Eq: 3) in Dioxane (4.97 ml)/Water (1.24 ml) was heated to 93° C. for 1 hr. Dried onto silica gel for purification using a 10-30% EtOAc/Hex gradient. Obtained 4-[2-(2,6-Difluoro-phenyl)-3-methyl-1H-indol-5-yl]-3,N,N-trimethyl-benzenesulfonamide (84 mg, 68.3% yield) as an off-white solid; MS (M+H)=442

### Example 229

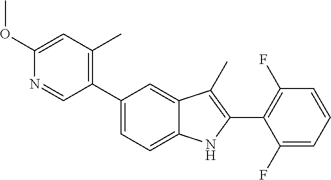

### 2-(2,6-Difluoro-phenyl)-5-(6-methoxy-4-methyl-pyridin-3-yl)-3-methyl-1H-indole

bromo-2-(2,6-difluorophenyl)-3-methyl-1H-indole (90 mg, 279 μmmol, Eq: 1.00), 6-methoxy-4-methylpyridin-3-ylboronic acid (60.6 mg, 363 μmmol, Eq: 1.3), tetrakis(triphenylphosphine)palladium (0)) (32.3 mg, 27.9 μmmol, Eq: 0.1) and potassium carbonate (116 mg, 838 μmmol, Eq: 3) in Dioxane (4.97 ml)/Water (1.24 ml) was heated to 93° C. for 1 hr. Dried onto silica gel for purification using a 10-25% EtOAc/Hex gradient. Obtained 2-(2,6-Difluoro-phenyl)-5-(6-methoxy-4-methyl-pyridin-3-yl)-3-methyl-1H-indole (43 mg, 42.2%) as a crystalline white solid; MS (M+H)=365

### Example 230

### Formulations

Pharmaceutical preparations for delivery by various routes are formulated as shown in the following Tables. “Active ingredient” or “Active compound” as used in the Tables means one or more of the Compounds of Formula I.

Composition for Oral Administration

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration

| Ingredient | Amount | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation

| Ingredient | % wt./wt. |
|---|---|
| Active ingredient | 0.25 g |
| Sodium Chloride | qs to make isotonic |
| Water for injection | 100 ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

Suppository Formulation

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

Topical Formulation

| Ingredients | Grams |
|---|---|
| Active compound | 0.2-2 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy anisole) | 0.01 |
| Water | q.s. 100 |

All of the ingredients, except water, are combined and heated to about 60° C. with stirring. A sufficient quantity of water at about 60° C. is then added with vigorous stirring to emulsify the ingredients, and water then added q.s. about 100 g.

### Nasal Spray Formulations

Several aqueous suspensions containing from about 0.025-0.5 percent active compound are prepared as nasal spray formulations. The formulations optionally contain inactive ingredients such as, for example, microcrystalline cellulose, sodium carboxymethylcellulose, dextrose, and the like. Hydrochloric acid may be added to adjust pH. The nasal spray formulations may be delivered via a nasal spray metered pump typically delivering about 50-100 microliters of formulation per actuation. A typical dosing schedule is 2-4 sprays every 4-12 hours.

### Example 231

### Jurkat IL-2 Production Assay

Cell: Jurkat cell (ATCC) was grown in RPMI 1640 with 10% FBS and 1% penicillin/streptomycin. The cell density was kept at 1.2-1.8×10⁶/mL in culture flask before seeding into culture plate, and the cell density in the plate was 0.5×10⁶/2004/well.

Culture media: RPMI 1640 with 1% FBS or 30% FBS for high serum assay.

Test compound: serial dilution was done in 100% DMSO, and intermediate dilution was done with RPMI 1640 medium with 1% FBS. The DMSO final concentration in culture well was 0.25%.

Stimulant: PHA (Sigma#L9017-10MG) was used for the assay with 1% FBS in culture medium, and added after 10 minutes exposure of cell to compound/DMSO. The PHA final concentration in culture well was 5 μg/mL. PMA (Sigma# P-8139 5MG)/Ionomycin (Sigma# 10634-5MG) was used for the assay with 30% FBS in culture medium, and added at same time point as the 1% FBS culture assay. The final concentration of PMA was 50 ng/mL, and Ionomycin final concentration was 500 ng/mL.

Incubation: at 37° C. with 5% CO₂ and 95% humidity for 18 h˜20 h.

IC50: IC50 was calculated with the data analysis software XLfit4, General Pharmacology model 251.

Using the above procedure, IC₅₀ values for compounds of the invention were calculated and are shown in Table 1:

| | IC50 (nM) | MS |
|---|---|---|
| | Jurkat | (M + H) |
| Example 1 | 46 | 378 |
| Example 2 | 830 | 444 |
| Example 3 | 954 | 426 |
| Example 4 | 219 | 404 |
| Example 5 | 223 | 402 |
| Example 6 | 611 | 382 |
| Example 7 | 800 | 402 |
| Example 8 | 287 | 382 |
| Example 9 | 908 | 387 |
| Example 10 | 190 | 383 |
| Example 11 | 169 | 386 |
| Example 12 | 86 | 402 |
| Example 13 | 66 | 404 |
| Example 14 | 337 | 360 |
| Example 15 | 468 | 400 |
| Example 16 | 211 | 401 |
| Example 17 | 57 | 387 |
| Example 18 | 102 | 401 |
| Example 19 | 198 | 387 |
| Example 20 | 101 | 401 |
| Example 21 | 90 | 404 |
| Example 22 | 150 | 402 |
| Example 23 | 210 | 387 |
| Example 24 | 427 | 373 |
| Example 25 | 224 | 403 |
| Example 26 | 639 | 366 |
| Example 27 | 97 | 345 |
| Example 28 | 123 | 350 |
| Example 29 | 168 | 334 |
| Example 30 | 85 | 378 |
| Example 31 | 174 | 353 |
| Example 32 | 71 | 388 |
| Example 33 | 741 | 394 |
| Example 34 | 184 | 410 |
| Example 35 | 116 | 393 |
| Example 36 | 131 | 377 |
| Example 37 | 120 | 375 |
| Example 38 | 83 | 387 |
| Example 39 | 148 | 414 |
| Example 40 | 728 | 400 |
| Example 41 | 486 | 410 |
| Example 42 | 204 | 394 |
| Example 43 | 87 | 407 |
| Example 44 | 169 | 361 |
| Example 45 | 375 | 362 |
| Example 46 | 44 | 392 |
| Example 47 | 40 | 408 |
| Example 48 | 12 | 394 |
| Example 49 | 13 | 417 |
| Example 50 | 13 | 420 |
| Example 51 | 524 | 403 |
| Example 52 | 62 | 403 |
| Example 53 | 14 | 394 |
| Example 54 | 33 | 382 |
| Example 55 | 79 | 458 |
| Example 56 | 132 | 424 |
| Example 57 | 301 | 420 |
| Example 58 | 689 | 420 |
| Example 59 | 105 | 376 |
| Example 60 | 140 | 356 |
| Example 61 | 848 | 349 |
| Example 62 | 964 | 328 |
| Example 63 | 368 | 350 |
| Example 64 | 81 | 387 |
| Example 65 | 71 | 440 |
| Example 66 | 33 | 434 |
| Example 67 | 63 | 420 |
| Example 68 | 152 | 412 |
| Example 69 | 949 | 367 |
| Example 70 | 356 | 369 |
| Example 71 | 584 | 365 |
| Example 72 | 397 | 367 |
| Example 73 | 60 | 394 |
| Example 74 | 668 | 411 |
| Example 75 | 310 | 369 |
| Example 76 | 108 | 383 |
| Example 77 | 169 | 385 |
| Example 78 | 95 | 399 |
| Example 79 | 139 | 365 |
| Example 80 | 91 | 379 |
| Example 81 | 501 | 385 |
| Example 82 | 367 | 403 |
| Example 83 | 358 | 403 |
| Example 84 | 399 | 387 |
| Example 85 | 149 | 419 |
| Example 86 | 118 | 403 |
| Example 87 | 135 | 419 |
| Example 88 | 908 | 398 |
| Example 89 | 451 | 363 |
| Example 90 | 177 | 366 |
| Example 91 | 456 | 338 |
| Example 92 | 188 | 442 |
| Example 93 | 921 | 368 |
| Example 94 | 175 | 408 |
| Example 95 | 994 | 367 |
| Example 96 | 177 | 452 |
| Example 97 | 72 | 427 |
| Example 98 | 279 | 370 |
| Example 99 | 344 | 404 |
| Example | 307 | 404 |
| 100 | | |
| Example | 593 | 351 |
| 101 | | |
| Example | 74 | 387 |
| 102 | | |
| Example | 176 | 403 |
| 103 | | |
| Example | 178 | 405 |
| 104 | | |
| Example | 664 | 405 |
| 105 | | |
| Example | 488 | 453 |
| 106 | | |
| Example | 32 | 403 |
| 107 | | |
| Example | 601 | 376 |
| 108 | | |
| Example | 399 | 382 |
| 109 | | |
| Example | 190 | 377 |
| 110 | | |
| Example | 318 | 388 |
| 111 | | |
| Example | 843 | 333 |
| 112 | | |
| Example | 60 | 351 |
| 113 | | |
| Example | 39 | 388 |
| 114 | | |
| Example | 38 | 404 |
| 115 | | |
| Example | 12 | 404 |
| 116 | | |
| Example | 19 | 395 |
| 117 | | |
| Example | 180 | 394 |
| 118 | | |
| Example | 14 | 405 |
| 119 | | |
| Example | 19 | 419 |
| 120 | | |
| Example | 12 | 367 |
| 121 | | |
| Example | 260 | 367 |
| 122 | | |
| Example | 448 | 367 |
| 123 | | |
| Example | 11 | 418 |
| 124 | | |
| Example | 49 | 410 |
| 125 | | |
| Example | 106 | 376 |
| 126 | | |
| Example | 991 | 375 |
| 127 | | |
| Example | 776 | 361 |
| 128 | | |
| Example | 237 | 343 |
| 129 | | |
| Example | 16 | 426 |
| 130 | | |
| Example | 149 | 414 |
| 131 | | |
| Example | 319 | 373 |
| 132 | | |
| Example | 976 | 405 |
| 133 | | |
| Example | 163 | 352 |
| 134 | | |
| Example | 283 | 375 |
| 135 | | |
| Example | 370 | 327 |
| 136 | | |
| Example | 132 | 348 |
| 137 | | |
| Example | 101 | 368 |
| 138 | | |
| Example | 162 | 354 |
| 139 | | |
| Example | 142 | 374 |
| 140 | | |
| Example | 660 | 345 |
| 141 | | |
| Example | 167 | 375 |
| 142 | | |
| Example | 182 | 354 |
| 143 | | |
| Example | 96 | 324 |
| 144 | | |
| Example | 89 | 324 |
| 145 | | |
| Example | 150 | 329 |
| 146 | | |
| Example | 311 | 324 |
| 147 | | |
| Example | 547 | 344 |
| 148 | | |
| Example | 286 | 329 |
| 149 | | |
| Example | 618 | 313 |
| 150 | | |
| Example | 158 | 328 |
| 151 | | |
| Example | 731 | 358 |
| 152 | | |
| Example | 331 | 419 |
| 153 | | |
| Example | 586 | 405 |
| 154 | | |
| Example | 87 | 428 |
| 155 | | |
| Example | 130 | 419 |
| 156 | | |
| Example | 222 | 361 |
| 157 | | |
| Example | 16 | 361 |
| 158 | | |
| Example | 25 | 443 |
| 159 | | |
| Example | 18 | 371 |
| 160 | | |
| Example | 99 | 362 |
| 161 | | |
| Example | 34 | 362 |
| 162 | | |
| Example | 108 | 411 |
| 163 | | |
| Example | 33 | 381 |
| 164 | | |
| Example | 316 | 422 |
| 165 | | |
| Example | 518 | 366 |
| 166 | | |
| Example | 34 | 444 |
| 167 | | |
| Example | 44 | 429 |
| 168 | | |
| Example | 117 | 485 |
| 169 | | |
| Example | 170 | 499 |
| 170 | | |
| Example | 17 | 418 |
| 171 | | |
| Example | 15 | 377 |
| 172 | | |
| Example | 12 | 415 |
| 173 | | |
| Example | 22 | 386 |
| 174 | | |
| Example | 11 | 434 |
| 175 | | |
| Example | 14 | 421 |
| 176 | | |
| Example | 82 | 421 |
| 177 | | |
| Example | 59 | 434 |
| 178 | | |
| Example | 23 | 435 |
| 179 | | |
| Example | 28 | 448 |
| 180 | | |
| Example | 56 | 449 |
| 181 | | |
| Example | 142 | 502 |
| 182 | | |
| Example | 28 | 419 |
| 183 | | |
| Example | 148 | 405 |
| 184 | | |
| Example | 65 | 401 |
| 185 | | |
| Example | 34 | 435 |
| 186 | | |
| Example | 69 | 420 |
| 187 | | |
| Example | 44 | 406 |
| 188 | | |
| Example | 76 | 371 |
| 189 | | |
| Example | 111 | 401 |
| 190 | | |
| Example | 67 | 371 |
| 191 | | |
| Example | 149 | 375 |
| 192 | | |
| Example | 129 | 401 |
| 193 | | |
| Example | 139 | 421 |
| 194 | | |
| Example | 782 | 360 |
| 195 | | |
| Example | 24 | 362 |
| 196 | | |
| Example | 110 | 364 |
| 197 | | |
| Example | 95 | 364 |
| 198 | | |
| Example | 86 | 364 |
| 199 | | |
| Example | 220 | 405 |
| 200 | | |
| Example | 35 | 424 |
| 201 | | |
| Example | 57 | 391 |
| 202 | | |
| Example | 31 | 438 |
| 203 | | |
| Example | 63 | 402 |
| 204 | | |
| Example | 24 | 402 |
| 205 | | |
| Example | 51 | 405 |
| 206 | | |
| Example | 26 | 418 |
| 207 | | |
| Example | 97 | 388 |
| 208 | | |
| Example | 54 | 414 |
| 209 | | |
| Example | 60 | 399 |
| 210 | | |
| Example | 124 | 381 |
| 211 | | |
| Example | 447 | 325 |
| 212 | | |
| Example | 105 | 340 |
| 213 | | |
| Example | 41 | 378 |
| 214 | | |
| Example | 235 | 334 |
| 215 | | |
| Example | 110 | 330 |
| 216 | | |
| Example | 100 | 436 |
| 217 | | |
| Example | 110 | 396 |
| 218 | | |
| Example | 158 | 396 |
| 219 | | |
| Example | 24 | 400 |
| 220 | | |
| Example | 95 | 400 |
| 221 | | |
| Example | 40 | 348 |
| 222 | | |
| Example | 54 | 398 |
| 223 | | |
| Example | 83 | 321 |
| 224 | | |
| Example | 153 | 424 |
| 225 | | |
| Example | 602 | 405 |
| 226 | | |
| Example | 284 | 392 |
| 227 | | |
| Example | 115 | 442 |
| 228 | | |
| Example | 164 | 365 |
| 229 | | |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

What is claimed is:

1. A compound of formula I: 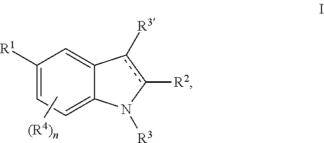
wherein:
R¹ is:
phenyl substituted one, two or three times with a group or groups independently selected from: C₁₋₆alkyl; C₁₋₆alkoxy; halo; halo-C₁₋₆alkyl; halo-C₁₋₆alkoxy; nitrile; acetyl; C₁₋₆alkoxycarbonyl; aminocarbonyl; aminosulfonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; hydroxy; sulfonylmorpholine; sulfonylmethylpiperazine; heterocyclyl; phenyl which may be optionally substituted; or heteroaryl which may be optionally substituted;
pyridinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; heterocyclyl; phenyl which may be optionally substituted; or heteroaryl which may be optionally substituted;
pyrimidinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; heterocyclyl; phenyl which may be optionally substituted; or heteroaryl which may be optionally substituted; or
a five-membered heteroaryl ring optionally substituted one, two or three times with a group or groups independently selected from: C₁₋₆alkyl; C₃₋₆cycloalkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; heterocyclyl; phenyl which may be optionally substituted; and heteroaryl which may be optionally substituted; or two of said substituents together with the atoms to which they are attached may form a phenyl fused to the five-membered heteroaryl ring;
R² is:
C₃₋₆cycloalkyl;
phenyl substituted one, two or three times with a group or groups independently selected from: C₁₋₆alkyl; C₁₋₆alkoxy; C₁₋₆alkoxyhydroxy; halo; halo-C₁₋₆alkyl; halo-C₁₋₆alkoxy; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; C₁₋₆alkylcarbonylhydroxy; C₁₋₆alkoxycyano; amino; hydroxy; phenyl which may be optionally substituted; or heteroaryl which may be optionally substituted;
pyridinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; phenyl which may be optionally substituted; or heteroaryl which may be optionally substituted;
pyrimidinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; phenyl which may be optionally substituted; or heteroaryl which may be optionally substituted; or
a five-membered heteroaryl ring optionally substituted once or twice with a group or groups independently selected from: C₁₋₆alkyl; C₁₋₆alkoxy; halo; halo-C₁₋₆alkyl; C₃₋₆cycloalkyl; halo-C₁₋₆alkoxy; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; phenyl which may be optionally substituted; and heteroaryl which may be optionally substituted; or two of said substituents together with the atoms to which they are attached may form a phenyl fused to said five-membered heteroaryl ring;
R³ is hydrogen;
R^{3′} is hydrogen or C₁₋₆alkyl;
n is from 0 to 3;
each R⁴ is independently selected from: hydrogen; C₁₋₆alkyl; C₁₋₆alkoxy; halo; and halo-C₁₋₆alkyl; and
said dashed line is a bond or absent,
or a pharmaceutically acceptable salt thereof.

2. The compound according to claim 1, wherein R¹ is phenyl substituted one, two or three times with a group or groups independently selected from: C₁₋₆alkyl; C₁₋₆alkoxy; halo; halo-C₁₋₆alkyl; halo-C₁₋₆alkoxy; nitrile; acetyl; C₁₋₆alkoxycarbonyl; aminocarbonyl; aminosulfonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; hydroxy; sulfonylmorpholine; sulfonylmethylpiperazine; heterocyclyl; phenyl which may be optionally substituted once or twice with a group or groups independently selected from halo, C₁₋₆alkyl, halo-C₁₋₆alkyl or C₁₋₆alkoxy; and heteroaryl which may be optionally substituted once or twice with a group or groups independently selected from halo, C₁₋₆alkyl, or halo-C₁₋₆alkyl.

3. The compound according to claim 1, wherein R¹ is phenyl substituted one, two or three times with a group or groups independently selected from: C₁₋₆alkyl; C₁₋₆alkoxy; halo; halo-C₁₋₆alkyl; halo-C₁₋₆alkoxy; nitrile; acetyl; C₁₋₆alkoxycarbonyl; aminocarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁-6alkyl; amino; hydroxy; sulfonylmorpholine; sulfonylmethylpiperazine; heterocyclyl selected from pyrrolidinyl, piperidinyl, piperazinyl, imidazolidinyl or isothiazolidinyl, said heterocyclyl being optionally substituted with oxo or C₁₋₆alkyl; phenyl which may be optionally substituted once or twice with a group or groups independently selected from halo, cyano, C₁₋₆alkyl, halo-C₁₋₆alkyl or C₁₋₆alkoxy; and heteroaryl selected from pyridinyl, pyrimidinyl, pyrazinyl, pyridazinyl, pyrazolyl, imidazolyl, oxazolyl, thiazolyl, isoxazolyl, isothiazolyl, furanyl or thienyl, said heteroaryl being optionally substituted once or twice with a group or groups independently selected from halo, oxo, C₁₋₆alkyl, or halo-C₁₋₆alkyl.

4. The compound according to claim 1, wherein R¹ is: 2-chloro-5-trifluoromethyl-phenyl, 3-trifluoromethyl-phenyl, 5-methoxycarbonyl-2-methyl-phenyl, 2-methanesulfanyl-phenyl, 4-chloro-phenyl, 3-cyano-phenyl, 3-chloro-4-fluoro-phenyl, 3-methylcarbonyl-amino-phenyl, 4-methoxycarbonyl-phenyl, 2,5-dimethoxy-phenyl, 2-methoxy-5-trifluoromethyl-phenyl, 2-trifluoromethyl-phenyl, 2-methyl-5-thiazol-2-yl-phenyl, 3-oxazol-2-yl-phenyl, 2-chloro-4-methoxycarbonyl-phenyl, 4-amino-2-methyl-phenyl, 2,4-dimethoxy-phenyl, 2-methyl-4-fluoro-phenyl, 2,4-di-trifluoromethyl-phenyl, 2-methyl-4-trifluoromethoxy-phenyl, 4-aminocarbonyl-2-methyl-phenyl, 4-methanesulfonyl-2-trifluoromethyl-phenyl, 4-amino-2-chloro-phenyl, 2-chloro-4-methoxy-phenyl, 2-methyl-4-trifluoromethyl-phenyl, 4-dimethylaminosulfonyl-2-methyl-phenyl, 4-hydroxy-2-methyl-phenyl, 4-methoxy-2-trifluoromethyl-phenyl, 2-chloro-4-trifluoromethyl-phenyl, 4-(2,4-dihydro-[1,2,4]triazol-3-one-1-yl)-2-methyl-phenyl, 2-methyl-4-(5-methyl-tetrazol-1-yl)-phenyl, 2-methyl-4-(pyrrolidin-3-one-1-yl-phenyl, 4-([1,3,5]triazin-2-yl)-2-methyl-phenyl, 2-methyl-4-(tetrazol-1-yl)-phenyl, 4-(1,1-dioxo-1lambda*6*-isothiazolidin-2-yl)-2-methyl-phenyl, 2-methyl-4-(piperidin-2-one-1-yl)-phenyl, 2-methyl-4-(piperidin-4-one-1-yl)-phenyl, 2-methyl-4-(piperidin-2,6-dione-1-yl)-phenyl, 2-methyl-4-(pyrrolidin-2-one-1-yl-phenyl, 2-methyl-4-(pyrrolidin-2,5-dione-1-yl-phenyl, 2-trifluoromethyl-4-(pyrrolidin-1-yl)-phenyl, 2-methyl-5-oxazol-2-yl-phenyl, 3-thiazol-2-yl-phenyl, 4-cyano-2-methyl-phenyl, 4-methoxy-2-methyl-phenyl,2,4-dimethyl-phenyl, 4-methoxycarbonyl-2-methyl-phenyl, 4-chloro-2-methyl-phenyl, 4-cyano-phenyl, 4-methyl-phenyl, or 4-chloro-phenyl.

5. The compound according to claim 1, wherein R¹ is substituted phenyl of formula A1 or A2 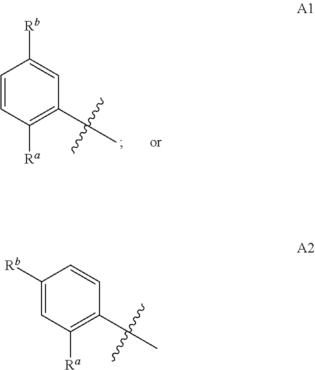
wherein:
R^{a} is: hydrogen; halo; C₁₋₆alkyl; halo-C₁₋₆alkyl; C₁₋₆alkylsulfanyl; or C₁₋₆alkoxy; and
R^{b} is: halo; halo-C₁₋₆alkyl; C₁₋₆alkoxy; halo-C₁₋₆alkoxy; cyano; amino; C₁₋₆alkoxy-carbonyl; amino; aminocarbonyl; aminosulfonyl; hydroxy; heterocyclyl; C₁₋₆alkylsulfonyl; hydroxy; or a 5-membered heteroaryl that is optionally substituted once or twice with a group or groups independently selected from halo, oxo, C₁₋₆alkyl, or halo-C₁₋₆alkyl.

6. The compound according to claim 1, wherein R¹ is pyridinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; heterocyclyl; phenyl which may be optionally substituted; or heteroaryl which may be optionally substituted.

7. The compound according to claim 1, wherein R¹ is a five-membered heteroaryl ring optionally substituted one, two or three times with a group or groups independently selected from: C₁₋₆alkyl; C₃₋₆cycloalkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁-6alkyl; hydroxy-C₁₋₆alkyl; amino; oxo; hydroxy; heterocyclyl; phenyl which may be optionally substituted; and heteroaryl which may be optionally substituted; or two of said substituents together with the atoms to which they are attached may form a phenyl fused to the five-membered heteroaryl ring.

8. The compound according to claim 1, wherein R¹ is a five-membered heteroaryl ring selected from: tetrazolyl; triazolyl; oxadiazolyl; thiadiazolyl; pyrazolyl; imidazolyl; thiazolyl; isothiazolyl; oxazolyl; isoxazolyl; pyrrolyl; furanyl; or thienyl; each optionally substituted one, two or three times with a group or groups independently selected from C₁₋₆alkyl, C₃₋₆cycloalkyl, C₁₋₆alkoxy, halo, halo-C₁₋₆alkyl, nitrile, acetyl, C₁₋₆alkoxycarbonyl, C₁₋₆alkylcarbonylamino, C₁₋₆alkyl-sulfanyl, C₁₋₆alkyl-sulfonyl, C₁₋₆alkoxy-C₁₋₆alkyl, hydroxy-C₁₋₆alkyl, oxo, phenyl which may be optionally substituted, and heteroaryl (such as pyridinyl) which may be optionally substituted, or two of said substituents together with the atoms to which they are attached may form a phenyl fused to said five-membered heteroaryl ring.

9. The compound according to claim 1, wherein R¹ is: 5-methyl-2-pyridin-2-yl-thiazol-4-yl; 4-methyl-2-phenyl-thiazol-5-yl; 5-methyl-2-pyridin-3-yl-thiazol-4-yl; 2-methyl-5-pyridin-2-yl-2H-pyrazol-3-yl; 2-ethyl-5-pyridin-2-yl-2H-pyrazol-3-yl; 2-methyl-5-pyridin-4-yl-2H-pyrazol-3-yl; 2-ethyl-5-phenyl-2H-pyrazol-3-yl; 2-methyl-5-pyridin-3-yl-2H-pyrazol-3-yl; 5-methyl-2-phenyl-thiazol-4-yl; 2-methyl-5-phenyl-2H-pyrazol-3-yl; 2-methyl-5-trifluoromethyl-2H-pyrazol-3-yl; 2-ethyl-5-phenyl-2H-pyrazol-3-yl; 2-ethyl-5-pyridin-3-yl-2H-pyrazol-3-yl; 2-ethyl-5-pyridin-2-yl-2H-pyrazol-3-yl; 2-ethyl-5-pyridin-4-yl-2H-pyrazol-3-yl; 2-methyl-5-phenyl-2H-pyrazol-3-yl; 2-methyl-5-pyridin-2-yl-2H-pyrazol-3-yl; 2-methyl-5-pyridin-3-yl-2H-pyrazol-3-yl; 2-methyl-5-pyridin-4-yl-2H-pyrazol-3-yl; 2-ethyl-5-methyl-thiazol-4-yl; 2-cyclopropyl-5-methyl-thiazol-4-yl; 2-isopropyl-5-methyl-thiazol-4-yl, 5-methyl-2-pyridin-4-yl-thiazol-4-yl, 1,4-dimethyl-1H-imidazol-2-yl, 2-methyl-5-pyridin-2-yl -2H-pyrazol-3-yl, 3-cyano-1-methyl-1H-pyrazol-4-yl, 1-methyl-3-trifluoromethyl-1H-pyrazol-4-yl, 5-methyl-2-oxazol-2-yl-thiazol-4-yl, 5-methyl-2-(tetrahydro-pyran-4-yl, 1,3-dimethyl-1H-pyrazol-4-yl, 5-cyclopropyl-2-methyl-2H-pyrazol-3-yl, or 2,5-dimethyl-2H-pyrazol-3-yl.

10. The compound according to claim 1, wherein R² is phenyl substituted one, two or three times with a group or groups independently selected from: C₁₋₆alkyl; C₁₋₆alkoxy; C₁₋₆alkoxyhydroxy; halo; halo-C₁₋₆alkyl; halo-C₁₋₆alkoxy; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; C₁₋₆alkylcarbonylhydroxy; C₁₋₆alkoxycyano; amino; hydroxy; phenyl which may be optionally substituted; or heteroaryl which may be optionally substituted.

11. The compound according to claim 1, wherein R² is halo-phenyl or dihalo-phenyl.

12. The compound according to claim 2, wherein R² is 2,6-difluoro-phenyl, 2-chloro-phenyl, 2-fluoro-phenyl, 4-chloro-phenyl, 2-chloro-6-fluoro-phenyl, 3-chloro-2-fluoro-phenyl, 2,5-dichloro-phenyl, 5-chloro-2-fluoro-phenyl, 2-chloro-4-fluoro-phenyl, 2-chloro-5-fluoro-phenyl, 2,6-dichlorophenyl, 2,3-difluoro-phenyl, 2,3-dichloro-phenyl, 2-methoxy-phenyl, 2-methyl-phenyl, 4-methoxycarbonyl-2-methyl-phenyl, or 4-trifluoromethoxy-phenyl.

13. The compound according to claim 1, wherein R² is pyridinyl optionally substituted once or twice with a group or groups independently selected from: C₁₋₆alkyl; C₁₋₆alkoxy; halo; halo-C₁₋₆alkyl; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; phenyl which may be optionally substituted; or heteroaryl which may be optionally substituted.

14. The compound according to claim 1, wherein R² is pyridin-4-yl, 3-fluoro-pyridin-4-yl, 3-methyl-pyridin-4-yl, 2-methyl-pyridin-3-yl, or 2-methoxy-pyridin-3-yl.

15. The compound according to claim 1, wherein R² is a five-membered heteroaryl ring optionally substituted once or twice with a group or groups independently selected from: C₁₋₆alkyl; C₁₋₆alkoxy; halo; halo-C₁₋₆alkyl; C₃₋₆cycloalkyl; halo-C₁₋₆alkoxy; nitrile; acetyl; C₁₋₆alkoxycarbonyl; C₁₋₆alkylcarbonylamino; C₁₋₆alkyl-sulfanyl; C₁₋₆alkyl-sulfonyl; C₁₋₆alkoxy-C₁₋₆alkyl; hydroxy-C₁₋₆alkyl; amino; oxo; hydroxy; phenyl which may be optionally substituted; and heteroaryl which may be optionally substituted; or two of said substituents together with the atoms to which they are attached may form a phenyl fused to said five-membered heteroaryl ring.

16. The compound according to claim 1, wherein R^{3′} is hydrogen.

17. The compound according to claim 1, wherein R^{3′} is methyl.

18. The compound according to claim 1, wherein n is 0.

19. The compound according to claim 1, wherein said dashed line is a bond.

20. The compound according to claim 1, wherein said compound is:
2-(2,6-Difluoro-phenyl)-5-(2-methyl-5-trifluoromethyl-2H-pyrazol-3-yl)-1H-indole;
1-[2-(2,6-Difluoro-phenyl)-1H-indol-5-yl]-5-methoxy-2-trifluoromethyl-1H-benzoimidazole;
5-(2-Methyl-5-trifluoromethyl-2H-pyrazol-3-yl)-2-(4-trifluoromethoxy-phenyl)-1H-indole;
2-(2,6-Difluoro-phenyl)-5-(5-methyl-2-pyridin-2-yl-thiazol-4-yl)-1H-indole;
2-(2-Chloro-phenyl)-5-(5-methyl-2-pyridin-2-yl-thiazol-4-yl)-1H-indole;
5-(5-Methyl-2-pyridin-2-yl-thiazol-4-yl)-2-o-tolyl-1H-indole;
2-(2-Chloro-phenyl)-5-(4-methyl-2-phenyl-thiazol-5-yl)-1H-indole;
5-(4-Methyl-2-phenyl-thiazol-5-yl)-2-(2-methyl-pyridin-3-yl)-1H-indole;
2-(3-Fluoro-pyridin-4-yl)-5-(5-methyl-2-pyridin-2-yl-thiazol-4-yl)-1H-indole;
2-(3-Methyl-pyridin-4-yl)-5-(5-methyl-2-pyridin-2-yl-thiazol-4-yl)-1H-indole;
2-(2-Fluoro-phenyl)-5-(5-methyl-2-pyridin-3-yl-thiazol-4-yl)-1H-indole;
2-(2-Chloro-phenyl)-5-(5-methyl-2-pyridin-3-yl-thiazol-4-yl)-1H-indole;
2-(2,6-Difluoro-phenyl)-5-(5-methyl-2-pyridin-3-yl-thiazol-4-yl)-1H-indole;
2-(2-Fluoro-phenyl)-5-(2-methyl-5-trifluoromethyl-2H-pyrazol-3-yl)-1H-indole;
2-(2,6-difluoro-phenyl)-5-(2-ethyl-5-phenyl-2H-pyrazol-3-yl)-1H-indole;
2-(2,6-Difluoro-phenyl)-5-(2-ethyl-5-pyridin-2-yl-2H-pyrazol-3-yl)-1H-indole;
2-(2,6-Difluoro-phenyl)-5-(2-methyl-5-pyridin-4-yl-2H-pyrazol-3-yl)-1H-indole;
2-(2,6-Difluoro-phenyl)-5-(2-ethyl-5-pyridin-4-yl-2H-pyrazol-3-yl)-1H-indole;
2-(2,6-Difluoro-phenyl)-5-(2-methyl-5-pyridin-3-yl-2H-pyrazol-3-yl)-1H-indole; or
2-(2,6-Difluoro-phenyl)-5-(2-ethyl-5-pyridin-3-yl-2H-pyrazol-3-yl)-1H-indole.

21. The compound according to claim 1, wherein said compound is:
2-(2,6-Difluoro-phenyl)-5-(5-methyl-2-pyridin-4-yl-thiazol-4-yl)-1H-indole;
2-(2-Chloro-phenyl)-5-(5-methyl-2-pyridin-4-yl-thiazol-4-yl)-1H-indole;
2-(2,6-Difluoro-phenyl)-5-(2-methyl-5-oxazol-2-yl-phenyl)-1H-indole;
2-(2,6-Difluoro-phenyl)-5-(3-oxazol-2-yl-phenyl)-1H-indole;
2-(2,6-Difluoro-phenyl)-5-(2-methyl-5-thiazol-2-yl-phenyl)-1H-indole;
2-(2,6-Difluoro-phenyl)-5-(2,5-dimethoxy-phenyl)-1H-indole;
4-[2-(2,6-Difluoro-phenyl)-1H-indol-5-yl]-3-methyl-benzonitrile;
2-(2,6-Difluoro-phenyl)-5-(4-methoxy-2-methyl-phenyl)-1H-indole;
2-(2,6-Difluoro-phenyl)-5-(2,4-dimethyl-phenyl)-1H-indole;
4-[2-(2,6-Difluoro-phenyl)-1H-indol-5-yl]-3-methyl-benzoic acid methyl ester;
5-(4-Chloro-2-methyl-phenyl)-2-(2,6-difluoro-phenyl)-1H-indole;
2-(2,6-Difluoro-phenyl)-5-(2-methyl-4-trifluoromethyl-phenyl)-1H-indole;
2-(5-Chloro-2-fluoro-phenyl)-5-(2-methyl-5-trifluoromethyl-2H-pyrazol-3-yl)-1H-indole;
2-(2,4-Dichloro-phenyl)-5-(2-methyl-5-trifluoromethyl-2H-pyrazol-3-yl)-1H-indole;
2-(2-Chloro-4-fluoro-phenyl)-5-(2-methyl-5-trifluoromethyl-2H-pyrazol-3-yl)-1H-indole;
2-(3-Chloro-pyridin-4-yl)-5-(2-methyl-5-trifluoromethyl-2H-pyrazol-3-yl)-1H-indole;
2-(3-Methyl-pyridin-4-yl)-5-(2-methyl-5-trifluoromethyl-2H-pyrazol-3-yl)-1H-indole;
2-(6-Methoxy-2-methyl-pyridin-3-yl)-5-(2-methyl-5-trifluoromethyl-2H-pyrazol-3-yl)-1H-indole;
Methyl-4-[5-(2-methyl-5-trifluoromethyl-2H-pyrazol-3-yl)-1H-indol-2-yl]-benzoic acid methyl ester; or
Methyl-4-[5-(2-methyl-5-trifluoromethyl-2H-pyrazol-3-yl)-1H-indol-2-yl]-benzoic acid methyl ester.

22. The compound according to claim 1, wherein said compound is:
2-(2,3-Dichloro-phenyl)-5-(2-methyl-5-trifluoromethyl-2H-pyrazol-3-yl)-1H-indole;
2-(2-Chloro-5-fluoro-phenyl)-5-(2-methyl-5-trifluoromethyl-2H-pyrazol-3-yl)-1H-indole;
2-(3-Chloro-2-methoxy-pyridin-4-yl)-5-(2-methyl-5-trifluoromethyl-2H-pyrazol-3-yl)-1H-indole;
2-(3-Fluoro-pyridin-4-yl)-5-(2-methyl-5-trifluoromethyl-2H-pyrazol-3-yl)-1H-indole;
2-(3,5-Dimethyl-isoxazol-4-yl)-5-(2-methyl-5-trifluoromethyl-2H-pyrazol-3-yl)-1H-indole;
2-(2,6-Difluoro-phenyl)-5-(2-ethyl-5-trifluoromethyl-2H-pyrazol-3-yl)-1H-indole;
2-(2-Chloro-6-fluoro-phenyl)-5-(2-ethyl-5-trifluoromethyl-2H-pyrazol-3-yl)-1H-indole;
2-(2-Chloro-6-fluoro-phenyl)-5-(2-methyl-5-trifluoromethyl-2H-pyrazol-3-yl)-1H-indole;
2-(2-Chloro-6-fluoro-phenyl)-5-(2-ethyl-5-pyridin-3-yl-2H-pyrazol-3-yl)-1H-indole;
2-(2-Chloro-6-fluoro-phenyl)-5-(5-methyl-2-pyridin-3-yl-thiazol-4-yl)-1H-indole;
2-(2-Chloro-6-fluoro-phenyl)-5-(2-methyl-5-pyridin-4-yl-2H-pyrazol-3-yl)-1H-indole;
2-(2-Chloro-6-fluoro-phenyl)-5-(2-methyl-4-oxazol-2-yl-phenyl)-1H-indole;
4-[2-(2-Chloro-6-fluoro-phenyl)-1H-indol-5-yl]-3-methyl-benzoic acid methyl ester;
2-(2-chloro-6-fluoro-phenyl)-5-(2,4-dimethoxy-phenyl)-1H-indole;
5-(2,4-Bis-trifluoromethyl-phenyl)-2-(2-chloro-6-fluoro-phenyl)-1H-indole;
2-(2-Chloro-6-fluoro-phenyl)-5-(2-chloro-4-trifluoromethyl-phenyl)-1H-indole;
2-(2-Chloro-4-fluoro-phenyl)-5-(5-methyl-2-pyridin-2-yl-thiazol-4-yl)-1H-indole;
2-(2-Chloro-5-fluoro-phenyl)-5-(5-methyl-2-pyridin-2-yl-thiazol-4-yl)-1H-indole;
2-(2-Chloro-phenyl)-5-(2-methyl-5-trifluoromethyl-2H-pyrazol-3-yl)-1H-indole; or
5-(2-Methyl-5-trifluoromethyl-2H-pyrazol-3-yl)-2-o-tolyl-1H-indole.

23. The compound according to claim 1, wherein said compound is:
2-(2-Chloro-phenyl)-5-(5-cyclopropyl-2-methyl-2H-pyrazol-3-yl)-1H-indole;
5-(5-Cyclopropyl-2-methyl-2H-pyrazol-3-yl)-2-o-tolyl-1H-indole;
5-(5-Cyclopropyl-2-methyl-2H-pyrazol-3-yl)-2-(2,6-difluoro-phenyl)-1H-indole;
2-(3-Fluoro-pyridin-4-yl)-5-(5-methyl-2-pyridin-3-yl-thiazol-4-yl)-1H-indole;
Methyl-4-[5-(5-methyl-2-pyridin-3-yl-thiazol-4-yl)-1H-indol-2-yl]-benzoic acid methyl ester;
2-(2,6-Difluoro-4-methoxy-phenyl)-5-(5-methyl-2-pyridin-3-yl-thiazol-4-yl)-1H-indole;
2-(2-Chloro-4-fluoro-phenyl)-5-(5-methyl-2-pyridin-3-yl-thiazol-4-yl)-1H-indole;
2-(4-Isopropyl-pyrimidin-5-yl)-5-(5-methyl-2-pyridin-3-yl-thiazol-4-yl)-1H-indole;
2-(2-Chloro-phenyl)-5-(2-isopropyl-5-methyl-thiazol-4-yl)-1H-indole;
2-(2,6-Difluoro-phenyl)-5-(2-isopropyl-5-methyl-thiazol-4-yl)-1H-indole;
2-(2,6-Difluoro-phenyl)-5-(2-isopropyl-5-methyl-thiazol-4-yl)-1H-indole;
5-(2-Cyclopropyl-5-methyl-thiazol-4-yl)-2-(2,6-difluoro-phenyl)-1H-indole;
2-(2,6-Difluoro-phenyl)-5-(5-methyl-2-oxazol-2-yl-thiazol-4-yl)-1H-indole;
2-(2,6-Difluoro-phenyl)-5-[5-methyl-2-(tetrahydro-pyran-4-yl)-thiazol-4-yl]-1H-indole;
2-(2-Fluoro-phenyl)-5-(2-methyl-5-pyridin-3-yl-2H-pyrazol-3-yl)-1H-indole;
2-(2-Fluoro-phenyl)-5-(2-ethyl-5-pyridin-3-yl-2H-pyrazol-3-yl)-1H-indole;
2-(2-Chloro-phenyl)-5-(2-methyl-5-pyridin-3-yl-2H-pyrazol-3-yl)-1H-indole;
2-(2-Chloro-phenyl)-5-(2-ethyl-5-pyridin-3-yl-2H-pyrazol-3-yl)-1H-indole;
5-(2-Methyl-5-pyridin-3-yl-2H-pyrazol-3-yl)-2-o-tolyl-1H-indole; or
5-(2-Ethyl-5-pyridin-3-yl-2H-pyrazol-3-yl)-2-o-tolyl-1H-indole.

24. The compound according to claim 1, wherein said compound is:
2-(2-Chloro-phenyl)-5-(2-methyl-5-pyridin-2-yl-2H-pyrazol-3-yl)-1H-indole;
2-(2-Chloro-5-fluoro-phenyl)-5-(2-methyl-5-pyridin-2-yl-2H-pyrazol-3-yl)-1H-indole;
2-(2-Chloro-4-fluoro-phenyl)-5-(2-methyl-5-pyridin-2-yl-2H-pyrazol-3-yl)-1H-indole;
2-(2,3-Difluoro-phenyl)-5-(2-methyl-5-pyridin-3-yl-2H-pyrazol-3-yl)-1H-indole;
2-(2,3-Dichloro-phenyl)-5-(2-methyl-5-pyridin-3-yl-2H-pyrazol-3-yl)-1H-indole;
2-(2-Chloro-4-fluoro-phenyl)-5-(2-methyl-5-pyridin-3-yl-2H-pyrazol-3-yl)-1H-indole;
2-(2,5-Dichloro-phenyl)-5-(2-methyl-5-pyridin-3-yl-2H-pyrazol-3-yl)-1H-indole;
4-[2-(2,6-Difluoro-phenyl)-1H-indol-5-yl]-3-chloro-benzoic acid methyl ester;
4-[2-(2,6-Difluoro-phenyl)-1H-indol-5-yl]-3-methyl-benzamide;
2-(2,6-Difluoro-phenyl)-5-(2,4-dimethoxy-phenyl)-1H-indole;
2-(2,6-Difluoro-phenyl)-5-(4-fluoro-2-methyl-phenyl)-1H-indole;
5-(2,4-Bis-trifluoromethyl-phenyl)-2-(2,6-difluoro-phenyl)-1H-indole;
2-(2,6-Difluoro-phenyl)-5-(2,4-dimethoxy-pyrimidin-5-yl)-1H-indole;
5-(2-Chloro-4-trifluoromethyl-phenyl)-2-(2,6-difluoro-phenyl)-1H-indole;
2-(2,6-Difluoro-phenyl)-5-(2,6-dimethoxy-pyridin-3-yl)-1H-indole;
2-(2,6-Difluoro-phenyl)-5-(4-methanesulfonyl-2-trifluoromethyl-phenyl)-1H-indole;
4-[2-(2,6-Difluoro-phenyl)-1H-indol-5-yl]-N,N-dimethyl-3-trifluoromethyl-benzenesulfonamide;
5-(2-Chloro-4-methoxy-phenyl)-2-(2,6-difluoro-phenyl)-1H-indole;
2-(2,6-Difluoro-phenyl)-5-(4-methoxy-2-trifluoromethyl-phenyl)-1H-indole; or
2-(2,6-Difluoro-phenyl)-5-(2-methyl-4-trifluoromethoxy-phenyl)-1H-indole.

25. The compound according to claim 1, wherein said compound is:
2-(2,6-Difluoro-phenyl)-5-(6-methoxy-2-methyl-pyridin-3-yl)-1H-indole;
2-(2,6-Difluoro-phenyl)-5-(2-methyl-4-oxazol-2-yl-phenyl)-1H-indole;
2-(2,6-Difluoro-phenyl)-5-(2-methoxy-4-oxazol-2-yl-phenyl)-1H-indole;
2-(2,6-Difluoro-phenyl)-5-(4-methyl-6-piperazin-1-yl-pyridin-3-yl)-1H-indole;
2-(2,6-Difluoro-phenyl)-5-(5-methyl-2-pyridazin-4-yl-thiazol-4-yl)-1H-indole;
2-(2,6-Difluoro-phenyl)-5-(2-iodo-5-methyl-thiazol-4-yl)-1H-indole;
5-{5-[2-(2,6-Difluoro-phenyl)-1H-indol-5-yl]-1-methyl-1H-pyrazol-3-yl}-pyrimidin-2-ylamine;
2-(2,6-Difluoro-phenyl)-5-(1-methyl-1H,1′H-[3,3′]bipyrazolyl-5-yl)-1H-indole;
5-[2-(2-Fluoro-6-methyl-phenyl)-1H-indol-5-yl]-1-methyl-1H-pyrazole-3-carboxylic acid dimethylamide;
2-(2,6-Difluoro-phenyl)-5-(2-methyl-5-oxazol-2-yl-2H-pyrazol-3-yl)-1H-indole;
5-(5-Bromo-2-methyl-2H-pyrazol-3-yl)-2-(2,6-difluoro-phenyl)-1H-indole;
2-(2-Fluoro-phenyl)-5-(6-methoxy-4-methyl-pyridin-3-yl)-1H-indole;
2-(2,6-Difluoro-phenyl)-5-(6-methoxy-4-methyl-pyridin-3-yl)-1H-indole;
2-(2,6-Difluoro-phenyl)-5-(2-methyl-5-pyridin-3-yl-2H-[1,2,4]triazol-3-yl)-1H-indole;
2-(2-Chloro-6-fluoro-phenyl)-5-(2-methyl-4-[1,3,4]oxadiazol-2-yl-phenyl)-1H-indole;
2-(2-Chloro-6-fluoro-phenyl)-5-(2-methyl-5-pyridin-3-yl-2H-[1,2,4]triazol-3-yl)-1H-indole;
5-[2-(2-Chloro-6-fluoro-phenyl)-1H-indol-5-yl]-4-methyl-pyridine-2-carboxylic acid methyl ester;
5-[2-(2-Chloro-6-fluoro-phenyl)-1H-indol-5-yl]-4-methyl-pyridine-2-carboxylic acid methylamide;
2-(2-Chloro-6-fluoro-phenyl)-5-(4-methyl-[1,3,4]oxadiazol-2-yl-pyridin-3-yl)-1H-indole; or
2-(2-Chloro-6-fluoro-phenyl)-5-[4-methyl-6-(5-methyl-[1,3,4]oxadiazol-2-yl)-pyridin-3-yl]-1H-indole.

26. The compound according to claim 1, wherein said compound is:
2-(2-Chloro-6-fluoro-phenyl)-5-(6-methoxy-4-methyl-pyridin-3-yl)-1H-indole;
2-(2-Chloro-6-fluoro-phenyl)-5-(5-methoxy-3-methyl-pyridin-2-yl)-1H-indole;
2-(2-Chloro-6-fluoro-phenyl)-5-(6-methoxy-2-methyl-pyridin-3-yl)-1H-indole;
2-(2-Chloro-6-fluoro-phenyl)-5-(2-ethyl-5-pyridin-3-yl-2H-[1,2,4]triazol-3-yl)-1H-indole;
2-(2-Chloro-6-fluoro-phenyl)-5-(5-methyl-2-oxazol-2-yl-thiazol-4-yl)-1H-indole;
4-[2-(2-Chloro-phenyl)-1H-indol-5-yl]-3-methyl-benzoic acid methyl ester;
4-[2-(2-Chloro-phenyl)-1H-indol-5-yl]-3,N-dimethyl-benzamide;
4-[2-(2-Chloro-phenyl)-1H-indol-5-yl]-3-methyl-benzamide;
4-[2-(2-Chloro-phenyl)-1H-indol-5-yl]-3-methyl-benzonitrile;
4-[2-(2-Chloro-phenyl)-1H-indol-5-yl]-3,N,N-trimethyl-benzenesulfonamide;
4-[5-(4-carbomethoxy-2-methyl-phenyl)-1H-indol-2-yl]-3-methyl-benzoic acid methyl ester;
4-[2-(2-Chloro-4-methoxy-phenyl)-1H-indol-5-yl]-3-methyl-benzonitrile;
4-[2-(2-Fluoro-4-methanesulfonyl-phenyl)-1H-indol-5-yl]-3-methyl-benzonitrile;
4-[2-(2-Fluoro-3-cyano-phenyl)-1H-indol-5-yl]-3-methyl-benzonitrile;
4-(2-(2,6-difluoro-4-methoxyphenyl)-1H-indol-5-yl)-3-methylbenzonitrile;
4-(2-(2-fluorophenyl)-1H-indol-5-yl)-3-methylbenzonitrile;
4-(2-(4-Cyano-2-methylphenyl)-1H-indol-5-yl)-3-methylbenzonitrile;
4-(2-(2-Chloro-5-cyanophenyl)-1H-indol-5-yl)-3-methylbenzonitrile;
4-(2-(6-methoxy-2-methylpyridin-3-yl)-1H-indol-5-yl)-3-methylbenzonitrile; or
4-(2-(3-chloro-2-methoxypyridin-4-yl)-1H-indol-5-yl)-3-methylbenzonitrile.

27. The compound according to claim 1, wherein said compound is:
4-(2-(2,4-difluorophenyl)-1H-indol-5-yl)-3-methylbenzonitrile;
4-(2-(2,6-difluoro-3-methoxyphenyl)-1H-indol-5-yl)-3-methylbenzonitrile;
4-(2-(6-methoxy-4-methylpyridin-3-yl)-1H-indol-5-yl)-3-methylbenzonitrile;
methyl-4-(2-(4-methylpyridin-3-yl)-1H-indol-5-yl)benzonitrile;
methyl-4-(2-(3-methylpyridin-4-yl)-1H-indol-5-yl)benzonitrile;
methyl-4-(2-(3-methylthiophen-2-yl)-1H-indol-5-yl)benzonitrile;
methyl-4-(2-(2-methylpyridin-3-yl)-1H-indol-5-yl)benzonitrile;
4-(2-(2,4-dimethylthiazol-5-yl)-1H-indol-5-yl)-3-methylbenzonitrile;
methyl-4-(2-(4-methylthiophen-3-yl)-1H-indol-5-yl)benzonitrile;
methyl-4-(2-(1-methyl-1H-pyrazol-5-yl)-1H-indol-5-yl)benzonitrile;
4-(2-(3,5-dimethylisoxazol-4-yl)-1H-indol-5-yl)-3-methylbenzonitrile;
fluoro-3-(5-(6-methoxy-4-methylpyridin-3-yl)-1H-indol-2-yl)benzonitrile;
4-(2-(2,6-difluoro-4-(2-methoxyethoxy)phenyl)-1H-indol-5-yl)-3-methylbenzonitrile;
4-(2-(2,6-difluoro-4-(2-hydroxyethoxy)phenyl)-1H-indol-5-yl)-3-methylbenzonitrile;
4-(2-(4-(3-cyanopropoxy)-2,6-difluorophenyl)-1H-indol-5-yl)-3-methylbenzonitrile;
4-(2-(2,6-difluoro-4-(3-hydroxypropoxy)phenyl)-1H-indol-5-yl)-3-methylbenzonitrile;
4-(2-(2,6-difluoro-4-hydroxyphenyl)-1H-indol-5-yl)-3-methylbenzonitrile;
4-[2-(2-chloro-6-fluorophenyl)-1H-indol-5-yl]-3-methylbenzonitrile;
4-[2-(2-Chloro-6-fluoro-phenyl)-1H-indol-5-yl]-3,N,N-trimethyl-benzenesulfonamide; or
2-(2-Chloro-6-fluoro-phenyl)-5-(6-chloro-4-methyl-pyridin-3-yl)-1H-indole.
6-(2-(2-chloro-6-fluorophenyl)-1H-indol-5-yl)-5-methylnicotinonitrile;
5-(2-(2-chloro-6-fluorophenyl)-1H-indol-5-yl)-4-methylpicolinonitrile;
2-(2-chloro-6-fluorophenyl)-5-(6-(2-methoxyethoxy)-4-methylpyridin-3-yl)-1H-indole;
2-(2-chloro-6-fluorophenyl)-5-(6-ethoxy-4-methylpyridin-3-yl)-1H-indole;
4-(5-(2-(2-chloro-6-fluorophenyl)-1H-indol-5-yl)-4-methylpyridin-2-yl)morpholine;
5-(2-(2-chloro-6-fluorophenyl)-1H-indol-5-yl)-N,4-dimethylpyridin-2-amine;
6-(2-(2-chloro-6-fluorophenyl)-1H-indol-5-yl)-N,N,5-trimethylpyridine-3-sulfonamide;
4-(2-(2-chloro-6-fluorophenyl)-1H-indol-5-yl)-N,3-dimethylbenzenesulfonamide;
4-(4-(2-(2-chloro-6-fluorophenyl)-1H-indol-5-yl)-3-methylphenylsulfonyl)morpholine;
2-(2-chloro-6-fluorophenyl)-5-(2-methyl-4-(4-methylpiperazin-1-ylsulfonyl)phenyl)-1H-indole;
2-(2-chloro-6-fluorophenyl)-5-(2-methyl-4-(2-methyl-2H-tetrazol-5-yl)phenyl)-1H-indole;
4-[2-(2-Chloro-6-fluoro-phenyl)-1H-indol-5-yl]-3-methoxy-benzonitrile;
2-(2-Chloro-6-fluoro-phenyl)-5-(6-methanesulfonyl-4-methyl-pyridin-3-yl)-1H-indole;
5-(6-Chloro-4-ethyl-pyridin-3-yl)-2-(2-chloro-6-fluoro-phenyl)-1H-indole;
4-[2-(2-chloro-6-fluorophenyl)-1H-indol-5-yl]-5-ethyl-2-(pyridin-3-yl)thiazole;
2-(2-Chloro-6-fluoro-phenyl)-5-(5-methyl-2-pyrazin-2-yl-thiazol-4-yl)-1H-indole;
2-(2-Chloro-6-fluoro-phenyl)-5-(5-methyl-2-pyrimidin-5-yl-thiazol-4-yl)-1H-indole;
2-(2-Chloro-6-fluoro-phenyl)-5-[5-methyl-2-(6-methyl-pyridin-3-yl)-thiazol-4-yl]-1H-indole;
2-(2-Chloro-6-fluoro-phenyl)-5-(5-ethyl-2-pyrazin-2-yl-thiazol-4-yl)-1H-indole;
2-(2-Chloro-6-fluoro-phenyl)-5-(5-isopropyl-2-pyridin-3-yl-thiazol-4-yl)-1H-indole; or
2-(2-Chloro-6-fluoro-phenyl)-5-(5-isopropyl-2-pyrazin-2-yl-thiazol-4-yl)-1H-indole.

28. The compound according to claim 1, wherein said compound is:
2-(2-chloro-6-fluoro-phenyl)-5-[2-pyridin-3-yl-5-(2,2,2-trifluoro-1-methyl-ethyl)-thiazol-4-yl]-1H-indole;
2-(2-chloro-6-fluorophenyl)-5-(1-ethyl-3-(pyrazin-2-yl)-1H-pyrazol-5-yl)-1H-indole;
2-(2-Chloro-6-fluoro-phenyl)-5-(2-methyl-5-pyridin-2-yl-2H-[1,2,4]triazol-3-yl)-1H-indole;
2-(2-Chloro-phenyl)-5-(2-ethyl-5-pyridin-3-yl-2H-[1,2,4]triazol-3-yl)-1H-indole;
2-(2,6-Dichloro-phenyl)-5-(2-ethyl-5-pyridin-3-yl-2H-[1,2,4]triazol-3-yl)-1H-indole;
2-(2-Chloro-6-fluoro-phenyl)-5-(2-ethyl-5-pyrazin-2-yl-2H-[1,2,4]triazol-3-yl)-1H-indole;
2-(2-Chloro-6-fluoro-phenyl)-5-(2-methyl-5-pyrimidin-5-yl-2H-[1,2,4]triazol-3-yl)-1H-indole;
5-(1-ethyl-3-(trifluoromethyl)-1H-pyrazol-5-yl)-2-(3-methylpyridin-4-yl)-1H-indole;
5-(1-ethyl-3-(trifluoromethyl)-1H-pyrazol-5-yl)-2-(6-methoxy-4-methylpyridin-3-yl)-1H-indole;
5-(1-ethyl-3-(trifluoromethyl)-1H-pyrazol-5-yl)-2-(4-methylpyridin-3-yl)-1H-indole;
5-(1-ethyl-3-(trifluoromethyl)-1H-pyrazol-5-yl)-2-(3-fluoropyridin-4-yl)-1H-indole;
5-(1-ethyl-3-(trifluoromethyl)-1H-pyrazol-5-yl)-2-(6-methoxy-2-methylpyridin-3-yl)-1H-indole;
2-(3-chloro-2-methoxypyridin-4-yl)-5-(1-ethyl-3-(trifluoromethyl)-1H-pyrazol-5-yl)-1H-indole;
cyclohexyl-5-(1-ethyl-3-(trifluoromethyl)-1H-pyrazol-5-yl)-1H-indole;
cyclohexenyl-5-(1-ethyl-3-(trifluoromethyl)-1H-pyrazol-5-yl)-1-(trifluoromethylsulfonyl)-1H-indole;
Cyclohexyl-5-(1-ethyl-3-(trifluoromethyl)-1H-pyrazol-5-yl)-1H-indole; or
[2-(2-Cyclohexyl-ethyl)-4-(2-ethyl-5-trifluoromethyl-2H-pyrazol-3-yl)-phenyl]-methyl-amine.

29. The compound according to claim 1, wherein said compound is:
[2-(2-Cyclohexyl-ethyl)-4-(2-ethyl-5-trifluoromethyl-2H-pyrazol-3-yl)-phenyl]-methyl-amine;
5-(1-ethyl-3-(trifluoromethyl)-1H-pyrazol-5-yl)-2-(tetrahydro-2H-pyran-4-yl)-1H-indole;
5-(1-ethyl-3-(trifluoromethyl)-1H-pyrazol-5-yl)-2-(tetrahydro-2H-pyran-3-yl)-1H-indole;
1-(4-(5-(1-ethyl-3-(trifluoromethyl)-1H-pyrazol-5-yl)-1H-indol-2-yl)piperidin-1-yl)ethanone;
2-(2-chloro-6-fluoro-4-methoxyphenyl)-5-(1-methyl-3-(trifluoromethyl)-1H-pyrazol-5-yl)-1H-indole;
4-(2-(2-chloro-6-fluoro-4-methoxyphenyl)-1H-indol-5-yl)-3-methylbenzonitrile;
2-(2-chloro-6-fluoro-4-methoxyphenyl)-5-(1-ethyl-3-(trifluoromethyl)-1H-pyrazol-5-yl)-1H-indole;
2-(2,6-difluorophenyl)-5-(1-ethyl-3-(pyrazin-2-yl)-1H-pyrazol-5-yl)-1H-indole;
2-(2,6-Difluoro-phenyl)-5-(2-ethyl-5-pyridin-3-yl-2H-[1,2,4]triazol-3-yl)-1H-indole;
2-(2,6-Difluoro-phenyl)-5-(5-methyl-2-pyrazin-2-yl-thiazol-4-yl)-1H-indole;
2-(2,6-Difluoro-phenyl)-5-(5-ethyl-2-pyridin-3-yl-thiazol-4-yl)-1H-indole;
2-(2,6-Difluoro-phenyl)-5-(4-methyl-6-oxazol-2-yl-pyridin-3-yl)-1H-indole;
5-{5-[2-(2,6-Difluoro-phenyl)-1H-indol-5-yl]-4-methyl-pyridin-2-yl}-pyrimidin-2-ylamine;
2-(2,6-Difluoro-phenyl)-5-(4-methyl-6-pyrimidin-5-yl-pyridin-3-yl)-1H-indole;
2-(4-Methyl-pyridin-3-yl)-5-(4,4,5,5-tetramethyl-1,3,2-dioxaborolan-2-yl)-1H-indole;
Methyl-5-[2-(4-methyl-pyridin-3-yl)-1H-indol-5-yl]-pyridine-2-carbonitrile;
Methoxy-5-[2-(4-methyl-pyridin-3-yl)-1H-indol-5-yl]-pyridine-2-carbonitrile;
5-(6-Methanesulfonyl-4-methyl-pyridin-3-yl)-2-(4-methyl-pyridin-3-yl)1indole;
5-(6-Chloro-4-methyl-pyridin-3-yl)-2-(4-methyl-pyridin-3-yl)-1H-indole;
5-(6-Methoxy-4-methyl-pyridin-3-yl)-2-(4-methyl-pyridin-3-yl)-1H-indole;
2-(2,6-Dichloro-phenyl)-5-(5-methyl-2-pyridin-2-yl-thiazol-4-yl)-1H-indole;
2-(2,6-Dimethyl-phenyl)-5-(5-methyl-2-pyridin-3-yl-thiazol-4-yl)-1H-indole;
2-(2,6-Dimethyl-phenyl)-5-(5-methyl-2-pyridin-2-yl-thiazol-4-yl)-1H-indole;
2-(2-Fluoro-6-methyl-phenyl)-5-(5-methyl-2-pyridin-3-yl-thiazol-4-yl)-1H-indole.
2-(2-Fluoro-6-methyl-phenyl)-5-(5-methyl-2-pyridin-2-yl-thiazol-4-yl)-1H-indole;
Cyclohexyl-5-(2,5-dimethyl-2H-pyrazol-3-yl)-1H-indole;
4-(2-cyclohexyl-1H-indol-5-yl)-N,N,3-trimethylbenzenesulfonamide;
cyclohexyl-5-(6-methoxy-4-methylpyridin-3-yl)-1H-indole;
4-(2-(2-fluorophenyl)-3-methyl-1H-indol-5-yl)-N,N,3-trimethylbenzenesulfonamide;
N,N,3-trimethyl-4-(3-methyl-2-phenyl-1H-indol-5-yl)benzenesulfonamide;
2-(2,6-Difluoro-phenyl)-5-(2,5-dimethyl-2H-pyrazol-3-yl)-3-methyl-1H-indole;
4-[2-(2,6-Difluoro-phenyl)-3-methyl-1H-indol-5-yl]-3,N,N-trimethyl-benzenesulfonamide; or
2-(2,6-Difluoro-phenyl)-5-(6-methoxy-4-methyl-pyridin-3-yl)-3-methyl-1H-indole.

30. A pharmaceutical composition, comprising a therapeutically effective amount of a compound according to claim 1 and a pharmaceutically acceptable carrier.

31. A method for treating arthritis, comprising the step of administering a therapeutically effective amount of a compound accoriding to claim 1 to a subject in need thereof.

32. A method for treating a respiratory disorder selected from chronic obstructive pulmonary disorder (COPD), asthma, and bronchospasm, comprising the step of administering a therapeutically effective amount of a compound according to claim 1 to a subject in need thereof.
